(19) United States
(12) Patent Application Publication
(10) Pub. No.: US 2025/0122219 A1
(43) Pub. Date: Apr. 17, 2025

(54) BRIDGED TRICYCLIC CARBAMOYLPYRIDONE COMPOUNDS AND USES THEREOF

(71) Applicant: Gilead Sciences, Inc., Foster City, CA (US)

(72) Inventors: Megan K. Armstrong, San Francisco, CA (US); Chienhung Chou, Dublin, CA (US); Ana Z. Gonzalez Buenrostro, San Mateo, CA (US); Xiaochun Han, San Jose, CA (US); Lan Jiang, Foster City, CA (US); Jiayao Li, Foster City, CA (US); Gregg M. Schwarzwalder, Redwood City, CA (US); Qiaoyin Wu, Foster City, CA (US); Hai Yang, San Mateo, CA (US)

(21) Appl. No.: 18/911,404

(22) Filed: Oct. 10, 2024

Related U.S. Application Data
(60) Provisional application No. 63/543,699, filed on Oct. 11, 2023.
(60) Provisional application No. 63/641,589, filed on May 2, 2024.

(51) Int. Cl. C07D 498/22; A61K 31/55; A61K 45/06

(52) U.S. Cl. CPC C07D 498/22; A61K 31/55; A61K 45/06

(57) ABSTRACT

The present disclosure relates generally to compounds, of Formula I:

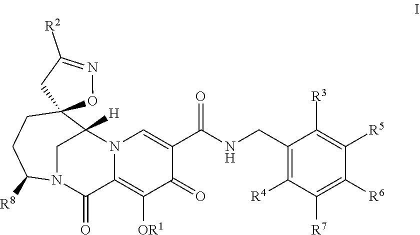

Also disclosed are pharmaceutical compositions comprising said compounds and methods of making said compounds. The compounds of the disclosure are useful in treating or preventing human immunodeficiency virus (HIV) infection.

### CROSS-REFERENCE TO RELATED APPLICATIONS

This application claims priority to U.S. Provisional Application No. 63/543,699, filed Oct. 11, 2023, and U.S. Provisional Application No. 63/641,589, filed May 2, 2024. The entire contents of these applications are hereby incorporated by reference in their entireties.

### FIELD

Compounds, compositions, and methods that may be used for treating or preventing human immunodeficiency virus (IV) infection are disclosed. In particular, novel spirocyclic substituted bridged tricyclic carbamoylpyridone compounds and methods for their preparation and use as therapeutic or prophylactic agents are disclosed.

### BACKGROUND

Human immunodeficiency virus infection and related diseases are a major public health problem worldwide. Human immunodeficiency virus encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains may limit their usefulness (Palella, et al. N. Eng. J Med. (1998) 338:853-860; Richman, D. D. Nature (2001) 410:995-1001). Accordingly, there is a need for new agents that inhibit the replication of HIV.

A goal of antiretroviral therapy is to achieve viral suppression in the HIV infected patient. Current treatment guidelines published by the United States Department of Health and Human Services provide that achievement of viral suppression requires the use of combination therapies, i.e., several drugs from at least two or more drug classes (Panel on Antiretroviral Guidelines for Adults and Adolescents. Guidelines for the Use of Antiretroviral Agents in Adults and Adolescents Living with HIV. Department of Health and Human Services. Available at http://www.aidsinfo.nih.gov/ContentFiles/AdultandAdolescentGL.pdf. Accessed Feb. 12, 2019). In addition, decisions regarding the treatment of HIV infected patients are complicated when the patient requires treatment for other medical conditions (Id. at F-8). Because the standard of care requires the use of multiple different drugs to suppress HIV, as well as to treat other conditions the patient may be experiencing, the potential for drug interaction is a criterion for selection of a drug regimen. As such, there is a need for antiretroviral therapies having a decreased potential for drug interactions.

In addition, the HIV virus is known to mutate in infected subjects (Tang, et al. Drugs (2012) 72 (9) e1-e25). Because of the proclivity of the HIV virus to mutate, there is a need for anti-HIV drugs to be effective against a range of known HIV variants (Hurt, et al. HIV/AIDS CID (2014) 58, 423-431).

For certain patients, for example, those with difficult or limited access to health care, adherence to daily oral treatment or prophylactic regimens can be challenging. Drugs that offer favorable pharmaceutical properties (for example, improved potency, long-acting pharmacokinetics, low clearance, and/or other properties) are amenable to less frequent administration and provide for better patient compliance. Such improvements can, in turn, optimize drug exposure and limit the emergence of drug resistance.

### SUMMARY

The present disclosure is directed to novel compounds having antiviral activity and pharmaceutically acceptable salts thereof. In some embodiments, the compounds may be used to treat HIV infections, to inhibit the activity of HIV integrase and/or to reduce HIV replication. In some embodiments, compounds disclosed herein may be effective against a range of known drug-resistant HIV mutants. In some embodiments, compounds disclosed herein may have a decreased propensity to cause drug-drug interactions when co-administered with other drugs. In some embodiments, compounds disclosed herein may be administered with less than daily frequency, for example, at weekly, monthly, once every three months, once every six months, or longer intervals.

In one embodinent, the disclosure provides a compound of Formula I:

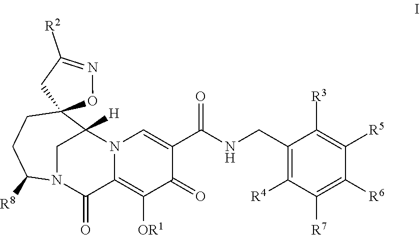

or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is an integer between 0 and 10, inclusive;
   p is 0 or 1;
   R^{1A} is H or C₁₋₃alkyl;
   R^{1B} is H or C₁₋₃alkyl;
   each R^{C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆ cycloalkyl, C₁₋₂₀alkyl or C₁₋₂₀alkenyl; wherein the C₃₋₆ cycloalkyl, C₁₋₂₀alkyl or C₁₋₂₀alkenyl is optionally substituted with a COOH, C₃₋₆ cycloalkyl, C₁₋₃ alkoxy, or —NHCOOR¹H;
   each R¹H is C₁₋₃alkyl;
R² is C₁₋₃ alkyl or C₁₋₃ alkoxy;
each R³, R⁴, R⁵, R⁶ and R⁷ is independently H or halo; and
R⁸ is H or C₁₋₃alkyl.

In one embodiment, the disclosure provides a compound of Formula I, or a pharmaceutically acceptable salt thereof, wherein:

or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is an integer between 0 and 10, inclusive;
   p is 0 or 1;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆ cycloalkyl, C₁₋₂₀alkyl or C₁₋₂₀alkenyl; wherein the C₃₋₆ cycloalkyl, C₁₋₂₀alkyl or C₁₋₂₀alkenyl is optionally substituted with a COOH, C₃₋₆ cycloalkyl, C₁₋₃ alkoxy, or —NHCOOR¹H;
   each R¹H is C₁₋₃alkyl;
R² is C₁₋₃ alkyl or C₁₋₃ alkoxy;
each R³, R⁴, R⁵, R⁶ and R⁷ is independently H or halo; and
R⁸ is H or C₁₋₃alkyl.

In one embodiment, a pharmaceutical composition is provided comprising a therapeutically effective amount of a compound of Formula I or a pharmaceutically acceptable salt thereof, and a pharmaceutically acceptable excipient.

In another embodiment, a kit or an article of manufacture is provided comprising a compound of Formula I, or a pharmaceutically acceptable salt thereof, and instructions for use.

In another embodiment, a method of treating an HIV infection in a human having or at risk of having the infection, by administering to the human a therapeutically effective amount of a compound of Formula I or a pharmaceutically acceptable salt thereof, or a pharmaceutical composition of a compound of Formula I, or a pharmaceutically acceptable salt thereof, is provided.

In another embodiment, use of a compound of Formula I, or a pharmaceutically acceptable salt thereof, or a pharmaceutical composition of a compound of Formula I or a pharmaceutically acceptable salt thereof, for treating an HIV infection in a human having or at risk of having the infection is provided.

In another embodiment, use of a compound of Formula I, or a pharmaceutically acceptable salt thereof, or a pharmaceutical composition of a compound of Formula I or a pharmaceutically acceptable salt thereof, in the manufacture of a medicament for treating an HIV infection in a human having or at risk of having the infection is provided.

In another embodiment, a compound of Formula I or a pharmaceutically acceptable salt thereof, or a pharmaceutical composition of a compound of formula I or a pharmaceutically acceptable salt thereof, for use in medical therapy is provided.

In another embodiment, a compound of Formula I or a pharmaceutically acceptable salt thereof, or a pharmaceutical composition of a compound of formula I or a pharmaceutically acceptable salt thereof, for use in treating an HIV infection is provided.

Other embodiments, objects, features, and advantages may be set forth in the detailed description of the embodiments that follows, and in part may be apparent from the description, or may be learned by practice, of the claimed embodiments. These objects and advantages may be realized and attained by the processes and compositions particularly pointed out in the description and claims thereof. The foregoing Summary has been made with the understanding that it is to be considered as a brief and general synopsis of some of the embodiments disclosed herein, is provided for the benefit and convenience of the reader, and is not intended to limit in any manner the scope, or range of equivalents, to which the appended claims are lawfully entitled.

### BRIEF DESCRIPTION OF THE DRAWINGS

FIG. 1 shows an overlay of the plasma concentrations of Intermediate B measured over time following a single subcutaneous injection of Compounds 8, 10, 15, 16, 17, or 22 in rats.

FIG. 2 shows the plasma concentrations of Intermediate C measured over time following a single subcutaneous injection of Compound 5 in rats.

### DETAILED DESCRIPTION

In the following description, certain specific details are set forth in order to provide a thorough understanding of various embodiments disclosed herein. However, one skilled in the art will understand that the embodiments disclosed herein may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### I. Definitions

Unless the context requires otherwise, throughout the present disclosure and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment disclosed herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments,

“Amino” refers to the —NH₂ radical.

“Hydroxy” or “hydroxyl” refers to the —OH radical.

The term “C₁₋ₙ alkyl” as used herein, wherein n is an integer, either alone or in combination with another radical, is intended to mean acyclic, straight or branched chain alkyl radicals containing from 1 to n carbon atoms. “C₁₋₆ alkyl” includes, but is not limited to, methyl, ethyl, propyl (n-propyl), butyl (n-butyl), 1methylethyl (isopropyl), 1methylpropyl (sec-butyl), 2methylpropyl (iso-butyl), 1,1dimethylethyl (tertbutyl), pentyl and hexyl. The abbreviation Me denotes a methyl group: Et denotes an ethyl group, Pr denotes a propyl group iPr denotes a 1-methylethyl group, Bu denotes a butyl group and tBu denotes a 1,1-dimethylethyl group.

“Alkyl” is hydrocarbon containing normal, secondary or tertiary atoms. For example, an alkyl group can have 1 to 20 carbon atoms (i.e., C₁₋₂₀ alkyl), 1 to 10 carbon atoms (i.e., C₁₋₁₀ alkyl), 1 to 8 carbon atoms (i.e., C₁₋₈ alkyl) or 1 to 6 carbon atoms (i.e. C₁₋₆ alkyl). Examples of suitable alkyl groups include, but are not limited to, methyl (Me, —CH₃), ethyl (Et, CH₂CH), 1-propyl (n—Pr, n-propyl, —CH₂CH₂CH-3), 2-propyl (iPr, ipropyl, CH(CH₃)₂), 1-butyl (n-Bu, n-butyl, —CH₂CH₂CH₂CH₃), 2methyl1-propyl (i-Bu, ibutyl, —CH₂CH(CH₃)₂), 2butyl (s-Bu, s-butyl, CH(CH₃)CH₂CH₃) 2-methyl-2-propyl (t-Bu, tbutyl, —C(CH₃)₃), 1-pentyl (n-pentyl, CH₂CH₂CH₂CH₂CH₃), 2-pentyl (CH(CH₃)CH₂CH₂CH₃), 3pentyl (CH(CH₂C₃)₂), 2-methyl-2-butyl (C(CH₃)₂CH₂CH₃), 3methyl2-butyl (CH(CH₃)CH(CH))₃methyl1butyl (CH₂CH₂CH(CH₃)₂), 2-methyl-1-butyl (CH₂CH(CH₃)CH₂CH₃), 1-hexyl (CH₂CH₂CH₂CH₂CH₂CH₃), 2-hexyl (CH(CH₃)CH₂CH₂CH₂CH₃), 3hexyl (CH(CH₂CH₃)(CF₂CH₂CH)), 2methyl-2-pentyl (C(CH₃)₂CH₂CH₃), 3methy2pentyl (CH(CH₃)CH(CH₃)CH₂CH₃), 4-methyl-2-pentyl (CH(CH₃)CH₂CH(CH₃)₂), 3-methyl-3-pentyl (—C(CH₃)(CH₂CH₃)₂), 2methyl-3-pentyl (—CH(CH₂CH₃)CH(CH₃)₂), 2,3-dimethyl-2-butyl (C(CH₃)₂CH(CH₃)₂), 3,3dimethyl2butyl (—CH(CH₃)C(CH₃)₃, and octyl ((CH₂)₇CH). “Alkyl” also refers to a saturated, branched or straight chain hydrocarbon radical having two monovalent radical centers derived by the removal of two hydrogen atoms from the same or two different carbon atoms of a parent alkane. For example, an alkyl group can have 1 to 10 carbon atoms (i.e., C₁-1a alkyl), or 1 to 6 carbon atoms (i.e. C₁₋₆ alkyl) or 1-3 carbon atoms (i.e., C₁₋₃ alkyl). Typical alkyl radicals include, but are not limited to, methylene (CH₂), 1,1ethyl (CH(CH₃)), 1,2ethyl (CH₂CH₂), 1,1-propyl (CH(CH₂CH₃)), 1,2-propyl (CH₂CH(CH₃)), 1,3propyl (CH₂CH₂CH₂), 1,4-butyl (CH₂CH₂CH₂CH₂), and the like.

The term “alkenyl” as used herein refers to a straight or branched hydrocarbon containing normal, secondary or tertiary carbon atoms with at least one site of unsaturation, i.e., a carbon-carbon, sp² double bond. For example, an alkenyl group can have 2 to 20 carbon atoms (i.e., C₂-C₂₀ alkenyl, or C₂₋₂₀ alkenyl), 2 to 8 carbon atoms (i.e., C₂-C₈ alkenyl or C₂₋₈ or 2 to 6 carbon atoms (i.e., C₂-C₆ alkenyl, or C₂₋₆ alkenyl). Examples of suitable alkenyl groups include, but are not limited to, ethylene or vinyl (CH═CH₂), allyl (CH₂CH₁═CH₂), cyclopentenyl (C₅H₇), and 5-hexenyl (CH₂CH₂CH₂CH₂CH═CH₂).

The term “C₂₋ₙalkenyl”, as used herein, wherein n is an integer, either alone or in combination with another radical, is intended to mean an unsaturated, acyclic straight or branched chain radical containing two to n carbon atoms, at least two of which are bonded to each other by a double bond. Examples of such radicals include, but are not limited to, ethenyl (vinyl), 1propenyl, 2propenyl, and 1butenyl. Unless specified otherwise, the term “C₂₋ₙalkenyl” is understood to encompass individual stereoisomers where possible, including but not limited to (E) and (Z) isomers, and mixtures thereof. When a C₂₋ₙalkenyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “halo” or “halogen” as used herein refers to fluoro, chloro, bromo and iodo.

The term “C₃₋ₘ cycloalkyl” as used herein, wherein m is an integer, either alone or in combination with another radical, is intended to mean a cycloalkyl substituent containing from 3 to m carbon atoms and includes, but is not limited to, cyclopropyl, cyclobutyl, cyclopentyl, cyclohexyl and cycloheptyl. The term includes fully saturated as well as partially unsaturated rings.

It is to be understood that when a variable is substituted, for example, as described by the phrase “C₁₋₆ alkyl, either alone or as part of a group, is optionally substituted”, the phrase means that the variable C₁₋₆ alkyl can be substituted when it is alone and that it can also be substituted when the variable “C₁₋₆ alkyl” is part of a larger group. Similarly, when stated, other variables can also be substituted “either alone or as part of a group.”

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers or axes of chirality and whose molecules are not mirror images of one another. Diastereomers typically have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “treatment” or “treating,” to the extent it relates to a disease or condition includes preventing the disease or condition from occurring, inhibiting or ameliorating the disease or condition (e.g., arresting or slowing its development), eliminating the disease or condition (e.g., causing regression or cure of the disease or condition), and/or relieving one or more symptoms of the disease or condition. In the case of HIV, treatment includes reducing the level of HIV viral load in a patient.

In some embodiments, the term “treatment” refers to the administration of a compound or composition according to the present invention to alleviate or eliminate symptoms of HIV infection and/or to reduce viral load in a patient. The term “treatment” also encompasses the administration of a compound or composition according to the present invention before the exposure of the individual to the virus, postexposure of the individual to the virus but before the appearance of symptoms of the disease, and/or prior to the detection of the virus in the blood, to prevent the appearance of symptoms of the disease and/or to prevent the virus from reaching detectible levels in the blood, and the administration of a compound or composition according to the present invention to prevent perinatal transmission of HIV from mother to baby, by administration to the mother before giving birth and to the child within the first days of life.

“Protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection. e.g., alcohols, be physiologically acceptable, although in general it is more desirable if the products are pharmacologically innocuous.

Protecting groups are available, commonly known and used, and are optionally used to prevent side reactions with the protected group during synthetic procedures, i.e., routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. PGs do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene (John Wiley & Sons, Inc., New York, 1991, ISBN 0-471-62301-6) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene as set forth below.

The term “solvate” refers to a crystalline solid containing amounts of a solvent incorporated within the crystal structure. As used herein, the term “solvate” includes hydrates.

The term “non-solvate” refers to a crystalline solid in which no solvent molecules occupy a specific crystallographic site.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “pharmaceutically acceptable salt” as used herein is intended to mean a salt of a compound according to the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Birge et al., J. Pharm. Sci., 1977, 66, pp. 1-19.

The term “pharmaceutically-acceptable acid addition salt” as used herein is intended to mean those salts which retain the biological effectiveness and properties of the free bases and which are not biologically or otherwise undesirable, formed with inorganic acids including but not limited to hydrochloric acid, hydrobromic acid, sulfuric acid, sulfamic acid, nitric acid, phosphoric acid and the like, and organic acids including but not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid and the like.

The term “pharmaceutically-acceptable base addition salt” as used herein is intended to mean those salts which retain the biological effectiveness and properties of the free acids and which are not biologically or otherwise undesirable, formed with inorganic bases including but not limited to ammonia or the hydroxide, carbonate, or bicarbonate of ammonium or a metal cation such as sodium, potassium, lithium, calcium, magnesium, iron, zinc, copper, manganese, aluminum and the like. Particularly preferred are the ammonium, potassium, sodium, calcium, and magnesium salts. Salts derived from pharmaceutically-acceptable organic nontoxic bases include but are not limited to salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine. N-methylmorpholine, dicyclohexylamine, dibenzylamine, N,N-dibenzylphenethylamine, 1-ephenamine, N,N′-dibenzylethylenediamine, polyamine resins and the like. Particularly preferred organic nontoxic bases are isopropylamine, diethylamine, ethanolamine, trimethylamine, dicyclohexylamine, choline, and caffeine.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal. The term “antiviral agent” includes, for example, an HIV integrase catalytic site inhibitor selected from the group consisting: raltegravir (ISENTRESS®; Merck); elvitegravir (Gilead); soltegravir (GSK; ViiV); GSK 1265744 (GSK; ViiV) and dolutegravir; an HIV nucleoside reverse transcriptase inhibitor selected from the group consisting of: abacavir (ZIAGEN®; GSK); didanosine (VIDEX®; BMS); tenofovir (VIREAD®; Gilead); emtricitabine (EMTRIVA®; Gilead); lamivudine (EPIVIR®; GSK/Shire); stavudine (ZERIT®; BMS); zidovudine (RETROVIR®; GSK); elvucitabine (Achillion); and festinavir (Oncolys); an HIV non-nucleoside reverse transcriptase inhibitor selected from the group consisting of: nevirapine (VIRAMUNE®; BI); efavirenz (SUSTIVA®; BMS); etravirine (INTELENCE®; J&J); rilpivirine (TMC278, R278474; J&J); fosdevirine (GSK/ViiV); and lersivirine (Pfizer ViiV); an HIV protease inhibitor selected from the group consisting of: atazanavir (REYATAZ®; BMS); darunavir (PREZISTA®; J&J); indinavir (CRIXIVAN)®; Merck); lopinavir (KELETRA®; Abbott); nelfinavir (VIRACEPT®; Pfizer); saquinavir (INVIRASEV®; Hoffmann-LaRoche); tipranavir (APTIVUS®; BI); ritonavir (NORVIR®; Abbott); and fosamprenavir (LEXIVA); GSK/Vertex); an HIV entry inhibitor selected from: maraviroc (SELZENTRY®; Pfizer); enfuvirtide (FUZEON®; Trimeris); and BMS-663068 (BMS); and an HIV maturation inhibitor selected from: bevirimat (Myriad Genetics).

The term “inhibitor of HIV replication” as used herein is intended to mean an agent capable of reducing or eliminating the ability of HIV to replicate in a host cell, whether in vitro, ex vivo or in vivo.

The term “substituent”, as used herein and unless specified otherwise, is intended to mean an atom, radical or group which may be bonded to a carbon atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The term “heteroatom” as used herein is intended to mean O, S or N.

The terms “O—C₁₋ₙ alkyl” or “C₁₋ₙ alkoxy” as used herein interchangeably, wherein n is an integer, either alone or in combination with another radical, is intended to mean an oxygen atom further bonded to an alkyl radical having 1 to n carbon atoms as defined above. Examples of C₁₋ₙ alkoxy include but are not limited to methoxy (CH₃O—), ethoxy (CH₃CH₂O—), propoxy (CH₃CH₂CH₂O—), 1-methylethoxy (iso-propoxy; (CH₃)₂CHO) and 1,1-dimethylethoxy (tert-butoxy; (CH₃)₃CO). When an C₁₋ₙ alkoxy is substituted, it is understood to be substituted on the alkyl portion thereof, such that the substitution would give rise to a chemically stable compound, such as are recognized by those skilled in the art.

The term “mammal” as used herein is intended to encompass humans, as well as non-human mammals which are susceptible to infection by HIV. Non-human mammals include but are not limited to domestic animals, such as cows, pigs, horses, dogs, cats, rabbits, rats and mice, and non-domestic animals.

The embodiments disclosed herein are also meant to encompass all pharmaceutically acceptable compounds of Formula I being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶C, ¹²³I, and ¹²⁵I, respectively. In certain embodiments, these radiolabeled compounds are useful to help determine or measure the effectiveness of the compounds, by characterizing, for example, the site or mode of action, or binding affinity to pharmacologically important site of action. Certain isotopically-labeled compounds of Formula I, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

In certain embodiments, substitution with heavier isotopes such as deuterium, i.e., ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O, and ¹³N, can be useful in Positron Emission Topography (PET) studies for examining substrate receptor occupancy. Isotopically-labeled compounds of the compounds disclosed herein can be prepared by techniques known to those skilled in the art or by processes analogous to those described in the Examples as set out below using an appropriate isotopically-labeled reagent in place of the non-labeled reagent previously employed.

The methods, compositions, kits and articles of manufacture provided herein use or include compounds (e.g., a compound of Formula I) or pharmaceutically acceptable salts thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds increase resistance to metabolism, and thus are useful for increasing the half-life of compounds or pharmaceutically acceptable salts thereof, when administered to a mammal. See. e.g., Foster, “Deuterium Isotope Effects in Studies of Drug Metabolism”, Trends Pharmacol. Sci., 5(12):524-527 (1984). Such compounds can be synthesized by means known in the art, for example by employing starting materials in which one or more hydrogen atoms have been replaced by deuterium.

The embodiments disclosed herein are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the embodiments disclosed herein include compounds produced by a process comprising administering a compound according to the embodiments disclosed herein to a mammal for a period of time sufficient to yield a metabolic product thereof. Such products are typically identified by administering a radiolabeled compound according to the embodiments disclosed herein in a detectable dose to an animal, such as rat, mouse, guinea pig, monkey, or to human, allowing sufficient time for metabolism to occur, and isolating its conversion products from the urine, blood or other biological samples.

The compounds disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic, scalemic, and optically pure forms. Optically active (+) and (−), (R)- and (S)-, or (D)- and (L)-isomers may be prepared using chiral synthons or chiral reagents, or resolved using methods such as chromatography and fractional crystallization. Techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Optional” or “optionally” means that the subsequently described event or circumstances may or may not occur, and that the description includes instances where said event or circumstance occurs and instances in which it does not. For example, “optionally substituted heterocyclyl” means that the heterocyclyl radical may or may not be substituted and that the description includes both substituted heterocyclyl radicals and heterocyclyl radicals having no substitution.

### II. Compounds

In some embodiments, the disclosure provides a compound of Formula I:

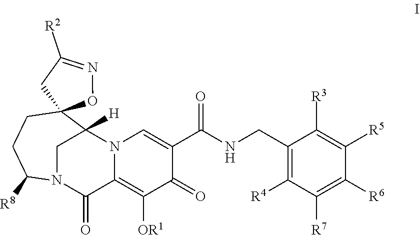

or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is an integer between 0 and 10, inclusive;
   p is 0 or 1;
   R^{1A} is H or C₁₋₃alkyl;
   R^{1B} is H or C₁₋₃alkyl;
   each R^{C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆ cycloalkyl, C₁₋₂₀alkyl or C₁₋₂₀alkenyl; wherein the C₃₋₆ cycloalkyl, C₁₋₂₀alkyl or C₁₋₂₀alkenyl is optionally substituted with a COOH, C₃₋₆ cycloalkyl, C₁₋₃ alkoxy, or —NHCOOR¹H;
   each R¹H is C₁₋₃alkyl;
R² is C₁₋₃ alkyl or C₁₋₃ alkoxy;
each R³, R⁴, R⁵, R⁶ and R⁷ is independently H or halo; and
R⁸ is H or C₁₋₃alkyl.

In some embodiments, the disclosure provides a compound of Formula I, or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is an integer between 0 and 10, inclusive;
   p is 0 or 1;
   R^{1A} is H or C₁₋₃alkyl;
   R^{1B} is H or C₁₋₃alkyl;
   each R^{C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆ cycloalkyl, C₁₋₂₀alkyl or C₁₋₂₀alkenyl; wherein the C₃₋₆ cycloalkyl, C₁₋₂₀alkyl or C₁₋₂₀alkenyl is optionally substituted with a COOH, C₃₋₆ cycloalkyl, C₁₋₃ alkoxy, or —NHCOOR¹H;
   each R¹H is C₁₋₃alkyl;
R² is C₁₋₃ alkyl or C₁₋₃ alkoxy;
each R³, R⁴, R⁵, R⁶ and R⁷ is independently H or halo; and
R⁸ is H or C₁₋₃alkyl.

In some embodiments, m is 0. In some embodiments, m is 1.

In some embodiments, n is an integer between 0 and 10, inclusive (i.e., n is 0, 1, 2, 3, 4, 5, 6, 7, 8, 9, or 10). In some embodiments, n is an integer between 0 and 7, inclusive (i.e., n is 0, 1, 2, 3, 4, 5, 6, or 7). In some embodiments, n is an integer between 1 and 7, inclusive (i.e., n is 1, 2, 3, 4, 5, 6, or 7). In some embodiments, n is 1, 2, 3, or 7.

In some embodiments, n is 0, 1, 2, 3, or 7. In some embodiments, n is 0, 1, 2, or 3. In some embodiments, n is 0. In some embodiments, n is 1, 2, or 3. In some embodiments, n is 1. In some embodiments, n is 2. In some embodiments, n is 3. In some embodiments, n is 7.

In some embodiments, p is 0. In some embodiments, p is 1.

In some embodiments, each R^{1A} is H. In some embodiments, each R^{1B} is H. In some embodiments, each R^{1A} and R^{1B} is H. R^{1B} is H or C₁-3alkyl. In some embodiments, R^{1A} is H and R^{1B} is C₁₋₃alkyl. In some embodiments, R^{1A} is H and R^{1B} is methyl.

In some embodiments, each R^{1C} and R^{1D} is H. In some embodiments, each R^{1C} is methyl and each R^{1D} is H. In some embodiments, each R^{1E} and R^{1F} is H. In some embodiments, each R^{1E} is methyl and each R^{1F} is H.

In some embodiments, each R^{1C} is H. In some embodiments, each R^{1D} is H. In some embodiments, each R^{1E} is H. In some embodiments, each R^{1F} is H. In some embodiments, each R^{1C}, R^{1D}, R^{1E}, and R^{1F} is H.

In some embodiments, R^{1G} is 5-6 cycloalkyl, C₁₋₂₀alkyl or C₁₋₂₀alkenyl; wherein the C₁₋₂₀alkyl or C₁₋₂₀alkenyl is optionally substituted with COOH, C₅₋₆ cycloalkyl, C₁₋₃ alkoxy, or —NHCOOR^{1H}. In some embodiments, R^{1G} is C₅₋₆ cycloalkyl, C₁₋₂₀alkyl or C₂₋₂₀ alkenyl; wherein the C₁₋₂₀alkyl or C₂₋₂₀alkenyl is optionally substituted with COOH, C₅₋₆ cycloalkyl, C₁₋₃ alkoxy, or —NHCOOR^{1H}.

In some embodiments, R^{1H} is methyl.

In some embodiments, R^{1G} is C₁₋₂₀alkyl or C₁₋₂₀alkenyl; wherein the C₁₋₂₀alkyl or C₁₋₂₀alkenyl is optionally substituted with a COOH or C₃₋₆ cycloalkyl. In some embodiments, R^{1G} is C₁₋₂₀alkyl or C₁₋₂₀alkenyl; wherein the C₁₋₂₀alkyl or C₁₋₂₀alkenyl is optionally substituted with a COOH.

In some embodiments, R^{1G} is C₁₋₂₀alkyl optionally substituted with a COOH, C₃₋₆ cycloalkyl, C₁₋₃ alkoxy, or —NHCOOR^{1H}. In some embodiments, R^{1G} is C₁₋₂₀alkyl optionally substituted with a COOH, cyclopentyl, methoxy, or —NHCOOCH₃. In some embodiments, R^{1G} is C₁₋₁₈alkyl optionally substituted with a COOH, cyclopentyl, methoxy, or —NHCOOCH₃.

In some embodiments, R^{1G} is C₁₋₂₀alkenyl optionally substituted with a COOH. In some embodiments, R^{1G} is C₂₋₂₀alkenyl optionally substituted with a COOH

In some embodiments, R^{1G} is C₃₋₆ cycloalkyl. In some embodiments, R^{1G} is C₅₋₆ cycloalkyl. In some embodiments, R^{1G} is cyclopentyl.

In some embodiments, R^{1G} is C₁₋₂₀alkyl or C₁₋₂₀alkenyl. In some embodiments, R^{1G} is C₁₋₂₀alkyl. In some embodiments. R^{1G} is C₁₋₂₀alkenyl. In some embodiments, R^{1G} is C₂₋₂₀alkenyl.

In some embodiments, R^{1G} is selected from the group consisting of —CH₃, —CH(CH₃)₂, —C(CH₃)₃, —(CH₂)₃CH₃, C₁₃H₂₇, C₁₅H₃₁, C₁₇H₃₅, C₁₇H₃₁, —CHCHCOOH, cyclopentyl, C₂H₅, C₃H₇, C₄H₉, C₅H₁₁, C₆H₁₃, C₇H₁₅, C₈H₁₇, C₉H₁₉, C₁₀H₂₁, C₁₁H₂₃, C₁₂H₂₅, C₁₄H₂₉, C₁₅H₃₁, C₁₆H₃₃, C₁₈H₃₇, —(CH₂)₂(cyclopentyl), —(CH₂)₅OCH₃, —(CH₂)₃COOH, —(CH₂)₅COOH, —(CH₂)₁₆COOH, —CH(CH₃)NHCOOCH₃, —CH(C₃H₇)NHCOOCH₃, C₁₃H₂₅, and C₁₉H₂₉.

In some embodiments, R^{1G} is selected from the group consisting of —CH₃, —CH(CH₃)₂, —C(CH₃)₃, —(CH₂)₃CH₃, C₁₃H₂₇, C₁₅H₃₁, —C₁₇R₃₅, —C₁₇H₃₁, and —CHCHCOOH.

In some embodiments, R^{1G} is selected from the group consisting of:

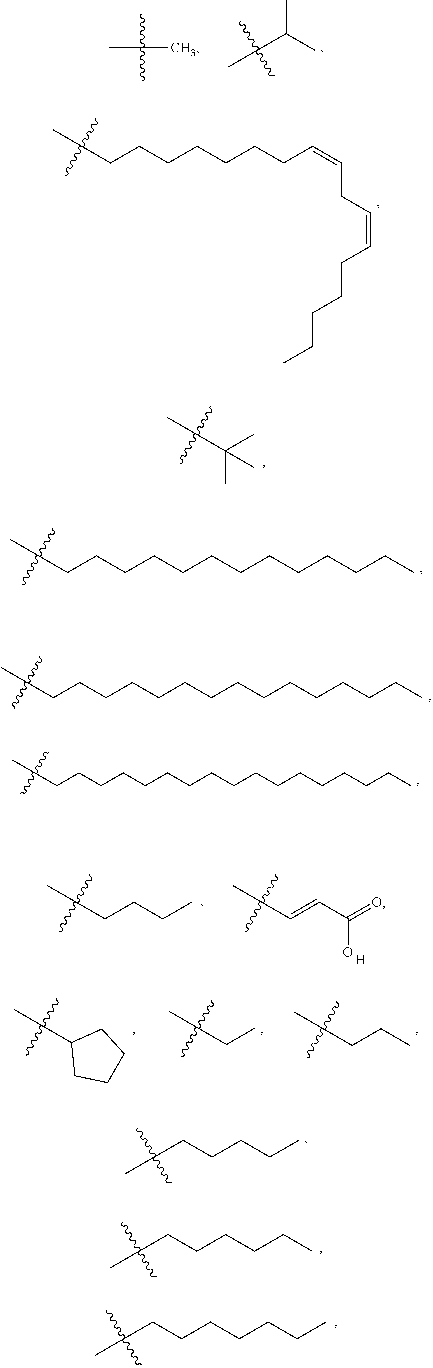

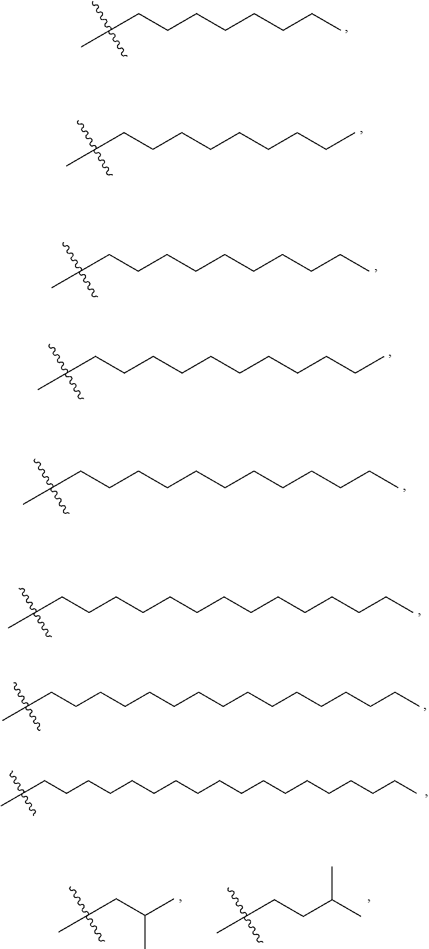

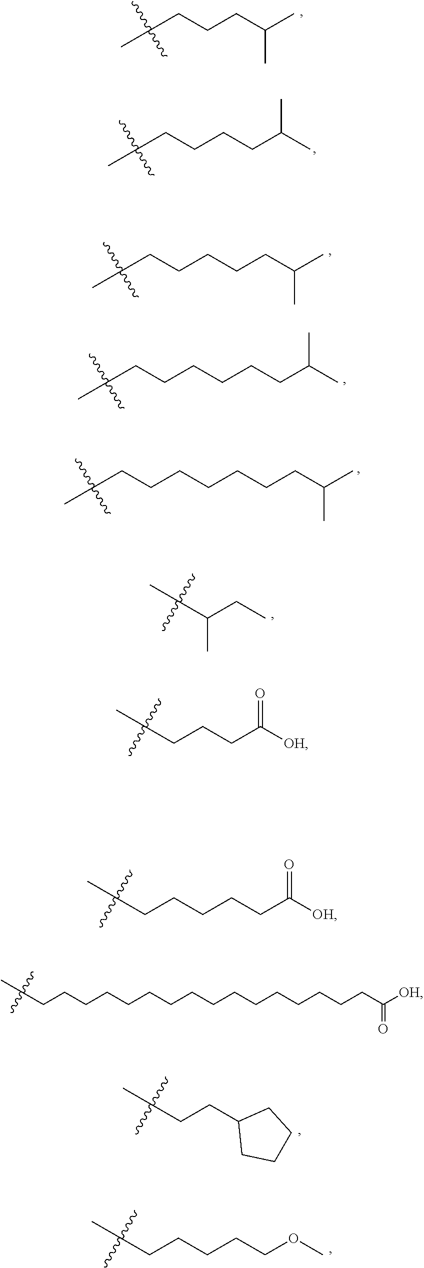

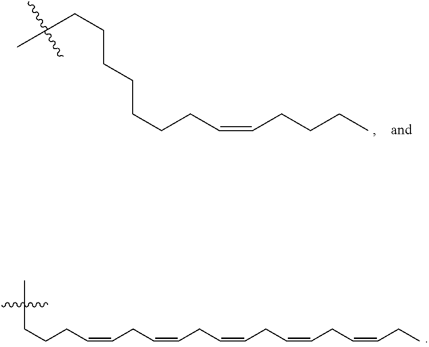 [SMILES: *CCCCCCC/C=C\CCCC ; *CCC/C=C\C/C=C\C/C=C\C/C=C\C/C=C\CC]

In some embodiments, R^{1G} is selected from the group consisting of:

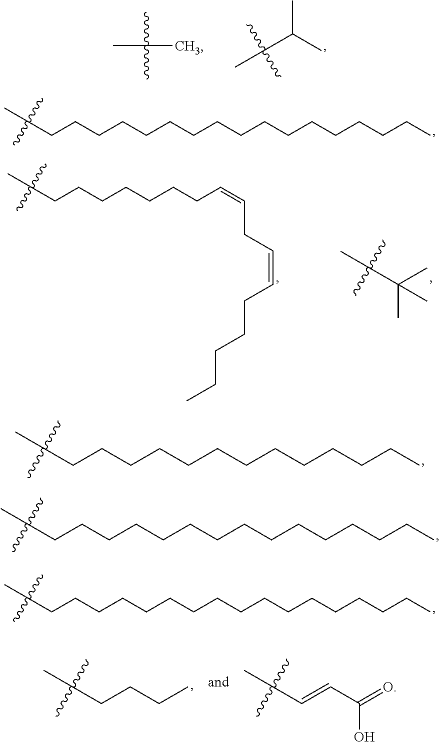

In some embodiment, R¹ is selected from the group consisting of:

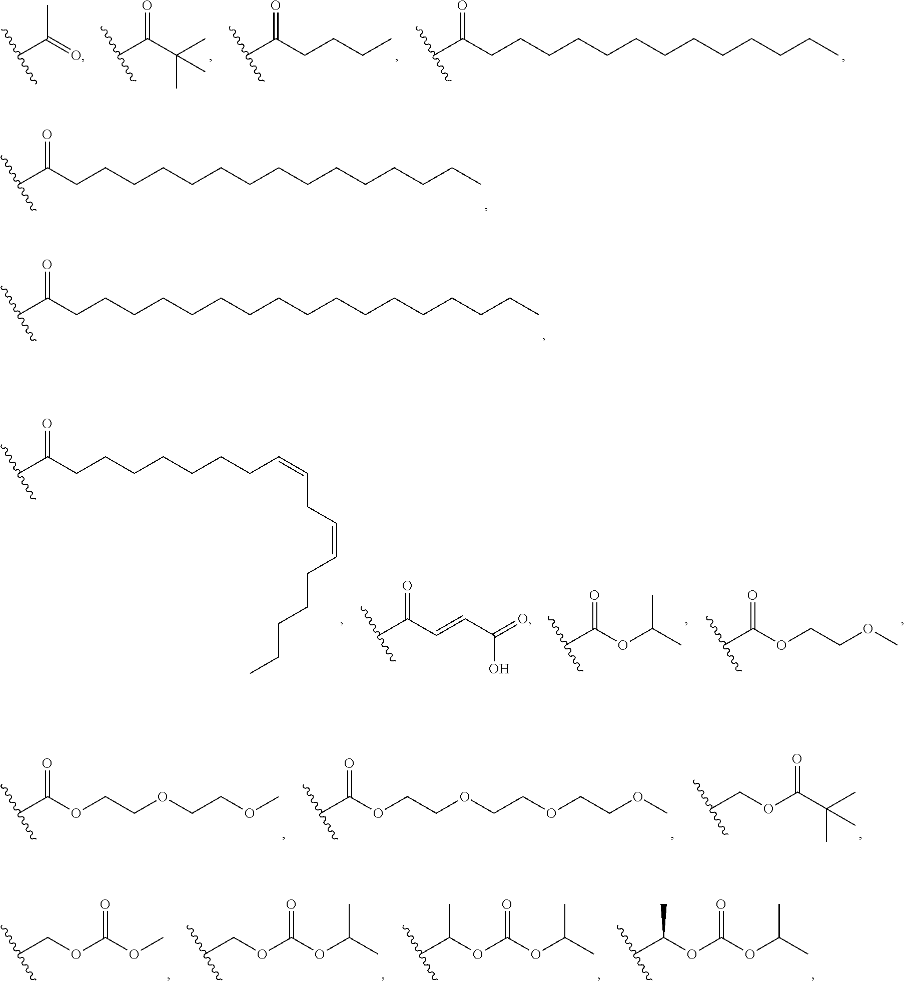

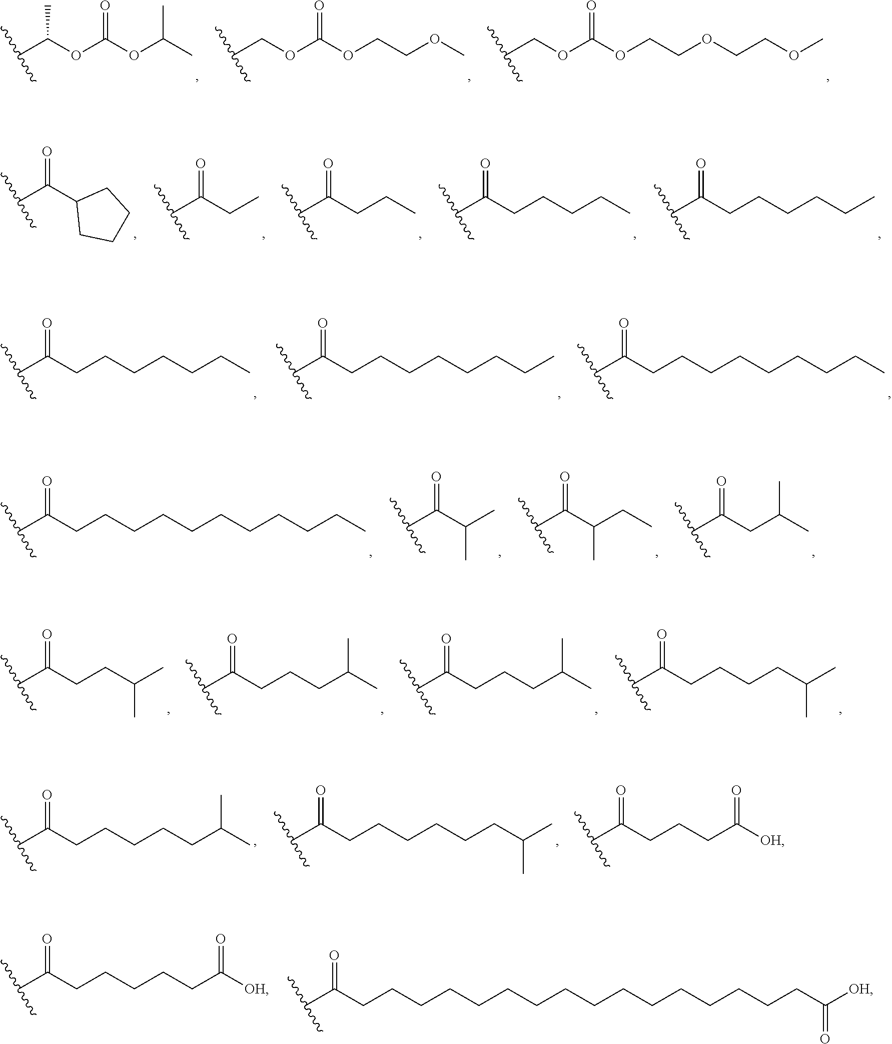

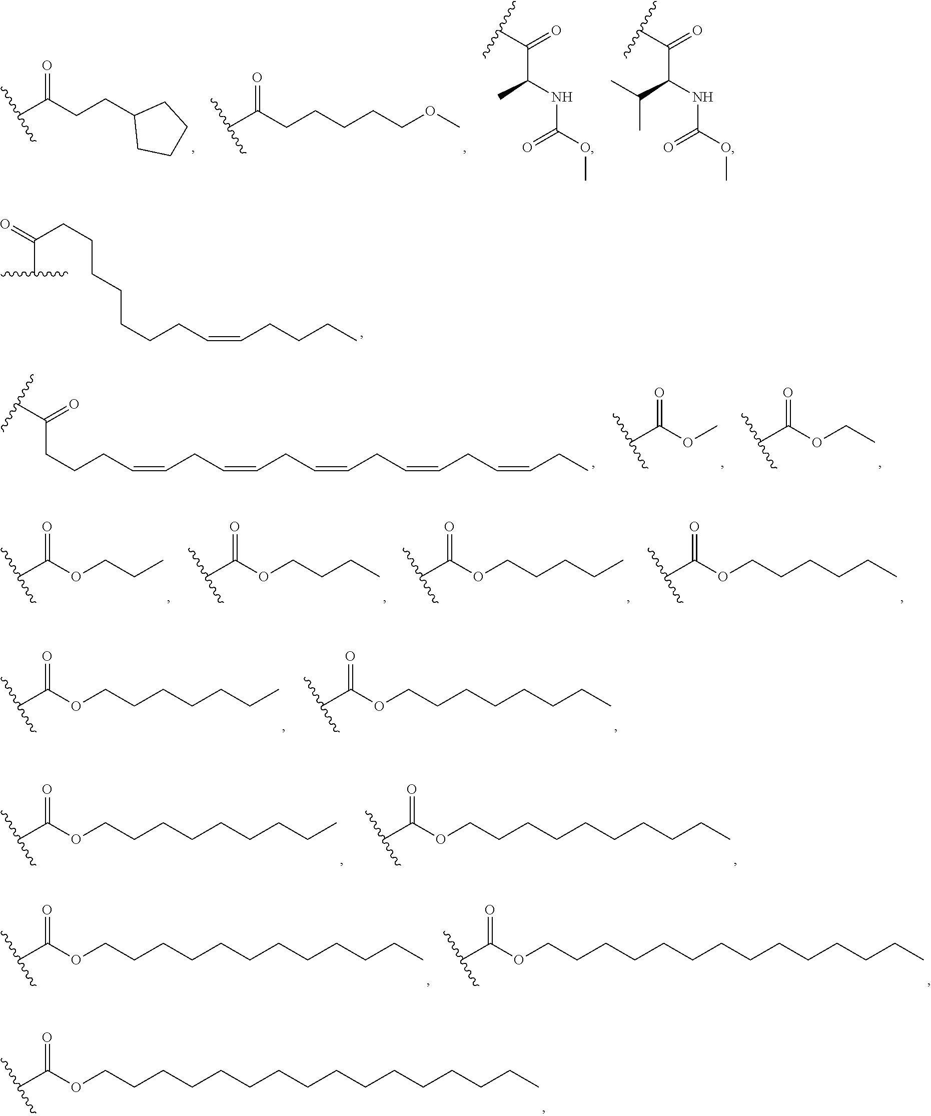

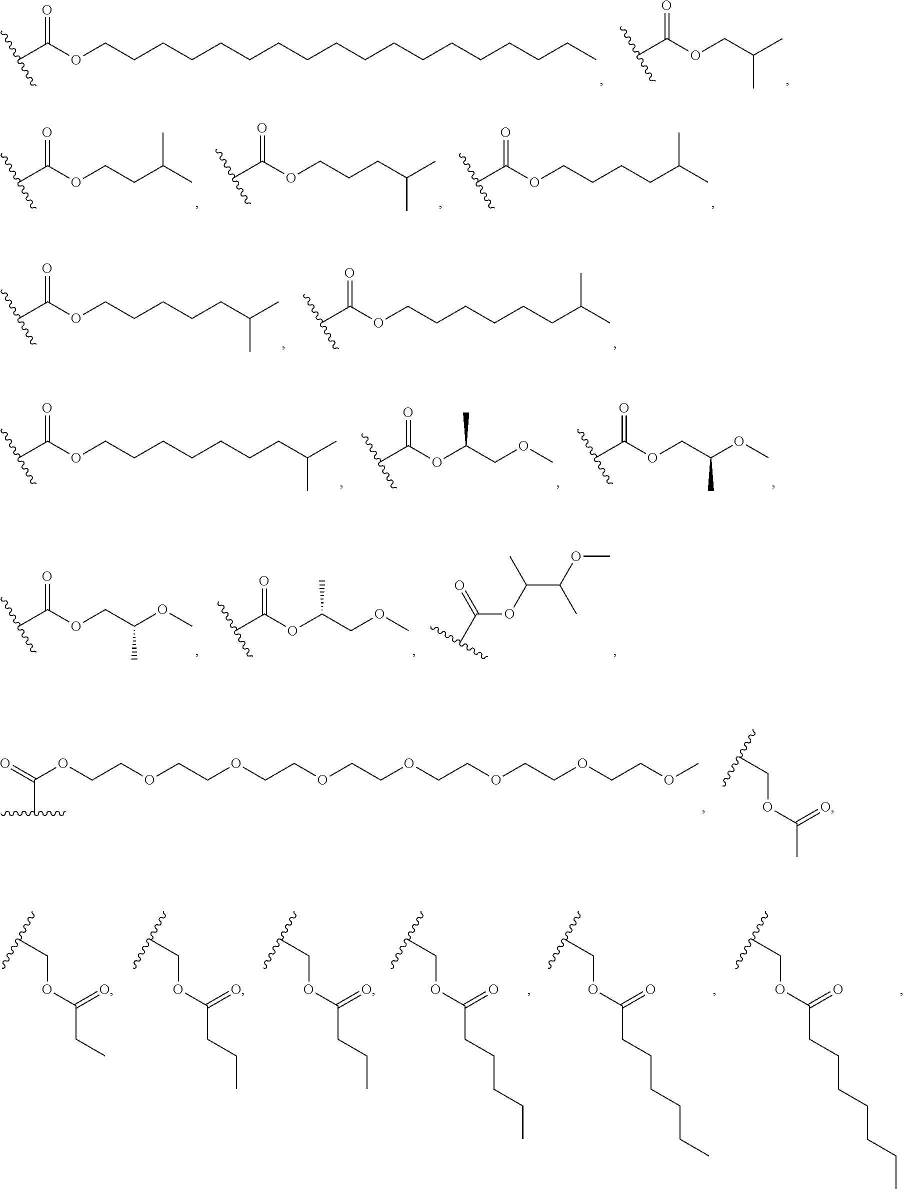

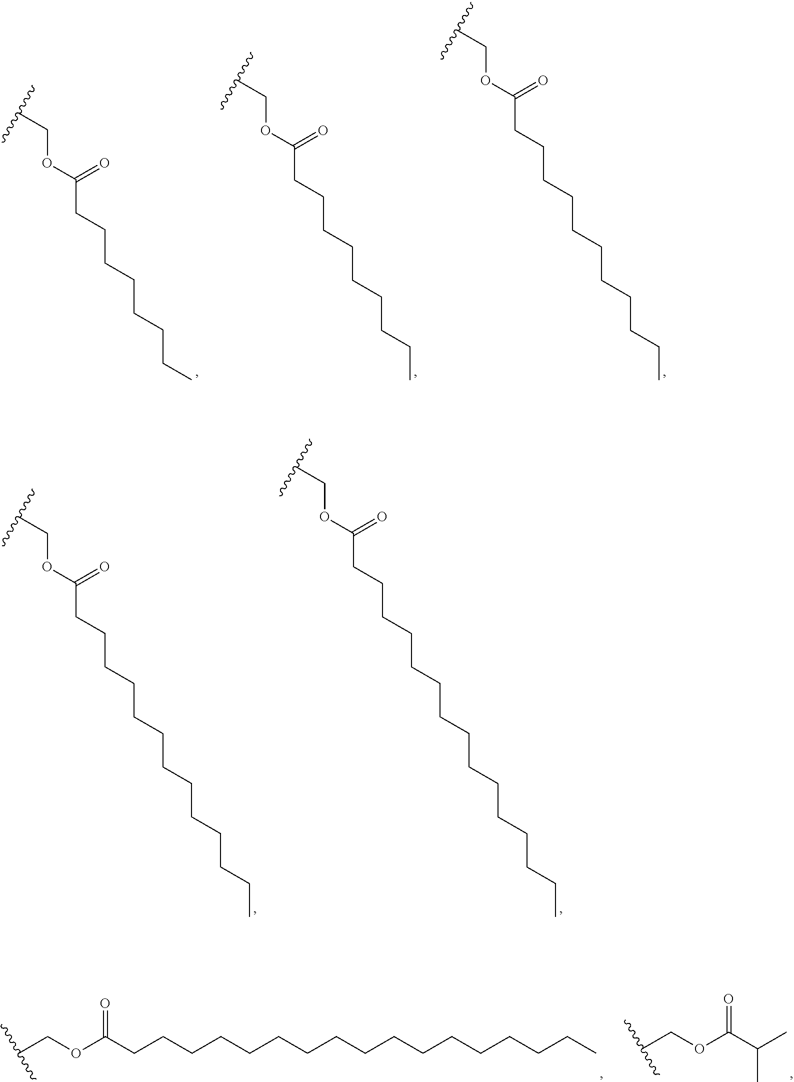

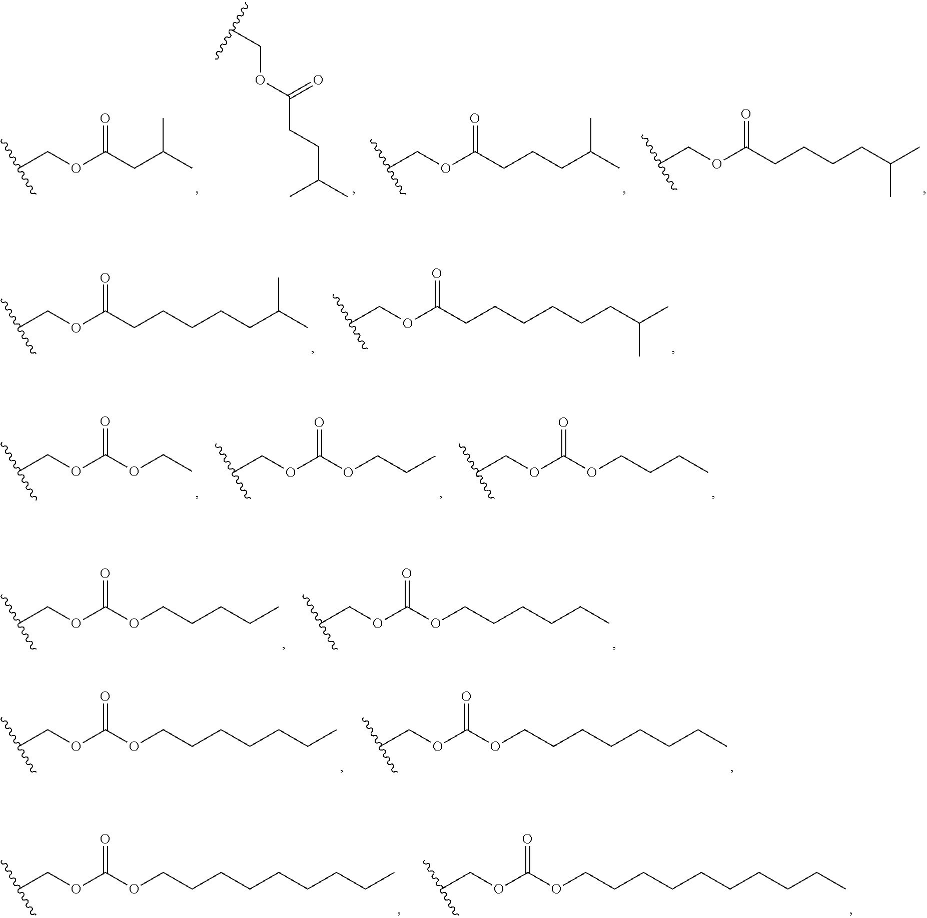

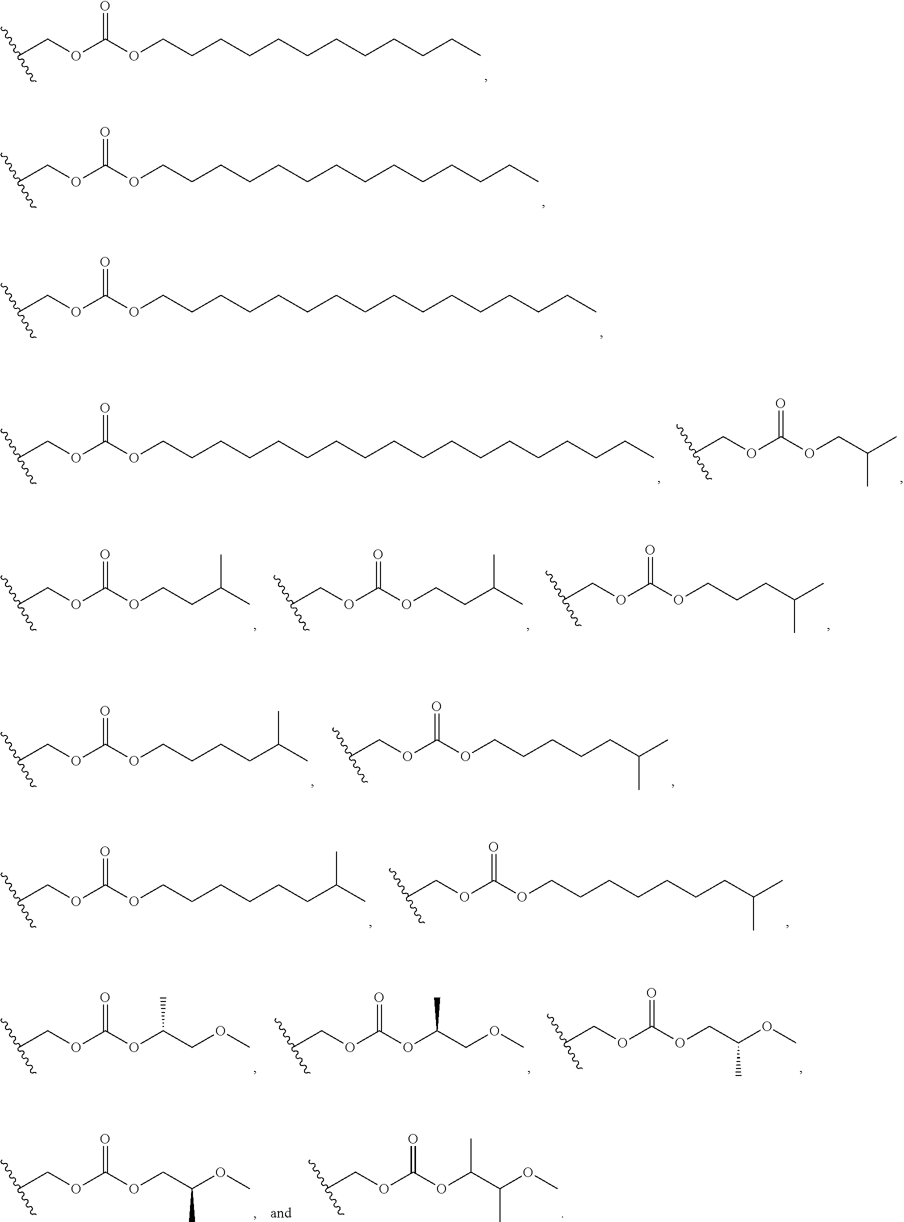

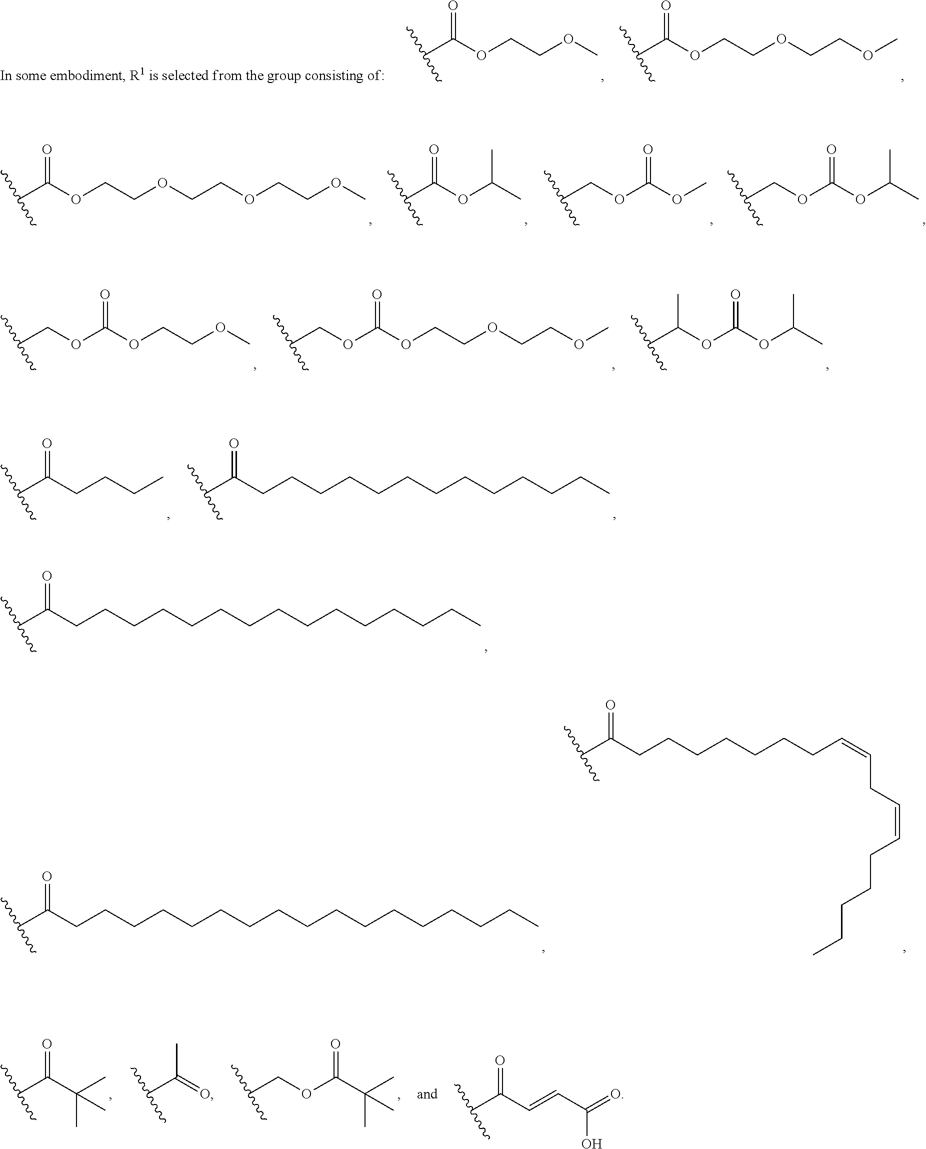

In some embodiment, R¹ is selected from the group consisting of:

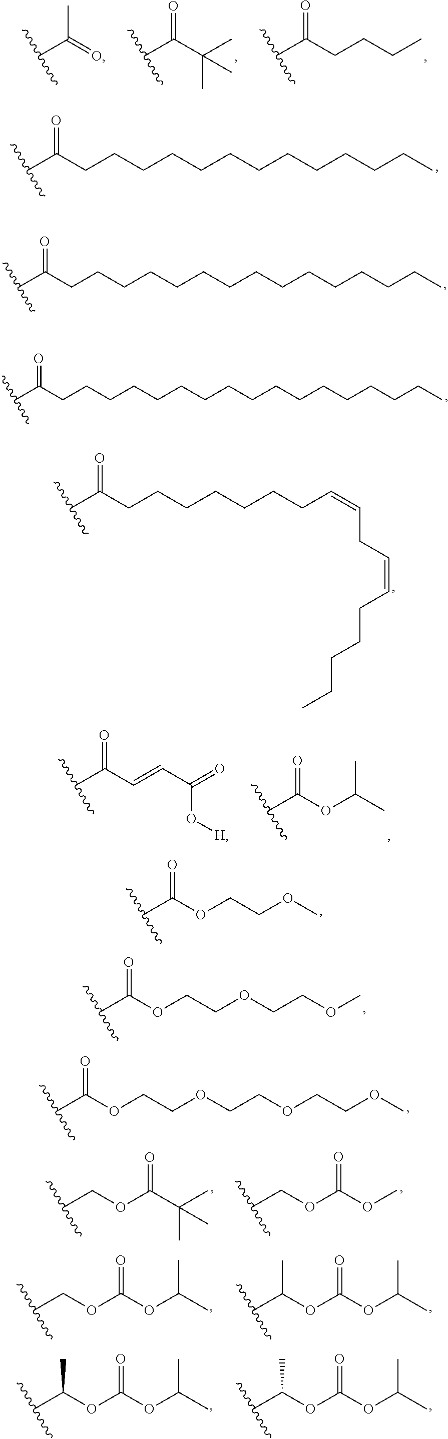

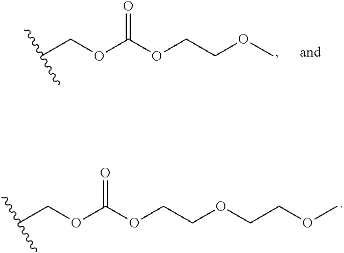

In some embodiments, R² is methyl or methoxy. In some embodiments, R² is methyl. In some embodiments, R² is methoxy.

In some embodiments, R³ and R⁶ are each independently a halo. In some embodiments. R³ and R⁶ are each F.

In some embodiments, R⁴, R⁵ and R⁷ are each H.

In some embodiments, R⁸ is C₁₋₃alkyl. In some embodiments, R⁸ is methyl.

In some embodiments of the compound of Formula I, or the pharmaceutically acceptable salt thereof, the compound has a Formula Ia:

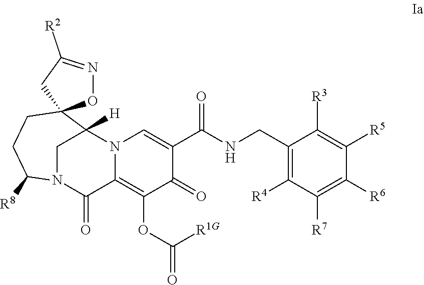

wherein variables R^{1G}, R², R³, R⁴, R⁵, R⁶, R⁷, and R⁸ are defined according to the definitions provided herein for compounds of Formula I.

In some embodiments of the compound of Formula I, or the pharmaceutically acceptable salt thereof, the compound has a Formula Ib.

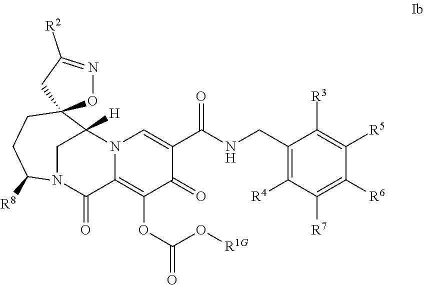

wherein variables R^{1G}, R², R³, R⁴, R⁵, R⁶, R⁷, and R⁸ are defined according to the definitions provided herein for compounds of Formula I.

In some embodiments of the compound of Formula I, or the pharmaceutically acceptable salt thereof, the compound has a Formula Ic:

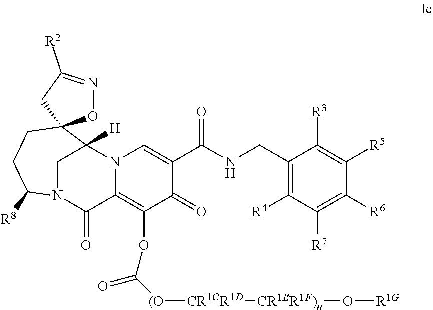

wherein n is an integer between 1 and 7, inclusive, and variables R^{1C}, R^{1D}, R^{1E}, R^{1F}, R^{1G}, R², R³, R⁴, R⁵, R⁶, R⁷, and R⁸ are defined according to the definitions provided herein for compounds of Formula I. In some embodiments, of the compound of Formula Ic, or the pharmaceutically acceptable salt thereof, n is 1, 2, 3, or 7. In some embodiments, of the compound of Formula Ic, or the pharmaceutically acceptable salt thereof, n is 1, 2, or 3.

In some embodiments of the compound of Formula I, or the pharmaceutically acceptable salt thereof, the compound has a Formula Id:

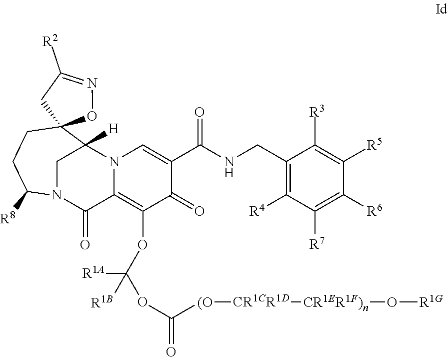

wherein n is 0, 1, 2 or 3; and variables R^{1A}, R^{1B}, R^{1C}, R^{1D}, R^{1E}, R^{1F}, R^{1G}, R², R³, R⁴, R¹, R⁶, R⁷, and R⁸ are defined according to the definitions provided herein for compounds of Formula I.

In some embodiments of the compound of Formula Id, or the pharmaceutically acceptable salt thereof, n is 1, 2, or 3.

In some embodiments of the compound of Formula I, or the pharmaceutically acceptable salt thereof, the compound has a Formula Ie:

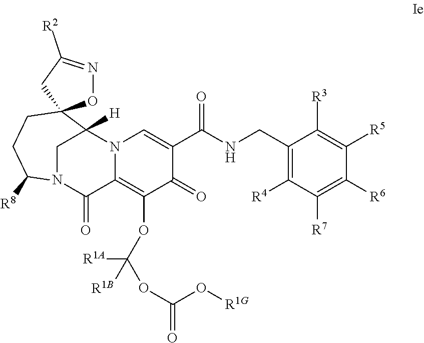

wherein variables R^{1A}, R^{1B}, R^{1G}, R², R³, R⁴, R⁵, R⁶, R⁷, and R⁸ are defined according to the definitions provided herein for compounds of Formula I.

In some embodiments of the compound of Formula I, or the pharmaceutically acceptable salt thereof, the compound has a Formula If:

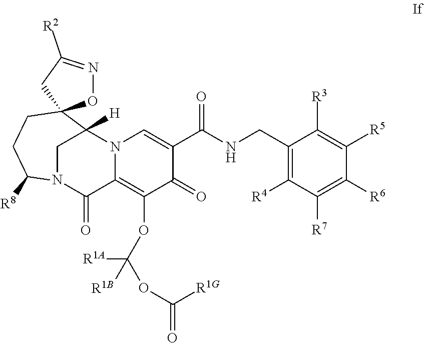

wherein variables R^{1A}, R^{1B}, R^{1G}, R², R³, R⁴, R⁵, R⁶, R⁷, and R⁸ are defined according to the definitions provided herein for compounds of Formula I.

In some embodiments of the compounds of Formula I, Ib, Ic, Id, or Ie, the compound is selected from the group consisting of:

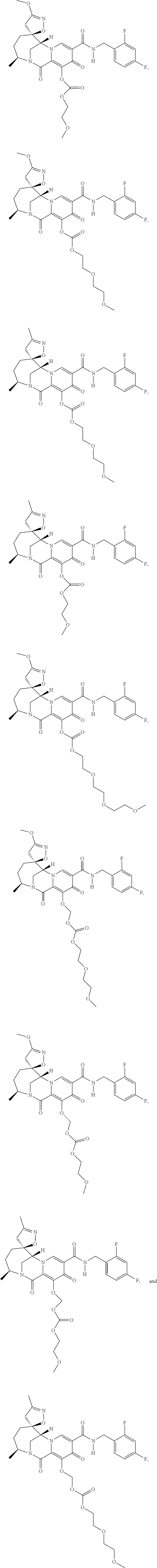

or a pharmaceutically acceptable salt thereof.

In some embodiments of the compounds of Formula I, Ia, or Ib, the compound is selected from the group consisting of

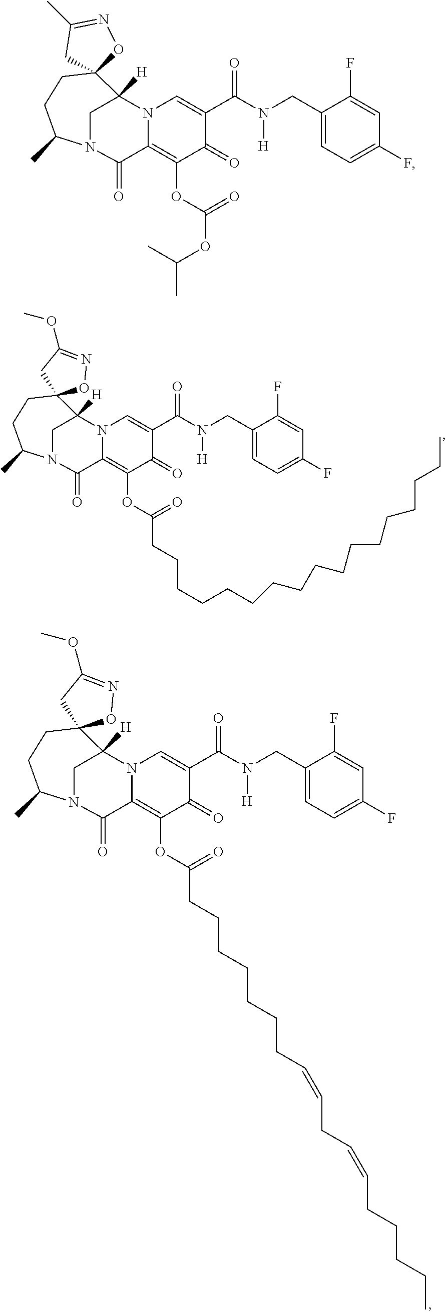

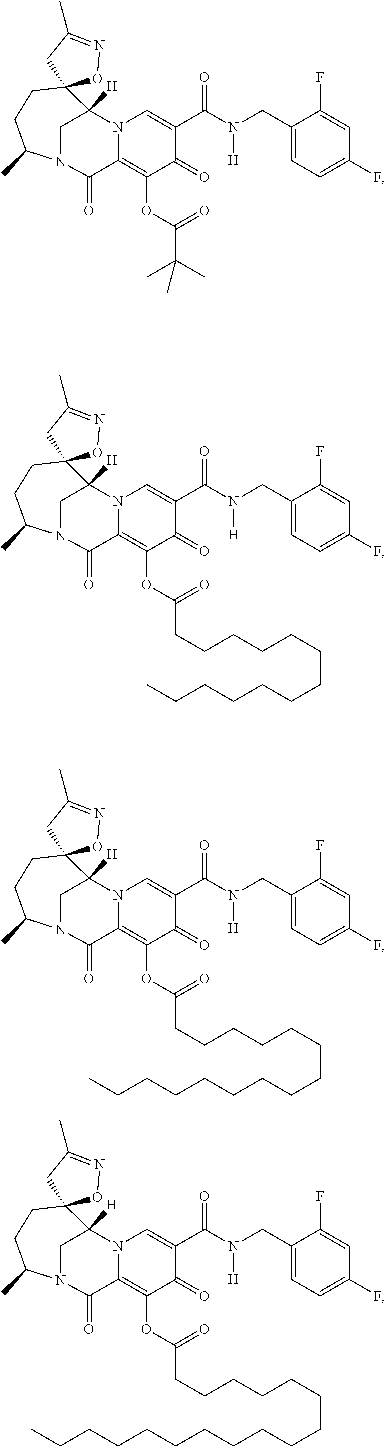 [SMILES: CC1=NO[C@@]2(CC[C@H](C)N3C[C@H]2n2cc(C(=O)NCc4ccc(F)cc4F)c(=O)c(OC(=O)C(C)(C)C)c2C3=O)C1 ; CCCCCCCCCCCCCC(=O)Oc1c2n(cc(C(=O)NCc3ccc(F)cc3F)c1=O)[C@@H]1CN(C2=O)[C@@H](C)CC[C@]12CC(C)=NO2 ; CCCCCCCCCCCCCCCC(=O)Oc1c2n(cc(C(=O)NCc3ccc(F)cc3F)c1=O)[C@@H]1CN(C2=O)[C@@H](C)CC[C@]12CC(C)=NO2 ; CCCCCCCCCCCCCCCCCC(=O)Oc1c2n(cc(C(=O)NCc3ccc(F)cc3F)c1=O)[C@@H]1CN(C2=O)[C@@H](C)CC[C@]12CC(C)=NO2]

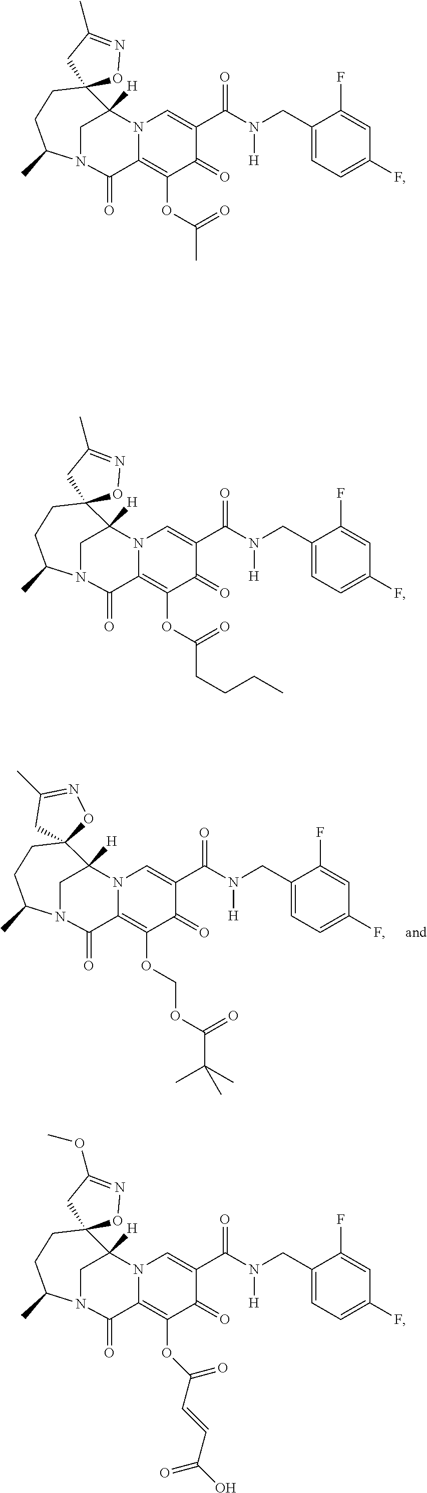 [SMILES: CC(=O)Oc1c2n(cc(C(=O)NCc3ccc(F)cc3F)c1=O)[C@@H]1CN(C2=O)[C@@H](C)CC[C@]12CC(C)=NO2 ; CCCCC(=O)Oc1c2n(cc(C(=O)NCc3ccc(F)cc3F)c1=O)[C@@H]1CN(C2=O)[C@@H](C)CC[C@]12CC(C)=NO2 ; CC1=NO[C@@]2(CC[C@H](C)N3C[C@H]2n2cc(C(=O)NCc4ccc(F)cc4F)c(=O)c(OCOC(=O)C(C)(C)C)c2C3=O)C1 ; COC1=NO[C@@]2(CC[C@H](C)N3C[C@H]2n2cc(C(=O)NCc4ccc(F)cc4F)c(=O)c(OC(=O)/C=C/C(=O)O)c2C3=O)C1]

or a pharmaceutically acceptable salt thereof.

In some embodiments of the compounds of Formula I, Id, or Ie, the compound is selected from the group consisting of:

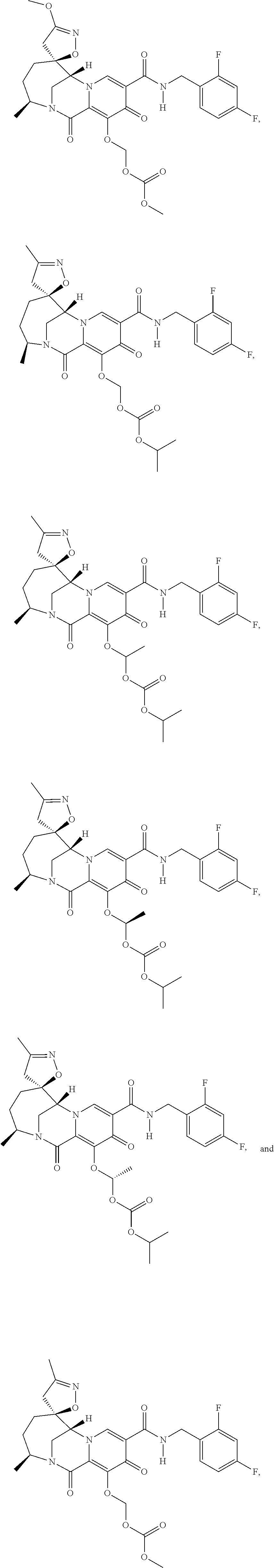

or a pharmaceutically acceptable salt thereof.

In some embodiments of the compounds of Formula I, Ia, or Ib, the compound is selected from the group consisting of

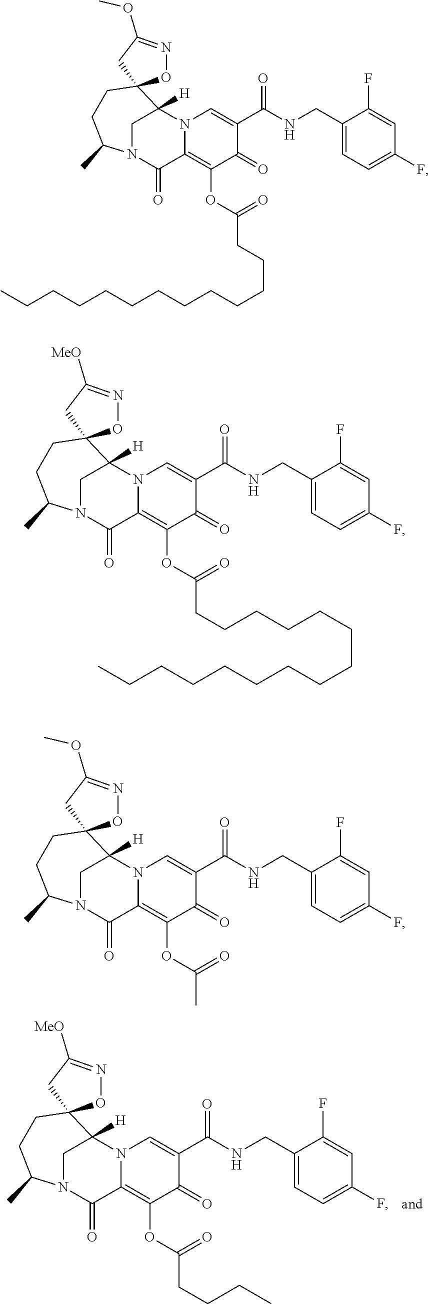

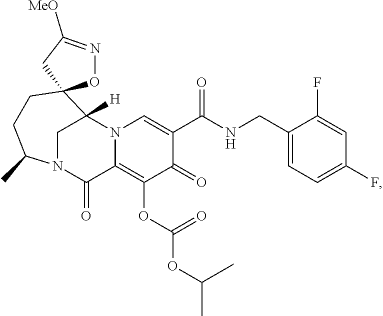 [SMILES: COC1=NO[C@@]2(CC[C@H](C)N3C[C@H]2n2cc(C(=O)NCc4ccc(F)cc4F)c(=O)c(OC(=O)OC(C)C)c2C3=O)C1]

or a pharmaceutically acceptable salt thereof.

In some embodiments of the compounds of Formula I or Ia, the compound is selected from the group consisting of:

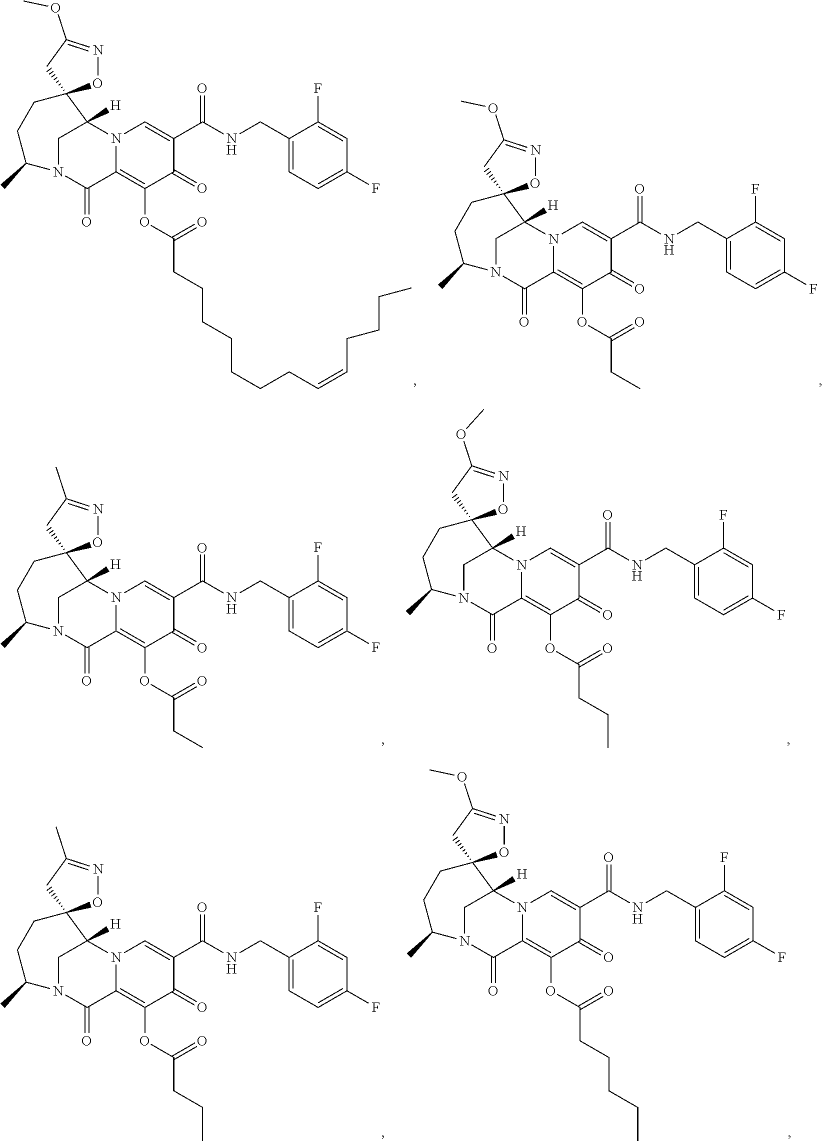

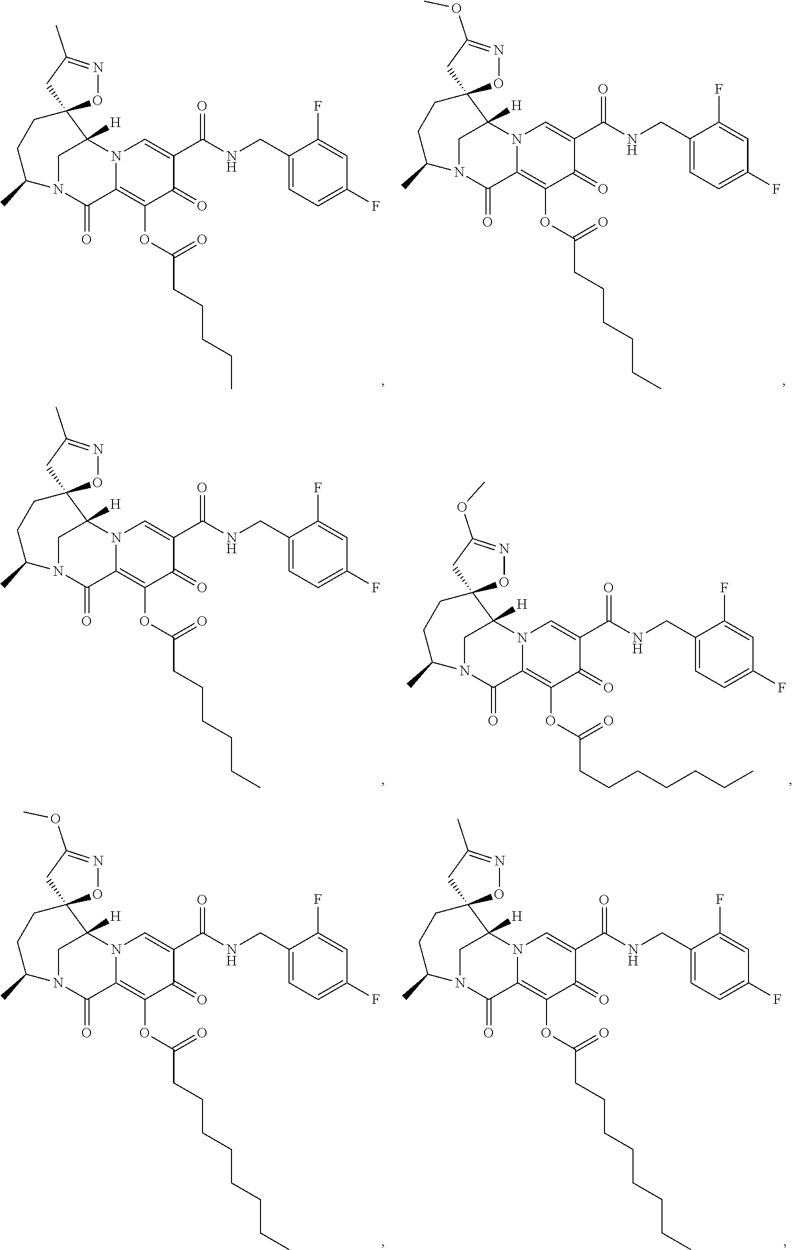

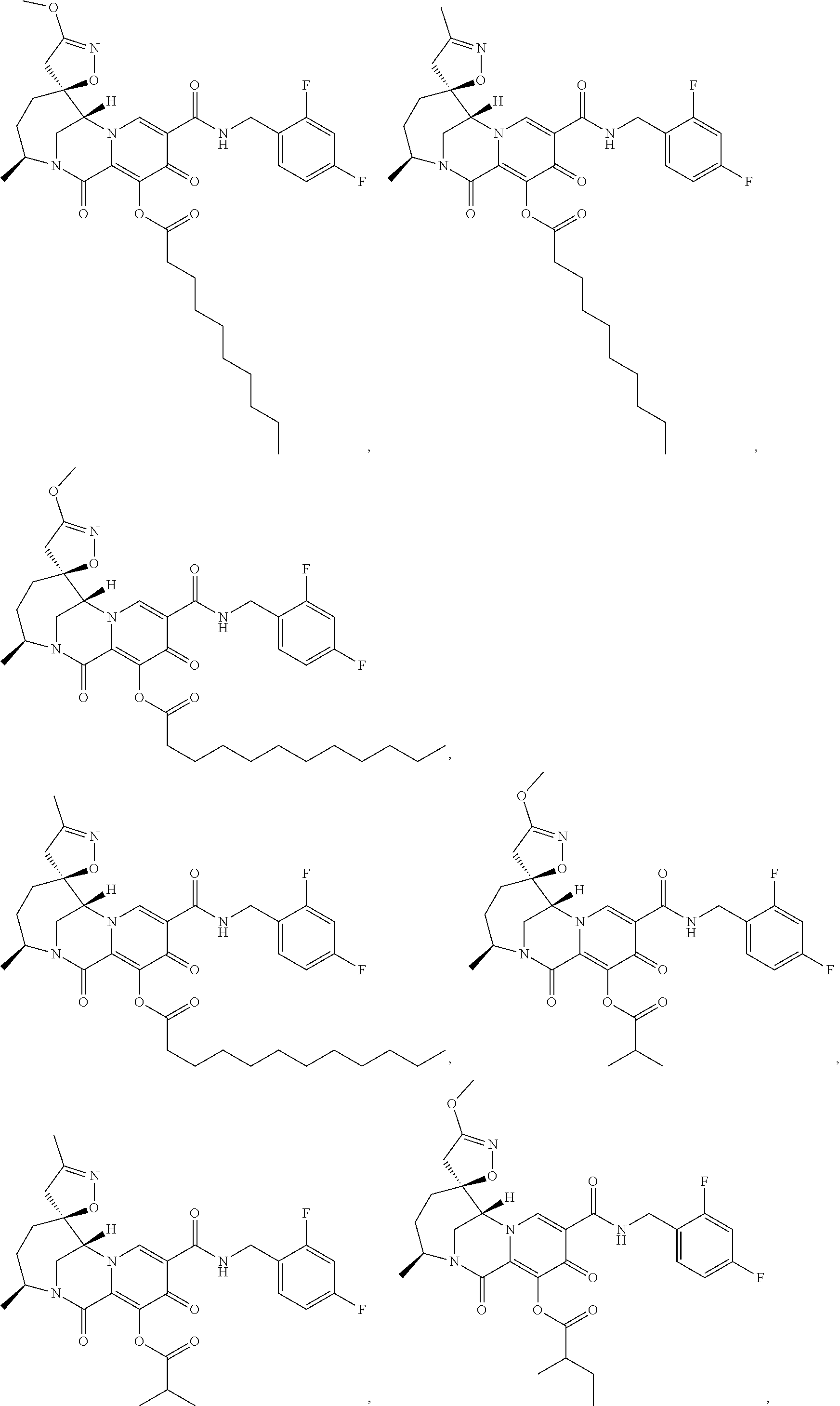

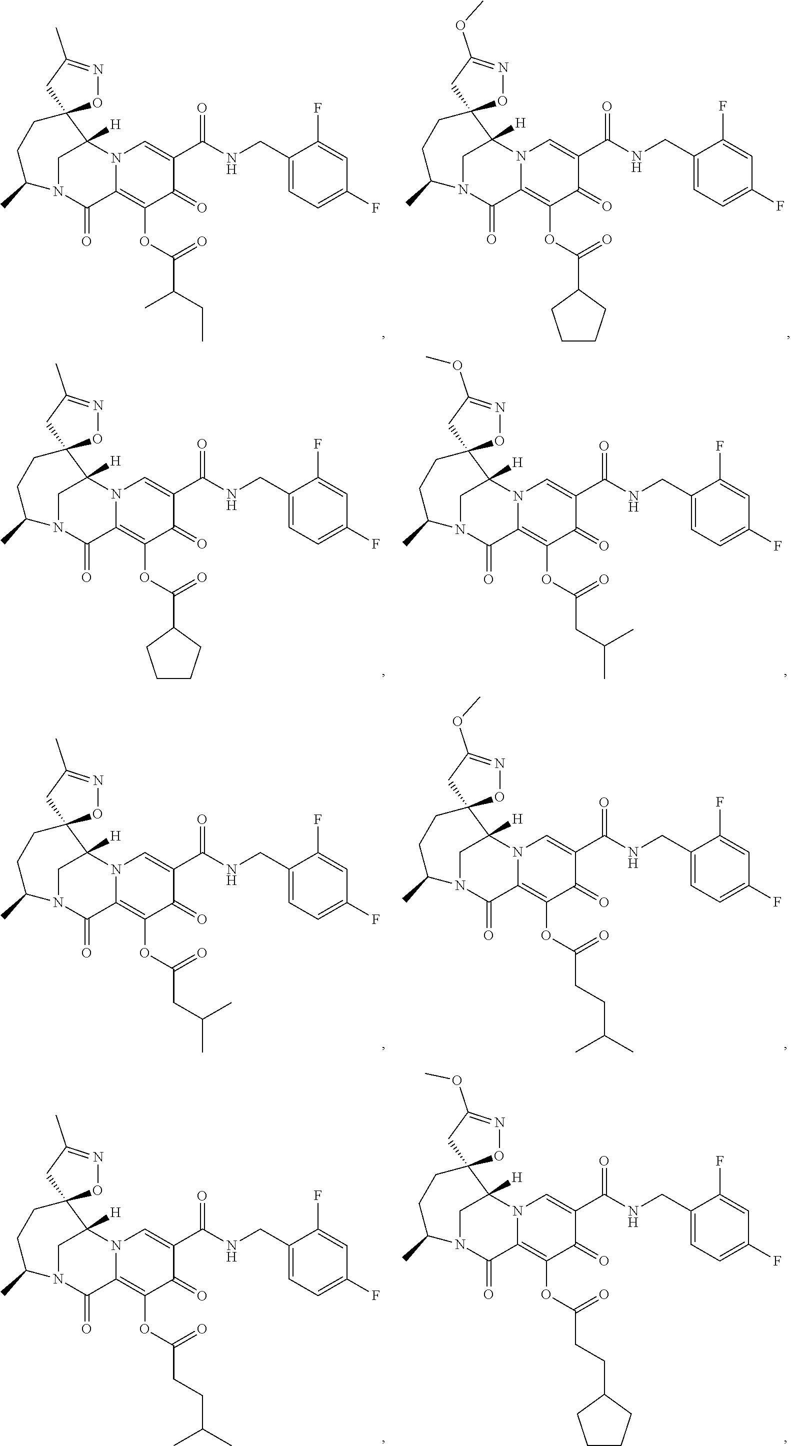

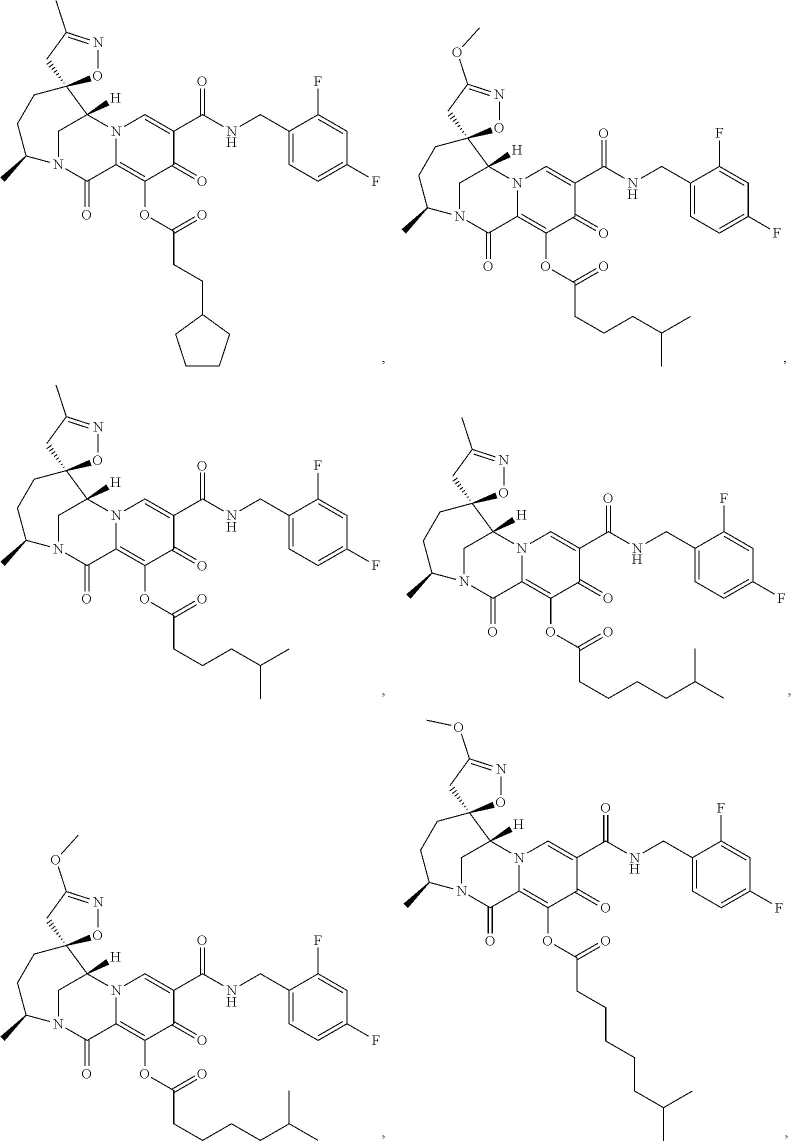

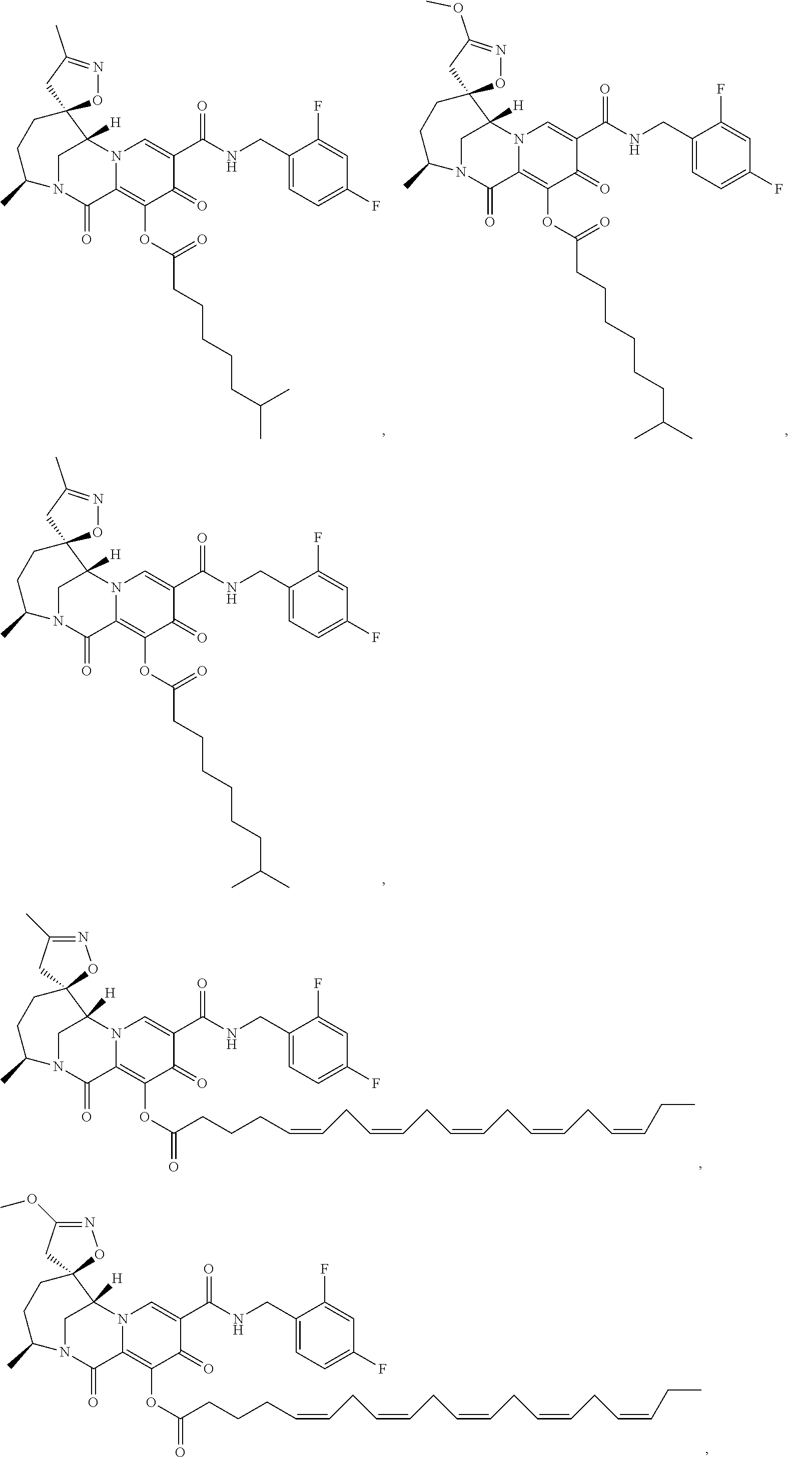

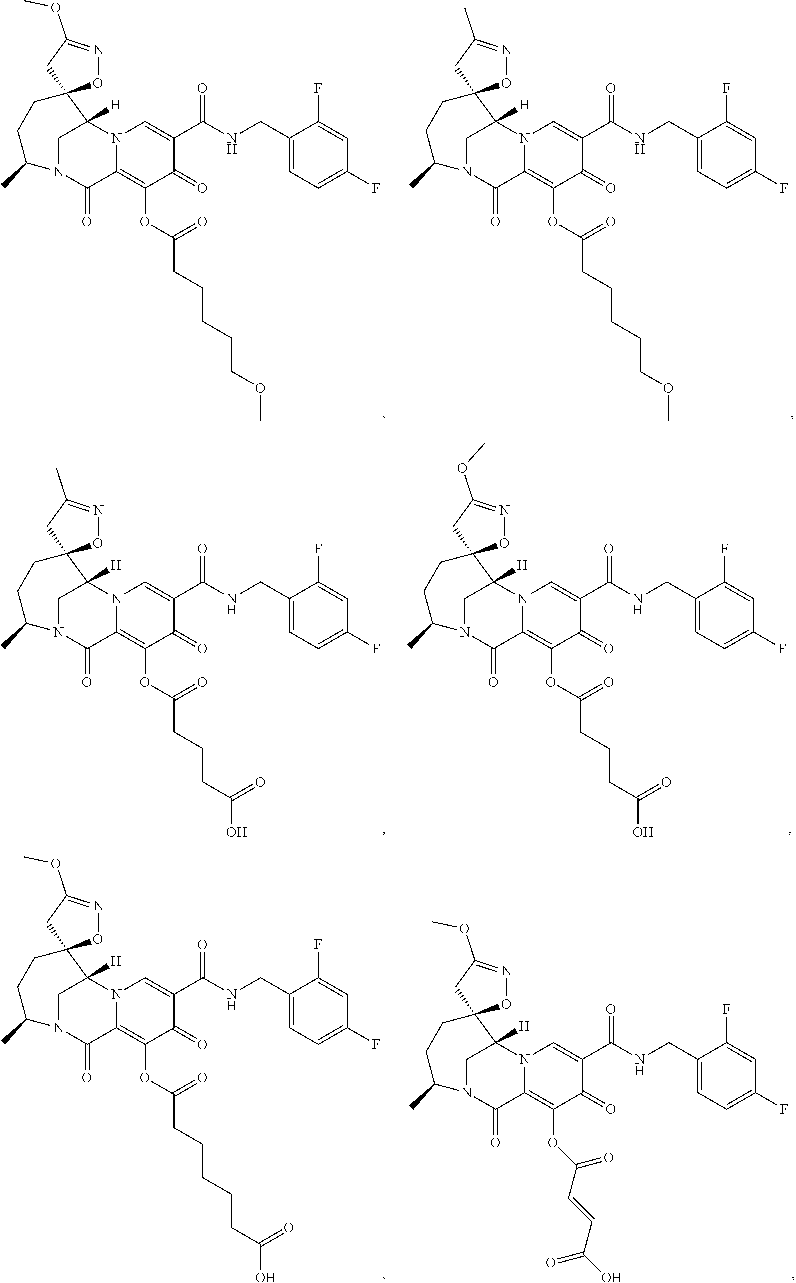

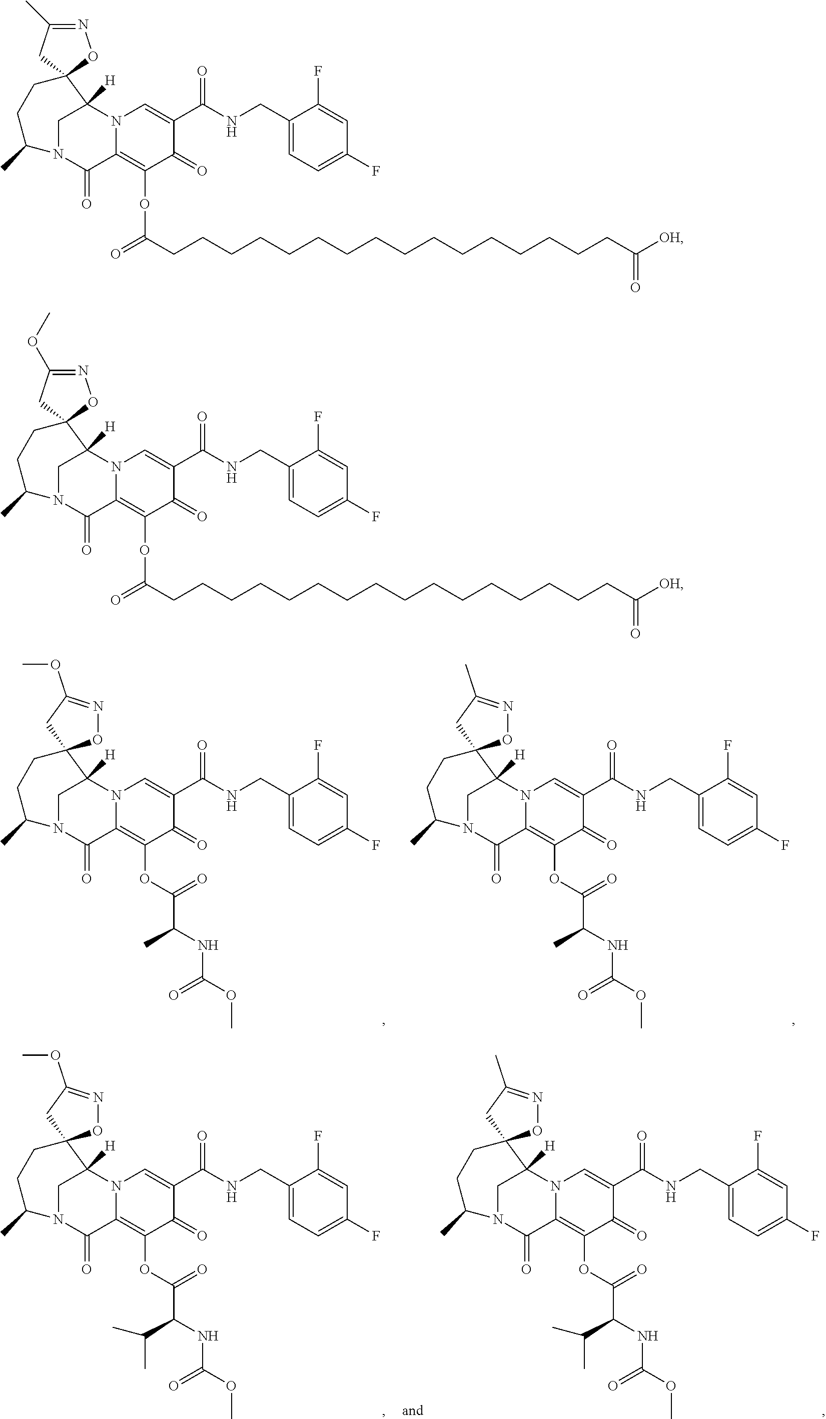

or a pharmaceutically acceptable salt thereof.

In some embodiments of the compounds of Formula I or If, the compound is selected from the group consisting of:

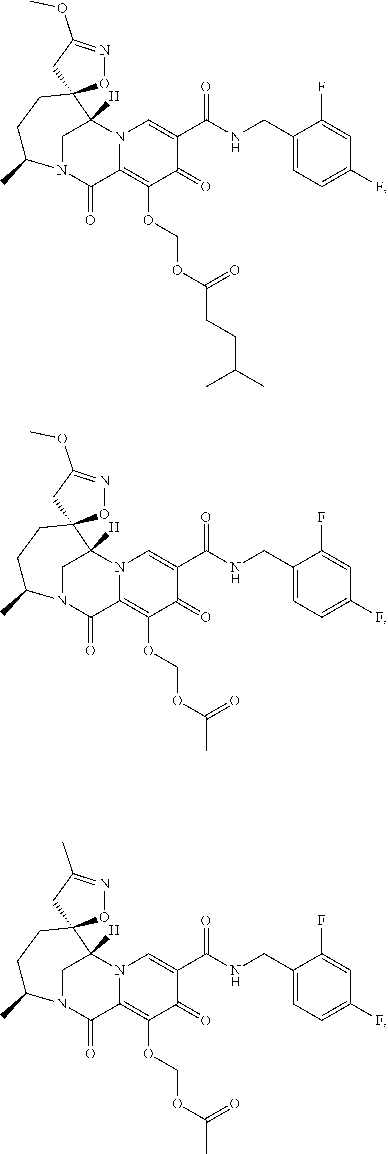 [SMILES: COC1=NO[C@@]2(CC[C@H](C)N3C[C@H]2n2cc(C(=O)NCc4ccc(F)cc4F)c(=O)c(OCOC(=O)CCC(C)C)c2C3=O)C1 ; COC1=NO[C@@]2(CC[C@H](C)N3C[C@H]2n2cc(C(=O)NCc4ccc(F)cc4F)c(=O)c(OCOC(C)=O)c2C3=O)C1 ; CC(=O)OCOc1c2n(cc(C(=O)NCc3ccc(F)cc3F)c1=O)[C@@H]1CN(C2=O)[C@@H](C)CC[C@]12CC(C)=NO2]

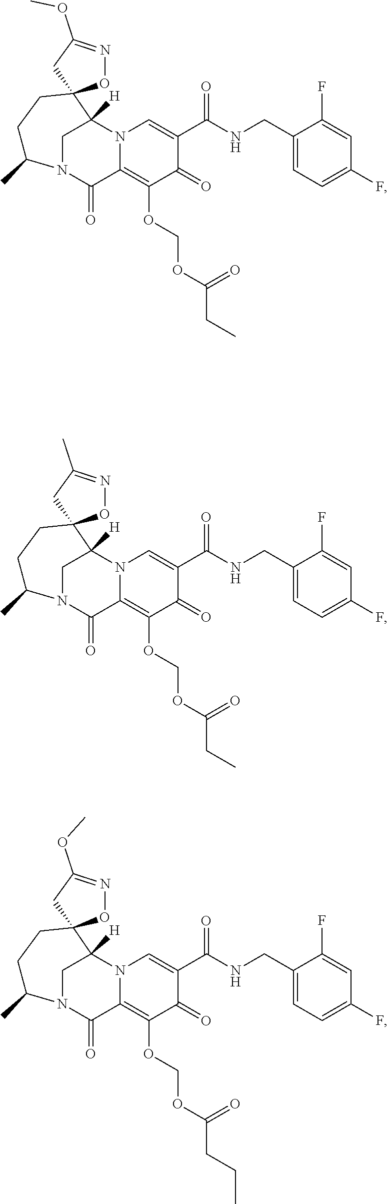 [SMILES: CCC(=O)OCOc1c2n(cc(C(=O)NCc3ccc(F)cc3F)c1=O)[C@@H]1CN(C2=O)[C@@H](C)CC[C@]12CC(OC)=NO2 ; CCC(=O)OCOc1c2n(cc(C(=O)NCc3ccc(F)cc3F)c1=O)[C@@H]1CN(C2=O)[C@@H](C)CC[C@]12CC(C)=NO2 ; CCCC(=O)OCOc1c2n(cc(C(=O)NCc3ccc(F)cc3F)c1=O)[C@@H]1CN(C2=O)[C@@H](C)CC[C@]12CC(OC)=NO2]

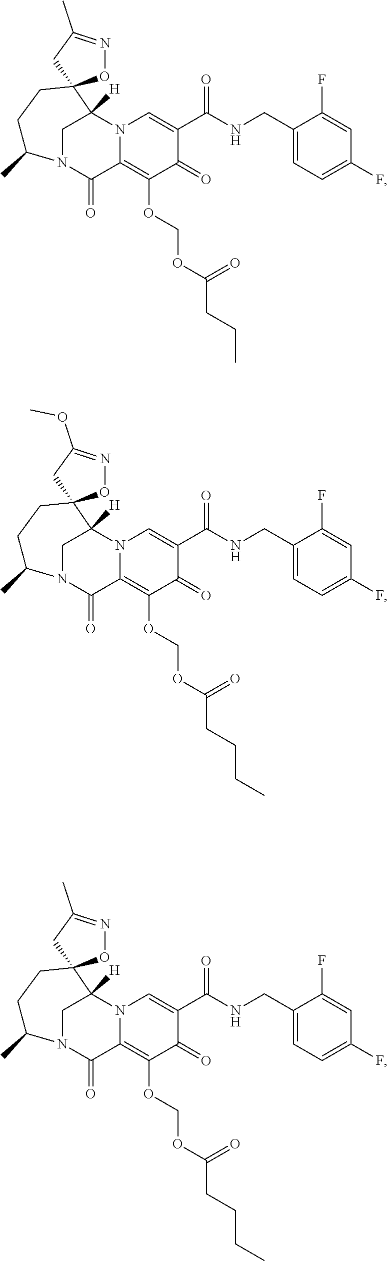 [SMILES: CCCC(=O)OCOc1c2n(cc(C(=O)NCc3ccc(F)cc3F)c1=O)[C@@H]1CN(C2=O)[C@@H](C)CC[C@]12CC(C)=NO2 ; CCCCC(=O)OCOc1c2n(cc(C(=O)NCc3ccc(F)cc3F)c1=O)[C@@H]1CN(C2=O)[C@@H](C)CC[C@]12CC(OC)=NO2 ; CCCCC(=O)OCOc1c2n(cc(C(=O)NCc3ccc(F)cc3F)c1=O)[C@@H]1CN(C2=O)[C@@H](C)CC[C@]12CC(C)=NO2]

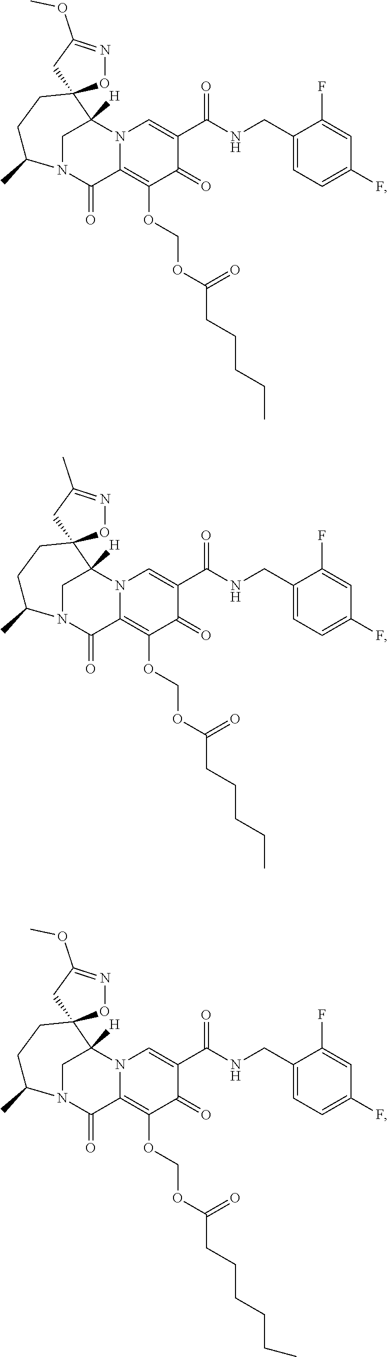 [SMILES: CCCCCC(=O)OCOc1c2n(cc(C(=O)NCc3ccc(F)cc3F)c1=O)[C@@H]1CN(C2=O)[C@@H](C)CC[C@]12CC(OC)=NO2 ; CCCCCC(=O)OCOc1c2n(cc(C(=O)NCc3ccc(F)cc3F)c1=O)[C@@H]1CN(C2=O)[C@@H](C)CC[C@]12CC(C)=NO2 ; CCCCCCC(=O)OCOc1c2n(cc(C(=O)NCc3ccc(F)cc3F)c1=O)[C@@H]1CN(C2=O)[C@@H](C)CC[C@]12CC(OC)=NO2]

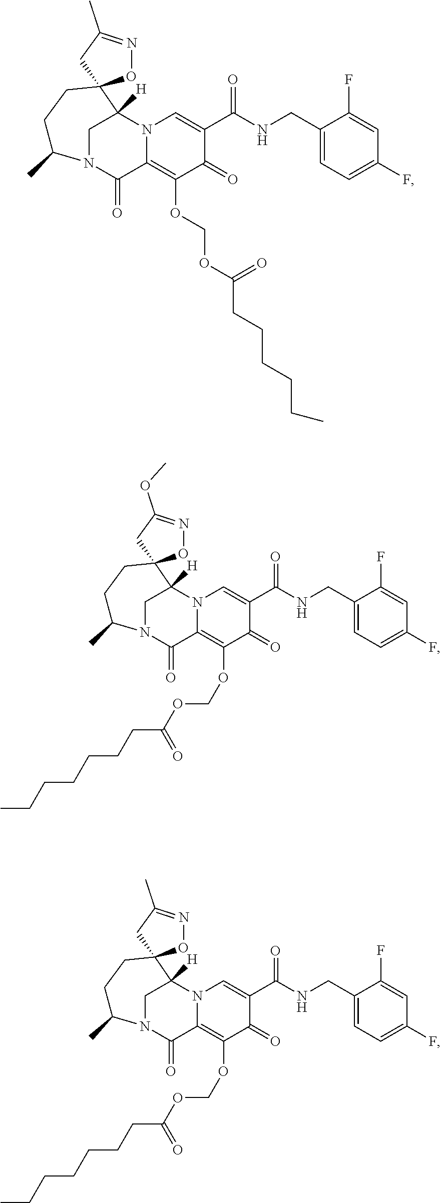 [SMILES: CCCCCCC(=O)OCOc1c2n(cc(C(=O)NCc3ccc(F)cc3F)c1=O)[C@@H]1CN(C2=O)[C@@H](C)CC[C@]12CC(C)=NO2 ; CCCCCCCC(=O)OCOc1c2n(cc(C(=O)NCc3ccc(F)cc3F)c1=O)[C@@H]1CN(C2=O)[C@@H](C)CC[C@]12CC(OC)=NO2 ; CCCCCCCC(=O)OCOc1c2n(cc(C(=O)NCc3ccc(F)cc3F)c1=O)[C@@H]1CN(C2=O)[C@@H](C)CC[C@]12CC(C)=NO2]

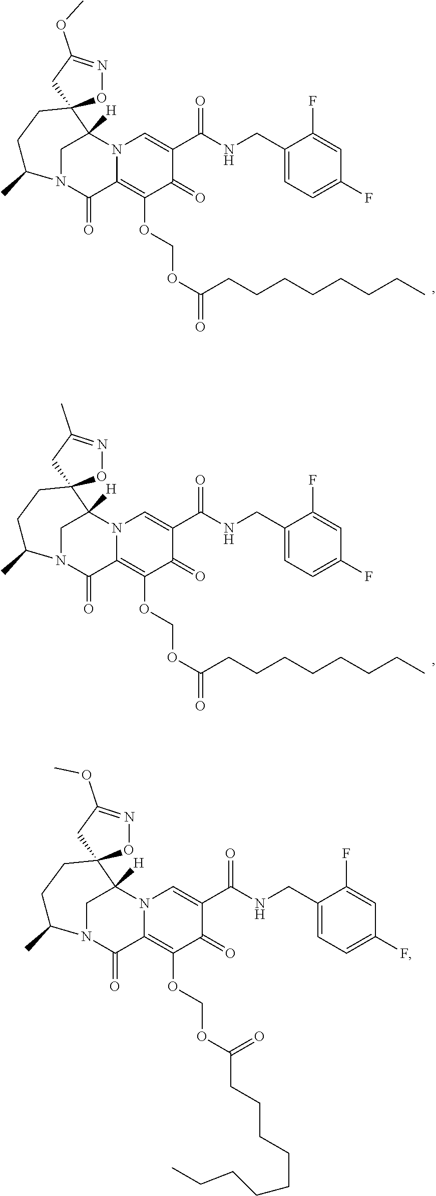

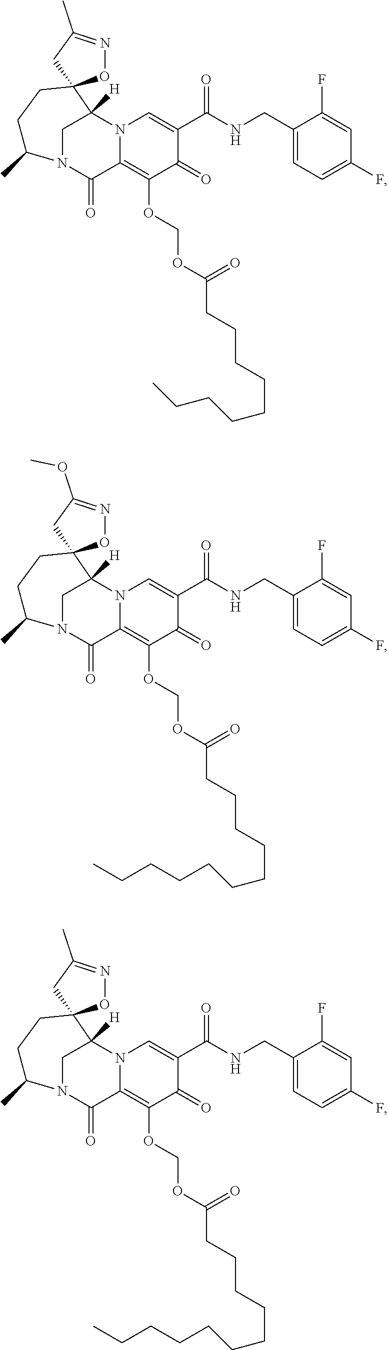 [SMILES: CCCCCCCCCC(=O)OCOc1c2n(cc(C(=O)NCc3ccc(F)cc3F)c1=O)[C@@H]1CN(C2=O)[C@@H](C)CC[C@]12CC(C)=NO2 ; CCCCCCCCCCCC(=O)OCOc1c2n(cc(C(=O)NCc3ccc(F)cc3F)c1=O)[C@@H]1CN(C2=O)[C@@H](C)CC[C@]12CC(OC)=NO2 ; CCCCCCCCCCCC(=O)OCOc1c2n(cc(C(=O)NCc3ccc(F)cc3F)c1=O)[C@@H]1CN(C2=O)[C@@H](C)CC[C@]12CC(C)=NO2]

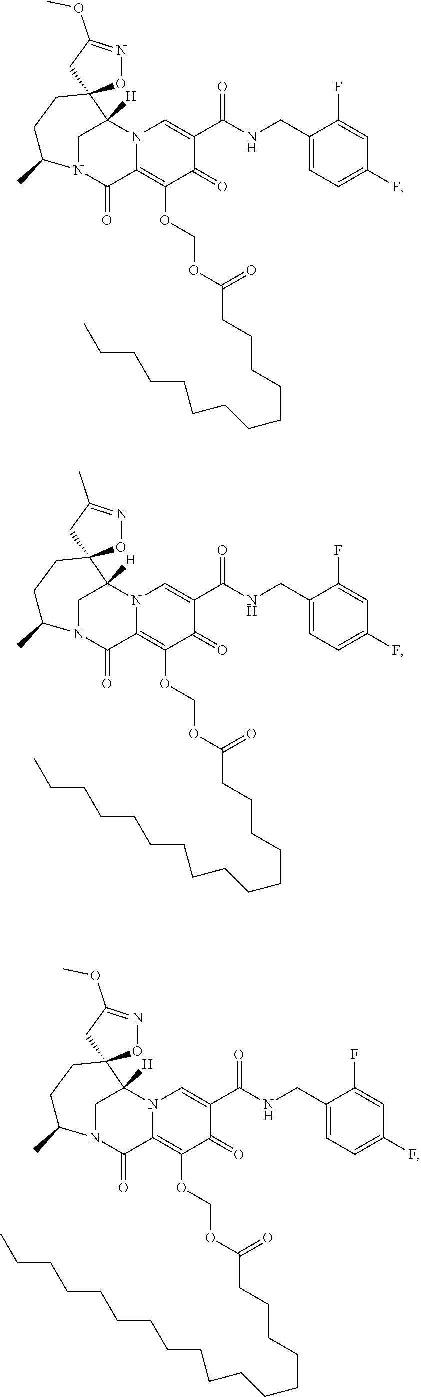 [SMILES: CCCCCCCCCCCCCC(=O)OCOc1c2n(cc(C(=O)NCc3ccc(F)cc3F)c1=O)[C@@H]1CN(C2=O)[C@@H](C)CC[C@]12CC(OC)=NO2 ; CCCCCCCCCCCCCCCC(=O)OCOc1c2n(cc(C(=O)NCc3ccc(F)cc3F)c1=O)[C@@H]1CN(C2=O)[C@@H](C)CC[C@]12CC(C)=NO2 ; CCCCCCCCCCCCCCCCCC(=O)OCOc1c2n(cc(C(=O)NCc3ccc(F)cc3F)c1=O)[C@@H]1CN(C2=O)[C@@H](C)CC[C@]12CC(OC)=NO2]

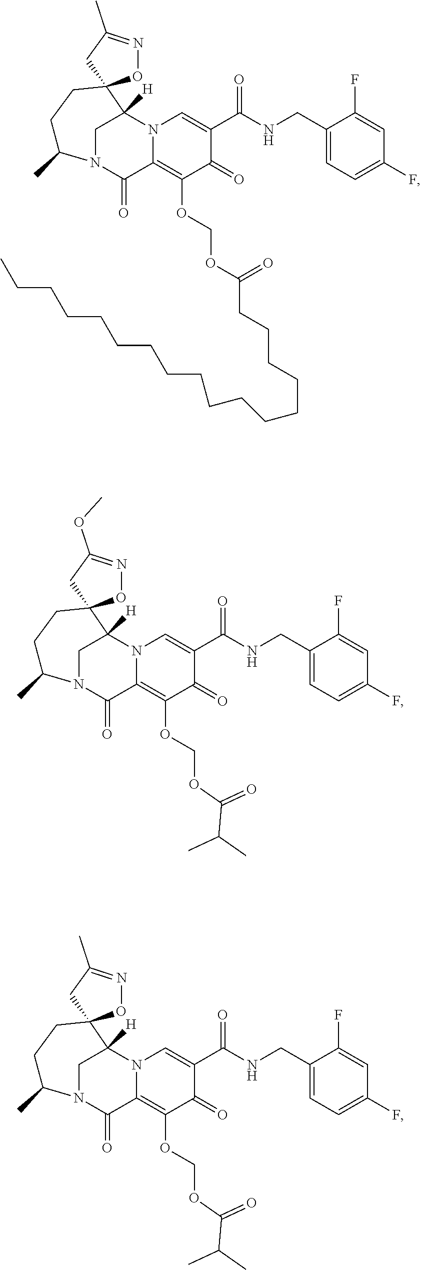 [SMILES: CCCCCCCCCCCCCCCCCC(=O)OCOc1c2n(cc(C(=O)NCc3ccc(F)cc3F)c1=O)[C@@H]1CN(C2=O)[C@@H](C)CC[C@]12CC(C)=NO2 ; COC1=NO[C@@]2(CC[C@H](C)N3C[C@H]2n2cc(C(=O)NCc4ccc(F)cc4F)c(=O)c(OCOC(=O)C(C)C)c2C3=O)C1 ; CC1=NO[C@@]2(CC[C@H](C)N3C[C@H]2n2cc(C(=O)NCc4ccc(F)cc4F)c(=O)c(OCOC(=O)C(C)C)c2C3=O)C1]

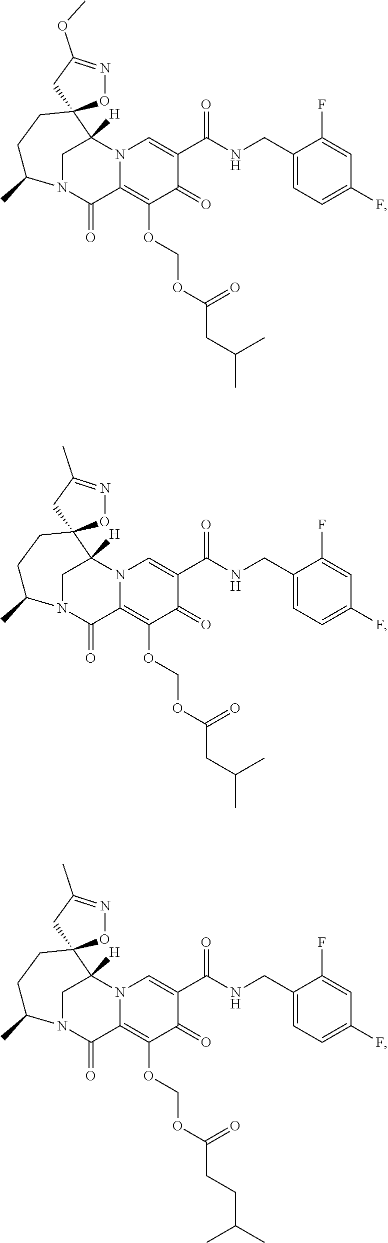 [SMILES: COC1=NO[C@@]2(CC[C@H](C)N3C[C@H]2n2cc(C(=O)NCc4ccc(F)cc4F)c(=O)c(OCOC(=O)CC(C)C)c2C3=O)C1 ; CC1=NO[C@@]2(CC[C@H](C)N3C[C@H]2n2cc(C(=O)NCc4ccc(F)cc4F)c(=O)c(OCOC(=O)CC(C)C)c2C3=O)C1 ; CC1=NO[C@@]2(CC[C@H](C)N3C[C@H]2n2cc(C(=O)NCc4ccc(F)cc4F)c(=O)c(OCOC(=O)CCC(C)C)c2C3=O)C1]

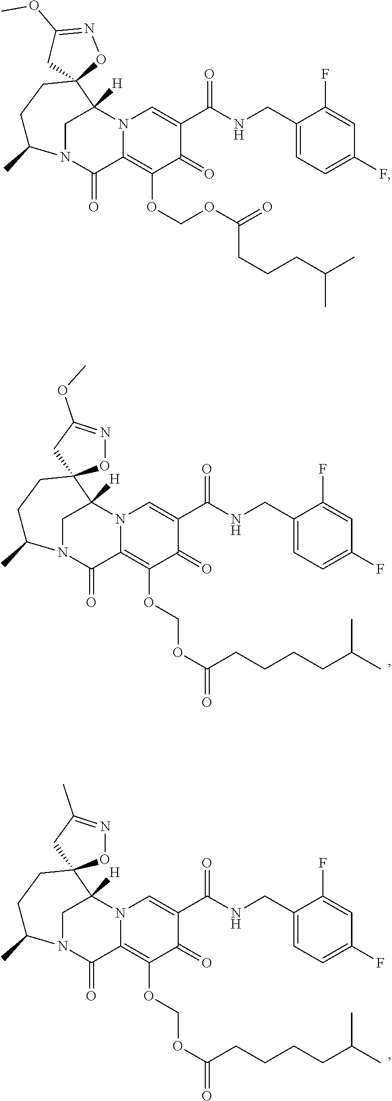

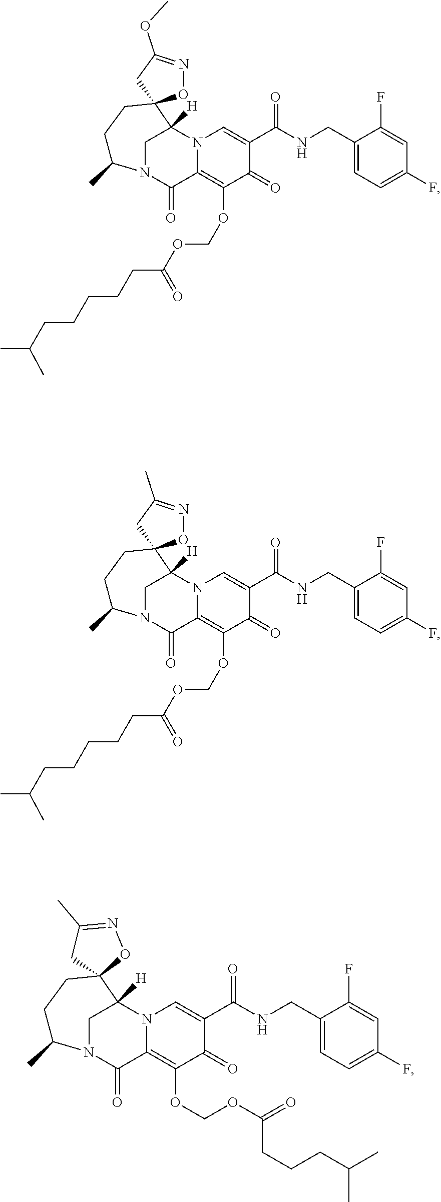 [SMILES: COC1=NO[C@@]2(CC[C@H](C)N3C[C@H]2n2cc(C(=O)NCc4ccc(F)cc4F)c(=O)c(OCOC(=O)CCCCCC(C)C)c2C3=O)C1 ; CC1=NO[C@@]2(CC[C@H](C)N3C[C@H]2n2cc(C(=O)NCc4ccc(F)cc4F)c(=O)c(OCOC(=O)CCCCCC(C)C)c2C3=O)C1 ; CC1=NO[C@@]2(CC[C@H](C)N3C[C@H]2n2cc(C(=O)NCc4ccc(F)cc4F)c(=O)c(OCOC(=O)CCCC(C)C)c2C3=O)C1]

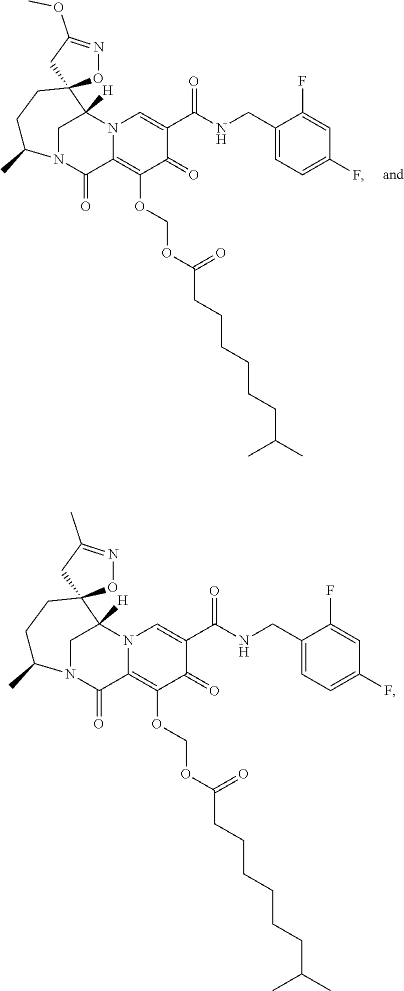 [SMILES: COC1=NO[C@@]2(CC[C@H](C)N3C[C@H]2n2cc(C(=O)NCc4ccc(F)cc4F)c(=O)c(OCOC(=O)CCCCCCC(C)C)c2C3=O)C1 ; CC1=NO[C@@]2(CC[C@H](C)N3C[C@H]2n2cc(C(=O)NCc4ccc(F)cc4F)c(=O)c(OCOC(=O)CCCCCCC(C)C)c2C3=O)C1]

or a pharmaceutically acceptable salt thereof.

In some embodiments of the compounds of Formula I or Ib, the compound is selected from the group consisting of;

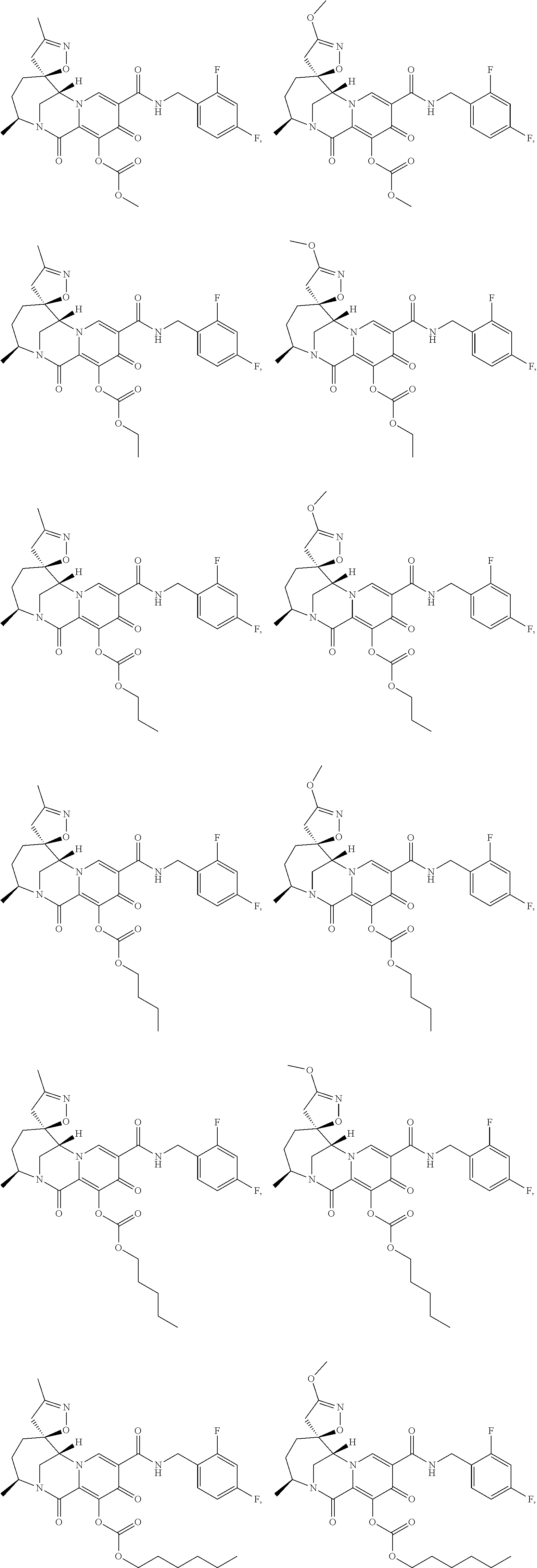

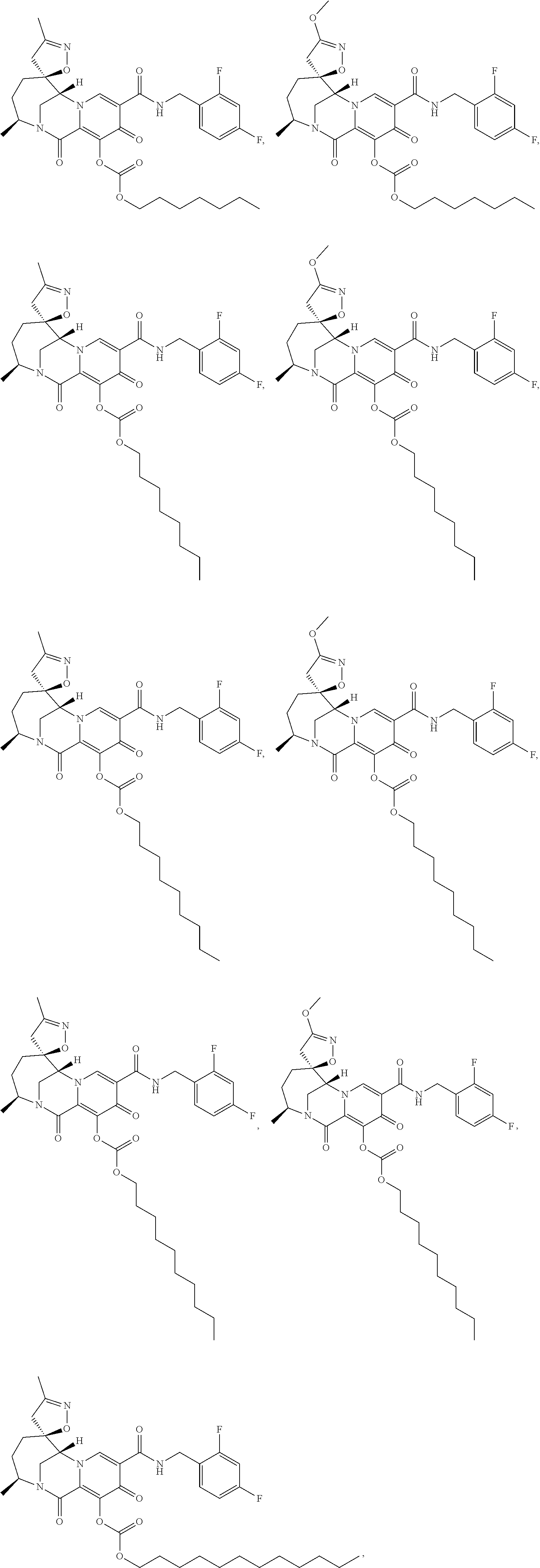

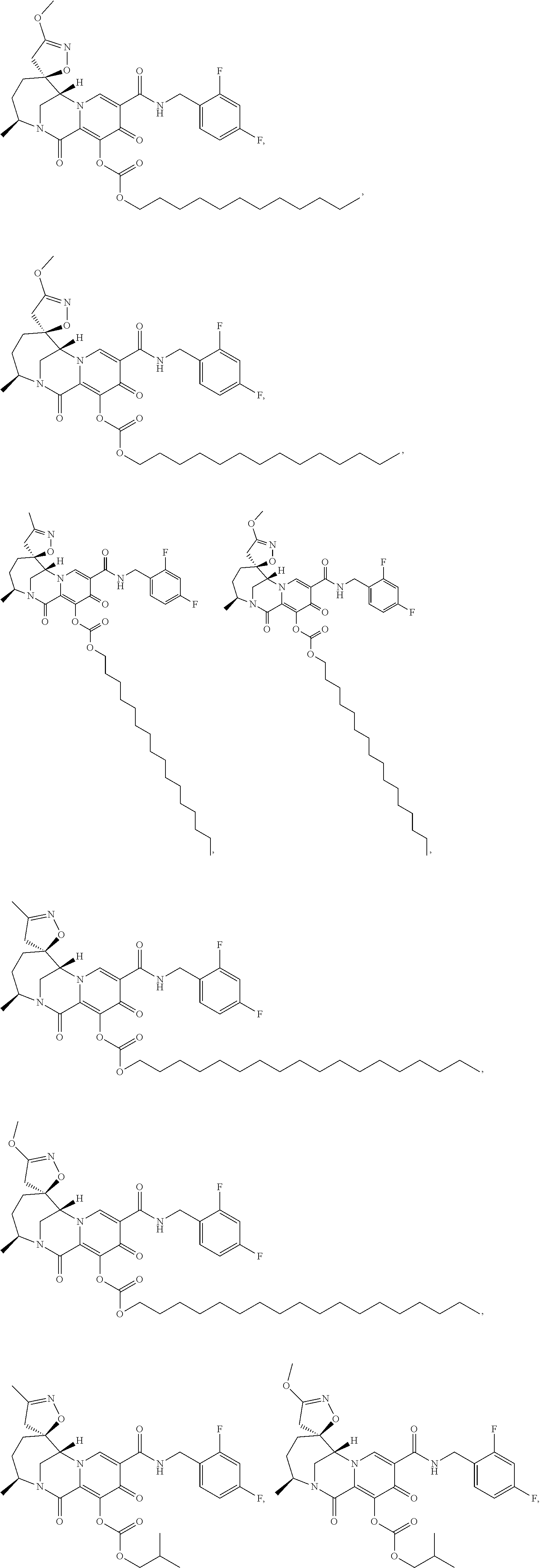

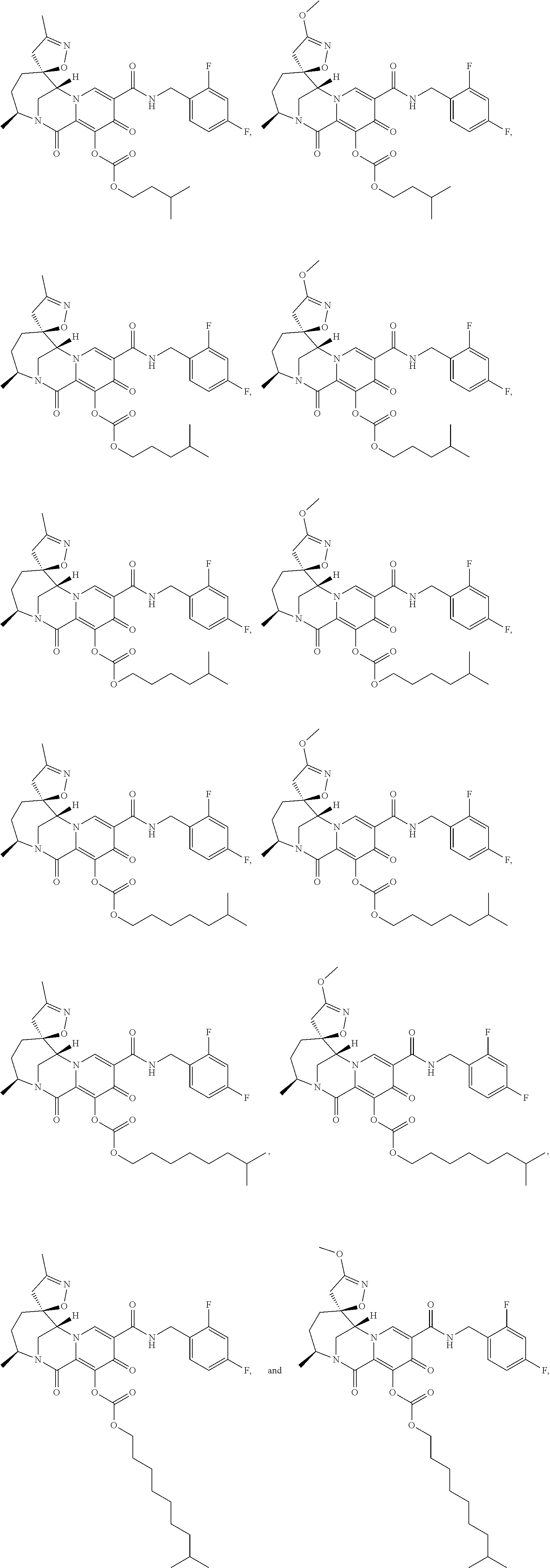

and or a pharmaceutically acceptable salt thereof.

In some embodiments of the compounds of Formula I or Ic, the compound is selected from the group consisting of:

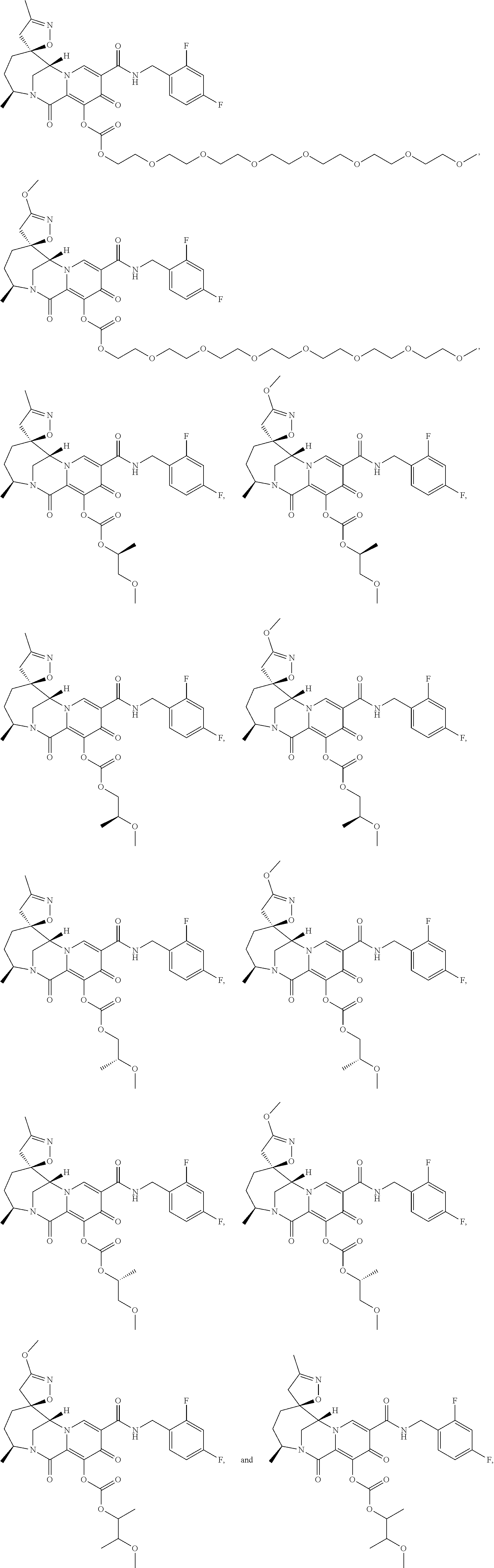

or a pharmaceutically acceptable salt thereof.

In some embodiments of the compounds of Formula I or Ie, the compound is selected from the group consisting of:

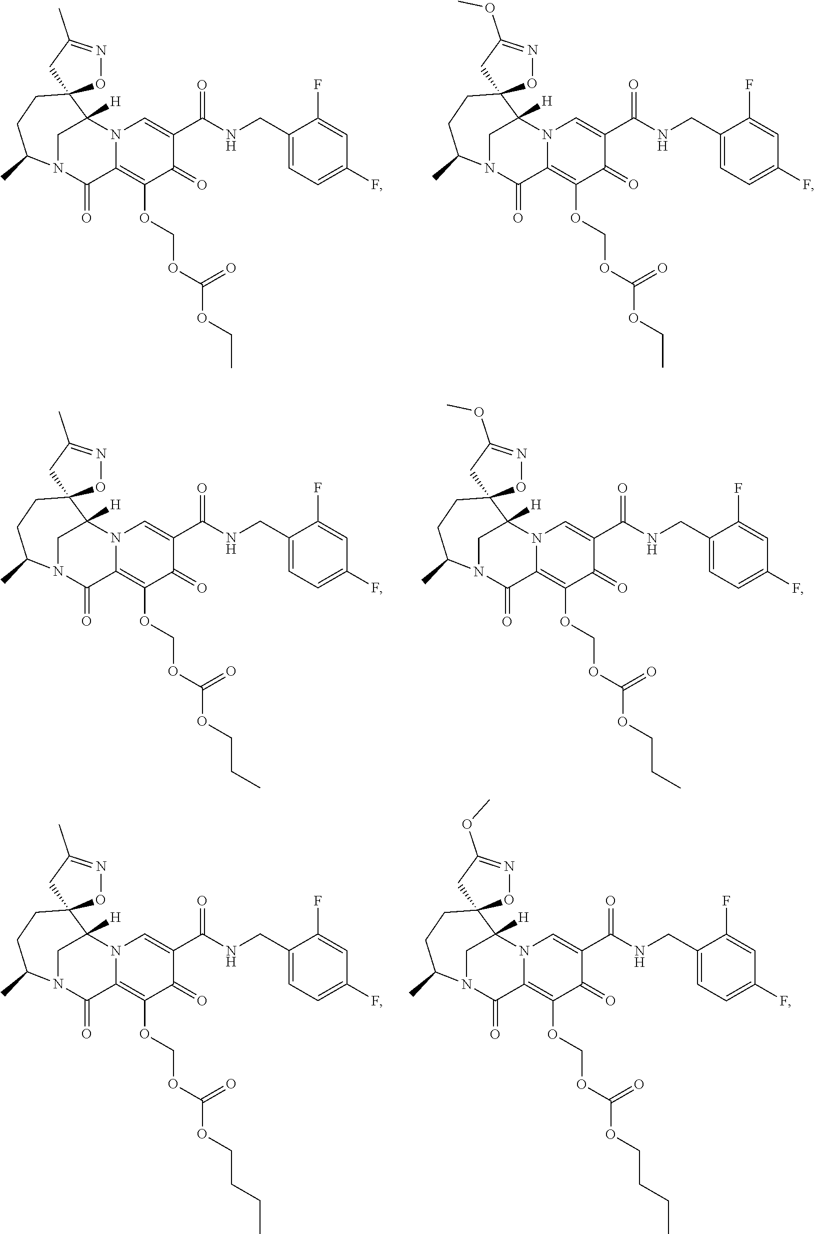

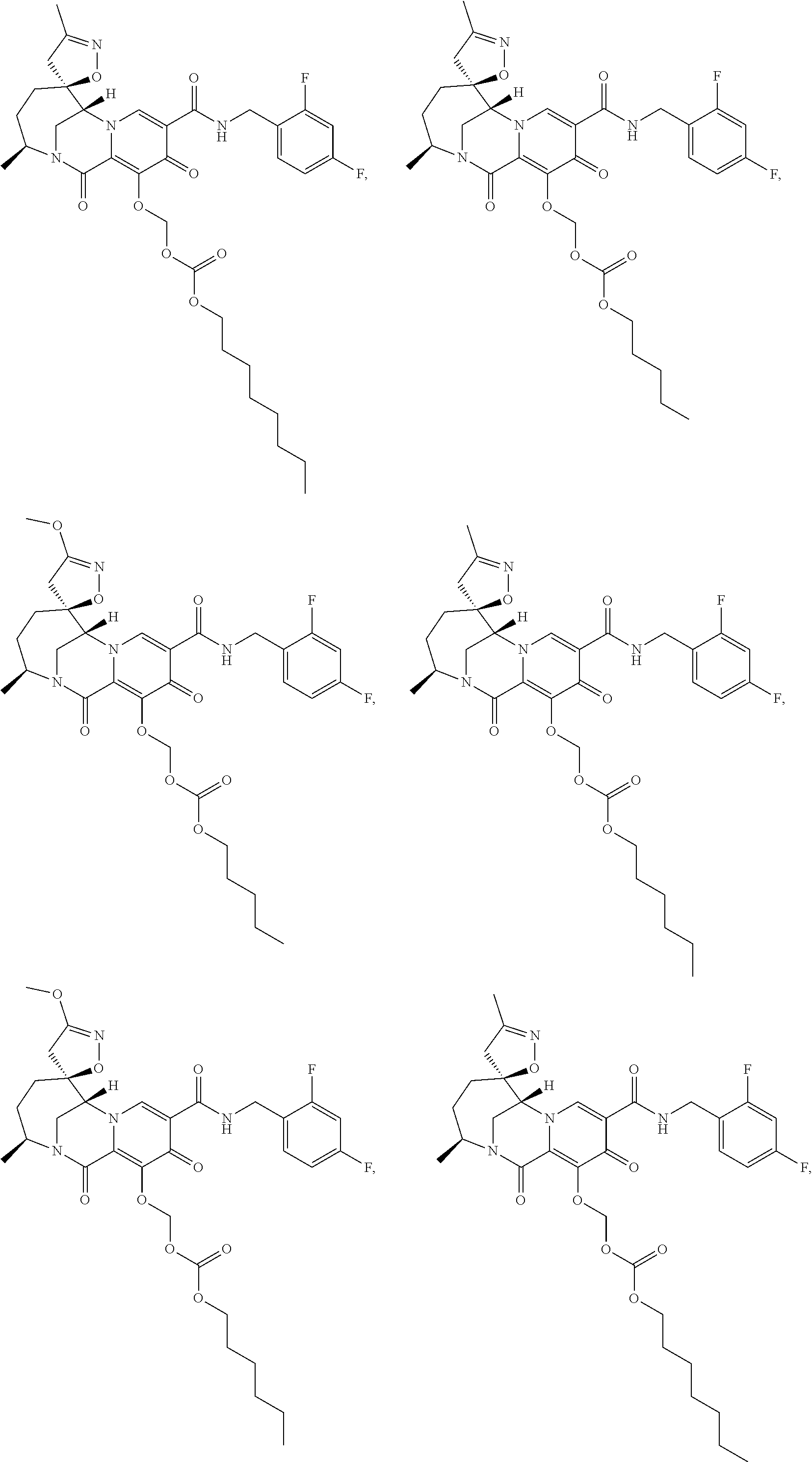

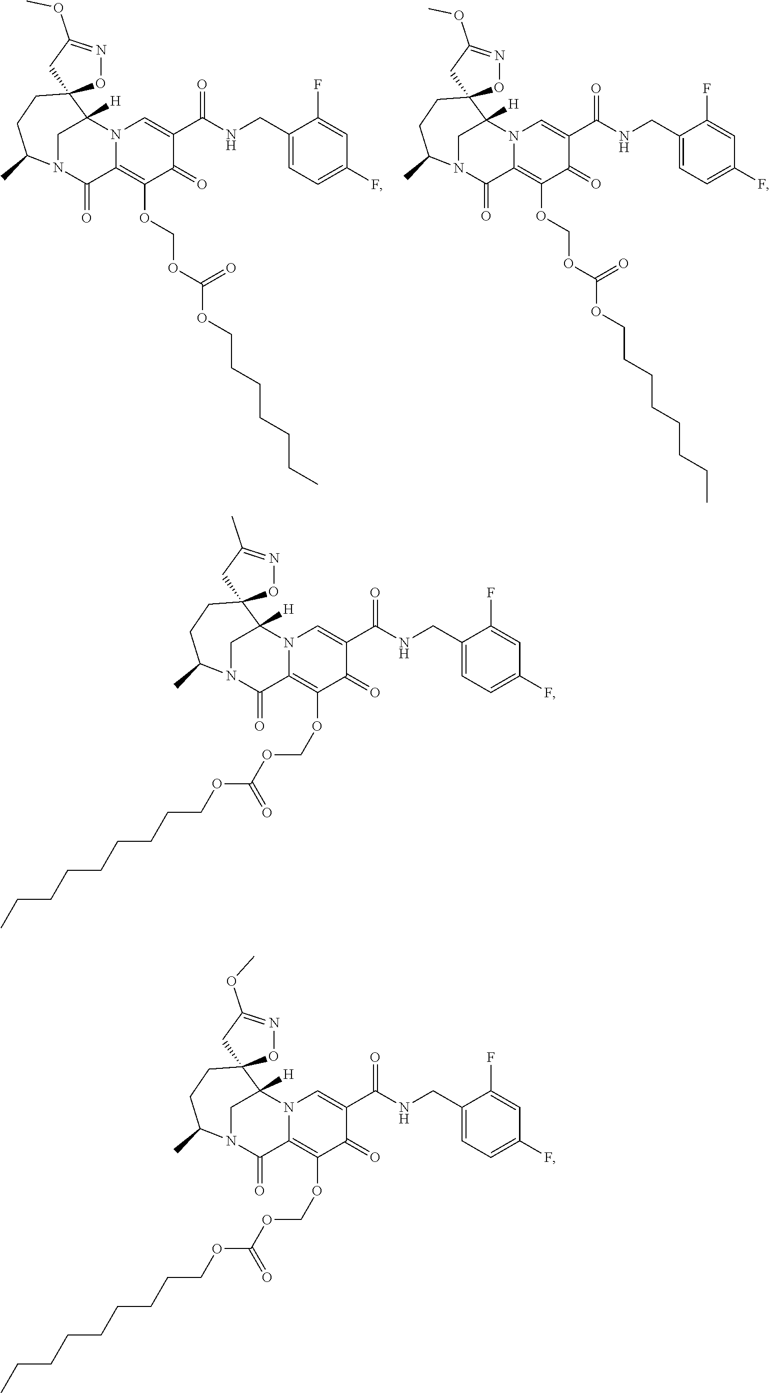

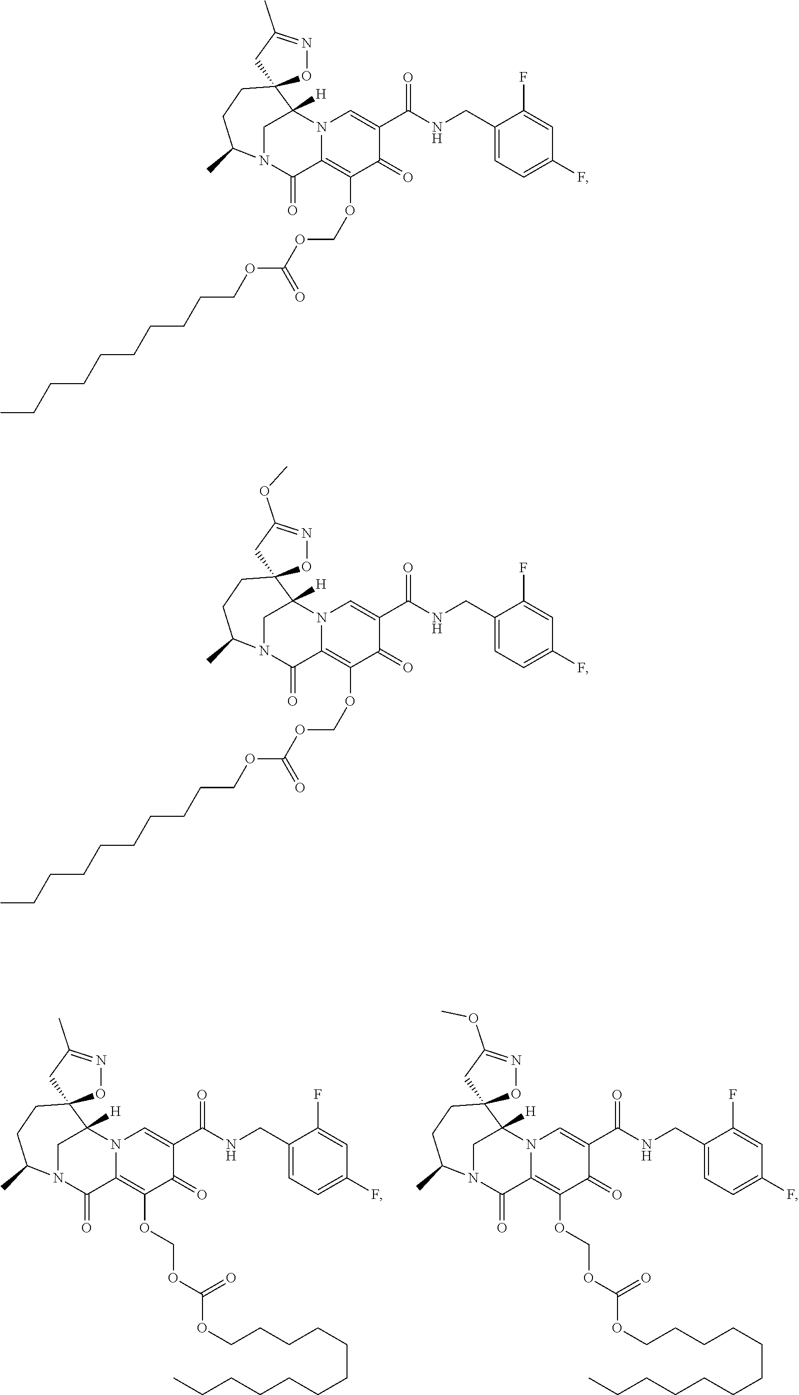

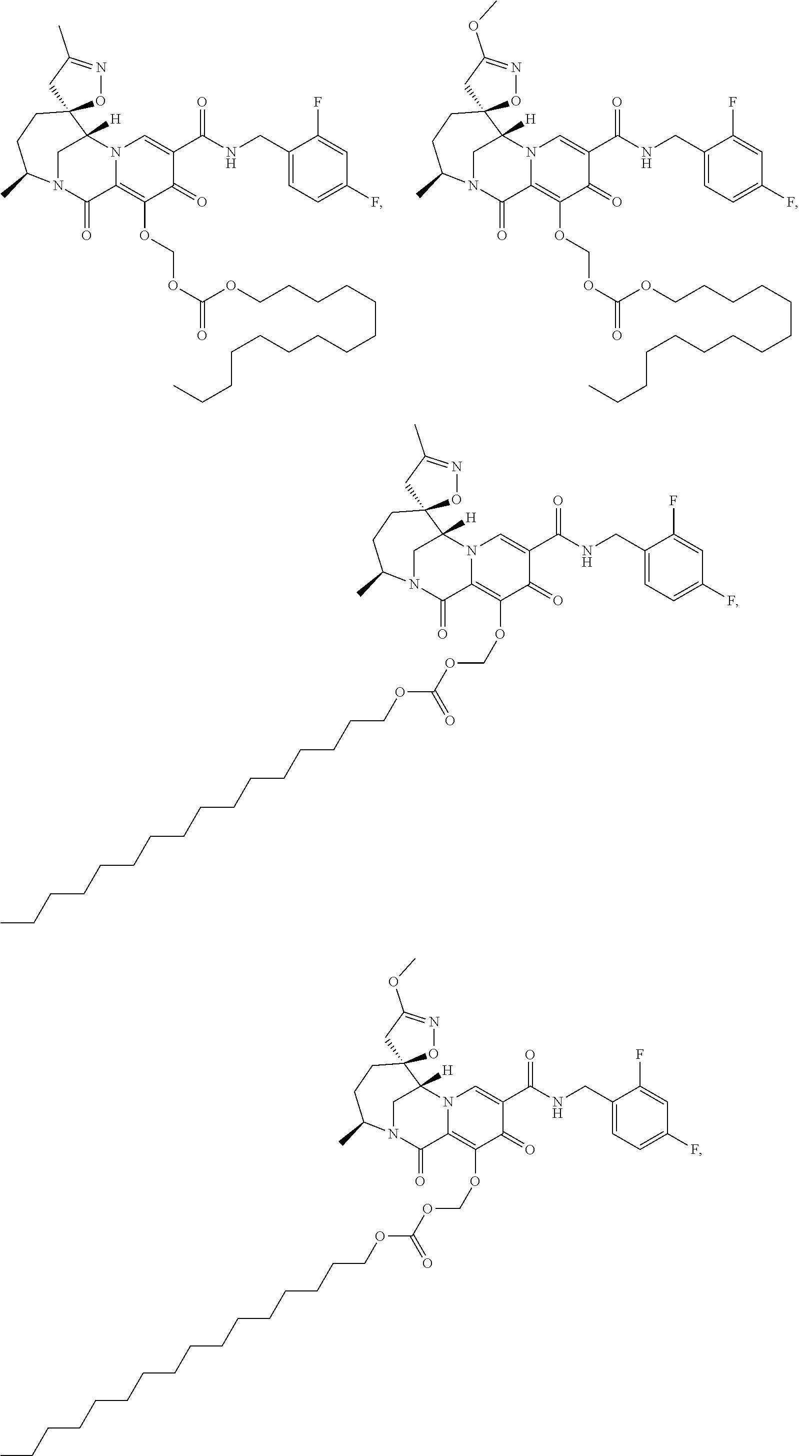

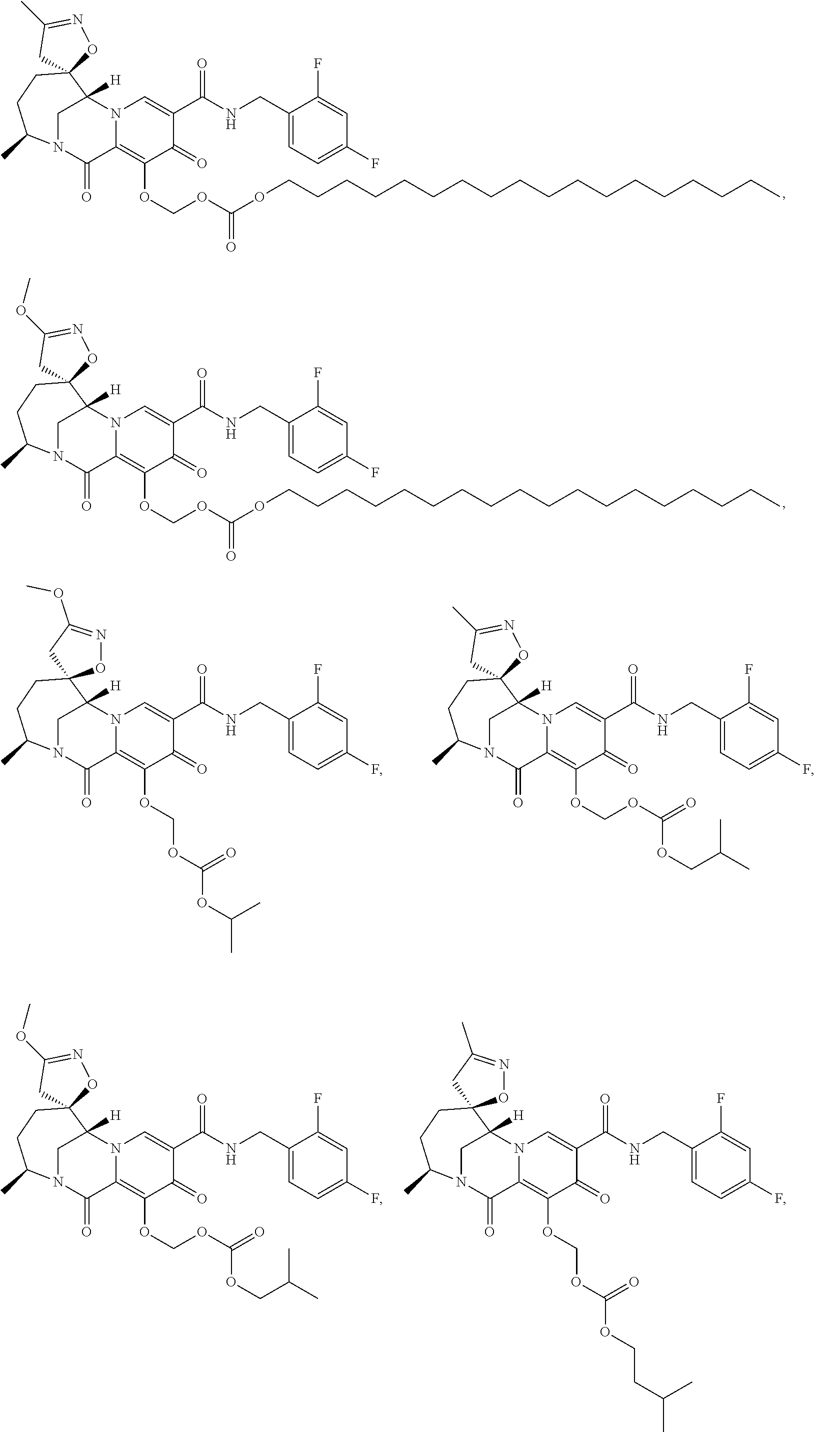

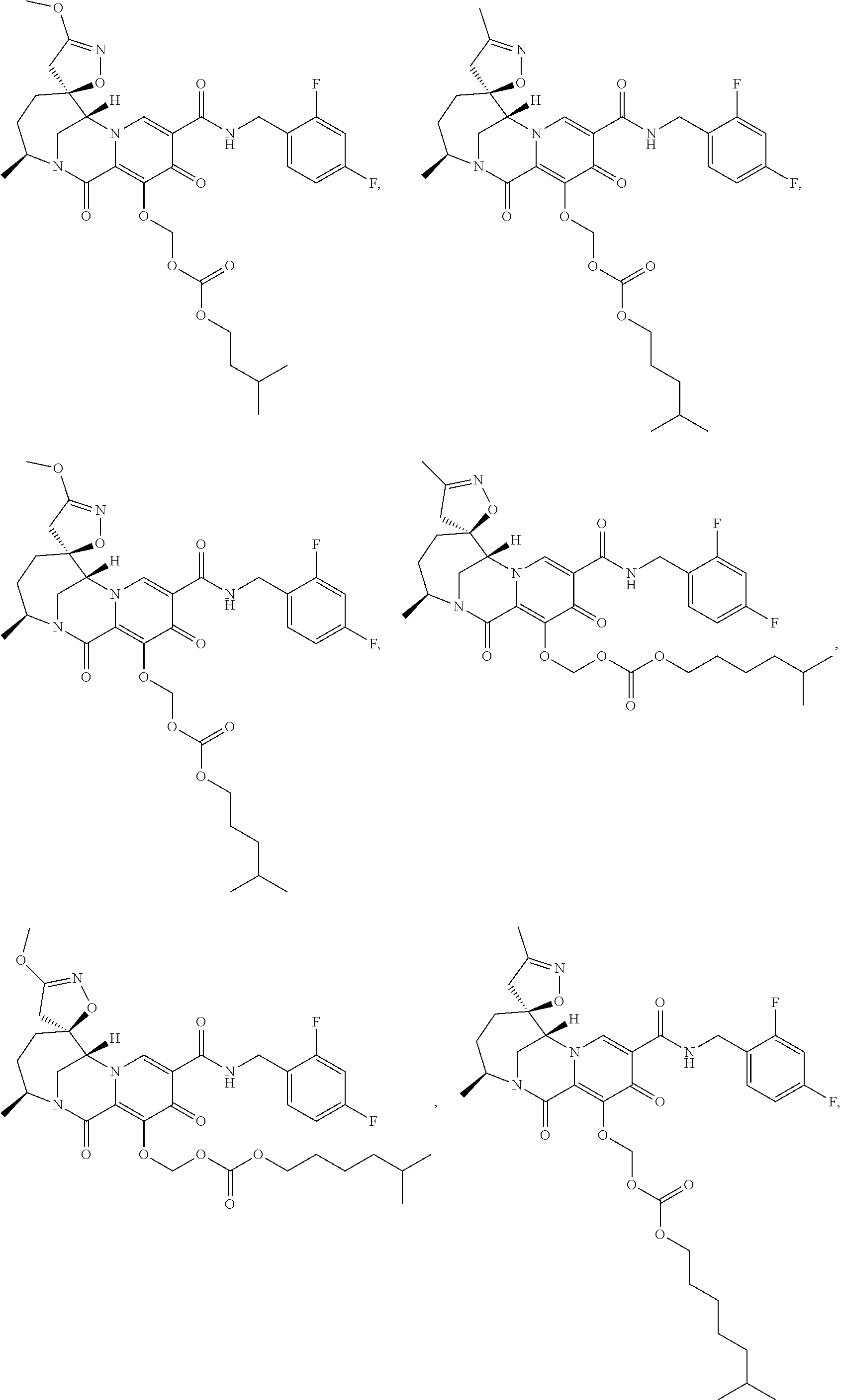

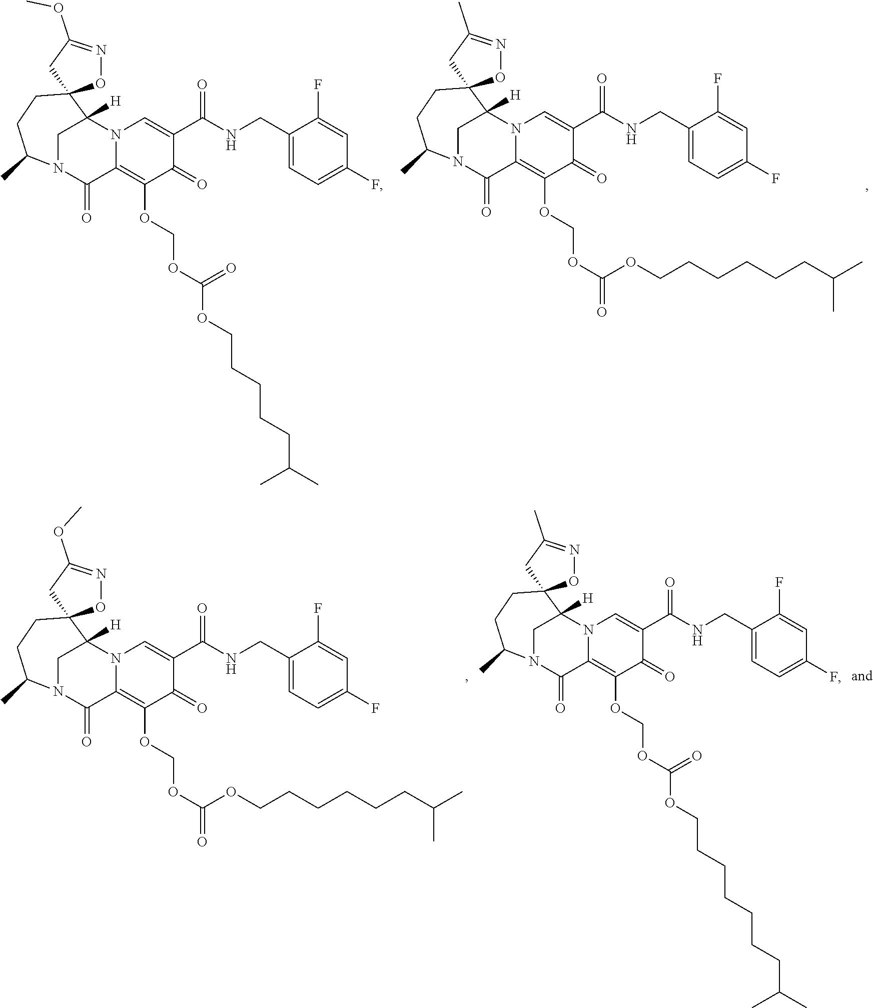

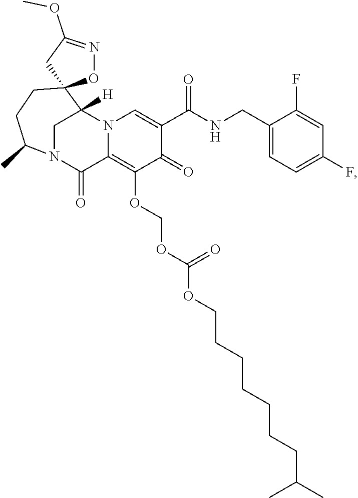 [SMILES: COC1=NO[C@@]2(CC[C@H](C)N3C[C@H]2n2cc(C(=O)NCc4ccc(F)cc4F)c(=O)c(OCOC(=O)OCCCCCCCC(C)C)c2C3=O)C1]

and or a pharmaceutically acceptable salt thereof.

In some embodiments of the compounds of Formula I or Id, the compound is selected from the group consisting of;

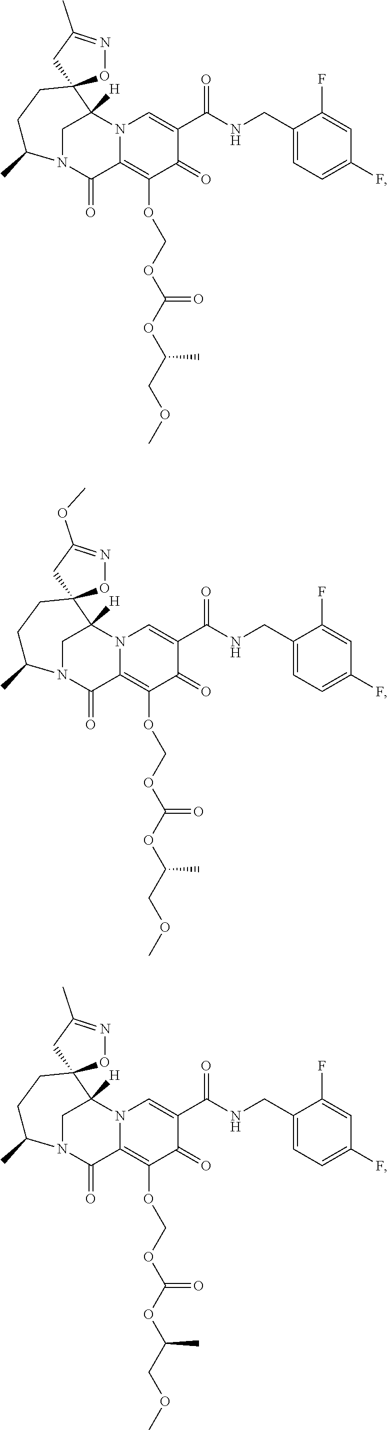 [SMILES: COC[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2 ; COC[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2 ; COC[C@H](C)OC(=O)OCOc1c2n(cc(C(=O)NCc3ccc(F)cc3F)c1=O)[C@@H]1CN(C2=O)[C@@H](C)CC[C@]12CC(C)=NO2]

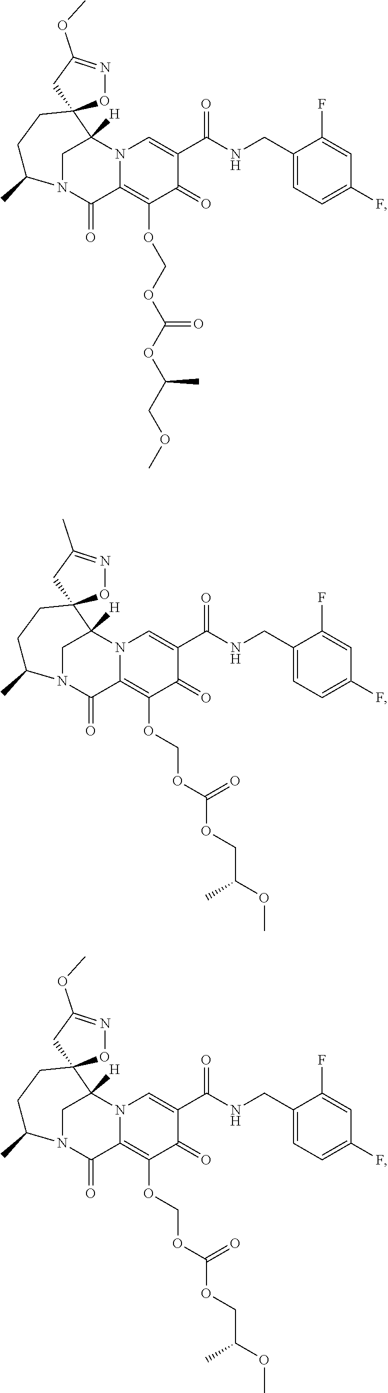 [SMILES: COC[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2 ; CO[C@H](C)COC(=O)OCOc1c2n(cc(C(=O)NCc3ccc(F)cc3F)c1=O)[C@@H]1CN(C2=O)[C@@H](C)CC[C@]12CC(C)=NO2 ; COC1=NO[C@@]2(CC[C@H](C)N3C[C@H]2n2cc(C(=O)NCc4ccc(F)cc4F)c(=O)c(OCOC(=O)OC[C@@H](C)OC)c2C3=O)C1]

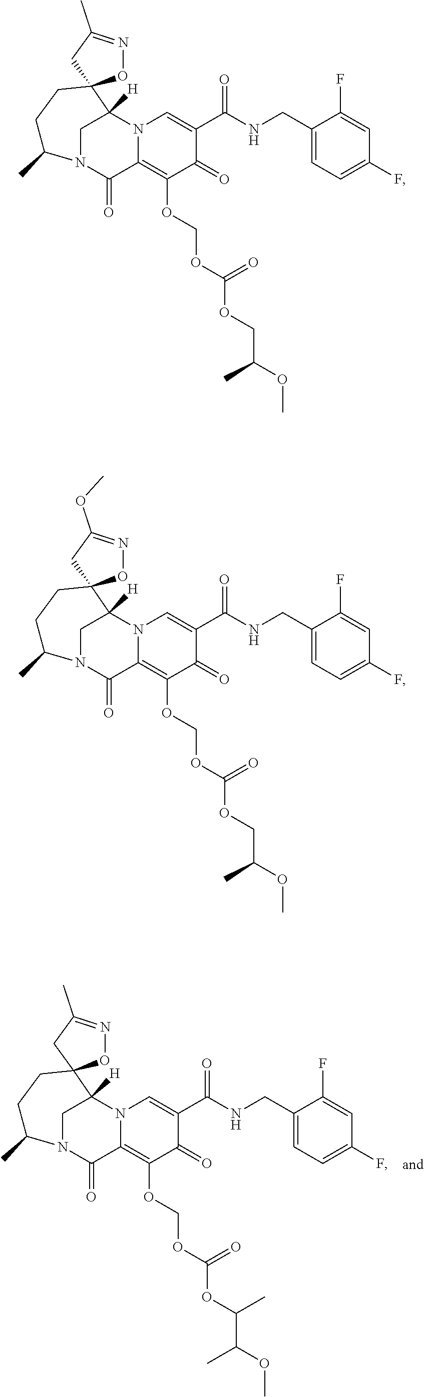 [SMILES: CO[C@@H](C)COC(=O)OCOc1c2n(cc(C(=O)NCc3ccc(F)cc3F)c1=O)[C@@H]1CN(C2=O)[C@@H](C)CC[C@]12CC(C)=NO2 ; COC1=NO[C@@]2(CC[C@H](C)N3C[C@H]2n2cc(C(=O)NCc4ccc(F)cc4F)c(=O)c(OCOC(=O)OC[C@H](C)OC)c2C3=O)C1 ; COC(C)C(C)OC(=O)OCOc1c2n(cc(C(=O)NCc3ccc(F)cc3F)c1=O)[C@@H]1CN(C2=O)[C@@H](C)CC[C@]12CC(C)=NO2]

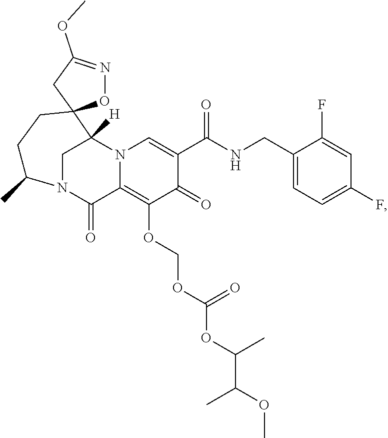 [SMILES: COC1=NO[C@@]2(CC[C@H](C)N3C[C@H]2n2cc(C(=O)NCc4ccc(F)cc4F)c(=O)c(OCOC(=O)OC(C)C(C)OC)c2C3=O)C1]

or a pharmaceutically acceptable salt thereof.

In some embodiments, the compounds disclosed herein have therapeutic activity. In some embodiments, the compounds disclosed herein are prodrugs, which upon administration to the human body can be converted to compounds having therapeutic activity. In some embodiments, the compounds disclosed herein are prodrugs of certain compounds disclosed in U.S. application Ser. Nos. 18/296,285, 18/334,588, and 18/334,611, and PCT Application No. PCT/US2023/065401, the disclosures of which are incorporated herein by reference in their entireties. For example, the compounds disclosed herein can be converted to compounds of Formula II:

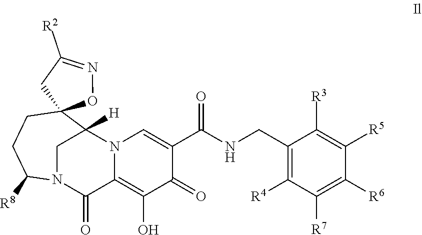

or pharmaceutically acceptable salts thereof, wherein R², R³, R⁴, R⁵, R⁶, R⁷, and R⁸ are as defined according to any other embodiment described herein. Accordingly, the present invention includes a method of converting a compound of Formula I, or a pharmaceutically acceptable salt thereof, to Formula II, or a pharmaceutically acceptable salt thereof, by (1) contacting the compound of Formula I, or a pharmaceutically acceptable salt thereof, with cell-containing aqueous media capable of converting —OR¹ to —OH (e.g., either enzymatically or chemically through acid or base hydrolysis); or (2) administering the compound to a patient whereby the compound of Formula I, or pharmaceutically acceptable salt thereof, is converted to a compound of Formula II, or a pharmaceutically acceptable salt thereof, through biologic pathways (e.g., enzymes) or through contact with biologic fluids and/or tissues conducive to converting —OR¹ to —OH (e.g., through acid or base hydrolysis).

The present invention further provides the treatment or prophylaxis of HIV infection in a patient in need thereof by contacting the patient with a compound of Formula II, or a pharmaceutically acceptable salt thereof, whereby the compound of Formula II, or a pharmaceutically acceptable salt thereof, is generated within the patient upon administration to the patient of a compound of Formula I, or a pharmaceutically acceptable salt thereof. In some embodiments, the compound of Formula II that is generated within the patient is Intermediate B or Intermediate C, as described below in the Example section.

### III. Pharmaceutical Compositions

Compounds provided herein are usually administered in the form of pharmaceutical compositions. Thus, provided herein are also pharmaceutical compositions that comprise one or more of the compounds provided herein or pharmaceutically acceptable salts, isomer, or a mixture thereof and one or more pharmaceutically acceptable vehicles selected from carriers, adjuvants and excipients. The compounds provided herein may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In one aspect, provided herein are pharmaceutical compositions comprising a compound provided herein (e.g., a compound of Formula I, Ia, Ib, Ic, Id, Ie, or If), or a pharmaceutically acceptable salt thereof, and a pharmaceutically acceptable excipient or carrier. In some embodiments, the pharmaceutical compositions comprise a therapeutically effective amount of a compound provided herein, or a pharmaceutically acceptable salt thereof, and a pharmaceutically acceptable excipient or carrier.

In some embodiments, the pharmaceutical compositions provided herein further comprise one or more (e.g., one, two, three, four, one or two, one to three, or one to four) additional therapeutic agents, or a pharmaceutically acceptable salt thereof. In some embodiments, the pharmaceutical compositions further comprise a therapeutically effective amount of the one or more (e.g., one, two, three, four, one or two, one to three, or one to four) additional therapeutic agents, or a pharmaceutically acceptable salt thereof. In some embodiments, the pharmaceutical compositions further comprise one, tow, three, or four additional therapeutic agents.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds provided herein. Administration may be via, for example, capsule or enteric coated tablets. In making the pharmaceutical compositions that include at least one compound provided herein or pharmaceutically acceptable salts, isomer, or a mixture thereof, the active ingredient (such as a compound provided herein) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose or any combinations thereof. The pharmaceutical compositions can additionally include lubricating agents such as talc, magnesium stearate, and mineral oil; wetting agents; emulsifying and suspending agents; preserving agents such as methyl and propylhydroxy-benzoates; sweetening agents; and flavoring agents; or any combinations thereof.

The pharmaceutical compositions that include at least one compound described herein or pharmaceutically acceptable salts, isomer, or a mixture thereof can be formulated so as to provide quick, sustained or delayed release of the active ingredient (such as a compound provided herein) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds provided herein in controlled amounts. The construction and use of transdermal patches for the delivery of pharmaceutical agents is well known in the art. See. e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or pharmaceutically acceptable salts, isomer, or a mixture thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with materials such as shellac, cetyl alcohol, and cellulose acetate.

Pharmaceutical compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### IV. Methods of Treatment

In one embodiment, methods of treating an HIV (e.g., HIV-1 and/or HIV-2) infection in a human having or at risk of having the infection comprising administering to the human a therapeutically effective amount of a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, are provided.

In some embodiments, the methods further comprise administering to the human a therapeutically effective amount of one, two, three, or four additional therapeutic agents. In certain embodiments, the additional therapeutic agent or agents are anti-HIV agents. In particular embodiments, the additional therapeutic agent or agents are HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs (broadly neutralizing HIV antibodies), TLR7 agonists, pharmacokinetic enhancers, other drugs for treating HIV, or combinations thereof.

In some embodiments, the additional therapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof. In one embodiment, the additional therapeutic agent or agents are abacavir, tenofovir alafenamide, tenofovir disoproxil, lenacapavir, or a pharmaceutically acceptable salt thereof. In one embodiment, the additional therapeutic agent or agents are abacavir, tenofovir alafenamide, tenofovir disoproxil, lenacapavir, GS-5894, islatravir, or a pharmaceutically acceptable salt thereof. In some embodiments, the additional therapeutic agent or agents are lenacapavir, islatravir. In some embodiments, the additional therapeutic agent is lenacapavir. In some embodiments, the additional therapeutic agent is islatravir.

In another embodiment, a use of a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, for treating an HIV (e.g., HIV-1 and/or HIV-2) infection in a human having or at risk of having the infection is provided.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, for use in medical therapy is provided.

In another embodiment, a compound of Formula I, Ia, Ib, Ic, Id, Ie, or If, or a pharmaceutically acceptable salt thereof, or a pharmaceutical composition of Formula I, Ia, Ib, Ic, Id, Ie, or If, or pharmaceutically acceptable salt thereof, for use in treating an HIV infection is provided.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided wherein said method further comprises administering to the human one, two, three, or four additional therapeutic agents.

In another embodiment, a compound of I, Ia, Ib, Ic, Id, Ie, or If, or a pharmaceutically acceptable salt thereof, or a pharmaceutical composition of a compound of Formula I, Ia, Ib, Ic, Id, Ie, or If or a pharmaceutically acceptable salt thereof for use in a method of treating an HIV infection in a human having or at risk of having the infection, is provided wherein said method further comprises administering to the human one, two, three, or four additional therapeutic agents selected from the group consisting of HIV protease inhibitors. HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof. In one embodiment, the one, two, three, or four additional therapeutic agents are selected from HIV protease inhibitors. HIV non-nucleoside inhibitors of reverse transcriptase, HIV nucleoside or nucleotide inhibitors of reverse transcriptase, latency reversing agents, HIV capsid inhibitors, HIV bNAbs, TLR7 agonists, and combinations thereof.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided wherein said method further comprises administering to the human a therapeutically effective amount of tenofovir disoproxil and emtricitabine.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided wherein said method further comprises administering to the human a therapeutically effective amount of tenofovir alafenamide and emtricitabine.

In another embodiment, a compound of Formula I, Ia, Ib, Ic, Id, Ie, or If, or a pharmaceutically acceptable salt thereof, or a pharmaceutical composition of a compound of I, Ia, Ib, Ic, Id, Ie, or If, or a pharmaceutically acceptable salt thereof for use in a method of treating an HIV infection in a human having or at risk of having the infection, is provided wherein said method further comprises administering to the human a therapeutically effective amount of tenofovir disoproxil.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided wherein said method further comprises administering to the human a therapeutically effective amount of tenofovir alafenamide.

In another embodiment, a method of using a compound of Formula I, Ia, Ib, Ic, Id, Ie, or If, or a pharmaceutically acceptable salt thereof in therapy is provided. In particular, a method of treating the proliferation of the HIV virus, treating AIDS, or delaying the onset of AIDS or ARC symptoms in a mammal (e.g., a human) is provided, comprising administering to the mammal a compound of Formula I, Ia, Ib, Ic, Id, Ie, or If, or a pharmaceutically acceptable salt thereof, and a pharmaceutically acceptable excipient.

In another embodiment, a composition comprising a compound of Formula I, Ia, Ib, Ic, Id, Ie, or If, or a pharmaceutically acceptable salt thereof, and a pharmaceutically acceptable excipient, for use in a method of treating the proliferation of the HIV virus, treating AIDS, or delaying the onset of AIDS or ARC symptoms in a mammal (e.g., a human) is provided.

In one embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, is provided for use in preventing HIV infection.

For example, in one embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, is provided for use in pre-exposure prophylaxis (PrEP), i.e., before the exposure of the individual to the HIV virus to prevent HIV infection from taking hold if the individual is exposed to the virus and/or to keep the virus from establishing a permanent infection and/or to prevent the appearance of symptoms of the disease and/or to prevent the virus from reaching detectable levels in the blood.

In another embodiment, the use of a compound of Formula I, Ia, Ib, Ic, Id, Ie, or If, or a pharmaceutically acceptable salt thereof, for the manufacture of a medicament for treating an HIV infection in a human being having or at risk of having the infection is disclosed.

In another embodiment, the use of a compound of Formula I, Ia, Ib, Ic, Id, Ie, or If, or a pharmaceutically acceptable salt thereof, as a research tool is disclosed.

In another embodiment, an article of manufacture comprising a composition effective to treat an HIV infection; and packaging material comprising a label which indicates that the composition can be used to treat infection by HIV is disclosed. Exemplary compositions comprise a compound of Formula I, Ia, Ib, Ic, Id, Ie, or If, or a pharmaceutically acceptable salt thereof.

In still another embodiment, a method of inhibiting the replication of HIV is disclosed. The method comprises exposing the virus to an effective amount of the compound of Formula I, Ia, Ib, Ic, Id, Ie, or If, or a pharmaceutically acceptable salt thereof, under conditions where replication of HIV is inhibited.

In another embodiment, the use of a compound of Formula I, Ia, Ib, Ic, Id, Ie, or If, or a pharmaceutically acceptable salt thereof, to inhibit the activity of the HIV integrase enzyme is disclosed.

In another embodiment, the use of a compound of Formula I, Ia, Ib, Ic, Id, Ie, or If, or a pharmaceutically acceptable salt thereof, to inhibit the replication of HIV is disclosed.

### V. Administration

The compounds of the present disclosure (for example, a compound of Formula I, Ia, Ib, Ic, Id, Ie, or If, or a pharmaceutically acceptable salt thereof, can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. In some embodiments, the administration is oral, intravenous, subcutaneous, or intramuscular. It will be appreciated that the preferred route may vary with, for example, the condition of the recipient. An advantage of certain compounds disclosed herein is that they are orally bioavailable and can be dosed orally.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound is administered on a daily or intermittent schedule for the duration of the individual's life.

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or w % ben converting an effective dosage in a non-human subject such as dog to a dosage suitable for a human subject.

The dosage may also be described as a total amount of a compound described herein administered per dose. Dosage of a compound of Formula I, Ia, Ib. Ic, Id, Ie, or If, or a pharmaceutically acceptable salt or pharmaceutically acceptable tautomer thereof, may be between about 1 mg and 4,000 mg, between about 2,000 to 4,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

The dosage or dosing frequency of a compound of the present disclosure may be adjusted over the course of the treatment, based on the judgment of the administering physician.

The compounds of the present disclosure may be administered to an individual (e.g., a human) in a therapeutically effective amount. In some embodiments, the compound is administered once daily. In some embodiments, the compound is administered once every week.

In some embodiments, the compound is administered once every month, once every two months, once every three months, once every four months, once every five months, once every six months, once every seven months, once every eight months, once every nine months, once every ten months, once every eleven months, once every twelve months, or longer.

In some embodiments, the compound is administered once every month. In some embodiments, the compound is administered every two months. In some embodiments, the compound is administered every three months. In some embodiments, the compound is administered every four months. In some embodiments, the compound is administered every five months. In some embodiments, the compound is administered every six months. In some embodiments, the compound is administered every seven months. In some embodiments, the compound is administered every eight months. In some embodiments, the compound is administered every nine months. In some embodiments, the compound is administered every ten months. In some embodiments, the compound is administered every eleven months. In some embodiments, the compound is administered every year.

The compounds provided herein can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein include from about 0.3 mg to about 30 mg per dose, or from about 30 mg to about 300 mg per dose, or from about 0.3 sg to about 30 mg per dose, or from about 30 μg to about 300 μg per dose.

A compound of the present disclosure may be combined with one or more additional therapeutic agents in any dosage amount of the compound of the present disclosure (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 10×) mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the compound of Formula I are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the compound of the present disclosure are about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, the methods described herein comprise administering to the subject an initial daily dose of about 1 to 5×) mg of a compound p herein and increasing the dose by increments until clinical efficacy is achieved. Increments of about 5, 10, 25, 50, or 100 mg can be used to increase the dose. The dosage can be increased daily, every other day, twice per week, once per week, once every two weeks, once every three weeks, or once a month.

When administered orally, the total daily dosage for a human subject may be between about 1 mg and 1,000 mg, between about 10-500 mg/day, between about 50-300 mg/day, between about 75-200 mg/day, or between about 100-150 mg/day. In some embodiments, the total daily dosage for a human subject may be about 100, 200, 300, 400, 500, 600, 700, 800, 900, or 1000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound disclosed herein is administered once daily in a method disclosed herein. In some embodiments, a compound disclosed herein is administered twice daily in a method disclosed herein.

In some embodiments, a compound disclosed herein is administered once every 10 days. In some embodiments, a compound disclosed herein is administered once every 15 days. In some embodiments, a compound disclosed herein is administered once every 20 days. In some embodiments, a compound disclosed herein is administered once every 10-15 days. In some embodiments, a compound disclosed herein is administered once every 15-20 days. In some embodiments, a compound disclosed herein is administered once every 10-20 days. In some embodiments, a compound disclosed herein is administered once every month. In some embodiments, a compound disclosed herein is administered once every 2 months. In some embodiments, a compound disclosed herein is administered once every 3 months. In some embodiments, a compound disclosed herein is administered once every 4 months. In some embodiments, a compound disclosed herein is administered once every 5 months. In some embodiments, a compound disclosed herein is administered once every 6 months. In some embodiments, a compound disclosed herein is administered once every 8 months. In some embodiments, a compound disclosed herein is administered once every 10 months. In some embodiments, a compound disclosed herein is administered once every year.

The frequency of dosage of the compound of the present disclosure will be determined by the needs of the individual patient and can be, for example, once per day, once per week, once per two weeks, once per month, once per two months, once per three months, once per four months, once per six months, or less. Administration of the compound continues for as long as necessary to treat the HIV infection.

### VI. Kits and Articles of Manufacture

In one aspect, provided herein are kits that comprise a compound provided herein, (e.g., a compound of Formula I, Ia, Ib, Ic, Id, Ie, or If), or a pharmaceutically acceptable salt, stereoisomer, or solvate thereof, and suitable packaging. In some embodiments, the kit further comprises instructions for use. In some embodiments, the kit comprises a compound provided herein (e.g., a compound of Formula I, Ia, Ib, Ic, Id, Ie, or If), or a pharmaceutically acceptable salt, stereoisomer, or solvate thereof, and a label and/or instructions for use of the compounds in the treatment of the indications, including the diseases or conditions, described herein.

In some embodiments, the kits further comprise one or more (e.g., one, two, three, four, one or two, one to three, or one to four) additional therapeutic agents, or a pharmaceutically acceptable salt thereof.

In one aspect, provided herein are articles of manufacture that comprise a compound described herein or pharmaceutically acceptable salts, isomer, or a mixture thereof in a suitable container. In some embodiments, the container may be a vial, jar, ampoule, preloaded syringe, or intravenous bag.

### VII. Combination Therapy

In certain embodiments, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents. In one embodiment, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents.

In one embodiment, pharmaceutical compositions comprising a compound disclosed herein, or a pharmaceutically acceptable salt thereof, in combination with one, two, three, or four additional therapeutic agents, and a pharmaceutically acceptable carrier, diluent, or excipient are provided.

In certain embodiments, the present disclosure provides a method for treating an HIV infection, comprising administering to a subject in need thereof a therapeutically effective amount of a compound disclosed herein, or a pharmaceutically acceptable salt thereof, in combination with a therapeutically effective amount of one, two, three, or four additional therapeutic agents which are suitable for treating an HIV infection.

In certain embodiments, a compound disclosed herein, or a pharmaceutically acceptable salt thereof, is combined with one, two, three, four, or more additional therapeutic agents. In certain embodiments, a compound disclosed herein, or a pharmaceutically acceptable salt thereof, is combined with one, two, three, or four additional therapeutic agents. In certain embodiments, a compound disclosed herein, or a pharmaceutically acceptable salt thereof, is combined with two additional therapeutic agents. In other embodiments, a compound disclosed herein, or a pharmaceutically acceptable salt thereof, is combined with three additional therapeutic agents. In further embodiments, a compound disclosed herein, or a pharmaceutically acceptable salt thereof, is combined with four additional therapeutic agents. The one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a compound disclosed herein is administered with one, two, three, or four additional therapeutic agents. Co-administration of a compound disclosed herein with one, two, three, or four additional therapeutic agents generally refers to simultaneous or sequential administration of a compound disclosed herein and one, two, three, or four additional therapeutic agents, such that therapeutically effective amounts of the compound disclosed herein and the one, two, three, or four additional therapeutic agents are both present in the body of the patient. When administered sequentially, the combination may be administered in two or more administrations.

Co-administration includes administration of unit dosages of the compounds disclosed herein before or after administration of unit dosages of one, two, three, or four additional therapeutic agents. For example, the compound disclosed herein may be administered within seconds, minutes, or hours of the administration of the one, two, three, or four additional therapeutic agents. In some embodiments, a unit dose of a compound disclosed herein is administered first, followed within seconds or minutes by administration of a unit dose of one, two, three, or four additional therapeutic agents. Alternatively, a unit dose of one, two, three, or four additional therapeutic agents is administered first, followed by administration of a unit dose of a compound disclosed herein within seconds or minutes. In other embodiments, a unit dose of a compound disclosed herein is administered first, followed, after a period of hours (e.g., 1-12 hours), by administration of a unit dose of one, two, three, or four additional therapeutic agents. In yet other embodiments, a unit dose of one, two, three, or four additional therapeutic agents is administered first, followed, after a period of hours (e.g., 1-12 hours), by administration of a unit dose of a compound disclosed herein.

In certain embodiments, a kit comprising a compound disclosed herein (e.g., a compound of Formula I, Ia, Ib, Ic, Id, Ie, or If, or a pharmaceutically acceptable salt thereof, and one or more (e.g., one, two, three, or four) additional therapeutic agents is provided.

In a specific embodiment, the kit includes a compound disclosed herein, or a pharmaceutically acceptable salt thereof, an HIV nucleoside or nucleotide inhibitor of reverse transcriptase and an HIV capsid inhibitor or an HIV capsid polymerization inhibitor.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators. HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors. CCR5 inhibitors, Nef inhibitors, latency reversing agents, HIV bNAbs, agonists of TLR7. TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are chosen from dolutegravir, cabotegravir, islatravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, H1viral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir), NOV-205. OB-002H, ODE-Bn-TFV, PA-1050040 (PA-040), PC-707, PGN-007, QF-036, 5-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR—CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911.

Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), doravirine+islatravir (fixed dose combination/oral tablet formulation, HIV-1 infection), elsulfavirine (long acting injectable nanosuspension, HIV infection), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, islatravir, MK-8583. VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710. NSC-699171, NSC-699172, NSC-699173, NSC-699174, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV capsid inhibitors include, but are not limited to, those described in U.S. Patent Application Publication Nos. US20200317689, US20210284642, US2014221356 and US2016016973.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-10), nifeviroc (TD-0232), anti-GP120/CD4 or CCR5 bispecific antibodies, B-07, MB-66, polypeptide C₂₅P. TD-0680, thioraviroc and vMIP (Haimipu).

Examples of gp4l inhibitors include, but are not limited to, albuvirtide, enfuvirtide, griffithsin (gp4l/gp120/gpl60 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, lipuvirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, TAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQI, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CAI, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L 1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CDI 12R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL 16 binding protein 1 (ULBP1); UL16 binding protein 2 (ULBP2); UL 16 binding protein 3 (ULBP3); retinoic acid early transcript 1E (RAET1E; ULBP4); retinoic acid early transcript IG (RAET1G; ULBP5); retinoic acid early transcript IL (RAET1L; ULBP6); lymphocyte activating 3 (CD223); killer cell immunoglobulin like receptor, three Ig domains and long cytoplasmic tail 1 (KIR, CD158E 1); killer cell lectin like receptor C1 (KLRC1, NKG2A, CD159A); killer cell lectin like receptor K1 (KLRK1, NKG2D, CD314); killer cell lectin like receptor C₂ (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DLI); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL 1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155), See, e.g., Xu et al., J Exp Clin Cancer Res. (2018) 37; 110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL 1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C₁ (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75: Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181(budigalimab), PD1-P1K, BAT-1306, (MSB0010718C), CX-072, CBT-502, TSR-042 (dostarlimab), MSB-2311, JTX-4014, BGB-A333, SHR-1316, CS-1001 (WBP-3155, KN-035, 1B1-308 (sintilimab), HLX-20, KL-A167, STI-A1014, STI-A1015 (IMC-001), BCD-135, FAZ-053, TQB-2450, MDX1105-01, GS-4224, GS-4416, 1NCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40: NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF1OB (CD262, DRS, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MED16383, MEDI0562 (tavolixizumab), MOXR0916, PF-04518600, RG-7888, GSK-3174998, INCAGN1949, BMS-986178, GBR-8383, ABBV-368, and those described in WO2016179517, WO20170%179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089%28.

### Bi- and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor. e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346: Fang et al., Semin Immunol. (2017) 31:37-54. Examples of trispecific NK cell engagers (TRiKE) include, but are not limited to, OXS-3550. HIV-TriKE, and CD16-IL-15-B7H3 TriKe.

Indoleamine-pyrrole-2,3-dioxygenase (IDO1) inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID. 7098), TLR4 (NCBI Gene ID: 7099), TLRS (NCBI Gene ID: 7100), TLR6 (NCBI Gene ID: 10333). TLR7 (NCBI Gene ID: 51284). TLR8 (NCBI Gene ID: 51311), TLR9 (NCBI Gene ID: 54106), and/or TLR 10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen). WO2014/023813 (Janssen). US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma). US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125. S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-(X)3-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1, TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-1 and NOD2 Modulators

In some embodiments, the agents described herein are combined with a stimulator of interferon genes (STING). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-1 modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CU1-101, Neo-2/15; examples of IL-15 agonists, such as ALT-803, NKTR-255, and hetIL-15, interleukin-15/Fc fusion protein, AM-0015, NIZ-985, SO—C₁₀₁, IL-15 Synthorin (pegylated 11-15), P-22339, and a IL-15-PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577. GSK-2636771, INCB-040093, LY-3023414. MLN-1117, PQR-309, RG-7666, RP-6530, RV-1729, SAR-245409, SAR-260301, SF-1126, TGR-1202, UCB-5857, VS-5584, XL-765, and ZSTK-474.

alpha-4/beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®9, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, IB2530, INC9, 8ANC195, 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acid Res., 42 (Database issue):D1 133-9 (2014), Mascola et al., Immunol Rev., 254(1):225-44 (2013), such as 2F5, 4E10, M66.6, CAP206—CH12, 10E81 (all of which bind the MPER of gp4l); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312: WO2012/158948: WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue):DI 133-9 (2014), Mascola et al., Immunol Rev., 254(1):225-44 (2013), such as 2F5, 4E10, M66.6, CAP206—CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiTA-SG, CH01, CH59, C₂F5, C4E10, C2F5+C2G12+C4E10, CAP256V2LS, 3BNC117, 3BNC117-LS, 3BNC60, DH270.1, DH270.6, D1D2, 10-1074-LS, C13hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1. N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDX010 (ipilimumab), VRC01, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7, VRC-HIVMAB080-00-AB, VRC-HIVMAB060-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LN01.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e., rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxyirus based vaccine (modified vaccinia virus Ankara (MVA), orthopoxyirus-derived NYVAC, and avipoxyirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHiVconsv1 vaccine, CMV-MVA triplex vaccine, ChAdOx1.HTI, Chimigen HIV vaccine, ConM SOSIP.v7 gp140, ALVAC HIV (vCP1521), AIDSV AX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C₅, VAC-3S, multiclade DNA recombinant adenovirus-5 (rAdS), rAd5 gag-pol env AB/C vaccine, Pennvax-G, Pennvax-GP, Pennvax-G, MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIV consv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines. ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C. A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVR1.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA®, (tenofovir disoproxil fumarate and emtricitabine; TDF+FTC); DESCOVY® (tenofovir alafenamide and emtricitabine); ODEFSEY®, (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestreno, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-B01, HSPCs overexpressing ALDH1 (LV-800, HIV infection), AGT103-T, and SupT1 cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C₃₄—CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells, for example, ImmTAV.

### B-Cell Therapy

In certain embodiments, the antibodies or antigen-binding fragments described herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

A compound as disclosed herein (e.g., any compound of Formula I) may be combined with one, two, three, or four additional therapeutic agents in any dosage amount of the compound of Formula I (e.g., from 1 mg to 500 mg of compound).

In one embodiment, kits comprising a compound disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases. TALENs), cell therapies (such as chimeric antigen receptor T-cell. CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors. HIV integrase inhibitors, HIV non-catalytic site (or allosteric) integrase inhibitors. HIV entry (fusion) inhibitors. HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, islatravir implant, doravirine, raltegravir, and long acting dolutegravir.

### PGP Inhibitors

In some embodiments, the compounds described herein can be used in combination with a P-glycoprotein (PGP) inhibitor. Examples of PGP inhibitors include, but are not limited to, verapamil, dexverapamil, cyclosporine, zosuquidar, laniquidar, elacridar, tariquidar, and encequidar. In some embodiments, the compounds described herein can be used in combination with encequidar. In some embodiments, the compounds described herein can be used in combination with a pharmaceutically acceptable salt of encequidar. In some embodiments, the compounds described herein can be used in combination with a mesylate salt of encequidar.

In some embodiments, the compounds described herein can be co-administered with a P-glycoprotein (PGP) inhibitor. Examples of PGP inhibitors include, but are not limited to, verapamil, dexverapamil, cyclosporine, zosuquidar, laniquidar, elacridar, tariquidar, and encequidar. In some embodiments, the compounds described herein can be co-administered with encequidar. In some embodiments, the compounds described herein can be co-administered with a pharmaceutically acceptable salt of encequidar. In some embodiments, the compounds described herein can be co-administered with a mesylate salt of encequidar.

### VII. Examples

### Intermediate A: (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (A)

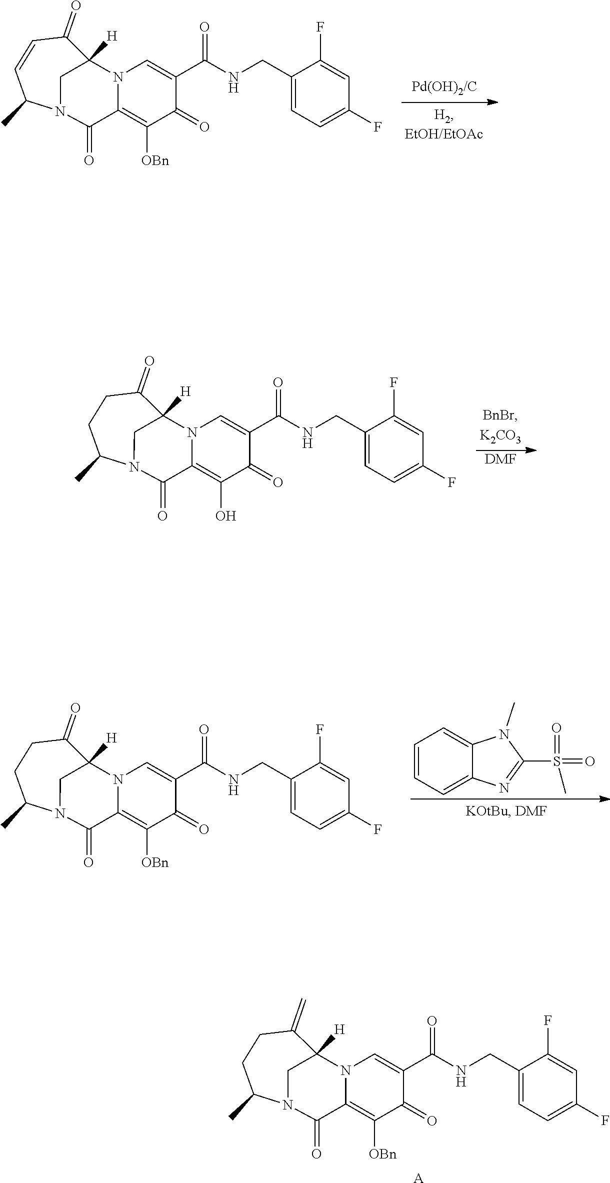

### Step 1: Synthesis of (3S,7R)—N-(2,4-difluorobenzyl)-12-hydroxy-3-methyl-1,6,11-trioxo-1,4,5,6,7,11-hexahydro-3H-2,7-methanopyrido[1,2-a][1,4]diazonine-10-carboxamide

To a solution of (3S,7R)-12-(benzyloxy)-N-(2,4-difluorobenzyl)-3-methyl-1,6,11-trioxo-1,6,7,11-tetrahydro-3H-2,7-methanopyrido[1,2-a][1,4]diazonine-10-carboxamide (17 g, 32.7 mmol) in EtOAc (200 mL) at room temperature was added EtOH (400 mL) followed by 20% Pd(OH)₂/C (50 wt % water, 7.6 g). The resulting mixture was degassed and flushed with nitrogen three times and then degassed and flushed with hydrogen three times before it was hydrogenated under hydrogen balloon for 4 hours. The reaction was then degassed and flushed with nitrogen, diluted with DCM, filtered through Celite®, concentrated and used directly in next step. MS (m/z) 432.124 [M+H]⁺.

### Step 2: Synthesis of (3S,7R)-12-(benzyloxy)-N-(2,4-difluorobenzyl-3-methyl-1,6,11-trioxo-1,4,5,6,7,11-hexahydro-3H-2,7-methanopyrido[1,2-a][1,4]diazonine-10-carboxamide

(3S,7R)—N-(2,4-difluorobenzyl)-12-hydroxy-3-methyl-1,6,11-trioxo-1,4,5,6,7,11-hexahydro-3H-2,7-methanopyrido[1,2-a][1,4]diazonine-10-carboxamide (33.1 g, 76.7 mmol) from Step 1 was dissolved in DMF (300 mL) at room temperature and K₂CO₃ (16.0 g, 115.0 mmol) and benzyl bromide (13.1 g, 76.7 mmol) were added. The resulting mixture was then heated to 50° C. for 4.5 hours and then cooled to room temperature. The mixture was filtered through a pad of Celite® and the filter cake was rinsed with DMF (100 mL). Combined filtrate was carried directly into the next step. MS (m/z) 554.086 [M+H+MeOH]⁺.

### Step 3: Synthesis of (3S,7S)-12-(benzyloxy-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide

The solution of (3S,7R)-12-(benzyloxy)-N-(2,4-difluorobenzyl)-3-methyl-1,6,11-trioxo-1,4,5,6,7,11-hexahydro-3H-2,7-methanopyrido[1,2-a][1,4]diazonine-10-carboxamide (39.7 g, 76.1 mmol) in DMF (350 mL) was immersed into a room temperature water bath. 1-methyl-2-(methylsulfonyl)-1H-benzo[d]imidazole (20.81 g, 99.0 mmol) was added in one portion followed by potassium tert-butoxide (21.36 g, 190 mmol) in 5 portions. The reaction was removed from the water bath and stirred at room temperature for 1.5 hours. The reaction was then quenched slowly with 0.5N HCl in water (180 mL) and extracted with EtOAc three times. The combined organic layer was washed with water and brine, dried over sodium sulfate, filtered and concentrated. The residue was purified by normal phase silica gel chromatography, eluting with 0-100% EtOAc/hexane, to afford intermediate A. MS (m/z) 520.060 [M+H]⁺.

### Intermediate B: Preparation of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (B)

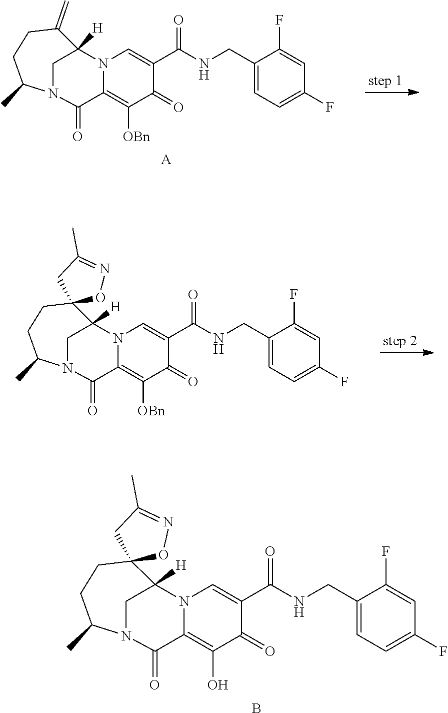

### Step 1: Preparation of (3′S,5S,7′R)-12′-(benzyloxy)-N-(2,4-difluorobenzyl)-3,3′-dimethyl-1,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

Into the solution of acetaldehyde oxime (666 mg, 11.3 mmol) in DMF (50 ml) was added N-chlorosuccinimide (1.51 g, 11.3 mmol) at room temperature, then heated to 60° C. for 1 h. After cooling to room temperature, (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (Intermediate A) (1.58 g, 3.04 mmol) and triethylamine (1.539 g, 15.2 mmol) were added at room temperature. After stirring at room temperature overnight, the reaction was quenched by adding sat. NaHCO₃ solution. The mixture was extracted with EtOAc, the organic phase was separated and dried over MgSO₄, filtered, concentrated down and purified by silica gel chromatography column (eluting with 0-100% EtOAc/hexane). MS (m/z) 577.135 [M+H]+

### Step 2: Preparation of (3′S,5S,7′R)—N-(2,4-difluorobenzyl)-12′-hydroxy-3,3′-dimethyl-1′,11′-dioxo-1′,4′,5′,1′-tetrahydro-3′H,4H,7′H-spiro[isoxazole-5,6′-[2,7]methanopyrido[1,2-a][1,4]diazonine]-10′-carboxamide (B)

To a solution of (3′S,5S,7′R)-12′-(benzyloxy)-N-(2,4-difluorobenzyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide ((4.7 mg, 0.112 mmol) in toluene (2 mL) was added TFA (0.5 mL). The reaction mixture was stirred at room temperature overnight. The reaction mixture was concentrated down, and the residue was purified by reverse phase HPLC, eluting with 10-90% acetonitrile in water to give intermediate B. MS (m/z) 487.12 [M+H]⁺. ¹H NMR (400 MHz, Chloroform-d) δ 10.53 (s, 1H), 8.43 (s, 1H), 7.37 (td, J=8.6, 6.3 Hz, 1H), 6.92-6.78 (m, 2H), 4.82-4.70 (m, 1H), 4.67 (t, J=4.8 Hz, 2H), 4.18 (d, J=2.2 Hz, 1H), 3.86 (dd, J=14.9, 1.9 Hz, 1H), 3.72 (dd, J=14.9, 2.7 Hz, 1H), 2.94 (d, J=17.8 Hz, 1H), 2.53 (d, J=17.7 Hz, 1H), 2.06 (s, 3H), 2.04-1.88 (m, 3H), 1.56 (dd, J=14.3, 11.2 Hz, 1H), 1.32 (d, J=6.6 Hz, 3H).

### Intermediate C: Synthesis of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

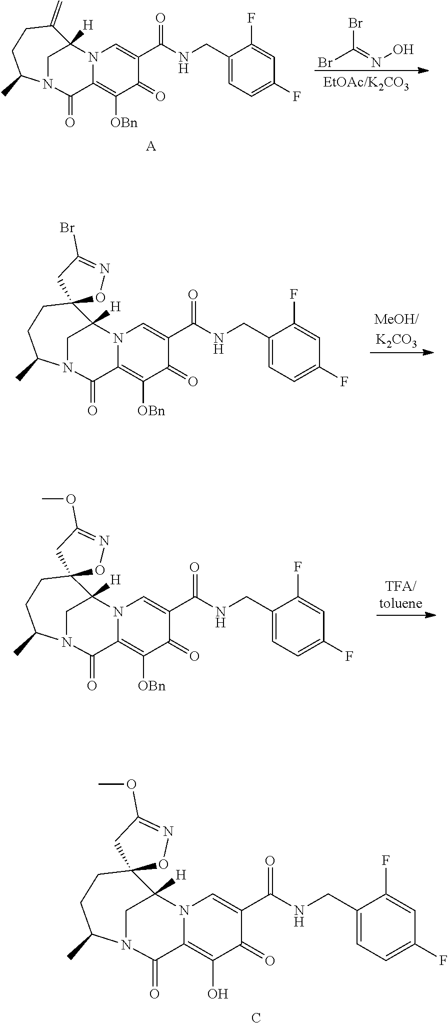

### Step 1: Synthesis of (3′S,5S,7′R)-12′-(benzyloxy)-3-bromo-N-(2,4-difluorobenzyl-3′-methyl-1′,11′-dioxo-1′,4′,5′,11′-tetrahydro-3′H,4H,7′H-spiro[isoxazole-5,6′-[2,7methanopyrido(1,2-a][1,4]diazonine]-10′-carboxamide

To a solution of (3S,7S)-12-(benzyloxy)-N-(2,4-difluorobenzyl)-3-methyl-6-methylene-1,11-dioxo-1,4,5,6,7,11-hexahydro-3H-2,7-methanopyrido[1,2-a][1,4]diazonine-10-carboxamide (Intermediate A) (18.1 g, 34.8 mmol) in EtOAc (350 mL) at 0° C. was added dibromomethanone oxime (21.199 g, 105 mmol) followed by potassium carbonate (28.846 g, 209 mmol). The resulting mixture was stirred overnight before it was diluted with water (3X) mL). The layers were separated. The aqueous layer was extracted with EtOAc (200 mL) and the combined organic phase was washed with brine, dried over sodium sulfate, filtered and concentrated. The residue was purified by normal phase silica gel chromatography, eluting with 0-80% EtOAc/Hexane to afford the title compound. MS (m/z) 640.904 [M+H]⁺. 1H NMR (400 MHz, DMSO) δ 10.41 (t, J=6.0 Hz, 1H), 8.89 (s, 1H), 7.54-7.21 (m, 7H), 7.13-7.05 (m, 1H), 5.30 (d, J=10.5 Hz, 1H), 5.04 (d, J=10.5 Hz, 1H), 4.78 (s, 1H), 4.72-4.54 (m, 3H), 3.75-3.62 (m, 2H), 3.39 (d, J=17.6 Hz, 1H), 3.07 (d, J=17.7 Hz, 1H), 1.92-1.73 (m, 3H), 1.41 (t, J=12.9 Hz, 1H), 1.15 (d, J=6.7 Hz, 3H).

### Step 2: Synthesis of (3′S,5S,7′R)-12′-(benzyloxy)-N-(2,4-difluorobenzyl)-3-methoxy-3′-methyl-1′,11′-dioxo-1′,4′,5′,11′-tetrahydro-3′H,4H, 7′H-spiro[isoxazole-5,6′-[2,7]methanopyrido[1,2-a][1,4]diazonine]-10′-carboxamide

To a mixture of (3′S,5S,7′R)-12′-(benzyloxy)-3-bromo-N-(2,4-difluorobenzyl)-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (23.2 g, 36.2 mmol) in MeOH (450 mL) and DMF (100 mL) at 55° C. was added potassium carbonate (12.496 g, 90.4 mmol). The reaction was stirred for 3 hrs and then cooled to room temperature. To this stirred mixture was added water (1100 mL) and stirring continued for 30 minutes. The mixture was filtered and the filter cake was rinsed with water, collected, and dried to give the title compound. MS (m/z) 593.207 [M+H]⁺. 1H NMR (400 MHz, DMSO) δ 10.41 (t, J=6.0 Hz, 1H), 8.73 (s, 1H), 7.53-7.48 (m, 2H), 7.47-7.30 (m, 4H), 7.25 (ddd, J=10.5, 9.3, 2.6 Hz, 1H), 7.08 (tdd, J=8.5, 2.6, 1.0 Hz, 1H), 5.30 (d, J=10.5 Hz, 1H), 5.03 (d, J=10.5 Hz, 1H), 4.73-4.63 (m, 2H), 4.63-4.49 (m, 2H), 3.82 (s, 3H), 3.65 (d, J=2.0 Hz, 2H), 3.08 (d, J=16.8 Hz, 1H), 2.77 (d, J=16.9 Hz, 1H), 1.96-1.71 (m, 3H), 1.38 (dd, J=15.5, 10.2 Hz, 1H), 1.15 (d, J=6.6 Hz, 3H).

### Step 3: Synthesis of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide

To a solution of (3′S,5S,7′R)-12′-(benzyloxy)-N-(2,4-difluorobenzyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (19 g, 32.1 mmol) in toluene (64 mL) at room temperature was added TFA (32 mL) and stirred for 16 hrs. The solvents were removed by rotatory evaporator. The resulting residue was treated with EtOAc (60 mL) and concentrated, repeating three times. The residue was stirred with a mixture of toluene (42 mL)/MeOH (42 mL)/EtOAc (42 mL) at 70° C. for 1 hr and room temperature for 2 hours. The mixture was then filtered and the filter cake was rinsed with EtOAc, collected and dried to give title compound. MS (m/z) 503.263 [M+H]⁺. 1H NMR (400 MHz, DMSO) δ 10.99 (s, 1H), 10.34 (t, J=5.9 Hz, 1H), 8.64 (s, 1H), 7.42 (td, J=8.7, 6.6 Hz, 1H), 7.25 (ddd, J=10.5, 9.3, 2.6 Hz, 1H), 7.08 (tdd, J=8.6, 2.6, 1.0 Hz, 1H), 4.74 (s, 1H), 4.63-4.47 (m, 3H), 3.81 (s, 3H), 3.80-3.67 (m, 2H), 2.93 (d, J=16.9 Hz, 1H), 2.70 (d, J=16.9 Hz, 1H), 1.95-1.76 (m, 3H), 1.41-1.31 (m, 1H), 1.19 (d, J=6.8 Hz, 3H).

### Intermediate D: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate

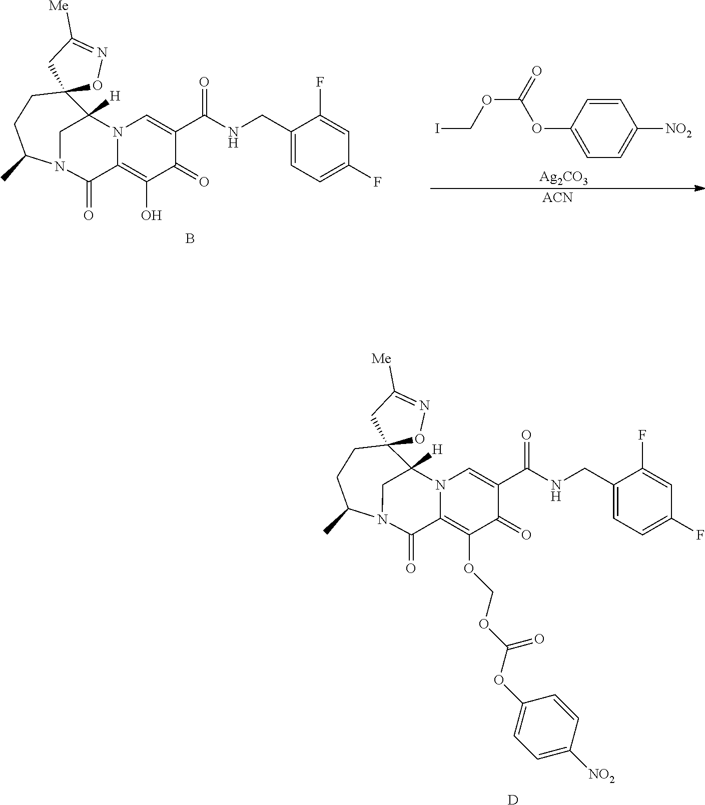

(3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 3.06 g, 6.29 mol) and iodomethyl (4-nitrophenyl) carbonate (4.23 g, 13.1 mmol), prepared according to WO2010011812, were mixed with acetonitrile (73 mL). Ag₂CO₃ (5.29 g, 19.2 mmol) was added in one portion. The slurry was stirred at room temperature overnight and filtered through celite. The filtrate was collected and concentrated to dryness. The residue was purified on silica gel column with 0-20% MeOH in DCM to afford the crude material upon concentration. The crude product was then dissolved in EtOAc (200 mL) and was treated with water (200 mL) with agitation. Organic phase was separated, dried over Na₂SO₄ and filtered. The filtrate was concentrated to afford Intermediate D. Calculated for C₃₂H₂₉F₂N₅O₁₀: 681.19, Found MS (ESI+): 682.01 [M+H]+.

### Intermediate E: Synthesis of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate

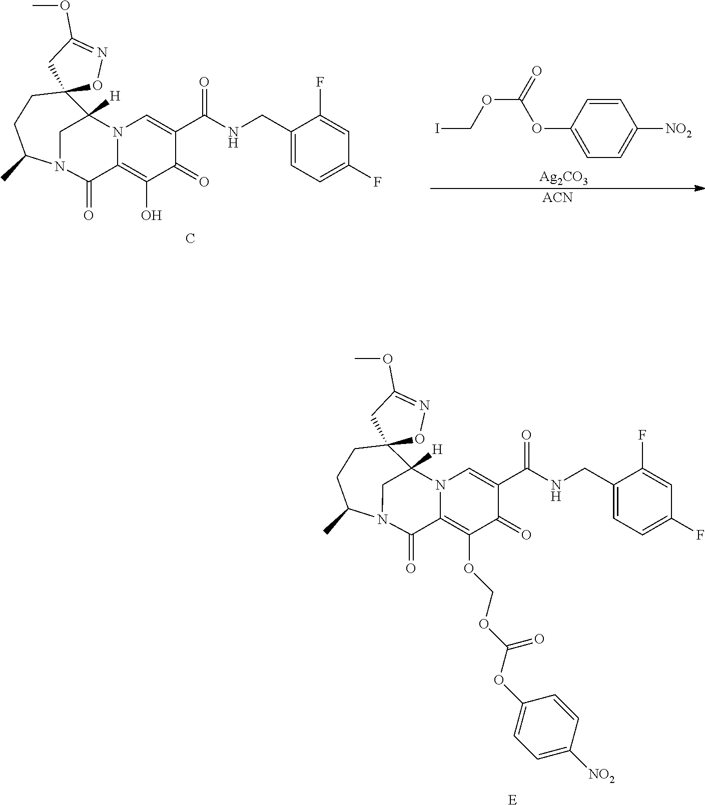

The title compound was prepared following a similar method for Intermediate D, except using Intermediate C instead of Intermediate B. LCMS-ESI+ (m/z): calcd H+ for C₃₂H29F2N5O11, Theoretical:697.18, Found: 697.845.

### Example 1: (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1],4diazonin]-12′-yl (2-methoxyethyl) carbonate (1)

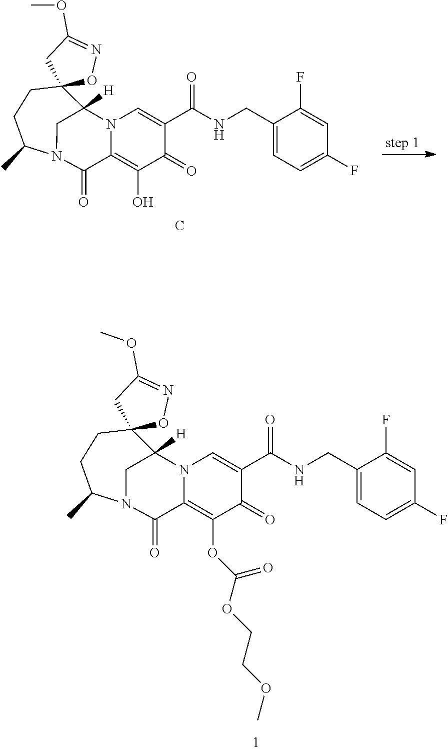

To a mixture of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C, 20 mg, 0.0398 mmol) in DMF (3.0 mL) at room temperature was added N,N-diisopropylethylamine (7.72 mg, 0.0597 mmol) and DMAP (4.86 mg, 0.0398 mmol) followed by 2-methoxyethyl carbonochloridate (6.62 mg, 0.0478 mmol). The resulting mixture was stirred at room temperature for 16 hours before it was diluted with EtOAc, washed with water, saturated ammonium chloride, brine, dried over sodium sulfate, filtered, concentrated and purified by normal phase silica gel chromatography to give the title compound. ¹H NMR (400 MHz, Chloroform-d) δ 10.20 (t, J=5.9 Hz, 1H), 8.72 (s, 1H), 7.44-7.32 (m, 1H), 6.90-6.74 (m, 2H), 4.92-4.78 (m, 1H), 4.70 (dd, J=15.1, 6.1 Hz, 1H), 4.55 (dd, J=15.1, 5.6 Hz, 1H), 4.49-4.38 (m, 3H), 3.87 (s, 31H), 3.79 (dd, J=15.2, 1.9 Hz, 1H), 3.75-3.63 (m, 3H), 3.43 (s, 3H), 3.09 (d, J=17.1 Hz, 1H), 2.65 (d, J=17.2 Hz, 1H), 2.05-1.87 (m, 3H), 1.67-1.56 (m, 1H), 1.25 (d, J=6.7 Hz, 3H). LCMS-ESI+ (m/z): calcd H+ for C28H30F2N4O9, Theoretical:604.20, Found: 604.985.

### Example 2: Synthesis of (3′S,5S,7′R)-1O′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2-(2-methoxyethoxy)ethyl) carbonate (2)

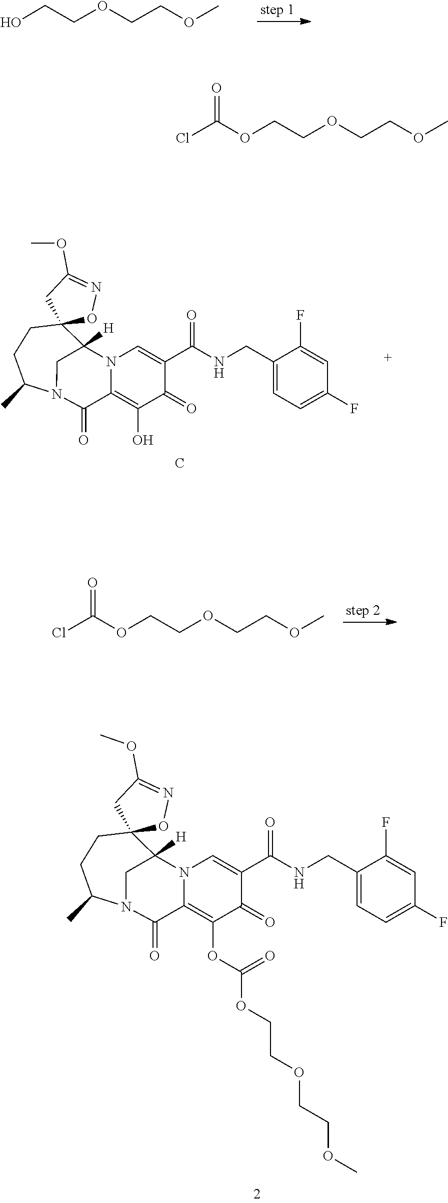

### Step 1: Synthesis of 2-(2-methoxyethoxy)ethyl carbonochloridate

To a solution of bis(trichloromethyl) carbonate (1235 mg, 4.16 mmol) in THF (60 mL) at 0° C. was added pyridine (856 mg, 10.8 mmol) followed by a solution of 2-(2-methoxyethoxy)ethanol (1000 mg, 8.32 mmol) in THF (20 mL). The resulting suspension was removed from cooling bath and allowed to warm to room temperature and stir for 3 hours. The reaction mixture was filtered through Celite® and rinsed with DCM. The filtrate was washed with 1 N HCl in water, dried over Na₂SO₄, filtered, and concentrated. The product was used directly in the next step.

Step 2: Synthesis of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl(2-(2-methoxyethoxy)ethyl) carbonate (2)

To a mixture of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-11′-carboxamide (Intermediate C, 25 mg, 0.0498 mmol) in DMF (0.5 mL) at 0° C. was added N,N-diisopropylethylamine (19.3 mg, 0.149 mmol) followed by 2-(2-methoxyethoxy)ethyl carbonochloridate (18.2 mg, 0.0995 mmol). The resulting suspension was removed from cooling bath and allowed to warm to room temperature and stir for 16 hours. The reaction mixture was then diluted with EtOAc, washed with 1 N HCl (×2), water (×2), brine, dried over Na₂SO₄, filtered, concentrated and purified by normal phase silica gel chromatography to give the title compound. 1H NMR (400 MHz, Chloroform-d) δ 10.22 (t, J=5.9 Hz, 1H), 8.83 (s, 1H), 7.36 (td, J=8.4, 6.3 Hz, 1H), 6.89-6.75 (m, 2H), 4.89-4.77 (m, 1H), 4.70 (dd, J=15.1, 6.2 Hz, 1H), 4.58-4.49 (m, 2H), 4.49-4.43 (m, 2H), 3.85-3.74 (m, 6H), 3.73-3.65 (m, 3H), 3.61-3.54 (m, 2H), 3.40 (s, 3H), 3.10 (d, J=17.1 Hz, 1H), 2.62 (d, J=17.0 Hz, 1H), 2.05-1.87 (m, 3H), 1.66-1.55 (m, 1H), 1.25 (d, J=6.7 Hz, 3H). LCMS-ESI+ (m/z): calcd H+ for C₃₀H34F2N4O10, Theoretical: 648.22, Found: 648.986.

### Example 3: (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2-(2-(2-methoxyethoxy)ethoxy)ethyl) carbonate (3)

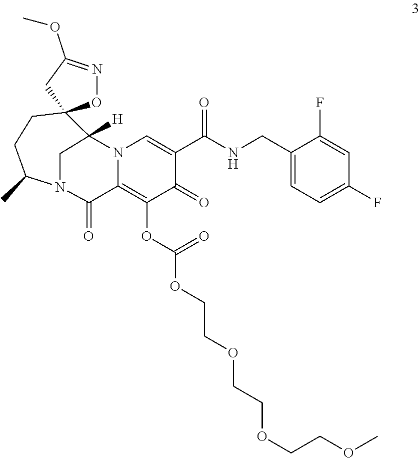 [SMILES: COCCOCCOCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared following a similar method for the preparation of (3′S,5S,7′R)-10′-((2,4-difluorobenzyl)carbamoyl)-3-methoxy-3′-methyl-1′,11′-dioxo-1′,4,5′,11′-tetrahydro-3′H,4H,7′H-spiro[isoxazole-5,6′-[2,7]methanopyrido[1,2-a][1,4]diazonin]-12′-yl (2-(2-methoxyethoxy)ethyl) carbonate (Example 2) except using 2-[2-(2-methoxyethoxy)ethoxy]ethanol instead of 2-(2-methoxyethoxy)ethanol in step 1. ¹H NMR (400 MHz, Chloroform-d) δ 10.22 (t, J=5.9 Hz, 1H), 8.83 (s, 1H), 7.35 (td, J=8.3, 6.2 Hz, 1H), 6.91-6.72 (m, 2H), 4.89-4.76 (m, 1H), 4.70 (dd, J=15.1, 6.2 Hz, 1H), 4.57-4.48 (m, 2H), 4.47-4.41 (m, 2H), 3.85-3.79 (m, 5H), 3.74-3.64 (m, 8H), 3.58-3.53 (m, 2H), 3.39 (s, 3H), 3.10 (d, J=17.0 Hz, 1H), 2.62 (d, J=17.1 Hz, 1H), 2.09-1.86 (m, 3H), 1.68-1.53 (m, 1H), 1.25 (d, J=6.7 Hz, 3H). LCMS-ESI+ (m/z): calcd H+ for C₃₂H38F2N4O11, Theoretical: 692.25, Found: 693.137.

### Example 4: Synthesis of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-(2-methoxyethoxy)ethyl) carbonate (4)

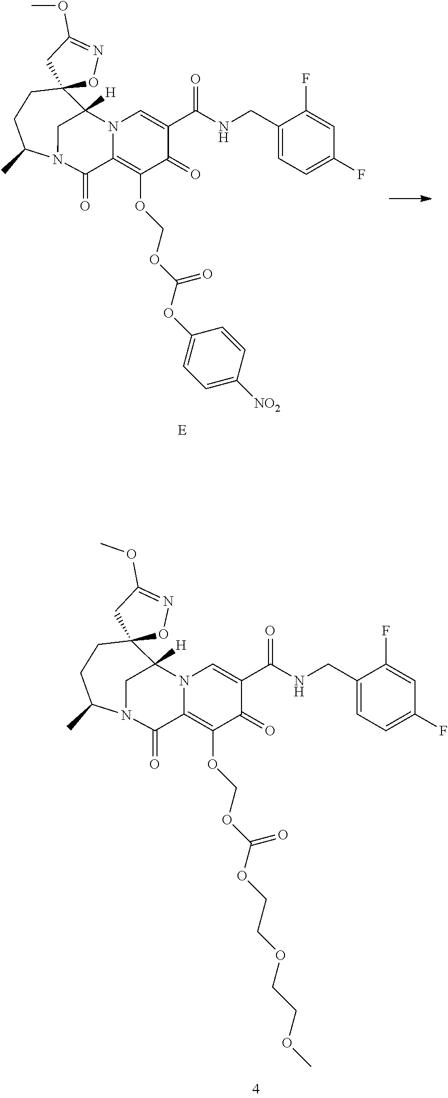

To a mixture of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate E, 100 mg, 0.143 mmol) in acetonitrile (2.0 mL) at room temperature was added 2-(2-methoxyethoxy)ethan-1-ol (86.04 mg, 0.717 mmol) followed by triethylamine (72.5 mg, 0.717 mmol) and DMAP (2.62 mg, 0.0215 mmol). The resulting mixture was stirred at room temperature for 2 hours before it was diluted with EtOAc, washed with water, brine, dried over sodium sulfate, filtered, concentrated and purified by normal phase chromatography to give the title compound. ¹H NMR (400 MHz, Acetonitrile-d3) δ 10.35-10.26 (m, 1H), 8.51 (s, 1H), 7.45 (td, J=9.3, 8.8, 6.6 Hz, 1H), 7.05-6.96 (m, 2H), 5.87 (d, J=6.4 Hz, 1H), 5.70 (d, J=6.4 Hz, 1H), 4.83-4.71 (m, 1H), 4.61 (d, J=5.9 Hz, 2H), 4.38 (s, 1H), 4.31-4.18 (m, 2H), 3.88 (s, 3H), 3.76-3.64 (m, 4H), 3.58 (dd, J=5.9, 3.4 Hz, 2H), 3.48 (dd, J=5.8, 3.4 Hz, 2H), 3.31 (s, 3H), 3.01 (d, J=17.2 Hz, 1H), 2.70 (d, J=17.2 Hz, 1H), 2.04-1.82 (m, 3H), 1.50 (ddd, J=15.3, 10.5, 2.4 Hz, 1H), 1.21 (d, J=6.7 Hz, 3H). LCMS-ESI+ (m/z): calcd H+ for C₃₁H36F2N4O11, Theoretical:678.23, Found: 678.948.

### Example 5: (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-methoxyethyl) carbonate (5)

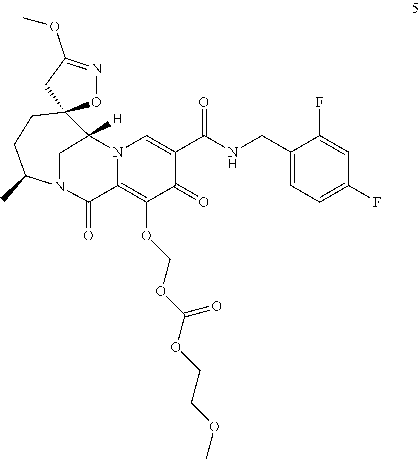 [SMILES: COCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was synthesized following a similar method for the preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-(2-methoxyethoxy)ethyl) carbonate (Example 4) except 2-(2-methoxyethoxy)ethan-1-ol was replaced by 2-methoxyethanol. ¹H NMR (400 MHz, Acetonitrile-d3) δ 10.31 (t, J=6.0 Hz, 1H), 8.51 (s, 1H), 7.45 (td, J=9.2, 8.8, 6.5 Hz, 1H), 7.07-6.92 (m, 2H), 5.86 (d, J=6.4 Hz, 1H), 5.70 (d, J=6.4 Hz, 1H), 4.83-4.69 (m, 1H), 4.60 (d, J=5.8 Hz, 2H), 4.37 (d, J=2.6 Hz, 1H), 4.30-4.16 (m, 2H), 3.88 (s, 3H), 3.76-3.63 (m, 2H), 3.58 (dd, J=5.0, 4.3 Hz, 2H), 3.32 (s, 3H), 3.01 (d, J=17.2 Hz, 1H), 2.68 (s, 1H), 2.05-1.76 (m, 3H), 1.58-1.43 (m, 1H), 1.21 (d, J=6.7 Hz, 3H). LCMS-ESI+ (m/z): calcd H+ for C₂₉H32F2N4O10, Theoretical: 634.21, Found: 634.941.

### Example 6: Synthesis of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl stearate (6)

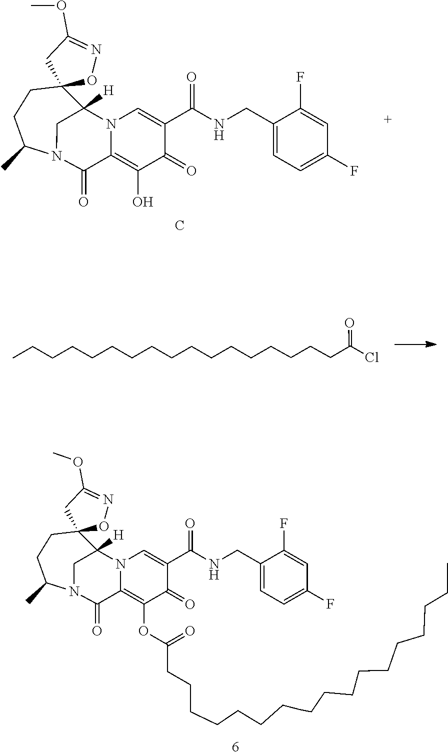

To a mixture of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-11′-carboxamide (Intermediate C, 50 mg, 0.0995 mmol) in DMF (0.5 mL) at room temperature was added N,N-diisopropylethylamine (51.4 mg, 0.398 mmol) followed by octadecanoyl chloride (60.3 mg, 0.199 mmol). The resulting mixture was stirred at room temperature for 48 hours before it was diluted with EtOAc, washed with water, brine, dried over Na₂SO₄, filtered, concentrated and purified by normal phase silica gel chromatography to give the title compound. ¹H NMR (400 MHz, Chloroform-d) δ 10.26 (t, J=5.9 Hz, 1H), 8.83 (s, 1H), 7.41-7.31 (m, 1H), 6.89-6.75 (m, 2H), 4.89-4.77 (m, 1H), 4.72 (dd, J=15.1, 6.2 Hz, 1H), 4.58-4.44 (m, 2H), 3.84 (s, 3H), 3.81-3.63 (m, 2H), 3.10 (d, J=17.0 Hz, 1H), 2.71-2.57 (m, 3H), 2.05-1.86 (m, 3H), 1.83-1.73 (m, 2H), 1.61 (ddd, J=15.4, 10.8, 2.0 Hz, 1H), 1.47-1.37 (m, 2H), 1.37-1.21 (m, 29H), 0.93-0.85 (m, 3H). LCMS-ESI+ (m/z): calcd H+ for C₄₂H58F2N4O7, Theoretical: 768.43, Found: 769.806.

### Example 7: Synthesis of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (9Z,12Z)-octadeca-9,12-dienoate (7)

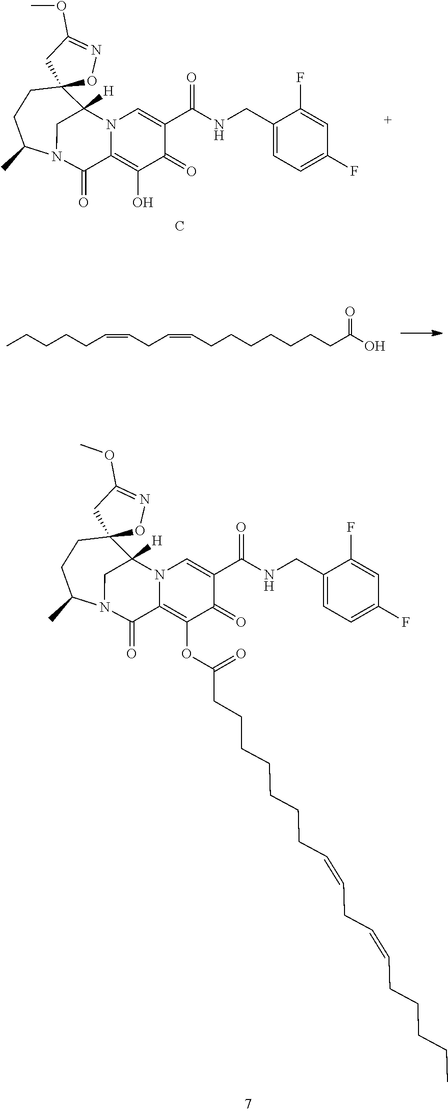

To a mixture of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (intermediate C, 20 mg, 0.0398 mmol) in DMF (0.5 mL) at room temperature was added (9Z,12Z)-octadeca-9,12-dienoic acid (22.3 mg, 0.0796 mmol) followed by HATU (37.8 mg, 0.0995 mmol) and N,N-diisopropylethylamine (15.4 mg, 0.119 mmol). The resulting mixture was stirred at room temperature for 16 hours before it was diluted with EtOAc, washed with water, brine, dried over Na₂SO₄, filtered, concentrated and purified by normal phase chromatography to give the title compound. 1H NMR (400 MHz, Chloroform-d) δ 10.25 (t, J=5.9 Hz, 1H), 8.89 (s, 1H), 7.38-7.29 (in, 1H), 6.86-6.73 (m, 2H), 5.45-5.24 (m, 4H), 4.87-4.64 (m, 2H), 4.59-4.42 (m, 2H), 3.83-3.58 (m, 5H), 3.09 (d, J=17.0 Hz, 1H), 2.82-2.72 (m, 2H), 2.64 (dd, J=8.5, 6.8 Hz, 2H), 2.57 (d, J=17.1 Hz, 1H), 2.09-1.84 (m, 7H), 1.81-1.67 (m, 2H), 1.63-1.52 (m, 1H), 1.46-1.25 (m, 14H), 1.23 (d, J=6.7 Hz, 3H), 0.92-0.84 (nm, 3H). LCMS-ESI+ (m/z); calcd H+ for C₄₂H54F2N4O7, Theoretical: 764.40, Found: 764.456.

### Example 8: Synthesis of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2-(2-methoxyethoxy)ethyl) carbonate (8)

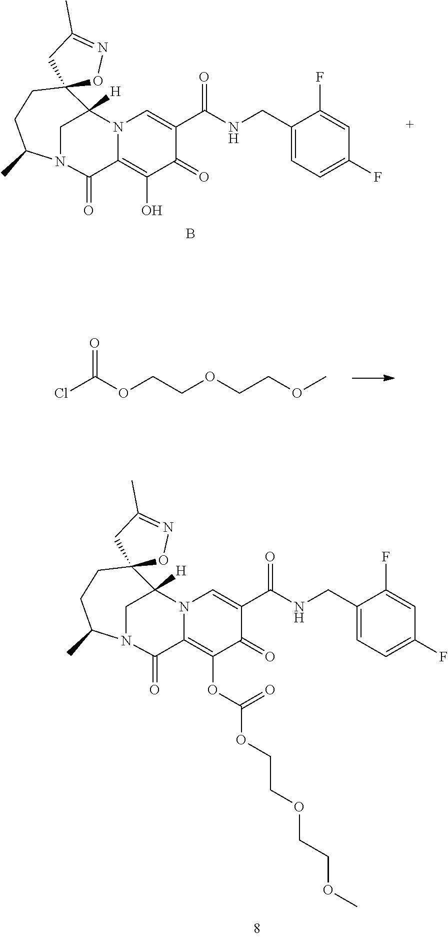

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 56 mg, 0.115 mmol) in DMF (0.5 mL) at 0° C. was added N,N-diisopropylethylamine (44.6 mg, 0.345 mmol) followed by 2-(2-methoxyethoxy)ethyl carbonochloridate (42.0 mg, 0.23 mmol). The resulting mixture was removed from cooling bath after addition and stirred at room temperature for 16 hours before it was diluted with EtOAc, washed with IN HCl, water, brine, dried over Na₂SO₄, filtered, concentrated and purified by normal phase silica gel chromatography to give the title compound. ¹H NMR (400 MHz, Chloroform-d) δ 10.16 (t, J=5.9 Hz, 1H), 8.44 (s, 1H), 7.35 (td, J=8.7, 6.5 Hz, 1H), 6.88-6.76 (m, 2H), 4.88-4.76 (m, 1H), 4.67 (dd, J=15.2, 6.1 Hz, 1H), 4.57 (dd, J=15.1, 5.8 Hz, 1H), 4.50-4.43 (m, 2H), 4.15-4.09 (m, 1H), 3.86-3.76 (m, 3H), 3.74-3.66 (m, 3H), 3.60-3.53 (m, 2H), 3.40 (s, 3H), 3.05 (d, J=17.8 Hz, 1H), 2.65-2.56 (m, 1H), 2.09 (s, 3H), 2.00-1.85 (m, 3H), 1.61 (ddd, J=15.0, 9.0, 3.7 Hz, 1H), 1.33-1.20 (m, 3H). LCMS-ESI+ (m/z): calcd H+ for C₃₀H34F2N4O9, Theoretical:632.23, Found: 632.973.

### Example 9: Synthesis of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2-methoxyethyl) carbonate (9)

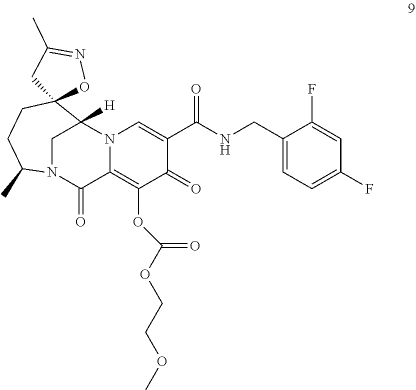 [SMILES: COCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was synthesized following a similar method for the preparation of (3′S,5S,7′R)-10′-((2,4-difluorobenzyl)carbamoyl)-3,3′-dimethyl-1′,11′-dioxo-1′,4′,5′,11′-tetrahydro-3′H,4H7′H-spiro[isoxazole-5,6′-[2,7]methanopyrido[1,2-a][1,4]diazonin]-12′-yl (2-(2-methoxyethoxy)ethyl) carbonate (Example 8) except 2-(2-methoxyethoxy)ethyl carbonochloridate was replaced by 2-methoxyethyl carbonochloridate. ¹H NMR (400 MHz, Chloroform-d) δ 10.17 (t, J=5.9 Hz, 1H), 8.43 (s, 1H), 7.36 (td, J=8.7, 6.5 Hz, 1H), 6.91-6.76 (m, 2H), 4.89-4.77 (m, 1H), 4.67 (dd, J=15.1, 6.1 Hz, 1H), 4.58 (dd, J=15.1, 5.8 Hz, 1H), 4.49-4.42 (m, 2H), 4.13-4.08 (m, 1H), 3.81 (dd, J=15.1, 1.9 Hz, 1H), 3.75-3.65 (m, 3H), 3.43 (s, 3H), 3.10-3.00 (m, 1H), 2.61 (dd, J=17.8, 1.3 Hz, 1H), 2.10 (s, 3H), 2.00-1.86 (m, 3H), 1.67-1.57 (m, 1H), 1.25 (d, J=6.6 Hz, 3H). LCMS-ESI+ (m/z): calcd H+ for C₂₈H30F2N4O8, Theoretical: 588.20, Found: 588.986.

### Example 10: Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-methoxyethyl) carbonate (10)

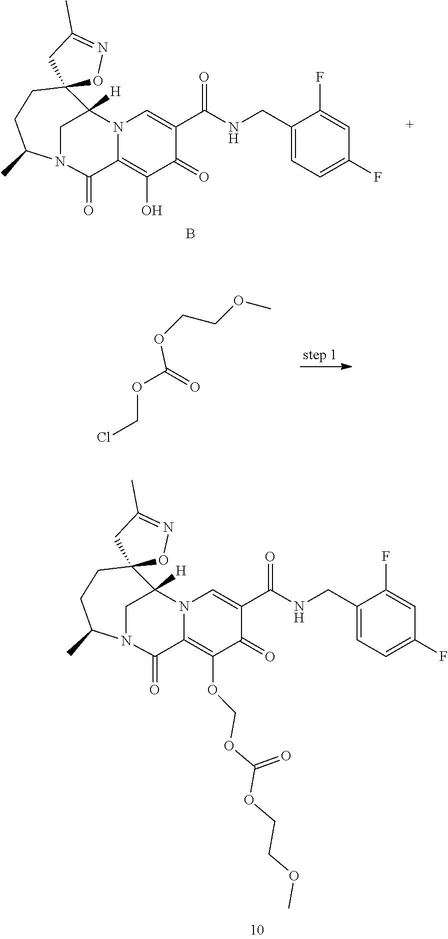

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 400 mg, 0.822 mmol) in DMF (9 mL) at room temperature was added potassium carbonate (284 mg, 2.06 mmol) and tetrabutylammonium iodide (304 mg, 0.822 mmol) followed by chloromethyl 2-methoxyethyl carbonate (222.0 mg, 1.32 mmol). The resulting mixture was heated at 55° C. for 1 hour and then at 80° C. for another hour before it was cooled to room temperature. The reaction was then diluted with EtOAc, washed with water, brine, dried over Na₂SO₄, filtered, concentrated and purified by normal phase chromatography to give the title compound (10). ¹H NMR (400 MHz, Acetone-d6) δ 10.36 (t, J=5.9 Hz, 1H), 8.58 (s, 1H), 7.50 (td, J=8.4, 6.4 Hz, 1H), 7.12-6.95 (m, 2H), 5.92 (d, J=6.4 Hz, 1H), 5.73 (d, J=6.3 Hz, 1H), 4.88-4.74 (m, 1H), 4.72-4.55 (m, 3H), 4.31-4.17 (m, 2H), 3.90 (dd, J=15.2, 2.7 Hz, 1H), 3.81 (dd, J=15.2, 2.0 Hz, 1H), 3.60 (t, J=4.8 Hz, 2H), 3.32 (s, 3H), 3.24 (dd, J=17.7, 1.1 Hz, 1H), 2.75 (dd, J=17.5, 1.1 Hz, 1H), 2.02 (d, J=1.0 Hz, 3H), 1.97-1.84 (m, 3H), 1.64-1.52 (m, 1H), 1.26 (d, J=6.7 Hz, 3H). LCMS-ESI+ (m/z): calcd H+ for C₂₉H32F2N4O9, Theoretical:618.21, Found: 618.796.

### Example 11: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12-yl)oxy)methyl methyl carbonate (11)

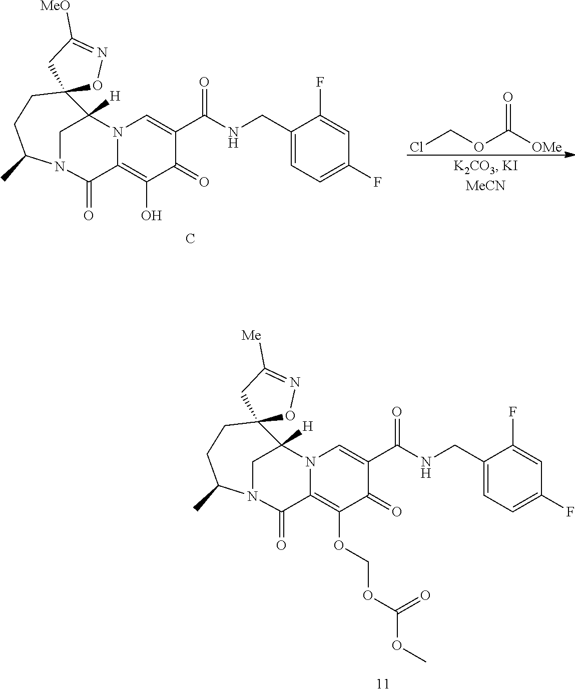

To a mixture of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C, 0.500 g, 0.995 mmol) in MeCN (10 mL) was added potassium carbonate (0.688 g, 4.98 mmol), potassium iodide (0.182 g, 1.09 mmol), and chloromethyl methyl carbonate (0.4% m, 3.98 mmol). The mixture was heated to 60° C. for 16 h, cooled to room temperature, and filtered. The filter cake was rinsed with MeCN and the filtrate was concentrated. The residue was purified by silica gel column chromatography (0-100% EtOAc/hexane) and reverse phase prep HPLC (5-100% MeCN/H₂O w/0.1% TFA) to afford the title compound after lyophilization. MS (m/z) 590.90 [M+H]⁺. ¹H NMR (400 MHz, DMSO-d6) δ 10.27 (t, J=6.0 Hz, 1H), 8.74 (s, 1H), 7.42 (td, J=8.7, 6.6 Hz, 1H), 7.24 (ddd, J=10.5, 9.3, 2.6 Hz, 1H), 7.07 (tdd, J=8.5, 2.6, 1.0 Hz, 1H), 5.78 (d, J=6.4 Hz, 1H), 5.58 (d, J=6.4 Hz, 1H), 4.70 (d, J=2.4 Hz, 1H), 4.62 (dt, J=10.2, 6.7 Hz, 1H), 4.58-4.49 (m, 2H), 3.81 (s, 3H), 3.68 (s, 3H), 3.67 (d, J=2.3 Hz, 2H), 3.05 (d, J=16.8 Hz, 1H), 2.75 (d, J=16.9 Hz, 1H), 1.96-1.70 (m, 3H), 1.40-1.26 (m, 1H), 1.15 (d, J=6.7 Hz, 3H).

### Example 12: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl methyl carbonate (12)

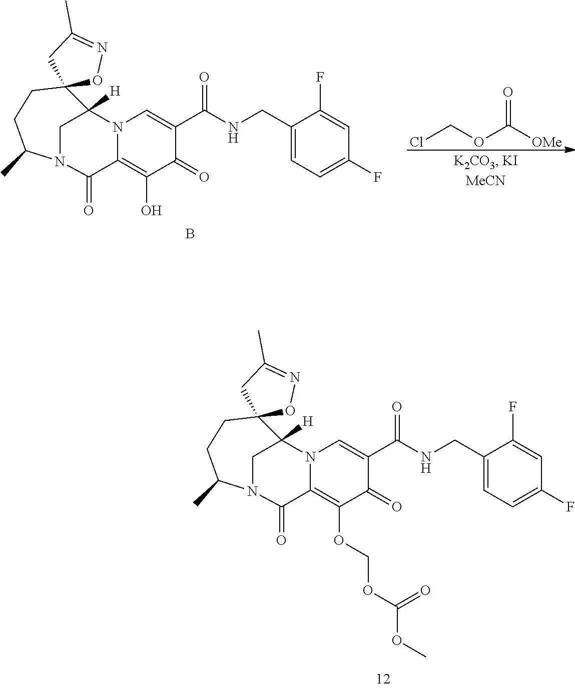

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.100 g, 0.206 mmol) in MeCN (2 mL) was added potassium carbonate (0.142 g, 1.03 mmol), potassium iodide (0.038 g, 0.226 mmol), and chloromethyl methyl carbonate (0.102 g, 0.822 mmol). The mixture was heated to 60° C. for 16 h, cooled to room temperature, and filtered. The filter cake was rinsed with MeCN and the filtrate was concentrated. The residue was purified by reverse phase prep HPLC (5-100% MeCN/H₂O w/0.1% TFA) to afford the title compound after lyophilization. MS (m/z) 574.94 [M+H]⁺. ¹H NMR (400 MHz, DMSO-d6) δ 10.27 (t, J=5.9 Hz, 1H), 8.69 (s, 1H), 7.42 (td, J=8.6, 6.6 Hz, 1H), 7.25 (ddd, J=10.5, 9.3, 2.6 Hz, 1H), 7.13-7.02 (m, 1H), 5.79 (d, J=6.5 Hz, 1H), 5.59 (d, J=6.4 Hz, 1H), 4.69-4.59 (m, 2H), 4.55 (t, J=5.4 Hz, 2H), 3.68 (s, 5H), 3.00 (d, J=17.6 Hz, 1H), 2.63 (d, J=17.6 Hz, 1H), 1.94 (s, 3H), 1.88-1.68 (m, 3H), 1.36-1.21 (m, 1H), 1.15 (d, J=6.7 Hz, 3H).

### Example 13: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl tetradecanoate (13)

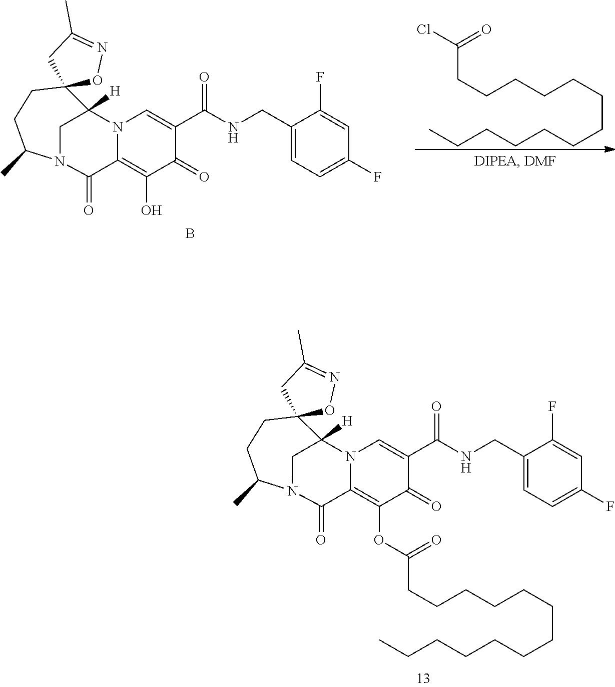

To a solution of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.250 g, 0.514 mmol) in DMF (2.5 mL) was added DIPEA (0.36 mL, 2.0)6 mmol) and cooled to 0° C. Myristoyl chloride (0.35 mL, 1.28 mmol) was added and the reaction mixture was allowed to warm to rt and stir for 48 h. The reaction mixture was diluted with EtOAc and washed with water and brine. The organic phase was dried over Na₂SO₄, filtered, and concentrated. The residue was purified by silica gel column chromatography (0-100% EtOAc/hexane) and fractions containing product were pooled, concentrated, and lyophilized from 1:1 MeCN/water to afford the title compound. MS (m/z) 696.83 [M+H]⁺. ¹H NMR (400 MHz, CDCl₃) δ 10.18 (t, J=6.0 Hz, 1H), 8.38 (s, 1H), 7.39-7.30 (m, 1H), 6.86-6.75 (m, 2H), 4.88-4.74 (m, 1H), 4.66 (dd, J=15.0, 5.8 Hz, 1H), 4.61-4.49 (m, 1H), 4.05 (s, 1H), 3.79 (dd, J=15.0, 1.9 Hz, 1H), 3.67 (d, J=15.4 Hz, 1H), 3.02 (d, J=17.8 Hz, 1H), 2.65 (t, J=7.7 Hz, 2H), 2.59 (d, J=17.8 Hz, 1H), 2.09 (s, 3H), 1.92 (q, J=14.9, 13.1 Hz, 3H), 1.77 (p. J=7.7 Hz, 2H), 1.61 (d, J=9.8 Hz, 1H), 1.24 (d, J=8.3 Hz, 23H), 0.88 (t, J=6.8 Hz, 3H).

### Example 14: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl palmitate (14)

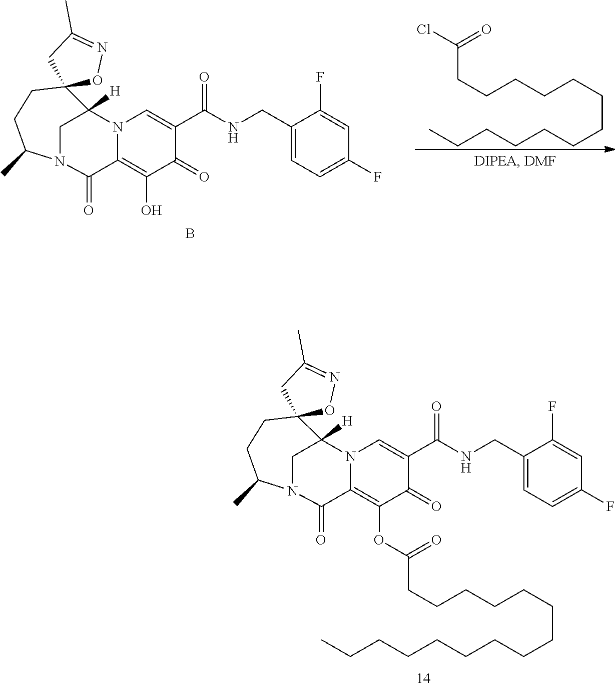

(3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl palmitate (14) was prepared in a manner similar to (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl tetradecanoate (Example 13), except using palmitoyl chloride instead of myristoyl chloride. MS (m/z) 724.84 [M+H]+. ¹H NMR (400 MHz, CDCl₃) δ 10.18 (t, J=5.9 Hz, 1H), 8.38 (s, 1H), 7.39-7.29 (m, 1H), 6.88-6.74 (m, 2H), 4.79 (dt, J=16.4, 6.8 Hz, 1H), 4.66 (dd, J=15.3, 5.7 Hz, 1H), 4.55 (d, J=15.3 Hz, 1H), 4.05 (s, 1H), 3.84-3.73 (m, 1H), 3.67 (d, J=15.2 Hz, 1H), 3.02 (d, J=17.8 Hz, 1H), 2.65 (t, J=7.7 Hz, 2H), 2.59 (d, J=17.8 Hz, 1H), 2.09 (s, 3H), 2.01-1.83 (m, 3H), 1.77 (p, J=7.7 Hz, 2H), 1.60 (s, 1H), 1.24 (d, J=8.0 Hz, 27H), 0.93-0.82 (m, 3H).

### Example 15: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl stearate (15)

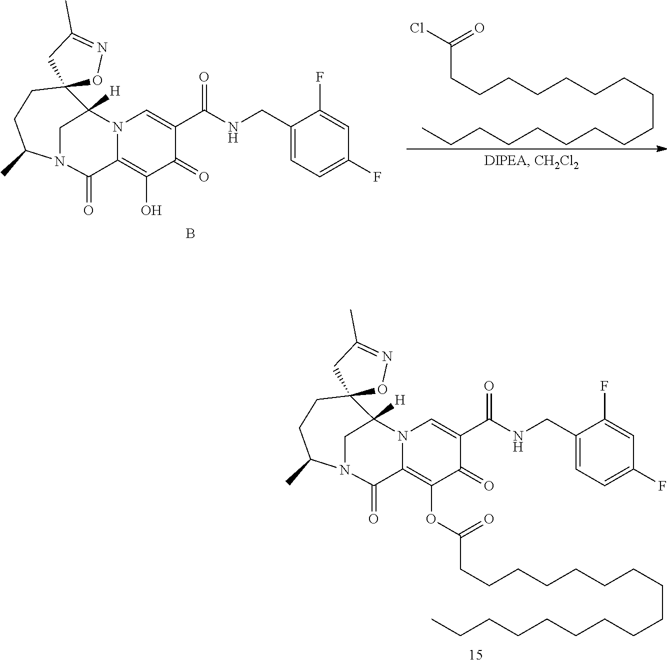

(3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl stearate (15) was prepared in a manner similar to (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl tetradecanoate (Example 13), except using stearoyl chloride instead of myristoyl chloride and CH₂Cl₂ instead of DMF. MS (m/z) 752.99 [M+H]+. ¹H NMR (400 MHz, CDCl₃) δ 10.18 (s, 1H), 8.37 (s, 1H), 7.34 (q, J=8.2 Hz, 1H), 6.86-6.74 (m, 2H), 4.87-4.73 (m, 1H), 4.66 (d, J=13.8 Hz, 1H), 4.56 (d, J=14.6 Hz, 1H), 4.05 (s, 1H), 3.83-3.73 (m, 1H), 3.67 (d, J=15.1 Hz, 1H), 3.02 (d, J=17.8 Hz, 1H), 2.65 (t, J=7.7 Hz, 2H), 2.59 (d, J=17.7 Hz, 1H), 2.09 (s, 3H), 1.99-1.84 (m, 3H), 1.77 (p, J=7.7 Hz, 2H), 1.62 (s, 1H), 1.24 (d, J=7.9 Hz, 31H), 0.92-0.82 (m, 3H).

### Example 16: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl acetate (16)

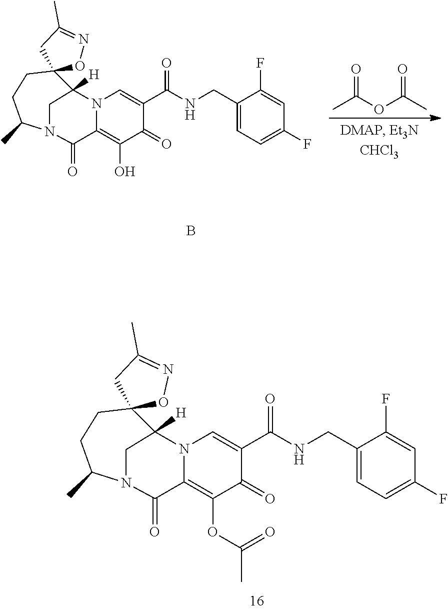

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.50 g, 1.03 mmol) in CHCl₃ (2 mL) was added DMAP (0.050 g, 0.411 mmol) and Et₃N (0.30 mL, 2.16 mmol). Acetic anhydride (0.16 mL, 1.75 mmol) was added and the reaction mixture was stirred at room temperature for 1 h. The mixture was concentrated and purified by silica gel column chromatography (0-100% EtOAc/hexane). Fractions containing product were pooled, concentrated, and lyophilized from 1:1 MeCN/water to afford the title compound. MS (m/z) 528.80 [M+H]+. ¹H NMR (400 MHz, CDCl₃) δ 10.17 (t, J=6.0 Hz, 1H), 8.39 (s, 1H), 7.34 (td, J=8.7, 6.5 Hz, 1H), 6.87-6.74 (m, 2H), 4.79 (dt, J=9.8, 6.8 Hz, 1H), 4.66 (dd, J=15.0, 5.9 Hz, 1H), 4.56 (dd, J=15.2, 5.5 Hz, 1H), 4.06 (d, J=2.2 Hz, 1H), 3.79 (dd, J=15.1, 1.9 Hz, 1H), 3.67 (d, J=15.4 Hz, 1H), 3.08-2.94 (m, 1H), 2.65-2.55 (m, 1H), 2.37 (s, 3H), 2.09 (d, J=1.0 Hz, 3H), 2.00-1.82 (m, 3H), 1.63-1.55 (m, 1H), 1.25 (d, J=6.7 Hz, 3H).

### Example 17: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl pentanoate (17)

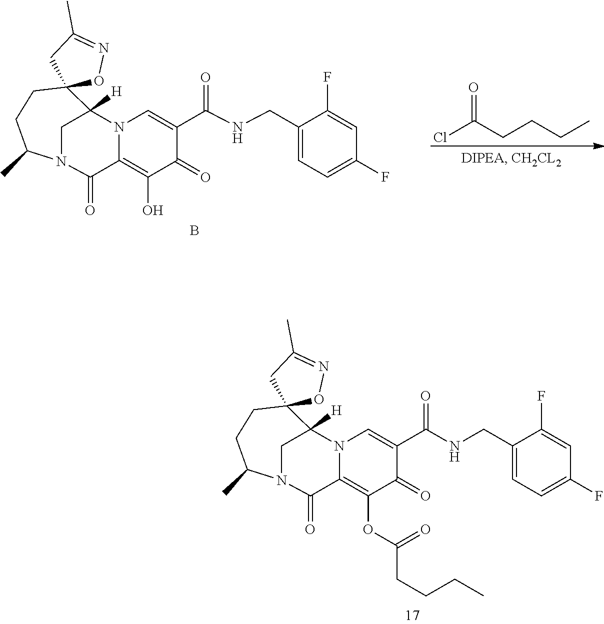

(3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl pentanoate (17) was prepared in a manner similar to (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl tetradecanoate (Example 13), except using valeroyl chloride instead of myristoyl chloride and CH₂Cl₂ instead of DMF. MS (m/z) 570.76 [M+H]⁺. ¹H NMR (400 MHz, CDCl₃) δ 10.17 (t, J=6.1 Hz, 1H), 8.38 (s, 1H), 7.39-7.29 (m, 1H), 6.86-6.73 (m, 2H), 4.80 (dt, J=9.8, 6.7 Hz, 1H), 4.66 (dd, J=15.2, 5.7 Hz, 1H), 4.60-4.47 (m, 1H), 4.05 (s, 1H), 3.79 (dd, J=15.1, 1.9 Hz, 1H), 3.67 (d, J=15.1 Hz, 1H), 3.02 (d, J=17.8 Hz, 1H), 2.66 (dd, J=8.5, 6.8 Hz, 2H), 2.59 (d, J=17.8 Hz, 1H), 2.09 (s, 3H), 2.02-1.83 (m, 3H), 1.76 (p. J=7.6 Hz, 2H), 1.65-1.58 (m, 1H), 1.44 (dt, J=14.7, 7.4 Hz, 2H), 1.24 (d, J=6.7 Hz, 3H), 0.95 (t, J=7.4 Hz, 3H).

### Example 18: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl pivalate (18)

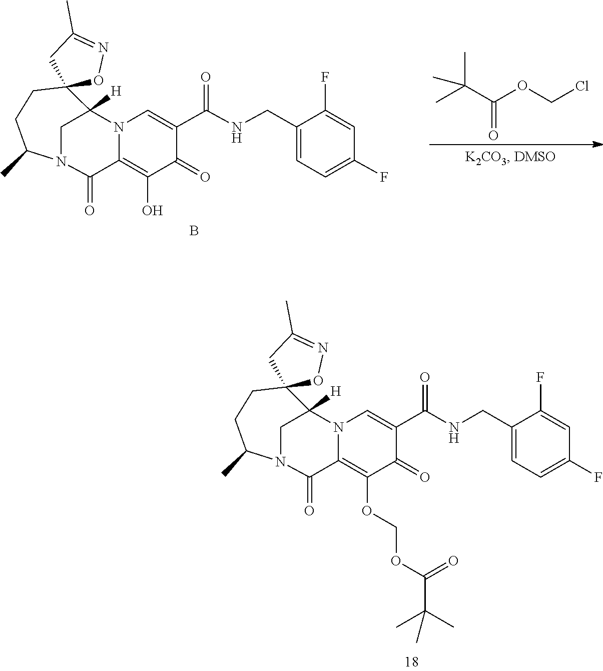

(3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.022 g, 0.0452 mmol) was dissolved in DMSO (1 ml), followed by the addition Cs₂CO₃ (0.031 g, 0.226 mmol). Chloromethyl pivalate (0.02 g, 0.136 mmol) was added to the mixture dropwise. The resulting mixture was stirred at room temperature overnight. The mixture was diluted with EtOAc, washed with water and brine, dried over sodium sulfate, filtered and concentrated. The crude material was purified by column chromatography (0-100% EtOAc/Hexane) to afford the title compound. MS (m/z) 600.9 [M+H]⁺. ¹H NMR (400 MHz, Chloroform-d) δ 10.33 (t, J=5.9 Hz, 1H), 8.33 (s, 1H), 7.44-7.35 (m, 1H), 6.87-6.74 (m, 2H), 5.92 (d, J=6.2 Hz, 1H), 5.83 (d, J=6.2 Hz, 1H), 4.88 (h, J=7.3 Hz, 1H), 4.78-4.54 (m, 2H), 4.04 (s, 1H), 3.77 (dd, J=15.0, 1.9 Hz, 1H), 3.63 (dd, J=15.0, 2.7 Hz, 1H), 3.04 (d, J=17.8 Hz, 1H), 2.58 (dd, J=17.8, 1.3 Hz, 1H), 2.11 (d, J=1.0 Hz, 3H), 1.99-1.82 (m, 4H), 1.60 (d, J=25.0 Hz, 3H), 1.20 (s, 9H).

### Example 19: Preparation (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl pivalate (19)

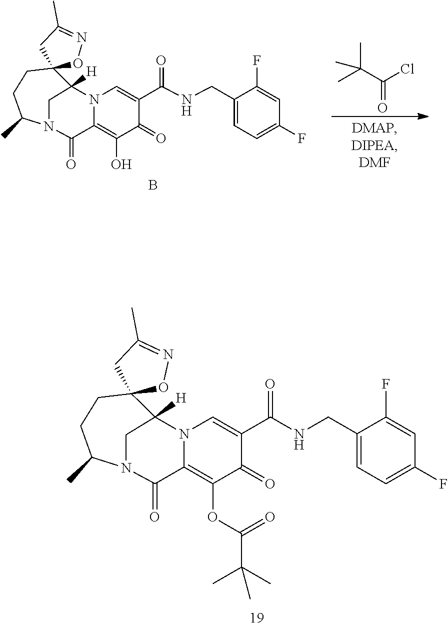

(3′S,5S,7′R)—N-(2,4-difluorobenzyl)-12-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 0.03 g, 0.0617 mmol) was dissolved in DMF (1 mL). The mixture was cooled to 0° C. followed by the addition of DIPEA (0.0322 ml, 0.185 mmol) and DMAP (0.0023 g, 0.0185 mmol). Pivaloyl chloride (0.015 g, 0.123 mmol) was added to the mixture slowly. The resulting mixture was stirred at rt for 2 hr. The mixture was diluted with EtOAc, washed with HCl (1N in H₂O) and brine, dried over sodium sulfate, filtered and concentrated. The resulting residue was purified by column chromatography (0-100% EtOAc/Hexane) to afford the title compound. MS (m/z) 571.08 [M+H]+. 1H NMR (400 MHz, Chloroform-d) δ 10.18 (t, J=6.0 Hz, 1H), 8.39 (s, 1H), 7.44-7.33 (m, 1H), 6.93-6.75 (m, 2H), 4.83 (dt, J=9.8, 6.7 Hz, 1H), 4.65 (m, 2H), 4.07 (m, 1H), 3.87-3.60 (m, 1H), 3.12-3.02 (m, 1H), 2.99-2.87 (m, 1H), 2.62 (m, 1H), 2.11 (d, 3H), 2.05-1.78 (m, 4H), 1.74-1.50 (m, 3H), 1.43 (s, 9H).

### Example 20 and 21: Preparation (1S)-1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl isopropyl carbonate (20) and (1R)-1-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl isopropyl carbonate (21)

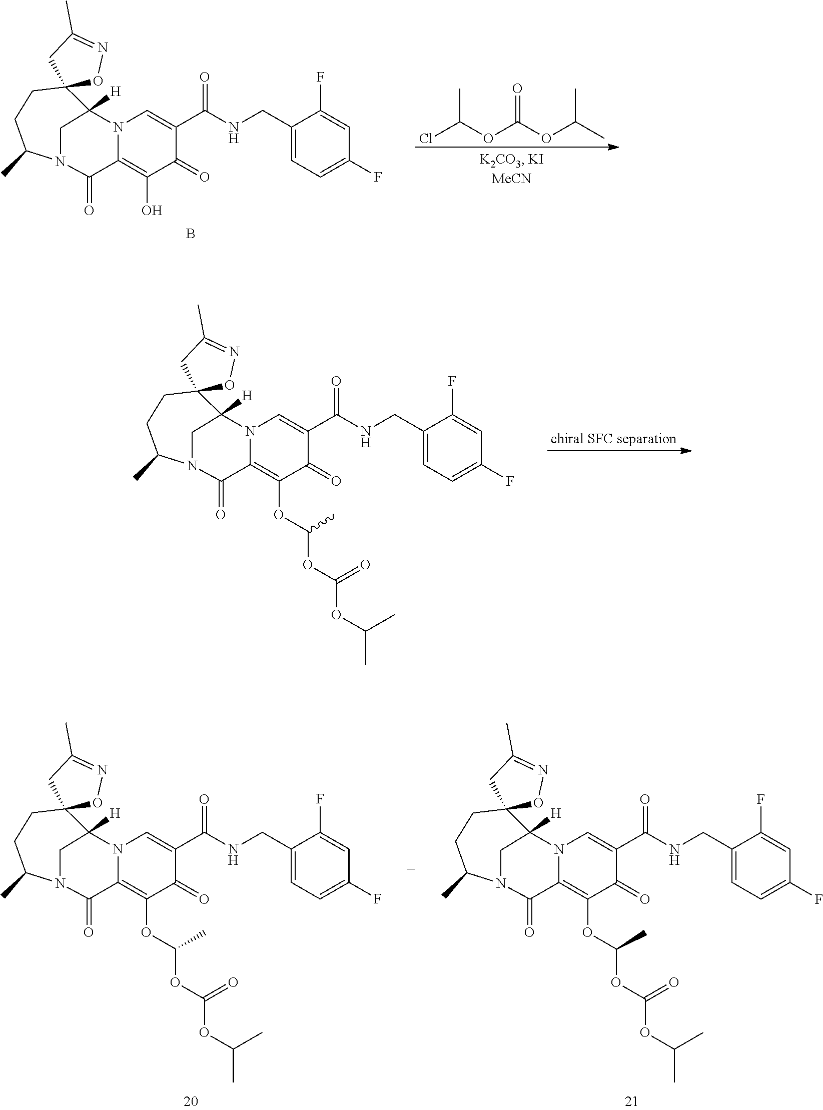

### Step 1: Synthesis of 1-(((3′S,5S,7′R)-10′-((24-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl isopropyl carbonate

To a mixture of (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 45 mg, 0.009 mmol) in MeCN (20 mL) was added potassium carbonate (25.6 mg, 0.185 mmol), potassium iodide (20.0 mg, 0.12 mmol), and 1-chloroethyl isopropyl carbonate (18.5 mg, 0.11 mmol). The mixture was heated to 70° C. for 3 h, cooled to room temperature, and filtered. The filter cake was rinsed with MeCN and the filtrate was concentrated. The residue was purified by reverse phase prep HPLC (5-100% MeCN/H2O w/0.1% TFA) to afford the title compound as a mixture of diastereomers. MS (m/z) 616.68 [M+H]⁺.

### Step 2: Chiral SFC separation to provide (1S)-1-(((3′S,5S,7′R)-10′-((2,4-difluorobenzyl)carbamoyl)-3,3′-dimethyl-1,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)ethyl isopropyl carbonate (20) and (IR)-1-(((3′S,5S,7′R)-10′-((2,4-di fluorobenzyl)carbamoyl)-3,3′-dimethyl-1′,11′-dioxo-1′4′5′,11′-tetrahydro-3′H,4H7′H-spiro[isoxazole-5,6′-(2,71methanopyrido[1,2-a][1,4]diazonin]-12′-yl)oxy)ethyl isopropyl carbonate (21)

The mixture of diastereomers obtained from step 1 was subjected to chiral SFC separation (SFC column: AD-H 4.6×100 mm, eluted with EtOH-NH3, 50%). The earlier eluted peak was arbitrary assigned to be Example 20, stereochemistry on promoiety not confirmed. MS (m/z) 616.32 [M+H]⁺. ¹H NMR (400 MHz, Chloroform-d) δ 10.34 (s, 1H), 8.32 (s, 1H), 7.38 (q, J=8.1 Hz, 1H), 6.92-6.76 (m, 2H), 6.67 (q, J=5.3 Hz, 1H), 4.88 (dd, J=10.5, 6.7 Hz, 1H), 4.77-4.55 (m, 2H), 4.03 (m, 1H), 3.76 (d, J=15.1 Hz, 1H), 3.62 (dd, J=15.0, 2.6 Hz, 1H), 3.07 (d, J=17.8 Hz, 1H), 2.59 (d, J=17.7 Hz, 1H), 2.11 (s, 3H), 2.00-1.84 (m, 2H), 1.79 (d, J=5.3 Hz, 2H), 1.55 (m, 4H), 1.31-1.14 (m, 9H).

The later eluted peak was arbitrary assigned to be Example 21, stereochemistry on promoiety not confirmed. MS (m/z) 616.72 [M+H]⁺. ¹H NMR (400 MHz, Chloroform-d) δ 10.35 (s, 1H), 8.33 (s, 1H), 7.38 (s, 1H), 6.91-6.78 (m, 2H), 6.39 (d, J=5.3 Hz, 1H), 4.90 (s, 1H), 4.80-4.49 (m, 2H), 4.03 (m, 1H), 3.76 (m, 1H), 3.62 (m, 1H), 3.03 (m, 1H), 2.60 (m, 1H), 2.11 (s, 3H), 1.95 (m, 2H), 1.81 (d, J=4.9 Hz, 2H), 1.58 (m, 4H), 1.30-1.23 (m, 9H).

### Example 22: Preparation (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl isopropyl carbonate (22)

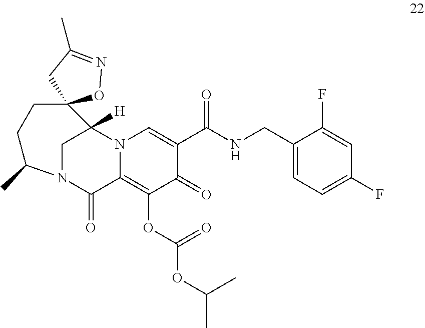 [SMILES: CC1=NO[C@@]2(CC[C@H](C)N3C[C@H]2n2cc(C(=O)NCc4ccc(F)cc4F)c(=O)c(OC(=O)OC(C)C)c2C3=O)C1]

The title compound was made by following a similar procedure to Example 19 except using isopropyl chloroformate (1.0 M in toluene) instead of using 2,2-dimethylpropanoyl chloride. MS (m/z) 572.8 [M+H]⁺. ¹H NMR (400 MHz, Chloroform-d) δ 10.20 (t, J=6.0 Hz, 1H), 8.40 (s, 1H), 7.43-7.33 (m, 1H), 6.94-6.73 (m, 2H), 5.03 (hept, J=6.2 Hz, 1H), 4.85 (dt, J=9.7, 6.8 Hz, 1H), 4.70 (d, J=9.1 Hz, 1H), 4.58 (dd, J=15.1, 5.4 Hz, 1H), 4.07 (s, 1H), 3.81 (dd, J=15.1, 1.9 Hz, 1H), 3.74-3.66 (m, 1H), 3.13-3.02 (m, 1H), 2.62 (dd, J=17.8, 1.3 Hz, 1H), 2.12 (d, J=1.0 Hz, 3H), 2.00-1.86 (m, 3H), 1.72-1.47 (m, 1H), 1.44 (t, J=6.2 Hz, 6H), 1.26 (d, J=6.7 Hz, 3H).

### Example 23: Preparation (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (2-(2-methoxyethoxy)ethyl) carbonate (23)

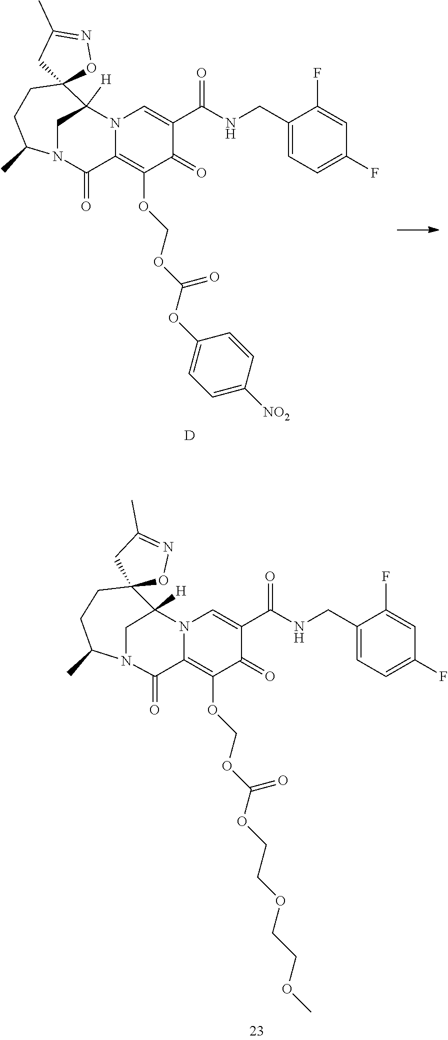

To a solution of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-nitrophenyl) carbonate (Intermediate D) (0.094 g, 1.38 mmol) in MeCN (1 ml) was added Et₃N (0.06 ml, 0.414 mmol) and DMAP (0.0017 g, 0.014 mmol), followed by the addition of 2-(2-methoxyethoxy)ethanol (0.033 g, 0.28 mmol). The reaction mixture was stirred at room temperature overnight. The solvent was removed under vacuo. The residue was purified by silica gel chromatography (0-100% EtOAc/hexane) to afford the title compound (23). MS (m/z) 662.8 [M+H]⁺. ¹H NMR (400 MHz, Chloroform-d) δ 10.33 (t, J=6.0 Hz, 1H), 8.37 (s, 1H), 7.36 (dd, J=8.8, 6.6 Hz, 1H), 6.91-6.77 (m, 2H), 6.06 (d, J=6.4 Hz, 1H), 5.87 (d, J=6.4 Hz, 1H), 4.86 (q, J=7.9 Hz, 1H), 4.71-4.58 (m, 2H), 4.36 (q, J=5.1 Hz, 2H), 4.05 (s, 1H), 3.82-3.76 (m, 2H), 3.67-3.64 (m, 2H), 3.60-3.51 (m, 3H), 3.39 (s, 3H), 3.05 (d, J=17.8 Hz, 1H), 2.67-2.57 (m, 1H), 2.12-2.10 (m, 4H), 2.03-1.85 (m, 3H), 1.68-1.53 (m, 1H), 1.24 (d, J=6.7 Hz, 3H).

### Example 24: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl isopropyl carbonate (24)

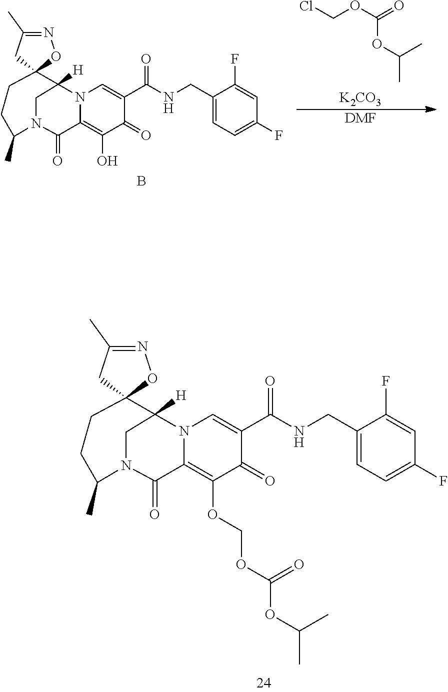

In a dry 100 mL reaction flask, (3′S,5S,7′R)—N-(2,4-difluorobenzyl)-12′-hydroxy-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate B, 302 mg, 0.62 mmol) was dissolved in DMSO (27 mL) at rt. K₂CO₃ (214 mg, 1.55 mmol) was added. The reaction mixture was heated under argon balloon to 50° C., then chloromethyl isopropyl carbonate (189 mg, 1.24 mmol) was added dropwise. The reaction mixture was then heated up to 80° C. and kept at that temperature with stirring for 2 hr. The reaction mixture was then diluted with EtOAc (50 mL) and washed sequentially with saturated aqueous NH₄Cl solution (50 mL) and water (50 mL). The aqueous phase was extracted with EtOAc (1×50 mL). The combined organic phase was washed with water (50 mL) and brine (50 mL) and then was concentrated to dryness. The residue was purified on silica gel column with EtOAc/hexane (0-100%) to afford the title compound (24). Calculated for C₂₉H₃₂F₂N₄O₈: 602.22, Found MS (ESI+) (m/z): 603.05 [M+H]⁺; ¹H NMR (400 MHz, CD3CN) δ 10.32 (d, J=6.3 Hz, 1H), 8.50 (s, 1H), 7.44 (td. J=8.8, 6.5 Hz, 1H), 6.98 (ddt, J=12.8, 8.4, 2.9 Hz, 2H), 5.84 (d, J=6.5 Hz, 1H), 5.68 (d, J=6.4 Hz, 1H), 4.90-4.70 (m, 2H), 4.60 (d, J=5.9 Hz, 2H), 4.34-4.28 (m, 1H), 3.75-3.62 (m, 2H), 3.04 (d, J=17.8 Hz, 1H), 2.59 (dd, J=17.7, 1.1 Hz, 1H), 1.99 (s, 3H), 1.96-1.79 (m, 3H), 1.51-1.40 (m, 1H), 1.26 (d, J=6.2 Hz, 6H), 1.21 (d, J=6.8 Hz, 3H).

### Example 25: Synthesis of (E)-4-(((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-4-oxobut-2-enoic acid (25)

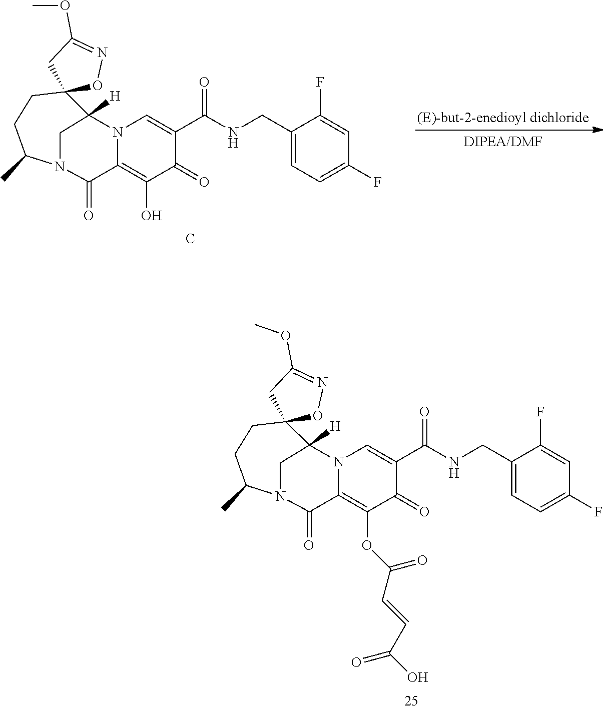

To a solution of (3′S,5S,7′R)—N-(2,4-difluorobenzyl)-12′-hydroxy-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonine]-10′-carboxamide (Intermediate C, 50 mg, 0.0995 mmol) in DMF (0.5 mL) at 0° C. was added DIPEA (25.7 mg, 0.199 mmol) followed by (E)-but-2-enedioyl dichloride (22.8 mg, 0.149 mmol) slowly. The reaction was removed from cooling bath after addition and diluted with DMF, filtered and purified by reverse phase preparative HPLC, eluted with 5-90% ACN/water containing 0.1% TFA. MS (m/z): 601.001 [M+H]⁺. 1H NMR (400 MHz, Acetonitrile-d3) δ 10.22-10.11 (m, 1H), 8.61 (s, 1H), 7.49-7.38 (m, 1H), 7.05-6.91 (m, 4H), 4.76-4.65 (m, 1H), 4.60 (d, J=5.9 Hz, 2H), 4.44 (s, 1H), 3.88 (s, 3H), 3.75 (d, J=2.2 Hz, 2H), 3.03 (d, J=17.2 Hz, 1H), 2.72 (d, J=17.2 Hz, 1H), 2.04-1.99 (m, 1H), 1.92-1.84 (m, 3H), 1.53-1.42 (m, 1H), 1.20 (d, J=6.7 Hz, 3H).

### Example 26: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl tetradecanoate (26)

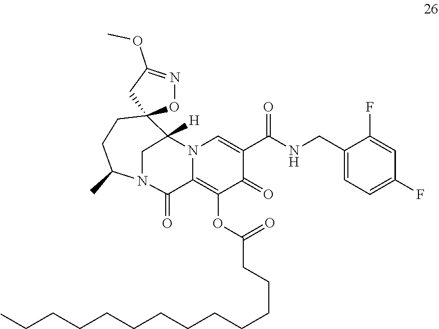 [SMILES: CCCCCCCCCCCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 6, except using tetradecanoyl chloride instead of octadecanoyl chloride and CH₂Cl₂ instead of DMF. MS (m/z) 713.82 [M+H]+. ¹H NMR (400 MHz, DMSO) δ 10.16 (t, J=6.0 Hz, 1H), 8.86 (s, 1H), 7.48-7.32 (m, 1H), 7.32-7.13 (m, 1H), 7.13-6.98 (m, 1H), 4.77 (s, 1H), 4.70-4.41 (m, 3H), 3.82 (s, 3H), 3.79-3.64 (m, 2H), 3.08 (d, J=16.7 Hz, 1H), 2.78 (d, J=16.8 Hz, 1H), 2.04-1.86 (m, 1H), 1.86-1.73 (m, 2H), 1.71-1.53 (m, 2H), 1.47-1.18 (m, 23H), 1.16 (d, J=6.7 Hz, 3H), 0.95-0.67 (m, 3H).

### Example 27: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl palmitate (27)

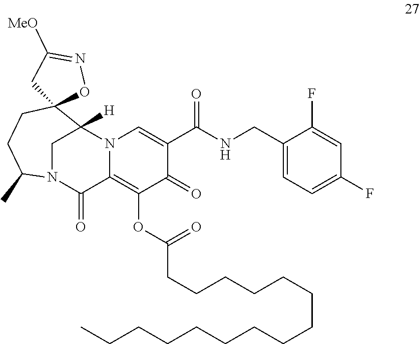 [SMILES: CCCCCCCCCCCCCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was made following the same method as Example 6, except using hexadecanoyl chloride instead of octadecanoyl chloride. MS (m/z) 742.1 [M+H]+. 1H NMR (400 MHz, Methanol-d4) δ 10.37 (t, J=5.9 Hz, 1H), 8.76 (s, 1H), 7.42 (td, J=8.7, 6.3 Hz, 1H), 6.91 (ddt, J=11.2, 8.3, 2.8 Hz, 2H), 4.75 (dq, J=10.3, 6.8 Hz, 1H), 4.70-4.59 (m, 3H), 3.88 (s, 3H), 3.81 (t, J=1.8 Hz, 2H), 3.18 (d, J=17.0 Hz, 1H), 2.73 (d, J=17.1 Hz, 1H), 2.12-1.85 (m, 3H), 1.75 (p, J=7.4 Hz, 2H), 1.56 (ddd, J=15.7, 10.7, 2.2 Hz, 1H), 1.28 (q, J=6.7 Hz, 28H), 0.97-0.84 (m, 3H).

### Example 28: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl acetate (28)

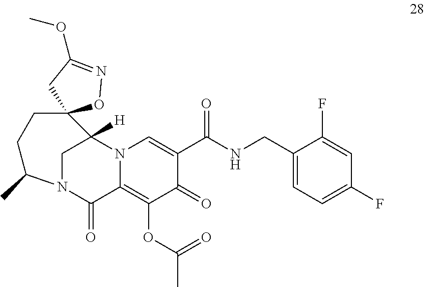 [SMILES: COC1=NO[C@@]2(CC[C@H](C)N3C[C@H]2n2cc(C(=O)NCc4ccc(F)cc4F)c(=O)c(OC(C)=O)c2C3=O)C1]

The title compound was prepared in a manner similar to Example 6, except using acetyl chloride instead of octadecanoyl chloride and CH₂Cl₂ instead of DMF. MS (m/z) 545.12 [M+H]+. ¹H NMR (400 MHz, DMSO) δ 10.17 (t, J=5.9 Hz, 1H), 8.86 (s, 1H), 7.42 (td, J=8.7, 6.6 Hz, 1H), 7.25 (ddd, J=10.6, 9.4, 2.6 Hz, 1H), 7.17-6.97 (m, 1H), 4.77 (s, 1H), 4.70-4.43 (m, 3H), 3.82 (s, 3H), 3.77 (s, 1H), 3.69 (d, J=15.1 Hz, 1H), 3.08 (d, J=16.7 Hz, 1H), 2.79 (d, J=16.8 Hz, 1H), 2.23 (s, 3H), 1.98-1.88 (m, 1H), 1.82 (d, J=8.5 Hz, 2H), 1.37-1.22 (m, 1H), 1.16 (d, J=6.7 Hz, 3H).

### Example 29: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl pentanoate (29)

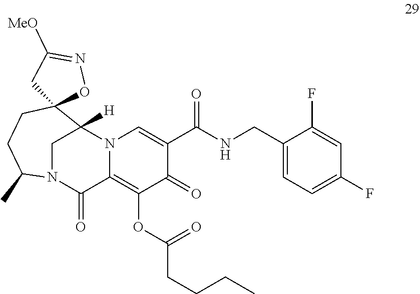 [SMILES: CCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was made following the same method as Example 6, except using pentanoyl chloride instead of octadecanoyl chloride. MS (m/z) 587.7 [M+H]+. 1H NMR (400 MHz, Methanol-d4) δ 10.34 (t, J=5.9 Hz, 1H), 9.46-8.83 (m, 1H), 7.44 (q, J=7.9 Hz, 1H), 6.93 (q, J=10.4, 9.7 Hz, 2H), 4.83-4.68 (m, 2H), 4.68-4.53 (m, 2H), 3.85 (d, J=17.8 Hz, 2H), 3.79 (s, 3H), 3.17 (d, J=17.0 Hz, 1H), 2.70 (d, J=17.0 Hz, 1H), 2.64 (t, J=7.5 Hz, 1H), 1.95 (tdd, J=20.9, 14.1, 6.0 Hz, 3H), 1.71 (p, J=7.4 Hz, 2H), 1.49 (qd, J=15.2, 9.2 Hz, 3H), 1.25 (d, J=6.6 Hz, 3H), 0.97 (t, J=7.3 Hz, 3H).

### Example 30: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl isopropyl carbonate (30)

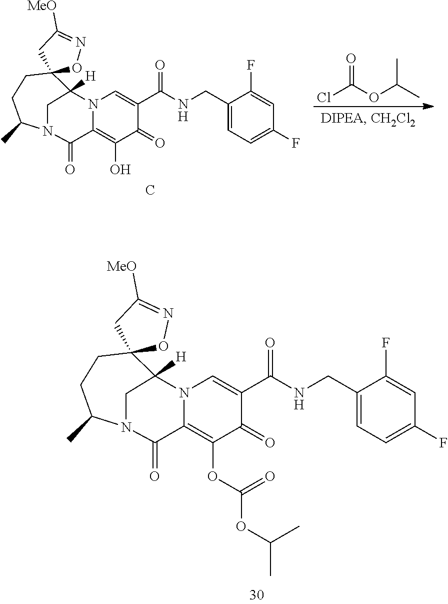

Intermediate C (1.5 g, 2.99 mmol) was dissolved in DCM (30 mL). The mixture was cooled to 0° C. followed by the addition of DIPEA (1.56 ml, 8.96 mmol). Isopropyl chloroformate (732 mg, 5.97 mmol) was added to the mixture slowly. The resulting mixture was stirred at rt overnight. The mixture was diluted with EtOAc, washed with sat. NH₄Cl and brine, dried over sodium sulfate, filtered and concentrated. The resulting residue was purified by column chromatography (0-100% EtOAc/Hexane) to afford the title compound. MS (m/z) 588.80 [M+H]⁺. 1H NMR (400 MHz, Chloroform-d) δ 10.23 (t, J=5.9 Hz, 1H), 8.70 (s, 1H), 7.36 (td, J=8.4, 6.3 Hz, 1H), 6.90-6.63 (m, 2H), 5.02 (hept. J=6.3 Hz, 1H), 4.84 (dp, J=10.0, 6.8 Hz, 1H), 4.71 (dd, J=15.1, 6.2 Hz, 1H), 4.54 (dd, J=15.1, 5.5 Hz, 1H), 4.38 (d, J=2.3 Hz, 1H), 3.87 (s, 3H), 3.79 (dd, J=15.2, 1.8 Hz, 1H), 3.67 (dd, J=15.1, 2.7 Hz, 1H), 3.09 (d, J=17.1 Hz, 1H), 2.66 (d, J=17.1 Hz, 1H), 2.04-1.86 (m, 3H), 1.67-1.61 (m, 1H), 1.43 (t, J=6.6 Hz, 6H), 1.25 (d, J=6.6 Hz, 3H).

### Example 31: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (Z)-tetradec-9-enoate (31)

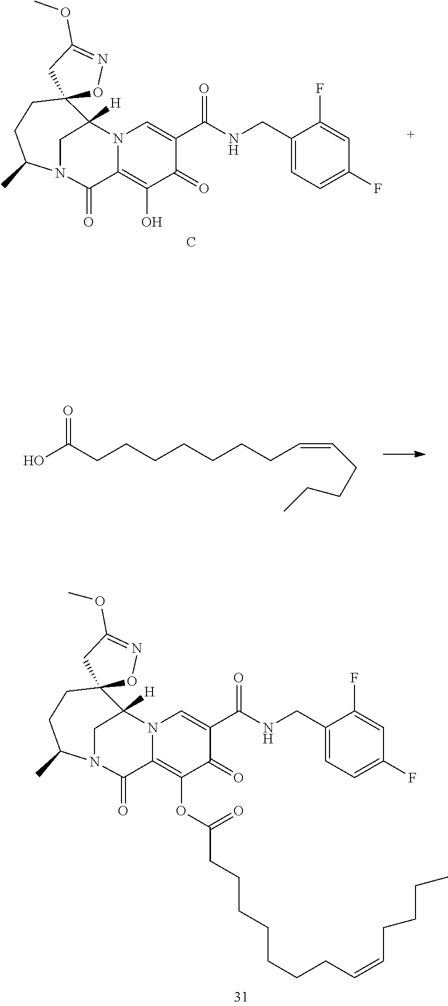

To a mixture of Intermediate C (0.2 g, 0.398 mmol) and (Z)-tetradec-9-enoic acid (225 mg, 0.995 mmol) in DMF (5.0 mL) at room temperature was added 1-(3-dimethylaminopropyl)-3-ethylcarbodiimide hydrochloride (191 mg, 0.995 mmol) followed by DMAP (24.3 mg, 0.199 mmol) and DIPEA (206.0 mg, 1.59 mmol). The resulting mixture was heated at 38° C. for 16 hours. The reaction was then diluted with EtOAc, washed with 5% aq LiCl, brine, dried over sodium sulfate, filtered and concentrated. The residue was purified by silica gel column chromatography, eluting with 0-100% EtOAc/Hexane, to provide the title compound. MS (m/z) 711.341 [M+H]⁺: ¹H NMR (400 MHz, Chloroform-d) δ 10.32 (t, J=5.8 Hz, 1H), 9.17 (s, 1H), 7.34 (td, J=8.4, 6.3 Hz, 1H), 6.88-6.73 (m, 2H), 5.41-5.27 (m, 2H), 4.88-4.67 (m, 3H), 4.47 (dd, J=15.2, 5.2 Hz, 1H), 3.78-3.65 (m, 5H), 3.13 (d, J=16.9 Hz, 1H), 2.65 (t, J=7.7 Hz, 2H), 2.52 (d, J=17.0 Hz, 1H), 2.05-1.85 (m, 7H), 1.82-1.71 (m, 2H), 1.62-1.51 (m, 1H), 1.44-1.29 (m, 12H), 1.24 (d, J=6.7 Hz, 3H), 0.93-0.83 (m, 3H).

### Example 32: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl propionate (32)

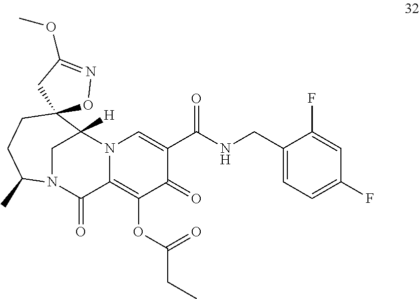 [SMILES: CCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 13, except using propionyl chloride instead of myristoyl chloride, Intermediate C instead of Intermediate B, and CH₂Cl₂ instead of DMF. MS (m/z) 559.207 [M+H]⁺. ¹H NMR (400 MHz, Chloroform-d) δ 10.29 (t, J=5.9 Hz, 1H), 9.08 (s, 1H), 7.34 (q, J=7.8 Hz, 1H), 6.89-6.69 (m, 2H), 4.87-4.64 (m, 3H), 4.57-4.40 (m, 1H), 3.84-3.62 (m, 5H), 3.12 (d, J=16.9 Hz, 1H), 2.69 (q, J=7.5 Hz, 2H), 2.55 (d, J=17.0 Hz, 1H), 2.04-1.84 (m, 4H), 1.31-1.22 (m, 6H).

### Example 33: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl propionate (33)

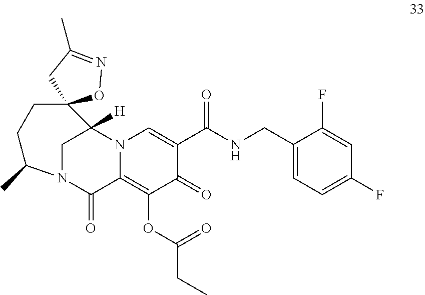 [SMILES: CCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 32 except using Intermediate B instead of Intermediate C. MS (m/z) 543.212 [M+H]⁺. ¹H NMR (400 MHz, Chloroform-d) δ 10.19 (t, J=6.0 Hz, 1H), 8.46 (s, 1H), 7.34 (td. J=8.7, 6.4 Hz, 1H), 6.90-6.75 (m, 2H), 4.80 (dt, J=9.4, 6.5 Hz, 1H), 4.67 (dd, J=15.1, 6.1 Hz, 1H), 4.56 (dd, J=15.1, 5.7 Hz, 1H), 4.14 (d, J=2.2 Hz, 1H), 3.84-3.62 (m, 2H), 3.06 (d, J=17.7 Hz, 1H), 2.71 (q, J=7.5 Hz, 2H), 2.59 (d, J=17.8 Hz, 1H), 2.08 (s, 3H), 1.98-1.82 (m, 4H), 1.31-1.24 (m, 6H).

### Example 34: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl butyrate (34)

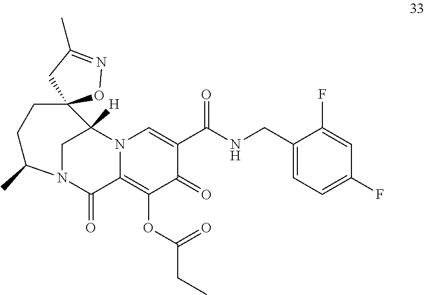 [SMILES: CCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 6, except using butanoyl chloride instead of octadecanoyl chloride. MS (m/z) 573.0 [M+H]⁺. ¹H NMR (400 MHz, Chloroform-d) δ 10.24 (t, J=5.9 Hz, 1H), 8.75 (s, 1H), 7.36 (td, J=8.4, 6.3 Hz, 1H), 6.97-6.67 (m, 2H), 4.95-4.79 (m, 1H), 4.71 (dd, 3=15.2, 6.2 Hz, 1H), 4.54 (dd, 3=15.2, 5.5 Hz, 1H), 4.43 (s, 1H), 3.86 (s, 3H), 3.77 (d, J=1.9 Hz, 1H), 3.72-3.59 (m, 1H), 3.10 (d, J=17.1 Hz, 1H), 2.74-2.52 (m, 3H), 2.03-1.87 (m, 3H), 1.82 (h, J=7.4 Hz, 2H), 1.61 (ddd, J=15.4, 10.6, 2.1 Hz, 1H), 1.28 (d, J=7.2 Hz, 1H), 1.25 (d, J=3.7 Hz, 2H), 1.07 (t, J=7.4 Hz, 3H).

### Example 35: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl butyrate (35)

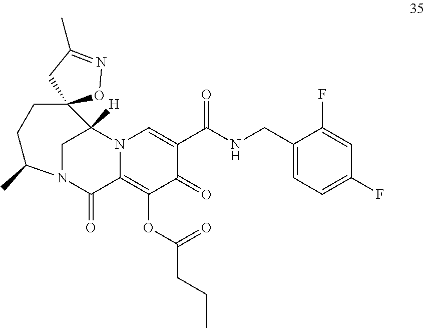 [SMILES: CCCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 6, except using butanoyl chloride instead of octadecanoyl chloride and Intermediate C instead of Intermediate B. MS (m/z) 557.0 [M+H]⁺. ¹H NMR (400 MHz, Chloroform-d) δ 10.19 (t, J=6.0 Hz, 1H), 8.45 (s, 1H), 7.34 (td, J=8.7, 6.4 Hz, 1H), 6.89-6.71 (m, 2H), 4.80 (dp, J=9.9, 6.8 Hz, 1H), 4.67 (dd, J=15.1, 6.2 Hz, 1H), 4.55 (dd, J=15.1, 5.7 Hz, 1H), 3.82-3.57 (m, 2H), 3.09-2.94 (m, 1H), 2.69-2.47 (m, 3H), 2.05 (s, 3H), 1.99-1.85 (m, 3H), 1.80 (p, J=7.4 Hz, 3H), 1.68-1.50 (m, 1H), 1.26-1.22 (m, 3H), 1.05 (t, J=7.4 Hz, 3H).

### Example 36: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl hexanoate (36)

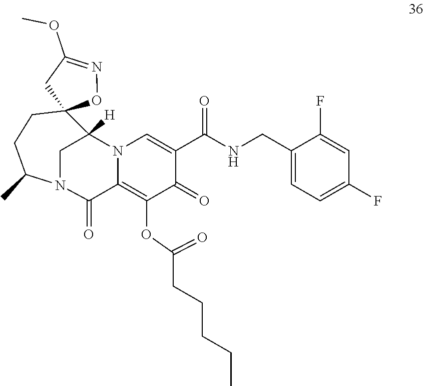 [SMILES: CCCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 6, except using hexanoyl chloride instead of octadecanoyl chloride and CH₂Cl₂ instead of DMF. MS (m/z) 601.4 [M+H]⁺. ¹H NMR (400 MHz, DMSO-d6) δ 10.16 (t, J=6.0 Hz, 1H), 8.86 (s, 1H), 7.41 (td, J=8.7, 6.6 Hz, 1H), 7.24 (ddd, J=10.5, 9.3, 2.6 Hz, 1H), 7.07 (tdd, J=8.5, 2.6, 1.0 Hz, 1H), 4.77 (s, 1H), 4.55 (qt, J=14.9, 6.9 Hz, 3H), 3.82 (s, 3H), 3.80-3.61 (m, 2H), 3.08 (d, J=16.7 Hz, 1H), 2.79 (d, J=16.8 Hz, 1H), 2.58-2.52 (m, 1H), 2.02-1.88 (m, 1H), 1.81 (d, J=8.2 Hz, 2H), 1.62 (p, J=7.4 Hz, 2H), 1.45-1.21 (m, 6H), 1.16 (d, J=6.7 Hz, 3H), 0.88 (t, J=7.0 Hz, 3H).

### Example 37: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl hexanoate (37)

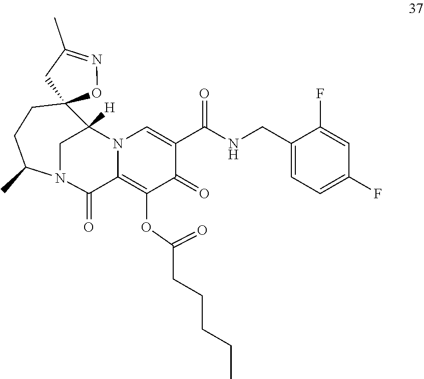 [SMILES: CCCCCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 13, except using hexanoyl chloride instead of myristoyl chloride and CH₂Cl₂ instead of DMF. MS (m/z) 585.1 [M+H]⁺. ¹H NMR (400 MHz, DMSO-d6) δ 10.17 (t, J=5.9 Hz, 1H), 8.81 (s, 1H), 7.41 (td, J=8.7, 6.6 Hz, 1H), 7.25 (ddd, J=10.5, 9.3, 2.6 Hz, 1H), 7.08 (tdd, J=8.6, 2.6, 1.0 Hz, 1H), 4.70 (s, 1H), 4.66-4.43 (m, 3H), 3.87-3.57 (m, 2H), 3.02 (d, J=17.5 Hz, 1H), 2.66 (d, J=17.4 Hz, 1H), 2.57-2.52 (m, 1H), 1.95 (s, 3H), 1.88-1.72 (m, 3H), 1.63 (p, J=7.3 Hz, 2H), 1.50-1.20 (m, 6H), 1.16 (d, J=6.7 Hz, 3H), 0.89 (t, J=7.0 Hz, 3H).

### Example 38: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl heptanoate (38)

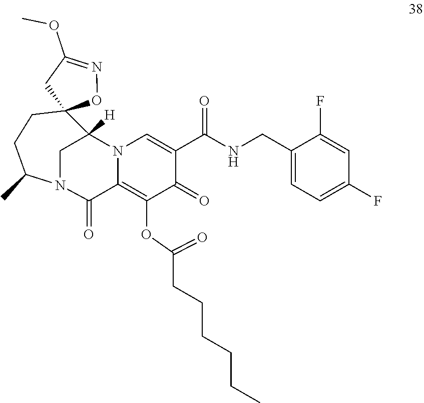 [SMILES: CCCCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 13, except using heptanoyl chloride instead of myristoyl chloride, Intermediate C instead of Intermediate B, and CH₂Cl₂ instead of DMF. MS (m/z) 615.249 [M+H]⁺. ¹H NMR (400 MHz, Chloroform-d) δ 10.27 (t, J=5.8 Hz, 1H), 9.08 (s, 1H), 7.32 (td, J=8.4, 6.3 Hz, 1H), 6.83-6.72 (m, 2H), 4.87-4.64 (m, 3H), 4.45 (dd, J=15.2, 5.3 Hz, 1H), 3.78-3.61 (m, 5H), 3.09 (d, J=16.9 Hz, 1H), 2.63 (dd, J=8.4, 7.0 Hz, 2H), 2.51 (d, J=17.0 Hz, 1H), 2.00-1.81 (m, 3H), 1.75 (q, J=7.6 Hz, 2H), 1.60-1.50 (m, 1H), 1.45-1.34 (m, 2H), 1.33-1.27 (m, 4H), 1.23-1.20 (m, 3H), 0.89-0.82 (m, 3H).

### Example 39: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl heptanoate (39)

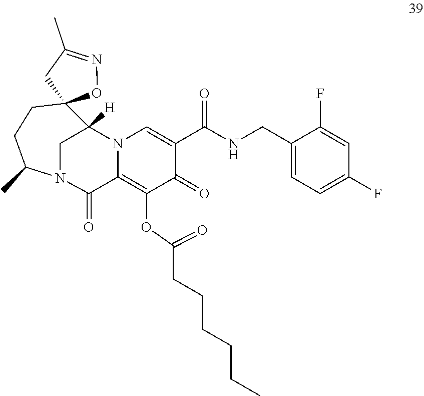 [SMILES: CCCCCCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 38 except that Intermediate B was used instead of Intermediate C. MS (m/z) 598.901 [M+H]⁺. ¹H NMR (400 MHz, Chloroform-d) δ 10.20 (t, J=6.0 Hz, 1H), 8.46 (s, 1H), 7.34 (td, J=8.7, 6.4 Hz, 1H), 6.90-6.70 (m, 2H), 4.79 (dq, J=9.4, 6.8 Hz, 1H), 4.67 (dd, J=15.1, 6.1 Hz, 1H), 4.55 (dd, J=15.2. 5.7 Hz, 1H), 4.14 (d, J=2.3 Hz, 1H), 3.82-3.62 (m, 2H), 3.05 (d, J=17.7 Hz, 1H), 2.66 (t, J=7.7 Hz, 2H), 2.58 (d, J=17.8 Hz, 1H), 2.07 (s, 3H), 1.97-1.89 (m, 2H), 1.88-1.85 (m, 1H), 1.77 (p, J=7.6 Hz, 2H), 1.59 (ddd, J=15.0, 8.9, 3.7 Hz, 1H), 1.46-1.39 (m, 2H), 1.36-1.29 (m, 4H), 1.25 (d, J=6.7 Hz, 3H), 0.93-0.85 (m, 3H).

### Example 40: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl octanoate (40)

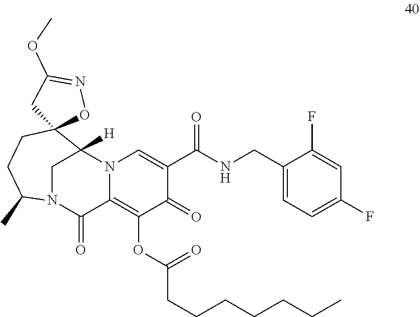 [SMILES: CCCCCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 6, except using octanoyl chloride instead of using octadecanoyl chloride. MS (m/z) 628.9 [M+H]⁺. ¹H NMR (400 MHz, Chloroform-d) δ 10.28 (t, J=5.9 Hz, 1H), 8.94 (s, 1H), 7.35 (td, J=8.3, 6.2 Hz, 1H), 6.88-6.75 (m, 2H), 4.82 (dt, J=10.3, 6.6 Hz, 1H), 4.72 (dd, J=15.2, 6.2 Hz, 1H), 4.59 (s, 1H), 4.51 (dd, J=15.3, 5.3 Hz, 1H), 3.80 (s, 3H), 3.77 (d, J=14.5 Hz, 1H), 3.73-3.64 (m, 1H), 3.11 (d, J=17.0 Hz, 1H), 2.66 (t, J=7.7 Hz, 2H), 2.59 (d, J=17.1 Hz, 1H), 2.08-1.84 (m, 3H), 1.78 (p, J=7.7 Hz, 2H), 1.59 (dd, J=15.9, 11.4 Hz, 1H), 1.43 (dd, J=10.7, 5.3 Hz, 2H), 1.29 (ddd, J=22.4, 14.2, 6.9 Hz, 9H), 0.89 (t, J=6.6 Hz, 3H).

### Example 41: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl octanoate (41)

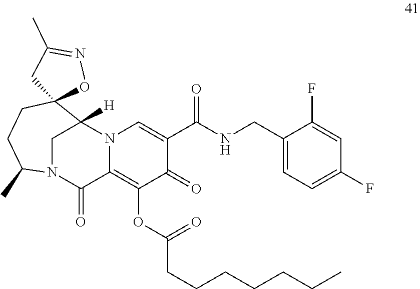 [SMILES: CCCCCCCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 6, except using octanoyl chloride instead of using octadecanoyl chloride and Intermediate B instead of Intermediate C. MS (m/z) 612.9 [M+H]⁺. ¹H NMR (400 MHz, Chloroform-d) δ 10.21 (t, J=6.0 Hz, 1H), 8.43 (s, 1H), 7.35 (td, J=8.7, 6.6 Hz, 1H), 6.89-6.76 (m, 2H), 4.81 (dt, J=9.5, 6.5 Hz, 1H), 4.68 (dd, J=15.1, 6.1 Hz, 1H), 4.57 (dd, J=15.1, 5.7 Hz, 1H), 4.10 (d, J=2.2 Hz, 1H), 3.80 (dd, J=15.2, 1.9 Hz, 1H), 3.69 (d, J=15.2 Hz, 1H), 3.05 (d, J=17.8 Hz, 1H), 2.67 (t, J=7.7 Hz, 2H), 2.60 (d, J=17.8 Hz, 1H), 2.10 (s, 3H), 2.04-1.85 (m, 3H), 1.79 (p, J=7.6 Hz, 2H), 1.67-1.52 (m, 1H), 1.49-1.34 (m, 2H), 1.38-1.22 (m, 9H), 0.89 (t, J=6.7 Hz, 3H).

### Example 42: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl nonanoate (42)

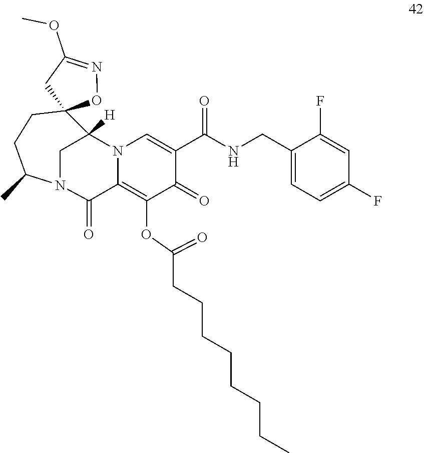 [SMILES: CCCCCCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 31, except using nonanoic acid instead of (Z)-tetradec-9-enoic acid and CH₂C₁₂ instead of DMF. MS (m/z) 643.3 [M+H]⁺. ¹H NMR (400 MHz, DMSO-d6) δ 10.28 (t, J=6.0 Hz, 1H), 8.70 (s, 1H), 7.42 (td, J=8.7, 6.6 Hz, 1H), 7.25 (ddd, J=10.4, 9.3, 2.6 Hz, 1H), 7.13-7.03 (m, 1H), 5.80 (d, J=6.4 Hz, 1H), 5.62 (d, J=6.5 Hz, 1H), 4.69-4.47 (m, 4H), 4.04 (tt, J=6.9, 3.5 Hz, 2H), 3.69 (d, J=2.3 Hz, 2H), 3.01 (d, J=17.5 Hz, 1H), 2.64 (d, J=17.6 Hz, 1H), 1.95 (s, 3H), 1.84-1.71 (m, 3H), 1.56 (p. J=6.7 Hz, 2H), 1.35-1.20 (m, 9H), 1.16 (d, J=6.7 Hz, 3H), 0.90-0.81 (m, 3H).

### Example 43: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl nonanoate (43)

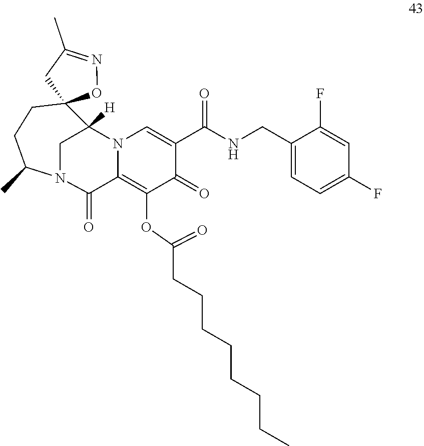 [SMILES: CCCCCCCCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 31, except using nonanoic acid instead of (Z)-tetradec-9-enoic acid, Intermediate B instead of Intermediate C, and CH₂Cl₂ instead of DMF. MS (m/z) 627.2 [M+H]⁺. ¹H NMR (400 MHz, DMSO-d6) δ 10.17 (t, J=6.0 Hz, 1H), 8.81 (s, 1H), 7.41 (td, J=8.7, 6.6 Hz, 1H), 7.24 (ddd, J=10.5, 9.3, 2.6 Hz, 1H), 7.16-6.98 (m, 1H), 4.69 (d, J=2.3 Hz, 1H), 4.66-4.40 (m, 3H), 3.89-3.58 (m, 2H), 3.02 (d, J=17.4 Hz, 1H), 2.66 (d, J=17.4 Hz, 1H), 2.53 (d, J=7.6 Hz, 1H), 1.95 (s, 3H), 1.89-1.71 (m, 3H), 1.62 (p, J=7.4 Hz, 2H), 1.38 (t, J=7.5 Hz, 2H), 1.27 (q, J=5.4 Hz, 10H), 1.16 (d, J=6.7 Hz, 3H), 0.95-0.70 (m, 3H).

### Example 44: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl decanoate (44)

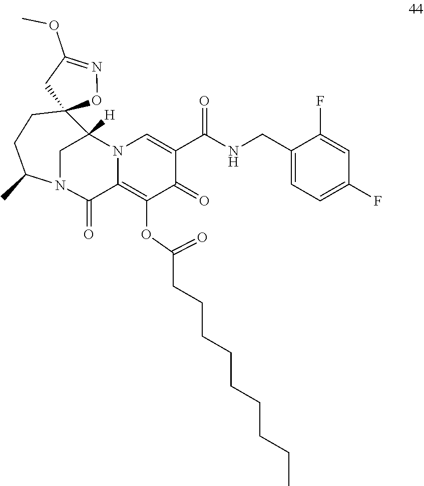 [SMILES: CCCCCCCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 13, except using decanoyl chloride instead of myristoyl chloride and CH₂Cl₂ instead of DMF. MS (m/z) 657.292 [M+H]⁺. ¹H NMR (400 MHz, Chloroform-d) δ 10.24 (t, J=6.0 Hz, 1H), 8.72 (s, 1H), 7.36 (td, J=8.4, 6.3 Hz, 1H), 6.91-6.71 (m, 2H), 4.82 (dt, J=10.2, 6.8 Hz, 1H), 4.71 (dd, J=15.1, 6.1 Hz, 1H), 4.55 (dd, J=15.2, 5.5 Hz, 1H), 4.40 (s, 1H), 3.87 (s, 3H), 3.82-3.63 (m, 2H), 3.09 (d, J=17.1 Hz, 1H), 2.73-2.57 (m, 3H), 2.08-1.87 (m, 3H), 1.78 (p, J=7.7 Hz, 2H), 1.64-1.56 (m, 1H), 1.44-1.23 (m, 15H), 0.93-0.85 (m, 3H).

### Example 45: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl decanoate (45)

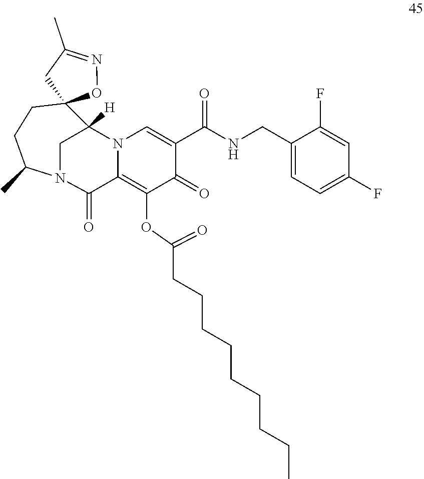 [SMILES: CCCCCCCCCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 44 except Intermediate B was used instead of Intermediate C. MS (m/z) 640.930 [M+H]⁺. ¹H NMR (400 MHz, Chloroform-d) δ 10.20 (t, J=6.0 Hz, 1H), 8.49 (s, 1H), 7.33 (td, J=8.7, 6.3 Hz, 1H), 6.85-6.75 (m, 2H), 4.78 (dq, J=9.9, 6.8 Hz, 1H), 4.66 (dd, J=15.2, 6.2 Hz, 1H), 4.54 (dd, J=15.2, 5.7 Hz, 1H), 4.18 (d, J=2.2 Hz, 1H), 3.71 (qd, J=15.0, 2.3 Hz, 2H), 3.04 (d, J=17.8 Hz, 1H), 2.64 (t, J=7.7 Hz, 2H), 2.56 (d, J=17.7 Hz, 1H), 2.05 (s, 3H), 1.97-1.83 (m, 3H), 1.77 (q, J=7.6 Hz, 2H), 1.58 (ddd, J=15.1, 9.1, 3.8 Hz, 1H), 1.44-1.21 (m, 15H), 0.91-0.83 (m, 3H).

### Example 46: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl dodecanoate (46)

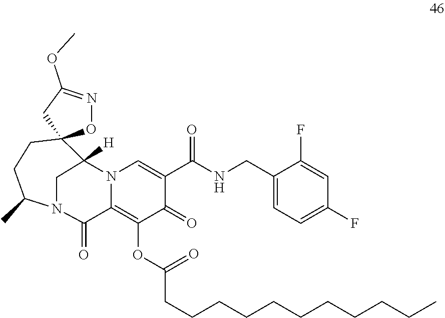 [SMILES: CCCCCCCCCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 6, except using dodecanoyl chloride instead of octadecanoyl chloride. MS (m/z) 684.9 [M+H]⁺. ¹H NMR (400 MHz, Chloroform-d) δ 10.27 (t, J=6.0 Hz, 1H), 8.89 (s, 1H), 7.36 (q, J=7.8 Hz, 1H), 6.82 (q, J=10.5, 10.0 Hz, 2H), 4.82 (q, J=7.9 Hz, 1H), 4.72 (dd, J=15.3, 6.2 Hz, 1H), 4.58-4.47 (m, 2H), 3.82-3.69 (m, 5H), 3.11 (d, J=17.0 Hz, 1H), 2.66 (t, J=7.7 Hz, 2H), 2.60 (d, J=17.0 Hz, 1H), 1.96 (qd, J=16.4, 15.3, 6.4 Hz, 3H), 1.76 (dd, J=18.3, 10.7 Hz, 3H), 1.60 (dd, J=15.3, 10.9 Hz, 1H), 1.29 (d, J=15.0 Hz, 18H), 0.89 (t, J=6.6 Hz, 3H).

### Example 47: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl dodecanoate (47)

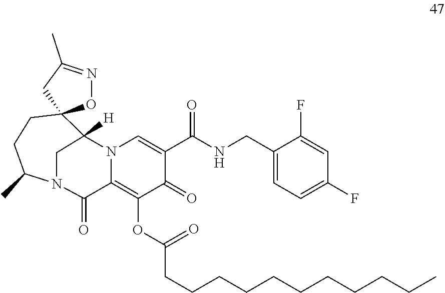 [SMILES: CCCCCCCCCCCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 6, except using dodecanoyl chloride instead of octadecanoyl chloride and Intermediate B instead of Intermediate C. MS (m/z) 669.9 [M+H]⁺. ¹H NMR (400 MHz, Chloroform-d) δ 10.20 (m, 1H), 8.52 (m, 1H), 7.32 (m, 1H), 6.80 (m, 2H), 4.78-4.21 (m, 4H), 3.71 (m, 2H), 3.04-2.58 (m, 4H), 2.03 (m, 3H), 1.89 (m, 4H), 1.25 (m, 21H), 0.86 (m, 3H).

### Example 48: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl isobutyrate (48)

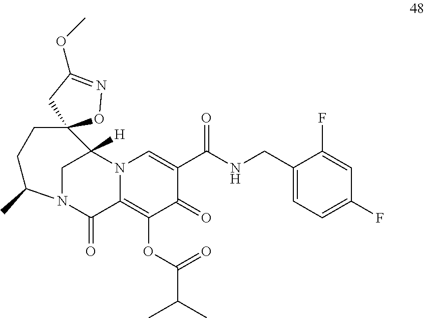 [SMILES: COC1=NO[C@@]2(CC[C@H](C)N3C[C@H]2n2cc(C(=O)NCc4ccc(F)cc4F)c(=O)c(OC(=O)C(C)C)c2C3=O)C1]

The title compound was prepared in a manner similar to Example 13, except using isobutyryl chloride instead of myristoyl chloride and Intermediate C instead of Intermediate B. MS (m/z) 573.0 [M+H]⁺. ¹H NMR (400 MHz, MeOD) δ 8.70 (s, 1H), 7.55-7.35 (m, 1H), 7.09-6.83 (m, 2H), 4.81-4.70 (m, 1H), 4.70-4.57 (m, 2H), 3.93-3.89 (m, 3H), 3.15-2.99 (m, 1H), 2.96-2.83 (m, 1H), 2.83-2.68 (m, 1H), 2.09-2.00 (m, 2H), 1.98-1.84 (m, 2H), 1.66-1.50 (m, 1H), 1.37-1.29 (m, 6H), 1.29-1.24 (m, 3H), 1.17-1.11 (m, 3H).

### Example 49: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl isobutyrate (49)

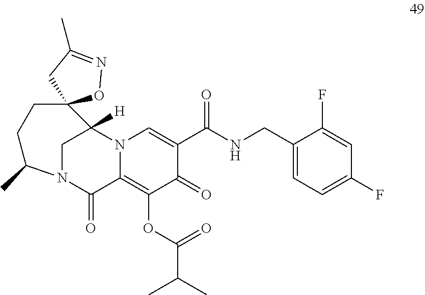 [SMILES: CC1=NO[C@@]2(CC[C@H](C)N3C[C@H]2n2cc(C(=O)NCc4ccc(F)cc4F)c(=O)c(OC(=O)C(C)C)c2C3=O)C1]

The title compound was prepared in a manner similar to Example 13, except using isobutyryl chloride instead of myristoyl chloride. MS (m/z) 557.0 [M+H]⁺. ¹H NMR (400 MHz, MeOD) δ 8.65 (s, 1H), 7.60-7.32 (m, 1H), 7.03-6.89 (m, 2H), 4.82-4.69 (m, 1H), 4.69-4.59 (m, 2H), 4.50 (s, 1H), 3.91-3.65 (m, 2H), 3.24-3.04 (m, 1H), 3.00-2.81 (m, 1H), 2.78-2.63 (m, 1H), 2.13-2.00 (m, 3H), 2.00-1.85 (m, 3H), 1.65-1.43 (m, 1H), 1.36-1.28 (m, 7H), 1.28-1.23 (m, 3H).

### Example 50: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-methylbutanoate (50)

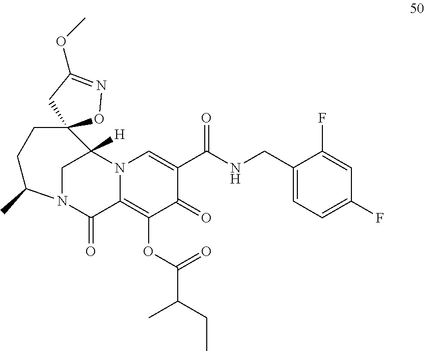 [SMILES: CCC(C)C(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 13, except using 2-methylbutanoyl chloride instead of myristoyl chloride and Intermediate C instead of Intermediate B. MS (m/z) 587.0 [M+H]⁺. ¹H NMR (400 MHz, MeOD) δ 8.70 (s, 1H), 7.53-7.37 (m, 1H), 7.09-6.89 (m, 2H), 4.79-4.71 (m, 1H), 4.67-4.63 (m, 3H), 3.91 (s, 4H), 3.89-3.72 (m, 2H), 3.25-3.09 (m, 1H), 2.83-2.63 (m, 2H), 2.18-1.79 (m, 4H), 1.79-1.46 (m, 2H), 1.36-1.23 (m, 6H), 1.10-1.02 (m, 3H).

### Example 51: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 2-methylbutanoate (51)

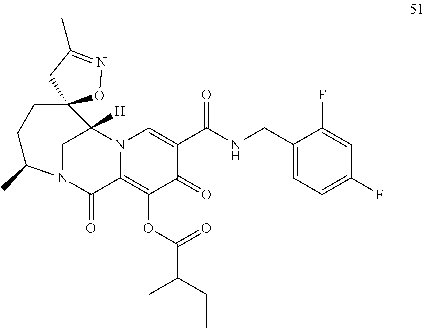 [SMILES: CCC(C)C(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 13, except using 2-methylbutanoyl chloride instead of myristoyl chloride. MS (m/z) 571.1 [M+H]⁺. ¹H NMR (400 MHz, MeOD) δ 8.63 (s, 1H), 7.51-7.33 (m, 1H), 7.09-6.80 (m, 2H), 4.78-4.39 (m, 4H), 3.93-3.72 (m, 2H), 3.19-3.10 (m, 1H), 2.87-2.64 (m, 2H), 2.12-2.01 (m, 3H), 2.01-1.76 (m, 5H), 1.76-1.46 (m, 2H), 1.39-1.19 (m, 6H), 1.11-0.97 (m, 3H).

### Example 52: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl cyclopentanecarboxylate (52)

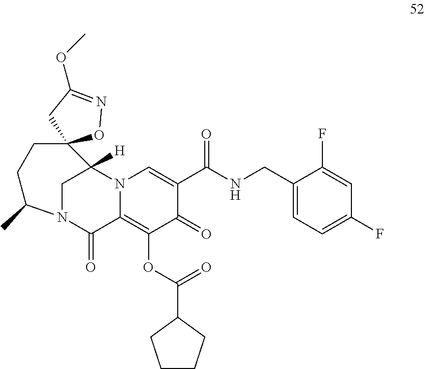 [SMILES: COC1=NO[C@@]2(CC[C@H](C)N3C[C@H]2n2cc(C(=O)NCc4ccc(F)cc4F)c(=O)c(OC(=O)C4CCCC4)c2C3=O)C1]

The title compound was prepared in a manner similar to Example 13, except using cyclopentanecarbonyl chloride instead of myristoyl chloride and Intermediate C instead of Intermediate B. MS (m/z) 599.0 [M+H]⁺. ¹H NMR (400 MHz, DMSO) δ 10.15 (t, J=5.9 Hz, 1H), 8.86 (s, 1H), 7.49-7.30 (m, 1H), 7.30-7.17 (m, 1H), 7.17-6.96 (m, 1H), 4.77 (s, 1H), 4.62-4.46 (m, 4H), 3.82 (s, 3H), 3.80-3.58 (m, 1H), 3.12-2.93 (m, 2H), 2.87-2.72 (m, 1H), 2.07-1.73 (m, 3H), 1.68-1.54 (m, 5H), 1.40-1.22 (m, 4H), 1.22-1.04 (m, 3H).

### Example 53: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl cyclopentanecarboxylate (53)

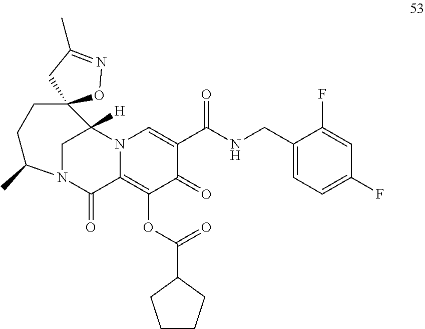 [SMILES: CC1=NO[C@@]2(CC[C@H](C)N3C[C@H]2n2cc(C(=O)NCc4ccc(F)cc4F)c(=O)c(OC(=O)C4CCCC4)c2C3=O)C1]

The title compound was prepared in a manner similar to Example 13, except using cyclopentanecarbonyl chloride instead of myristoyl chloride. MS (m/z) 582.9 [M+H]⁺. ¹H NMR (400 MHz, DMSO) δ 10.15 (t, J=5.9 Hz, 1H), 8.86 (s, 1H), 7.49-7.30 (m, 1H), 7.30-7.17 (m, 1H), 7.17-6.96 (m, 1H), 4.77 (s, 1H), 4.62-4.46 (m, 4H), 3.82 (s, 3H), 3.80-3.58 (m, 1H), 3.12-2.93 (m, 2H), 2.87-2.72 (m, 1H), 2.07-1.73 (m, 3H), 1.68-1.54 (m, 5H), 1.40-1.22 (m, 4H), 1.22-1.04 (m, 3H).

### Example 54: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 3-methylbutanoate (54)

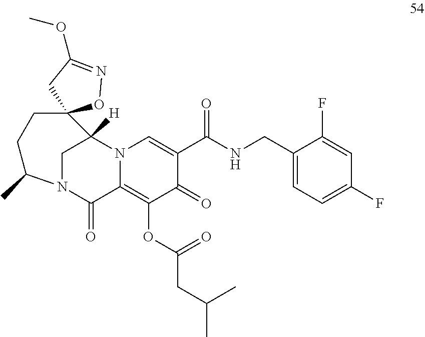 [SMILES: COC1=NO[C@@]2(CC[C@H](C)N3C[C@H]2n2cc(C(=O)NCc4ccc(F)cc4F)c(=O)c(OC(=O)CC(C)C)c2C3=O)C1]

The title compound was prepared in a manner similar to Example 6, except using 3-methylbutanoyl chloride instead of octadecanoyl chloride and CH₂Cl₂ instead of DMF. MS (m/z) 587.2 [M+H]⁺. ¹H NMR (400 MHz, DMSO-d6) δ 10.16 (t, J=6.0 Hz, 1H), 8.86 (s, 1H), 7.41 (td, J=8.7, 6.6 Hz, 1H), 7.24 (ddd, J=10.5, 9.3, 2.6 Hz, 1H), 7.07 (tdd, J=8.6, 2.9, 1.0 Hz, 1H), 4.77 (s, 1H), 4.68-4.39 (m, J=6.1 Hz, 3H), 3.82 (s, 3H), 3.81-3.61 (m, 2H), 3.08 (d, J=16.8 Hz, 1H), 2.79 (d, J=16.7 Hz, 1H), 2.46-2.31 (m, 2H), 2.09 (hept, J=6.7 Hz, 1H), 2.01-1.87 (m, 1H), 1.78 (q, J=14.6, 11.6 Hz, 2H), 1.26 (d, J=13.3 Hz, 1H), 1.16 (d, J=6.7 Hz, 3H), 1.01 (dd, J=6.7, 2.8 Hz, 6H).

### Example 55: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 3-methylbutanoate (55)

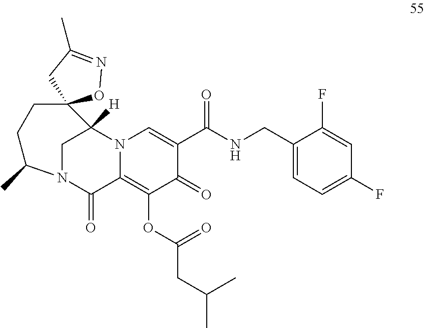 [SMILES: CC1=NO[C@@]2(CC[C@H](C)N3C[C@H]2n2cc(C(=O)NCc4ccc(F)cc4F)c(=O)c(OC(=O)CC(C)C)c2C3=O)C1]

The title compound was prepared in a manner similar to Example 13, except using 3-methylbutanoyl chloride instead of myristoyl chloride and CH₂Cl₂ instead of DMF. MS (m/z) 571.4 [M+H]⁺. ¹H NMR (400 MHz, DMSO-d6) δ 10.16 (t, J=6.0 Hz, 1H), 8.82 (s, 1H), 7.41 (td, J=8.7, 6.6 Hz, 1H), 7.24 (ddd, J=10.5, 9.3, 2.6 Hz, 1H), 7.08 (tdd, J=8.6, 2.6, 1.0 Hz, 1H), 4.69 (s, 1H), 4.55 (qd, J=14.8, 5.6 Hz, 3H), 3.87-3.61 (m, 2H), 3.02 (d, J=17.4 Hz, 1H), 2.67 (d, J=17.4 Hz, 1H), 2.47-2.33 (m, 2H), 2.09 (dt, J=13.5, 6.8 Hz, 1H), 1.95 (s, 3H), 1.89-1.70 (m, 3H), 1.33-1.11 (m, 4H), 1.02 (dd, J=6.7, 2.9 Hz, 6H).

### Example 56: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 4-methylpentanoate (56)

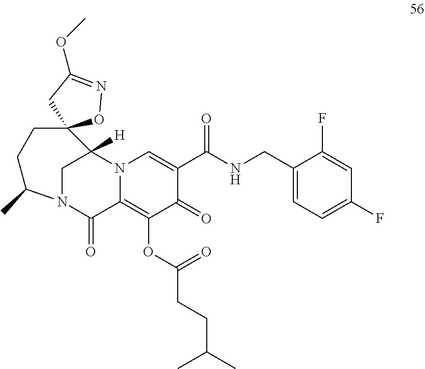 [SMILES: COC1=NO[C@@]2(CC[C@H](C)N3C[C@H]2n2cc(C(=O)NCc4ccc(F)cc4F)c(=O)c(OC(=O)CCC(C)C)c2C3=O)C1]

The title compound was prepared in a manner similar to Example 13, except using 4-methylpentanoyl chloride instead of myristoyl chloride and Intermediate C instead of Intermediate B. MS (m/z) 601.0 [M+H]⁺. ¹H NMR (400 MHz, DMSO) δ 10.26-10.07 (m, 1H), 8.86 (s, 1H), 7.50-7.35 (m, 1H), 7.35-7.16 (m, 1H), 7.16-6.87 (m, 1H), 4.77 (s, 1H), 4.69-4.43 (m, 3H), 3.82 (s, 5H), 3.07 (d, J=16.7 Hz, 1H), 2.79 (d, J=16.8 Hz, 1H), 1.97-1.88 (m, 2H), 1.85-1.73 (m, 3H), 1.73-1.60 (m, 1H), 1.60-1.43 (m, 2H), 1.42-1.20 (m, 1H), 1.16 (d, J=6.7 Hz, 3H), 0.90 (d, J=6.6 Hz, 6H).

### Example 57: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 4-methylpentanoate (57)

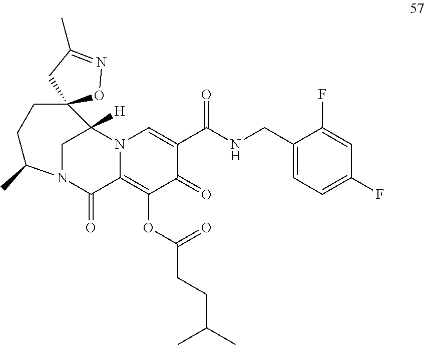 [SMILES: CC1=NO[C@@]2(CC[C@H](C)N3C[C@H]2n2cc(C(=O)NCc4ccc(F)cc4F)c(=O)c(OC(=O)CCC(C)C)c2C3=O)C1]

The title compound was prepared in a manner similar to Example 13, except using 4-methylpentanoyl chloride instead of myristoyl chloride. MS (m/z) 585.0 [M+H]⁺. ¹H NMR (400 MHz, DMSO) δ 10.26-10.08 (m, 1H), 8.81 (s, 1H), 7.48-7.33 (m, 1H), 7.32-7.17 (m, 1H), 7.17-6.90 (m, 1H), 4.70 (s, 1H), 4.64-4.43 (m, 3H), 3.90-3.59 (m, 2H), 3.02 (d, J=17.4 Hz, 1H), 2.66 (d, J=17.4 Hz, 1H), 1.95 (s, 3H), 1.84-1.74 (m, 4H), 1.74-1.59 (m, 1H), 1.58-1.48 (m, 3H), 1.29-1.10 (m, 4H), 0.90 (d, J=6.5 Hz, 6H).

### Example 58: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 3-cyclopentylpropanoate (58)

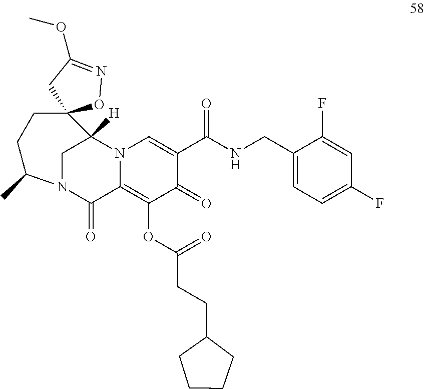 [SMILES: COC1=NO[C@@]2(CC[C@H](C)N3C[C@H]2n2cc(C(=O)NCc4ccc(F)cc4F)c(=O)c(OC(=O)CCC4CCCC4)c2C3=O)C1]

The title compound was prepared in a manner similar to Example 6, except using 3-cyclopentylpropanoyl chloride instead of octadecanoyl chloride and CH₂Cl₂ instead of DMF. MS (m/z) 627.4 [M+H]⁺. 1H NMR (400 MHz, DMSO-d6) δ 10.16 (t, J=6.0 Hz, 1H), 8.86 (s, 1H), 7.41 (td, J=8.7, 6.6 Hz, 1H), 7.24 (ddd, J=10.5, 9.3, 2.6 Hz, 1H), 7.07 (tdd, J=8.5, 2.6, 1.0 Hz, 1H), 4.77 (s, 1H), 4.69-4.42 (m, 4H), 3.82 (s, 5H), 3.07 (d, J=16.7 Hz, 1H), 2.78 (d, J=16.8 Hz, 1H), 2.01-1.70 (m, 7H), 1.70-1.39 (m, 6H), 1.40-0.99 (m, 6H).

### Example 59: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 3-cyclopentylpropanoate (59)

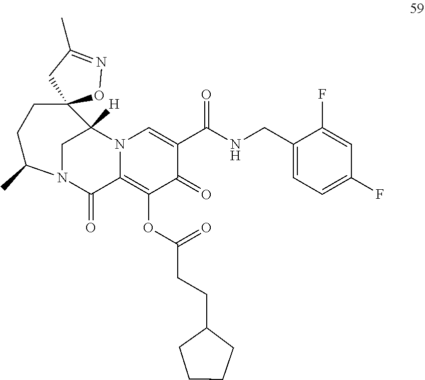 [SMILES: CC1=NO[C@@]2(CC[C@H](C)N3C[C@H]2n2cc(C(=O)NCc4ccc(F)cc4F)c(=O)c(OC(=O)CCC4CCCC4)c2C3=O)C1]

The title compound was prepared in a manner similar to Example 13, except using 3-cyclopentylpropanoyl chloride instead of myristoyl chloride and CH₂Cl₂ instead of DMF. MS (m/z) 611.2 [M+H]⁺. 1H NMR (400 MHz, DMSO-d6) δ 10.16 (t, J=6.0 Hz, 1H), 8.82 (s, 1H), 7.41 (td, 3=8.7, 6.6 Hz, 1H), 7.24 (ddd, 3=10.4, 9.3, 2.6 Hz, 1H), 7.08 (tdd, 3=8.5, 2.6, 1.0 Hz, 1H), 4.69 (d, J=2.4 Hz, 1H), 4.66-4.42 (m, 4H), 3.92-3.59 (m, 2H), 3.02 (d, J=17.5 Hz, 1H), 2.66 (d, J=17.4 Hz, 1H), 1.95 (s, 3H), 1.91-1.70 (m, 6H), 1.70-1.37 (m, 6H), 1.36-0.95 (m, 7H).

### Example 60: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 5-methylhexanoate (60)

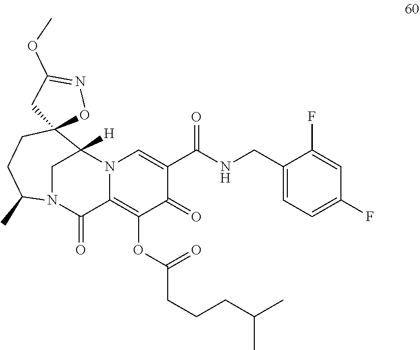 [SMILES: COC1=NO[C@@]2(CC[C@H](C)N3C[C@H]2n2cc(C(=O)NCc4ccc(F)cc4F)c(=O)c(OC(=O)CCCC(C)C)c2C3=O)C1]

The title compound was prepared in a manner similar to Example 6, except using 5-methylhexanoyl chloride instead of octadecanoyl chloride. MS (m/z) 614.9 [M+H]⁺. 1H NMR (400 MHz, Chloroform-d) δ 10.28 (t, J=5.9 Hz, 1H), 8.95 (s, 1H), 7.35 (td, J=8.4, 6.4 Hz, 1H), 6.88-6.75 (m, 2H), 4.89-4.75 (m, 1H), 4.72 (dd, J=15.2, 6.2 Hz, 1H), 4.60 (s, 1H), 4.51 (dd, J=15.3, 5.3 Hz, 1H), 3.82-3.64 (m, 5H), 3.11 (d, J=17.0 Hz, 1H), 2.96-2.89 (m, 1H), 2.65 (dd, J=8.5, 6.9 Hz, 2H), 2.58 (d, J=17.0 Hz, 1H), 1.96 (ddt, J=19.5, 10.6, 4.2 Hz, 2H), 1.84-1.67 (m, 2H), 1.66-1.47 (m, 2H), 1.38-1.27 (m, 2H), 1.26 (t, J=6.8 Hz, 3H), 0.91 (d, J=6.6 Hz, 6H).

### Example 61: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 5-methylhexanoate (61)

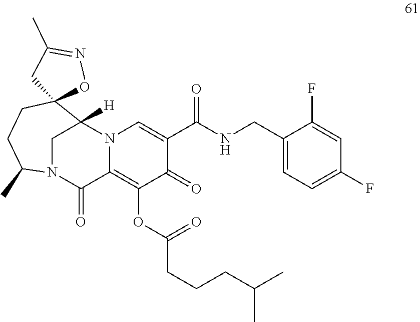 [SMILES: CC1=NO[C@@]2(CC[C@H](C)N3C[C@H]2n2cc(C(=O)NCc4ccc(F)cc4F)c(=O)c(OC(=O)CCCC(C)C)c2C3=O)C1]

The title compound was prepared in a manner similar to Example 6, except using 5-methylhexanoyl chloride instead of octadecanoyl chloride, and Intermediate B instead of Intermediate C. MS (m/z) 598.9 [M+H]⁺. ¹H NMR (400 MHz, Chloroform-d) δ 10.20 (t, J=5.9 Hz, 1H), 8.47 (s, 1H), 7.33 (td, J=8.7, 6.4 Hz, 1H), 6.87-6.74 (m, 2H), 4.79 (dt, J=9.5, 6.4 Hz, 1H), 4.66 (dd, J=15.1, 6.1 Hz, 1H), 4.54 (dd, J=15.4, 5.6 Hz, 1H), 4.16 (d, J=2.7 Hz, 1H), 3.76 (dd, J=15.1, 1.9 Hz, 1H), 3.73-3.63 (m, 1H), 3.05 (d, J=17.7 Hz, 1H), 2.95 (s, 1H), 2.87 (s, 1H), 2.64 (t, J=7.7 Hz, 2H), 2.57 (d, J=17.8 Hz, 1H), 2.06 (s, 3H), 1.98-1.82 (m, 3H), 1.76 (h, J=6.9, 6.2 Hz, 2H), 1.66-1.51 (m, 2H), 1.24 (d, J=6.7 Hz, 3H), 0.90 (d, J=6.6 Hz, 6H).

### Example 62: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 6-methylheptanoate (62)

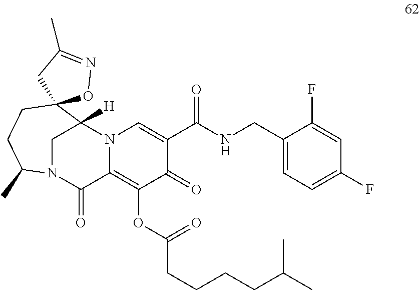 [SMILES: CC1=NO[C@@]2(CC[C@H](C)N3C[C@H]2n2cc(C(=O)NCc4ccc(F)cc4F)c(=O)c(OC(=O)CCCCC(C)C)c2C3=O)C1]

The title compound was prepared in a manner similar to Example 31, except using 6-methylheptanoic acid instead of (Z)-tetradec-9-enoic acid and Intermediate B instead of Intermediate C. MS (m/z) 613.0 [M+H]⁺. ¹H NMR (400 MHz, DMSO) δ 10.26-10.09 (m, 114), 8.81 (s, 1H), 7.46-7.36 (m, 1H), 7.36-7.18 (m, 1H), 7.18-7.00 (m, 1H), 4.78-4.65 (m, 1H), 4.65-4.47 (m, 3H), 4.03-3.59 (m, 2H), 3.02 (d, J=17.5 Hz, 1H), 2.66 (d, J=17.2 Hz, 1H), 1.95 (s, 3H), 1.89-1.72 (m, 4H), 1.72-1.47 (m, 3H), 1.50-1.31 (m, 3H), 1.31-1.11 (m, 6H), 0.87 (d, J=6.6 Hz, 6H).

### Example 63: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 6-methylheptanoate (63)

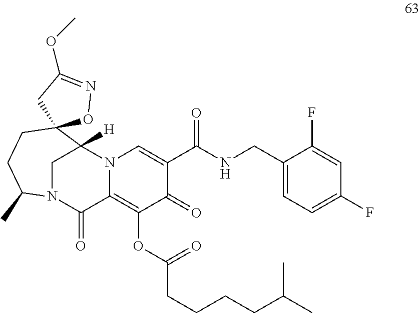 [SMILES: COC1=NO[C@@]2(CC[C@H](C)N3C[C@H]2n2cc(C(=O)NCc4ccc(F)cc4F)c(=O)c(OC(=O)CCCCC(C)C)c2C3=O)C1]

The title compound was prepared in a manner similar to Example 31, except using 6-methylheptanoic acid instead of (Z)-tetradec-9-enoic acid. MS (m/z) 629.0 [M+H]⁺. ¹H NMR (400 MHz, DMSO) δ 10.27-10.00 (m, 1H), 8.86 (s, 1H), 7.58-7.33 (m, 1H), 7.33-7.15 (m, 1H), 7.15-6.91 (m, 1H), 4.77 (s, 1H), 4.72-4.40 (m, 4H), 3.92-3.53 (m, 5H), 3.08 (d, J=16.7 Hz, 1H), 2.78 (d, J=16.8 Hz, 1H), 2.06-1.71 (m, 3H), 1.71-1.44 (m, 3H), 1.44-1.12 (m, 9H), 0.89-0.82 (m, 6H).

### Example 64: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 7-methyloctanoate (64)

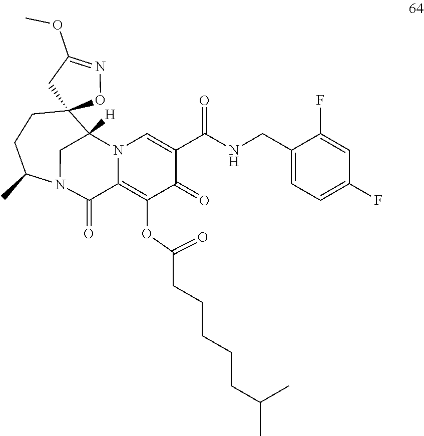 [SMILES: COC1=NO[C@@]2(CC[C@H](C)N3C[C@H]2n2cc(C(=O)NCc4ccc(F)cc4F)c(=O)c(OC(=O)CCCCCC(C)C)c2C3=O)C1]

The title compound was prepared in a manner similar to Example 31, except using 7-methyloctanoic acid instead of (Z)-tetradec-9-enoic acid and CH₂Cl₂ instead of DMF. MS (m/z) 643.3 [M+H]⁺. ¹H NMR (400 MHz, DMSO-d6) δ 10.16 (t, J=6.0 Hz, 1H), 8.86 (s, 1H), 7.41 (td, J=8.7, 6.6 Hz, 1H), 7.24 (ddd, J=10.5, 9.3, 2.6 Hz, 1H), 7.12-7.02 (m, 1H), 4.77 (s, 1H), 4.65-4.46 (m, 3H), 3.82 (s, 3H), 3.77 (s, 1H), 3.68 (dd, J=15.2, 1.9 Hz, 1H), 3.08 (d, J=16.7 Hz, 1H), 2.78 (d, J=16.8 Hz, 1H), 2.04-1.45 (m, 7H), 1.42-1.23 (m, 5H), 1.17 (t, J=6.5 Hz, 6H), 0.86 (d, J=6.6 Hz, 6H).

### Example 65: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 7-methyloctanoate (65)

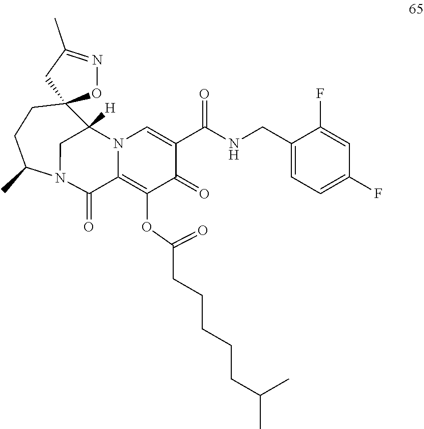 [SMILES: CC1=NO[C@@]2(CC[C@H](C)N3C[C@H]2n2cc(C(=O)NCc4ccc(F)cc4F)c(=O)c(OC(=O)CCCCCC(C)C)c2C3=O)C1]

The title compound was prepared in a manner similar to Example 31, except using 7-methyloctanoic acid instead of (Z)-tetradec-9-enoic acid, Intermediate B instead of Intermediate C, and CH₂Cl₂ instead of DMF. MS (m/z) 627.3 [M+H]⁺. ¹H NMR (400 MHz, DMSO-d6) δ 10.17 (t, J=5.9 Hz, 1H), 8.81 (s, 1H), 7.41 (td, J=8.7, 6.6 Hz, 1H), 7.24 (ddd, J=10.4, 9.3, 2.6 Hz, 1H), 7.08 (tdd, J=8.6, 2.6, 1.0 Hz, 1H), 4.70 (s, 1H), 4.66-4.38 (m, 3H), 3.88-3.51 (m, 2H), 3.02 (d, J=17.4 Hz, 1H), 2.66 (d, J=17.4 Hz, 1H), 2.53 (d, J=3.4 Hz, 1H), 1.95 (s, 3H), 1.79 (q, J=10.8, 10.0 Hz, 3H), 1.70-1.59 (m, 2H), 1.53 (qd, J=13.2, 12.3, 5.7 Hz, 1H), 1.43-1.22 (m, 5H), 1.16 (p, J=5.8 Hz, 6H), 0.86 (d, J=6.6 Hz, 6H).

### Example 66: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 8-methylnonanoate (66)

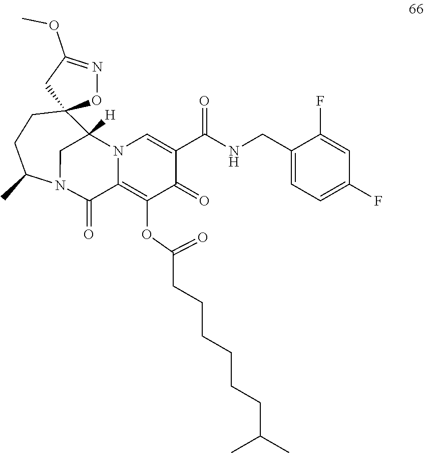 [SMILES: COC1=NO[C@@]2(CC[C@H](C)N3C[C@H]2n2cc(C(=O)NCc4ccc(F)cc4F)c(=O)c(OC(=O)CCCCCCC(C)C)c2C3=O)C1]

The title compound was prepared in a manner similar to Example 31, except using 8-methylnonanoic acid instead of (Z)-tetradec-9-enoic acid and CH₂Cl₂ instead of DMF. MS (m/z) 657.5 [M+H]⁺. ¹H NMR (400 MHz, DMSO-d6) δ 10.16 (t, J=6.0 Hz, 1H), 8.86 (s, 1H), 7.41 (td, J=8.7, 6.6 Hz, 1H), 7.24 (ddd, J=10.5, 9.3, 2.6 Hz, 1H), 7.07 (tdd, J=8.6, 2.6, 1.0 Hz, 1H), 4.77 (s, 1H), 4.55 (qt, J=14.9, 6.7 Hz, 4H), 3.82 (s, 5H), 3.08 (d, J=16.7 Hz, 1H), 2.78 (d, J=16.8 Hz, 1H), 1.86 (dt, J=50.6, 8.0 Hz, 3H), 1.55 (ddq, J=39.8, 13.2, 6.9 Hz, 3H), 1.44-1.22 (m, 8H), 1.16 (d, J=6.6 Hz, 5H), 0.85 (d, J=6.6 Hz, 6H).

### Example 67: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 8-methylnonanoate (67)

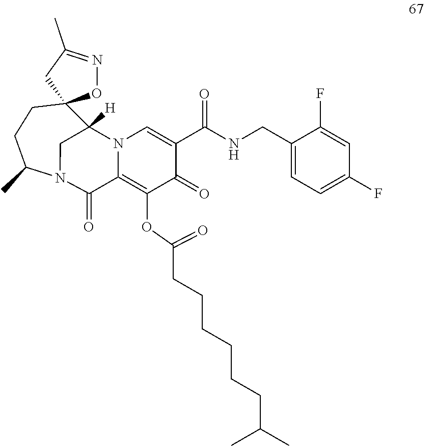 [SMILES: CC1=NO[C@@]2(CC[C@H](C)N3C[C@H]2n2cc(C(=O)NCc4ccc(F)cc4F)c(=O)c(OC(=O)CCCCCCC(C)C)c2C3=O)C1]

The title compound was prepared in a manner similar to Example 31, except using 8-methylnonanoic acid instead of (Z)-tetradec-9-enoic acid, Intermediate B instead of Intermediate C, and CH₂Cl₂ instead of DMF. MS (m/z) 641.3 [M+H]+. 1H NMR (400 MHz, DMSO-d6) δ 10.17 (t, J=5.9 Hz, 1H), 8.82 (s, 1H), 7.41 (td, J=8.7, 6.6 Hz, 1H), 7.24 (ddd, J=10.5, 9.3, 2.6 Hz, 1H), 7.07 (tdd, J=8.5, 2.6, 1.0 Hz, 1H), 4.69 (d, J=2.3 Hz, 1H), 4.65-4.42 (m, 4H), 3.89-3.53 (m, 2H), 3.02 (d, J=17.4 Hz, 1H), 2.66 (d, J=17.3 Hz, 1H), 1.95 (s, 3H), 1.89-1.69 (m, 4H), 1.55 (dtt, J=39.5, 13.1, 6.9 Hz, 3H), 1.44-1.14 (m, 12H), 0.85 (d, J=6.6 Hz, 6H).

### Example 68: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (5Z,8Z,11Z,14Z,17Z)-icosa-5,8,11,14,17-pentaenoate (68)

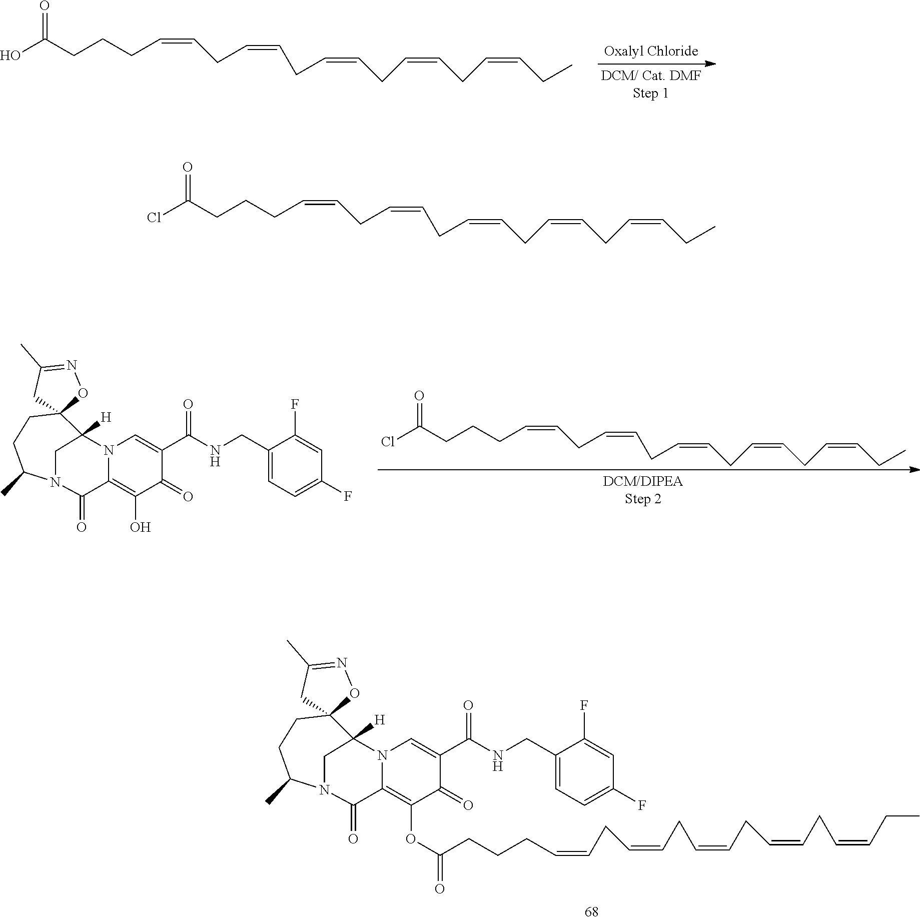

Step 1: Synthesis of (5Z,8Z,11Z,14Z,17Z)-icosa-5,8,11,14,17-pentaenoyl chloride

Into the solution of (5Z,8Z,11Z,14Z,7Z)-icosa-5,8,11,14,17-pentaenoic acid (605 mg, 2 mmol) in DCM (6 ml), oxalyl chloride (0.25 ml) was added at rt, then added one drop of DMF and the reaction was stirred at rt. After 2 h, the reaction mixture was concentrated under reduced pressure, the remaining residue was used for next step reaction without purification.

### Step 2: Synthesis of (3′S,5S,7′R)-1O′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (5Z,8Z,11Z,14Z,17Z)-icosa-5,8,11,14,17-pentaenoate

Into a mixture of Intermediate B (30) mg, 0.617 mmol) and (5Z,8Z,11Z,14Z,17Z)-icosa-5,8,11,14,17-pentaenoyl chloride (396 mg, 1.23 mmol) in DCM (20 ml) at 0° C. was added DIPEA (319 mg, 2.47 mmol). After addition, the reaction was allowed to warm to rt for overnight. The reaction mixture was extracted with ethyl acetate and washed with brine. The solvent was removed, and the residue was purified by column chromatography on silica gel to provide the title product. MS (m/z) 771.903 [M+H]⁺. ¹H NMR (400 MHz, Chloroform-d) δ 10.20 (t, J=6.0 Hz, 1H), 8.41 (s, 1H), 7.43-7.32 (m, 1H), 6.91-6.76 (m, 2H), 5.46-5.32 (m, 9H), 4.81 (dt, J=9.9, 6.8 Hz, 1H), 4.71-4.52 (m, 2H), 4.08 (d, J=2.2 Hz, 1H), 3.80 (dd, J=15.1, 1.9 Hz, 1H), 3.71 (s, 1H), 3.05 (d, J=17.8 Hz, 1H), 2.89-2.82 (m, 7H), 2.78-2.65 (m, 2H), 2.61 (d, J=17.8 Hz, 1H), 2.31-2.21 (m, 2H), 2.16-2.04 (m, 5H), 1.98-1.91 (m, 2H), 1.91-1.84 (m, 2H), 1.61 (s, 3H), 1.25 (dd, J=9.1, 6.9 Hz, 4H), 0.99 (t, J=7.5 Hz, 3H).

### Example 69: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (5Z,8Z,11Z,14Z,17Z)-icosa-5,8,11,14,17-pentaenoate (69)

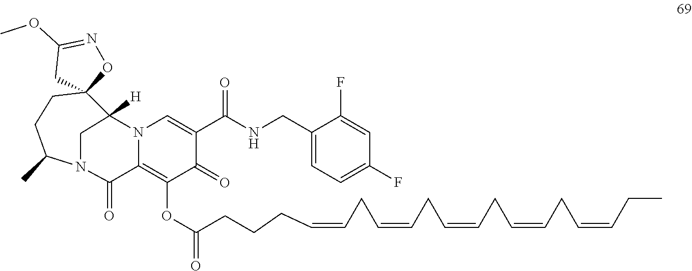 [SMILES: CC/C=C\C/C=C\C/C=C\C/C=C\C/C=C\CCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 68, except using Intermediate C instead of Intermediate B in Step 2. MS (m/z) 787.961 [M+H]⁺. ¹H NMR (400 MHz, Chloroform-d) δ 10.19 (t, J=6.0 Hz, 1H), 8.50 (s, 1H), 7.38 (d, J=8.0 Hz, 1H), 6.91-6.74 (m, 2H), 5.47-5.35 (m, 7H), 4.90-4.75 (m, 1H), 4.66 (d, J=6.1 Hz, 1H), 4.60 (d, J=5.7 Hz, 1H), 4.21 (s, 1H), 3.92 (s, 2H), 3.80 (d, J=15.1 Hz, 1H), 3.67 (d, J=15.0 Hz, 1H), 3.50 (q, J=7.0 Hz, 2H), 3.07 (d, J=17.2 Hz, 1H), 2.85 (p, J=6.8, 6.0 Hz, 6H), 2.73-2.66 (m, 2H), 2.24 (d, J=6.4 Hz, 1H), 2.10 (t, J=7.4 Hz, 2H), 1.91 (dt, J=22.9, 8.2 Hz, 3H), 1.58 (s, 7H), 1.24 (q, J=6.7 Hz, 5H), 0.99 (t, J=7.5 Hz, 3H).

### Example 70: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 6-methoxyhexanoate (70)

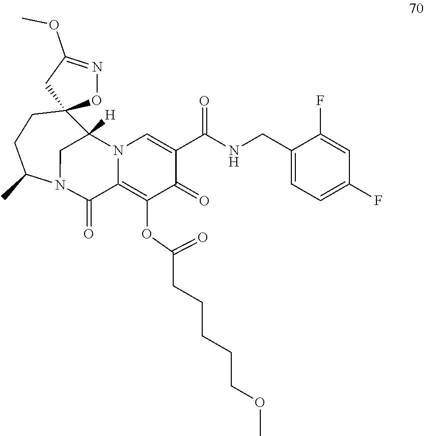 [SMILES: COCCCCCC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 31, except using 6-methoxyhexanoic acid instead of (Z)-tetradec-9-enoic acid and CH₂Cl₂ instead of DMF. MS (m/z) 631.6 [M+H]⁺. ¹H NMR (400 MHz, DMSO-d6) δ 10.16 (t, J=5.9 Hz, 1H), 8.86 (s, 1H), 7.41 (td, J=8.7, 6.6 Hz, 1H), 7.24 (ddd, J=10.5, 9.4, 2.6 Hz, 1H), 7.16-6.88 (m, 1H), 4.77 (s, 1H), 4.56 (tt, J=15.3, 8.0 Hz, 3H), 3.82 (s, 5H), 3.30 (d, J=6.2 Hz, 3H), 3.22 (s, 3H), 3.08 (d, J=16.7 Hz, 1H), 2.79 (d, J=16.8 Hz, 1H), 2.54 (d, J=7.5 Hz, 1H), 2.04-1.88 (m, 1H), 1.79 (q, J=14.4, 11.5 Hz, 2H), 1.63 (p, J=7.5 Hz, 2H), 1.52 (dt, J=13.8, 6.6 Hz, 2H), 1.40 (tt, J=10.6, 6.0 Hz, 2H), 1.26 (d, J=11.8 Hz, 1H), 1.16 (d, J=6.7 Hz, 3H). (71)

### Example 71: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl 6-methoxyhexanoate (71)

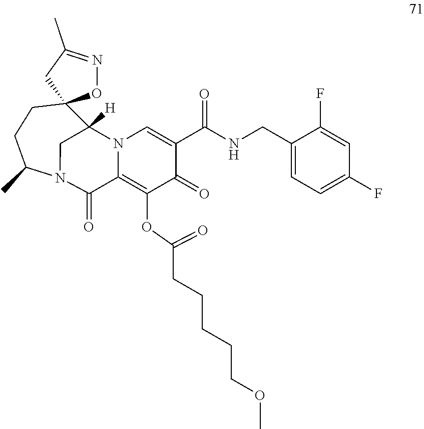 [SMILES: COCCCCCC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 31, except using 6-methoxyhexanoic acid instead of (Z)-tetradec-9-enoic acid, Intermediate B instead of Intermediate C, and CH₂C₁₂ instead of DMF. MS (m/z) 615.9 [M+H]⁺. ¹H NMR (400 MHz, DMSO-d6) δ 10.17 (t, J=6.0 Hz, 1H), 8.82 (s, 1H), 7.42 (td, J=8.6, 6.6 Hz, 1H), 7.36-7.18 (m, 1H), 7.08 (td, J=8.6, 2.6 Hz, 1H), 4.70 (s, 1H), 4.64-4.43 (m, 3H), 3.87-3.62 (m, 2H), 3.31 (d, J=6.5 Hz, 3H), 3.22 (s, 3H), 3.02 (d, J=17.3 Hz, 1H), 2.66 (d, J=17.4 Hz, 1H), 2.58-2.52 (m, 1H), 1.95 (s, 3H), 1.88-1.70 (m, 3H), 1.63 (p, J=7.4 Hz, 2H), 1.53 (p, J=6.6 Hz, 2H), 1.40 (ddt, J=14.4, 9.0, 5.4 Hz, 2H), 1.24 (s, 1H), 1.16 (d, J=6.6 Hz, 3H).

### Example 72: Preparation 5-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-5-oxopentanoic acid (72)

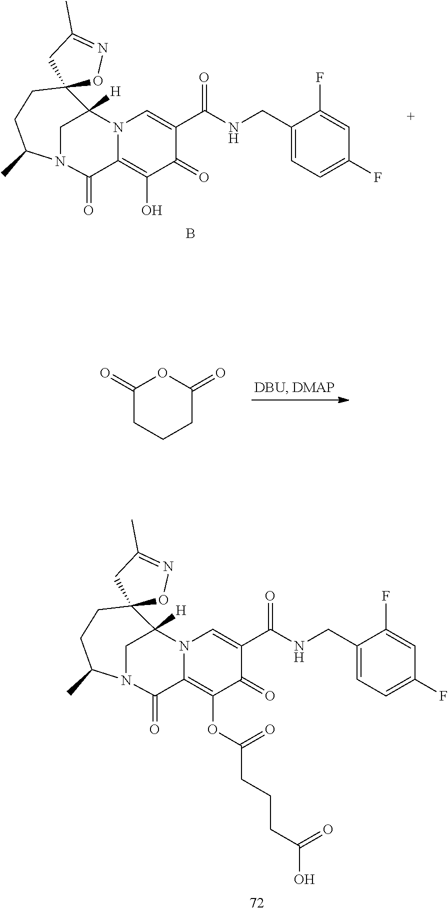

To a stirred solution of Intermediate B (0.14 g, 0.288 mmol) and tetrahydropyran-2,6-dione (0.043 g, 0.379 mmol) in acetonitrile (4 ml) was added 4-dimethylaminopyridine (0.007 g, 0.057 mmol) followed by DBU (0.11 g, 0.72 mmol) at room temperature. The resulting mixture was stirred at rt for 16 h. The solvent was removed and the crude material was purified by reverse phase preparative HPLC to afford the title compound. MS (m/z) 600.87 [M+H]⁺. ¹H NMR (400 MHz, Chloroform-d) δ 10.27 (t, J=6.0 Hz, 1H), 8.53 (s, 1H), 7.36 (td, J=8.5, 6.5 Hz, 1H), 6.90-6.78 (m, 2H), 4.80 (dt, J=9.8, 6.7 Hz, 1H), 4.68 (dd, J=15.0, 6.1 Hz, 1H), 4.60 (dd, J=15.1, 5.7 Hz, 1H), 4.14 (s, 1H), 3.90-3.52 (m, 2H), 3.07 (d, J=17.8 Hz, 1H), 2.79 (t, J=7.1 Hz, 2H), 2.60 (q, J=8.8, 7.3 Hz, 3H), 2.12 (d, J=12.9 Hz, 5H), 1.99-1.80 (m, 3H), 1.59 (dd, J=16.1, 7.9 Hz, 1H), 1.27 (d, J=6.7 Hz, 3H).

### Example 73: Preparation 5-(((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-5-oxopentanoic acid (73)

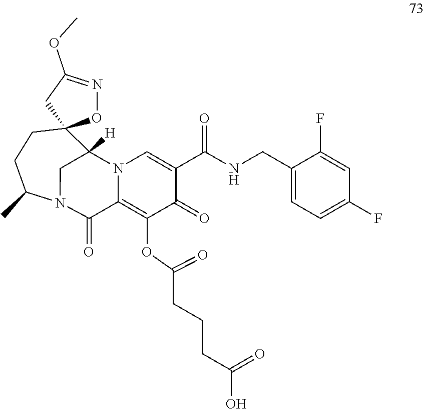 [SMILES: COC1=NO[C@@]2(CC[C@H](C)N3C[C@H]2n2cc(C(=O)NCc4ccc(F)cc4F)c(=O)c(OC(=O)CCCC(=O)O)c2C3=O)C1]

The title compound was prepared in a manner similar to Example 72, except using Intermediate C instead of Intermediate B. MS (m/z) 616.87 [M+H]⁺. ¹H NMR (400 MHz, Chloroform-d) δ 10.28 (t, J=5.9 Hz, 1H), 8.62 (s, 1H), 7.41-7.31 (m, 1H), 6.91-6.78 (m, 2H), 4.80 (dt, J=10.1, 6.9 Hz, 1H), 4.69 (dd, J=14.8, 6.2 Hz, 1H), 4.60 (dd, J=15.0, 5.8 Hz, 1H), 4.27 (s, 1H), 3.92 (s, 3H), 3.82 (dd, J=15.2, 1.9 Hz, 1H), 3.69 (d, J=15.2 Hz, 1H), 3.08 (d, J=17.1 Hz, 1H), 2.79 (t, J=7.1 Hz, 2H), 2.70 (d, J=17.2 Hz, 1H), 2.59 (t, J=7.3 Hz, 2H), 2.25-1.74 (m, 5H), 1.67-1.56 (m, 1H), 1.26 (d, J=6.7 Hz, 3H).

### Example 74: Preparation of 7-(((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-7-oxoheptanoic acid (74)

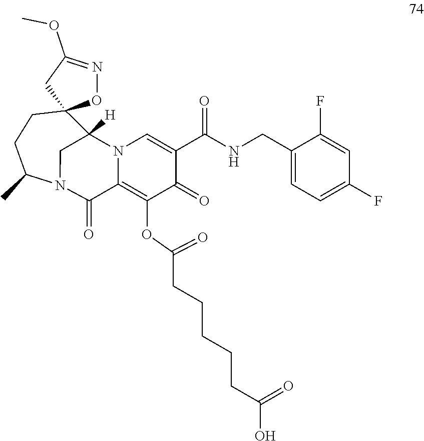 [SMILES: COC1=NO[C@@]2(CC[C@H](C)N3C[C@H]2n2cc(C(=O)NCc4ccc(F)cc4F)c(=O)c(OC(=O)CCCCCC(=O)O)c2C3=O)C1]

The title compound was prepared in a manner similar to Example 31, except using heptanedioic acid instead of (Z)-tetradec-9-enoic acid and CH₂Cl₂ instead of DMF. MS (m/z) 645.2 [M+H]⁺. ¹H NMR (400 MHz, DMSO-d6) δ 11.99 (s, 1H), 10.16 (t, J=6.0 Hz, 1H), 8.86 (s, 1H), 7.41 (td, J=8.6, 6.6 Hz, 1H), 7.24 (td, J=9.9, 2.6 Hz, 1H), 7.07 (td, J=8.6, 2.6 Hz, 1H), 4.77 (s, 1H), 4.71-4.42 (m, 3H), 3.92-3.54 (m, 5H), 3.07 (d, J=16.7 Hz, 1H), 2.78 (d, J=16.8 Hz, 1H), 2.21 (t, J=7.4 Hz, 2H), 2.02-1.72 (m, 4H), 1.57 (dp, J=35.7, 7.5 Hz, 5H), 1.44-1.20 (m, 3H), 1.16 (d, J=6.7 Hz, 3H).

### Example 75: Preparation of (E)-4-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-4-oxobut-2-enoic acid (75)

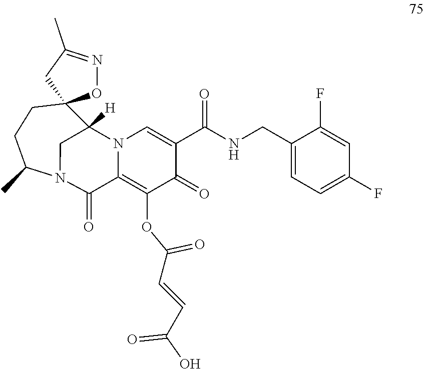 [SMILES: CC1=NO[C@@]2(CC[C@H](C)N3C[C@H]2n2cc(C(=O)NCc4ccc(F)cc4F)c(=O)c(OC(=O)/C=C/C(=O)O)c2C3=O)C1]

The title compound was prepared in a manner similar to Example 25, except using Intermediate B instead of Intermediate C. MS (m/z) 585.08 [M+H]⁺. ¹H NMR (400 MHz, DMSO-d6) δ 10.12 (t, J=6.0 Hz, 1H), 8.87 (s, 1H), 7.42 (td, J=8.6, 6.5 Hz, 1H), 7.24 (td, J=9.9, 2.6 Hz, 1H), 7.08 (td, J=8.6, 2.6 Hz, 1H), 6.88 (s, 2H), 4.73 (s, 1H), 4.63-4.46 (m, 3H), 3.95-3.48 (m, 2H), 3.03 (d, J=17.4 Hz, 1H), 2.67 (d, J=17.4 Hz, 1H), 1.95 (s, 3H), 1.86-1.68 (m, 3H), 1.32-1.09 (m, 4H).

### Example 76: Preparation of 18-(((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-18-oxooctadecanoic acid (76)

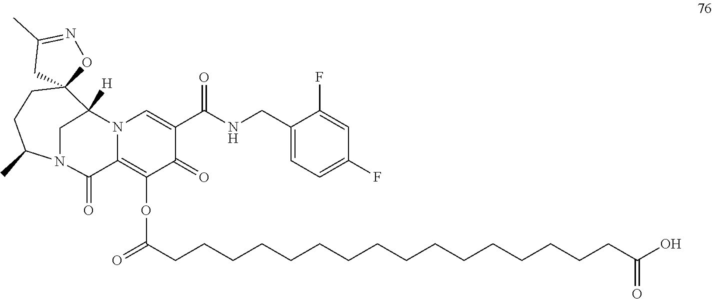 [SMILES: CC1=NO[C@@]2(CC[C@H](C)N3C[C@H]2n2cc(C(=O)NCc4ccc(F)cc4F)c(=O)c(OC(=O)CCCCCCCCCCCCCCCCC(=O)O)c2C3=O)C1]

The title compound was prepared in a manner similar to Example 68 except using octadecanedioic acid instead of (5Z,8Z,1Z,14Z,17Z)-icosa-5,8,11,14,17-pentaenoic acid in Step 1. MS (m/z) 783.932 [M+H]⁺. ¹H NMR (400 MHz, Chloroform-d) δ 10.27 (t, J=6.0 Hz, 1H), 8.71 (s, 1H), 7.36 (td, J=8.4, 6.3 Hz, 1H), 6.87-6.77 (m, 2H), 4.82 (dt, J=10.3, 6.8 Hz, 1H), 4.69 (dd, J=15.2, 6.1 Hz, 1H), 4.56 (dd, J=15.2, 5.7 Hz, 1H), 4.37 (s, 1H), 3.88 (s, 2H), 3.79 (dd, J=15.1, 1.9 Hz, 1H), 3.68 (d, J=15.3 Hz, 1H), 3.11 (d, J=17.1 Hz, 1H), 2.71-2.61 (m, 3H), 2.35 (t, J=7.5 Hz, 2H), 2.05-1.89 (m, 3H), 1.83-1.74 (m, 2H), 1.64 (t, J=7.4 Hz, 3H), 1.50-1.23 (m, 29H).

### Example 77: Preparation of 18-(((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)-18-oxooctadecanoic acid (77)

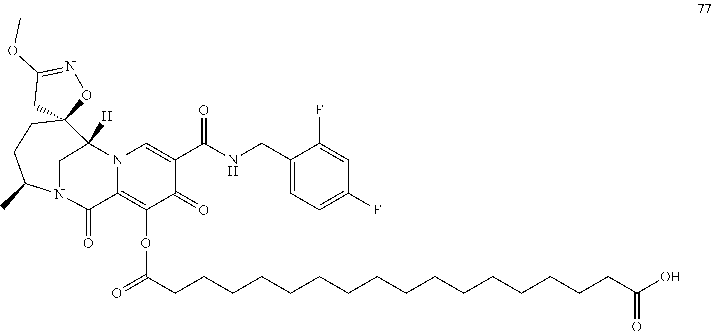 [SMILES: COC1=NO[C@@]2(CC[C@H](C)N3C[C@H]2n2cc(C(=O)NCc4ccc(F)cc4F)c(=O)c(OC(=O)CCCCCCCCCCCCCCCCC(=O)O)c2C3=O)C1]

The title compound was prepared in a manner similar to Example 68, except using octadecanedioic acid instead of (5Z,8Z,11Z,14Z,17Z)-icosa-5,8,11,14,17-pentaenoyl chloride and Intermediate C instead of Intermediate B in Step 2. MS (m/z) 799.958 [M+H]⁺. ¹H NMR (400 MHz, Chloroform-d) δ 10.24 (t, J=6.0 Hz, 1H), 8.48 (s, 1H), 7.42-7.31 (m, 1H), 6.89-6.77 (m, 2H), 4.82 (dt, J=9.9, 6.8 Hz, 1H), 4.68 (dd, J=15.1, 6.1 Hz, 1H), 4.58 (dd, J=15.2, 5.8 Hz, 1H), 4.11 (d, J=2.4 Hz, 1H), 3.81 (dd, J=15.1, 1.9 Hz, 1H), 3.74-3.65 (m, 1H), 3.06 (d, J=17.8 Hz, 1H), 2.68 (dd, J=8.4, 6.9 Hz, 2H), 2.60 (d, J=17.9 Hz, 1H), 2.36 (t, J=7.5 Hz, 2H), 2.10 (s, 3H), 1.99-1.87 (m, 3H), 1.83-1.74 (m, 2H), 1.64 (q, J=7.5 Hz, 3H), 1.39 (dd, J=28.2, 5.9 Hz, 4H), 1.28 (d, J=4.4 Hz, 23H).

### Example 78: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl(methoxycarbonyl)-L-alaninate (78)

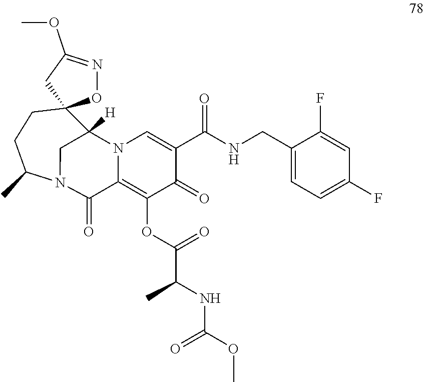 [SMILES: COC(=O)N[C@@H](C)C(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a manner similar to Example 31, except using (methoxycarbonyl)-L-alanine instead of (Z)-tetradec-9-enoic acid. MS (m/z) 632.83 [M+H]⁺. ¹H NMR (400 MHz, Chloroform-d) δ 10.34 (t, J=6.0 Hz, 1H), 8.79 (s, 1H), 7.35 (td, J=8.4, 6.3 Hz, 1H), 6.92-6.77 (m, 2H), 5.44 (d, J=7.9 Hz, 1H), 4.89-4.73 (m, 2H), 4.68 (dd, J=15.0, 6.3 Hz, 1H), 4.58 (dd, J=15.0, 5.8 Hz, 1H), 4.42 (s, 1H), 3.87 (s, 3H), 3.81 (dd, J=15.2, 1.9 Hz, 1H), 3.75-3.64 (m, 4H), 3.09 (d, J=17.1 Hz, 1H), 2.65 (d, J=17.1 Hz, 1H), 2.07-1.88 (m, 3H), 1.69-1.54 (m, 4H), 1.26 (d, J=6.7 Hz, 3H).

### Example 79: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl(methoxycarbonyl)-L-alaninate (79)

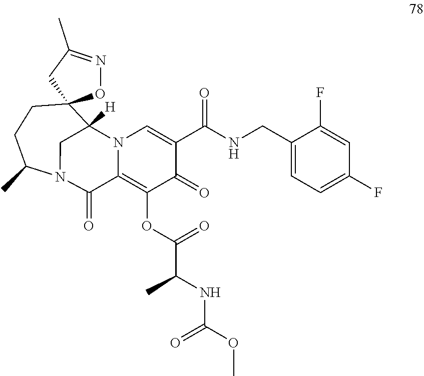 [SMILES: COC(=O)N[C@@H](C)C(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a manner similar to Example 78, except using Intermediate B instead of Intermediate C. MS (m/z) 615.83 [M+H]⁺. ¹H NMR (400 MHz, Chloroform-d) δ 10.23 (t, J=6.0 Hz, 1H), 8.50 (s, 1H), 7.35 (td, J=8.7, 6.6 Hz, 1H), 6.93-6.73 (m, 2H), 5.44 (d, J=8.0 Hz, 1H), 4.91-4.72 (m, 2H), 4.71-4.55 (m, 2H), 4.13 (d, J=2.3 Hz, 1H), 3.87-3.63 (m, 5H), 3.06 (d, J=17.8 Hz, 1H), 2.60 (d, J=17.8 Hz, 1H), 2.06 (s, 3H), 2.01-1.84 (m, 3H), 1.72-1.53 (m, 4H), 1.27 (d, J=6.7 Hz, 3H).

### Example 80: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl(methoxycarbonyl)-L-valinate (80)

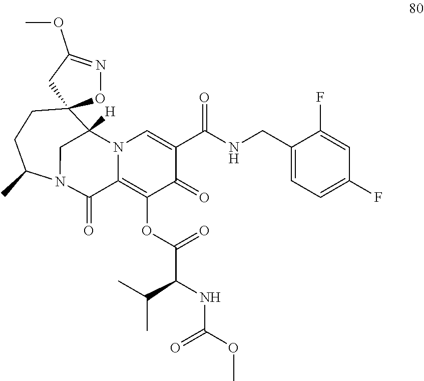 [SMILES: COC(=O)N[C@H](C(=O)Oc1c2n(cc(C(=O)NCc3ccc(F)cc3F)c1=O)[C@@H]1CN(C2=O)[C@@H](C)CC[C@]12CC(OC)=NO2)C(C)C]

The title compound was prepared in a manner similar to Example 31, except using (methoxycarbonyl)-L-valine instead of (Z)-tetradec-9-enoic acid. MS (m/z) 660.239 [M+H]⁺. ¹H NMR (400 MHz, Chloroform-d) δ 10.24 (t, J=5.9 Hz, 1H), 9.06 (s, 1H), 7.38-7.30 (m, 1H), 6.87-6.74 (m, 2H), 5.39 (d, J=9.3 Hz, 1H), 4.86-4.64 (m, 4H), 4.48 (dd, J=15.2, 5.6 Hz, 1H), 3.78-3.61 (m, 8H), 3.11 (d, J=17.0 Hz, 1H), 2.58-2.41 (m, 2H), 2.00-1.78 (m, 3H), 1.61-1.49 (m, 1H), 1.24-1.20 (m, 3H), 1.08 (t, J=6.5 Hz, 6H).

### Example 81: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl(methoxycarbonyl)-L-valinate (81)

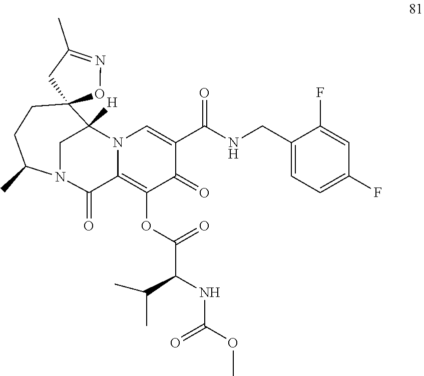 [SMILES: COC(=O)N[C@H](C(=O)Oc1c2n(cc(C(=O)NCc3ccc(F)cc3F)c1=O)[C@@H]1CN(C2=O)[C@@H](C)CC[C@]12CC(C)=NO2)C(C)C]

The title compound was prepared in a manner similar to Example 80, except using Intermediate B instead of Intermediate C. MS (m/z) 644.241 [M+H]⁺. ¹H NMR (400 MHz, Chloroform-d) δ 10.18 (t. J=6.0 Hz, 1H), 8.59 (s, 1H), 7.32 (td, J=8.7, 8.3, 6.4 Hz, 1H), 6.85-6.68 (m, 2H), 5.72-5.42 (m, 1H), 4.85-4.70 (m, 1H), 4.68-4.48 (m, 3H), 4.19 (s, 1H), 3.77-3.55 (m, 5H), 3.08 (d, J=17.7 Hz, 1H), 2.61-2.38 (m, 2H), 1.96 (s, 3H), 1.91-1.74 (m, 3H), 1.56-1.66 (m, 1H), 1.20 (d, J=6.7 Hz, 3H), 1.08 (d, J=6.8 Hz, 6H).

### Example 82: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-methylpentanoate (82)

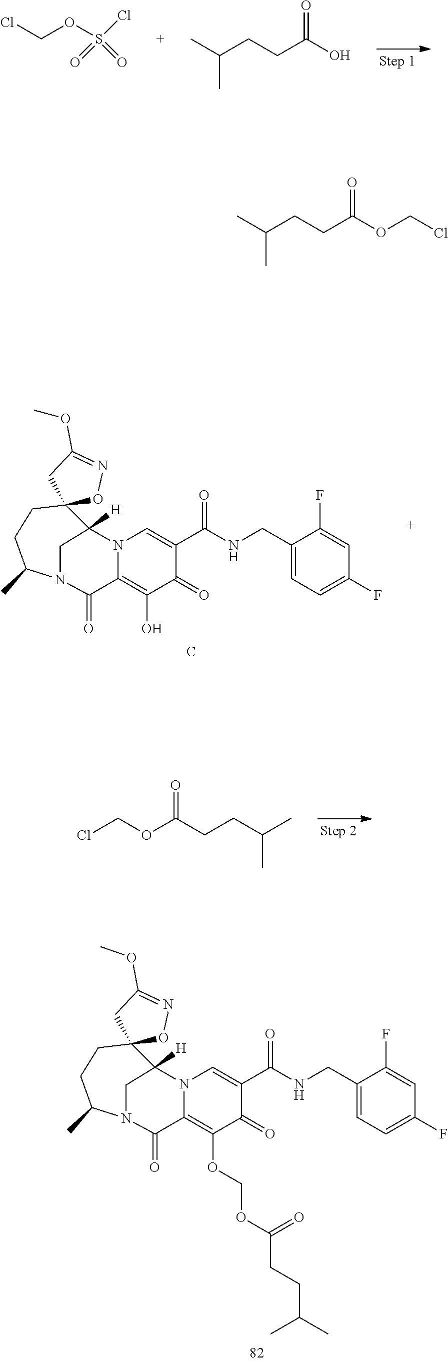

### Step 1: Synthesis of chloromethyl 4-methylpentanoate

The title compound was prepared in a manner similar to WO2023102239 Example 44, except 4-methylpentanoic acid was used and the resulting residue was used directly in next step without purification.

### Step 2: Synthesis of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-methylpentanoate

To a mixture of Intermediate C (400 mg, 0.796 mmol) and chloromethyl 4-methylpentanoate (236 mg, 1.43 mmol) in DCM (5.0 mL) at room temperature was added potassium carbonate (275 mg, 1.99 mmol) and tetrabutylammonium hydrogen sulfate (270 mg, 0.796 mmol) followed by water (5.0 mL). The resulting mixture was vigorously stirred at 40° C. for 16 hours. The reaction was then cooled to room temperature, diluted with DCM, washed sequentially with water and brine, dried over sodium sulfate, filtered and concentrated, purified by normal phase silica gel column chromatography, eluting with 20-80% EtOAc/Hexane, to provide the title compound. MS (m/z) 630.999 [M+H]⁺. ¹H NMR (400 MHz, Chloroform-d) δ 10.33 (s, 1H), 8.47 (s, 1H), 7.45-7.32 (m, 1H), 6.92-6.75 (m, 2H), 6.00 (d, J=6.2 Hz, 1H), 5.80 (d, J=6.2 Hz, 1H), 4.94-4.81 (m, 1H), 4.64 (qd, J=15.1, 5.9 Hz, 2H), 4.21 (s, 1H), 3.91 (s, 3H), 3.76 (dd, J=15.0, 1.9 Hz, 1H), 3.61 (dd, J=15.0, 2.7 Hz, 1H), 3.09 (d, J=17.2 Hz, 1H), 2.68 (d, J=17.2 Hz, 1H), 2.43-2.33 (m, 2H), 2.07-1.90 (m, 3H), 1.65-1.48 (m, 4H), 1.23 (d, J=6.7 Hz, 3H), 0.89 (d, J=6.4 Hz, 6H).

### Example 83: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl acetate (83)

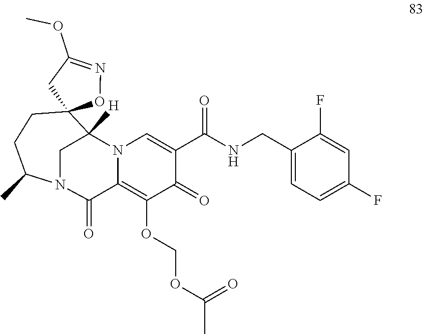 [SMILES: COC1=NO[C@@]2(CC[C@H](C)N3C[C@H]2n2cc(C(=O)NCc4ccc(F)cc4F)c(=O)c(OCOC(C)=O)c2C3=O)C1]

The title compound was prepared in a similar manner to Example 82, except using acetic acid in Step 1 instead of 4-methylpentanoic acid. MS (m/z) 575.009 [M+H]⁺. ¹H NMR (400 MHz, Chloroform-d) δ 10.31 (t, J=6.0 Hz, 1H), 8.48 (s, 1H), 7.38 (td, J=8.4, 6.4 Hz, 1H), 6.94-6.76 (m, 2H), 6.02 (d, J=6.3 Hz, 1H), 5.79 (d, J=6.3 Hz, 1H), 4.95-4.82 (m, 1H), 4.69 (dd, J=15.1, 6.0 Hz, 1H), 4.60 (dd, J=15.0, 5.8 Hz, 1H), 4.22 (s, 1H), 3.91 (s, 3H), 3.76 (dd, J=15.1, 1.9 Hz, 1H), 3.61 (dd, J=15.1, 2.7 Hz, 1H), 3.09 (d, J=17.1 Hz, 1H), 2.69 (d, J=17.2 Hz, 1H), 2.13 (s, 3H), 2.08-1.91 (m, 3H), 1.66-1.57 (m, 1H), 1.23 (d, J=6.7 Hz, 3H).

### Example 84: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl acetate (84)

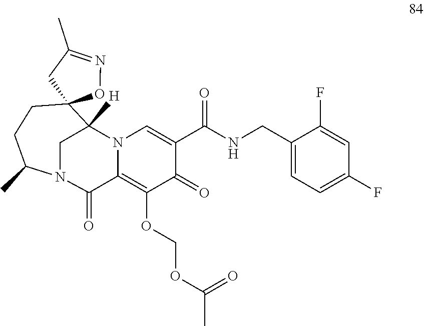 [SMILES: CC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 83, except using Intermediate B instead of Intermediate C. MS (m/z) 559.009 [M+H]+. 1H NMR (400 MHz, Chloroform-d) δ 10.31 (t, J=5.9 Hz, 1H), 8.34 (s, 1H), 7.43-7.31 (m, 1H), 6.94-6.72 (m, 2H), 6.02 (d, J=6.2 Hz, 1H), 5.79 (d, J=6.3 Hz, 1H), 4.95-4.83 (m, 1H), 4.68 (dd, J=15.1, 6.0 Hz, 1H), 4.61 (dd, J=15.1, 5.8 Hz, 1H), 4.04 (s, 1H), 3.77 (dd, J=15.1, 1.9 Hz, 1H), 3.62 (dd, J=15.0, 2.7 Hz, 1H), 3.06 (d, J=17.7 Hz, 1H), 2.61 (dd, J=17.7, 1.3 Hz, 1H), 2.13 (s, 3H), 2.11 (s, 3H), 1.99-1.88 (m, 3H), 1.55-1.62 (m, 1H), 1.24 (d, J=6.7 Hz, 3H).

### Example 85: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl propionate (85)

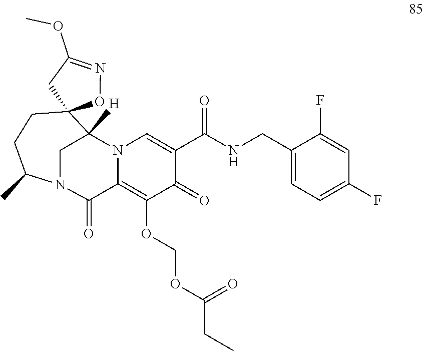 [SMILES: CCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 82 Step 2, except using chloromethyl propionate instead of chloromethyl 4-methylpentanoate. MS (m/z) 588.998 [M+H]+. 1H NMR (400 MHz, Chloroform-d) δ 10.32 (t, J=6.0 Hz, 1H), 8.47 (s, 1H), 7.38 (td, J=8.4, 6.4 Hz, 1H), 6.95-6.77 (m, 2H), 6.01 (d, J=6.3 Hz, 1H), 5.79 (d, J=6.3 Hz, 1H), 4.95-4.81 (m, 1H), 4.64 (qd, J=15.2, 5.6 Hz, 2H), 4.21 (s, 1H), 3.91 (s, 3H), 3.76 (dd, J=15.1, 1.9 Hz, 1H), 3.61 (dd, J=15.1, 2.7 Hz, 1H), 3.09 (d, J=17.2 Hz, 1H), 2.68 (d, J=17.1 Hz, 1H), 2.42 (q, J=7.5 Hz, 2H), 2.09-1.90 (m, 3H), 1.66-1.56 (m, 1H), 1.23 (d, J=6.7 Hz, 3H), 1.13 (t, J=7.5 Hz, 3H).

### Example 86: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl propionate (86)

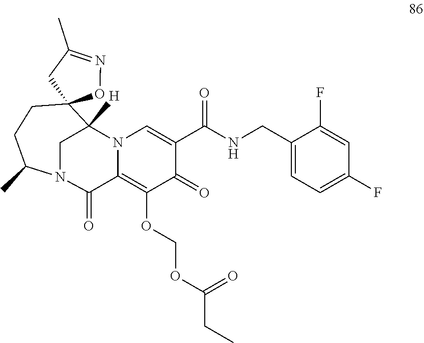 [SMILES: CCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 85, except using Intermediate B instead of Intermediate C. MS (m/z) 573.009 [M+H]+. 1H NMR (400 MHz, Chloroform-d) δ 10.32 (t, J=5.9 Hz, 1H), 8.34 (s, 1H), 7.44-7.32 (m, 1H), 6.92-6.78 (m, 2H), 6.01 (d, J=6.2 Hz, 1H), 5.80 (d, J=6.3 Hz, 1H), 4.95-4.81 (m, 1H), 4.74-4.57 (m, 2H), 4.04 (s, 1H), 3.77 (dd, J=15.0, 1.9 Hz, 1H), 3.63 (dd, J=15.1, 2.7 Hz, 1H), 3.06 (d, J=17.8 Hz, 1H), 2.60 (dd, J=17.8, 1.2 Hz, 1H), 2.47-2.37 (m, 2H), 2.11 (s, 3H), 1.99-1.86 (m, 3H), 1.56-1.66 (m, 1H), 1.24 (d, J=6.7 Hz, 3H), 1.13 (t, J=7.5 Hz, 3H).

### Example 87: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl butyrate (87)

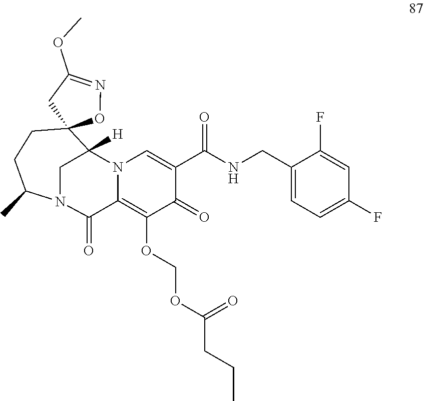 [SMILES: CCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 18, except using chloromethyl butanoate instead of chloromethyl pivalate and Intermediate C instead of Intermediate B. MS (m/z) 603.0 [M+H]⁺. 1H NMR (400 MHz, Chloroform-d) δ 10.32 (t, J=6.0 Hz, 1H), 8.46 (s, 1H), 7.38 (td, J=8.4, 6.4 Hz, 1H), 6.91-6.78 (m, 2H), 6.00 (d, J=6.3 Hz, 1H), 5.80 (d, J=6.2 Hz, 1H), 4.95-4.80 (m, 1H), 4.68 (dd, J=15.1, 6.1 Hz, 1H), 4.60 (dd, J=15.1, 5.9 Hz, 1H), 4.20 (d, J=2.6 Hz, 1H), 3.91 (s, 3H), 3.76 (dd, J=15.1, 1.9 Hz, 1H), 3.61 (dd, J=15.1, 2.7 Hz, 1H), 3.09 (d, J=17.2 Hz, 1H), 2.68 (d, J=17.2 Hz, 1H), 2.37 (t, J=7.4 Hz, 2H), 2.07-1.97 (m, 1H), 1.94 (ddd, J=9.8, 8.2, 3.0 Hz, 2H), 1.63 (dt, J=14.8, 7.4 Hz, 3H), 1.23 (d, J=6.8 Hz, 3H), 0.95 (t, J=7.4 Hz, 3H).

### Example 88: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl butyrate (88)

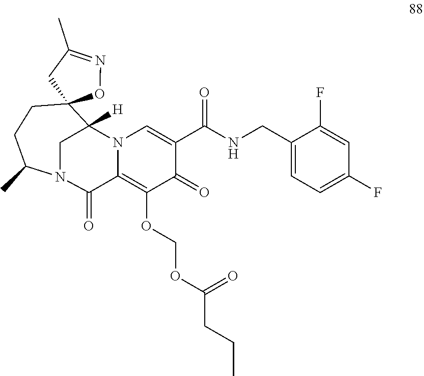 [SMILES: CCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 18, except using chloromethyl butanoate instead of chloromethyl pivalate. MS (m/z) 587.0 [M+H]⁺. 1H NMR (400 MHz, Chloroform-d) δ 10.31 (t, J=5.9 Hz, 1H), 8.34 (s, 1H), 7.37 (td, J=8.6, 6.5 Hz, 1H), 6.90-6.78 (m, 2H), 6.00 (d, J=6.3 Hz, 1H), 5.80 (d, J=6.3 Hz, 1H), 4.87 (q, J=7.8 Hz, 1H), 4.64 (qd, J=15.1, 6.0 Hz, 2H), 4.05 (d, J=2.3 Hz, 1H), 3.77 (dd, J=15.1, 1.9 Hz, 1H), 3.63 (dd, J=15.0, 2.7 Hz, 1H), 3.10-3.01 (m, 1H), 2.60 (dd, J=17.8, 1.3 Hz, 1H), 2.37 (t, J=7.4 Hz, 2H), 2.10 (s, 2H), 2.10 (d, J=2.1 Hz, 1H), 2.00-1.85 (m, 3H), 1.72-1.54 (m, 3H), 1.23 (d, J=6.7 Hz, 3H), 0.95 (t, J=7.4 Hz, 3H).

### Example 89: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl pentanoate (89)

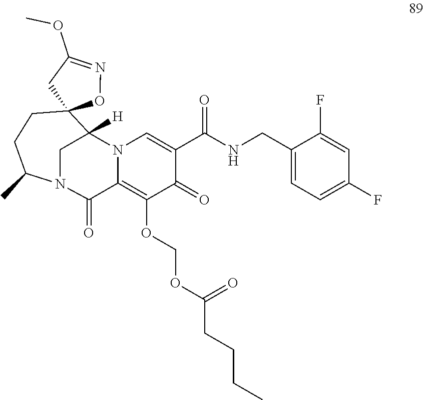 [SMILES: CCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 82, except using chloromethyl pentanoate instead of chloromethyl 4-methylpentanoate in Step 2. MS (m/z) 617.24 [M+H]⁺. 11H NMR (400 MHz, Chloroform-d) δ 10.32 (t, J=5.7 Hz, 1H), 8.61 (s, 1H), 7.34 (q, J=7.8 Hz, 1H), 6.80 (q, J=10.4, 9.7 Hz, 2H), 5.96 (d, J=6.2 Hz, 1H), 5.74 (d, J=6.3 Hz, 1H), 4.83 (p, J=7.4 Hz, 1H), 4.66 (dd, 3=15.4, 5.9 Hz, 1H), 4.54 (dd, J=15.2, 5.5 Hz, 11H), 4.35 (s, 1H), 3.83 (s, 3H), 3.71 (d, J=14.8 Hz, 1H), 3.59 (d, J=15.1 Hz, 1H), 3.08 (d, J=17.0 Hz, 1H), 2.60 (d, J=17.0 Hz, 1H), 2.35 (t, J=7.4 Hz, 2H), 1.90 (td, J=32.8, 30.8, 13.7 Hz, 4H), 1.65-1.48 (m, 3H), 1.39-1.20 (m, 4H), 0.87 (t, J=7.3 Hz, 3H).

### Example 90: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl pentanoate (90)

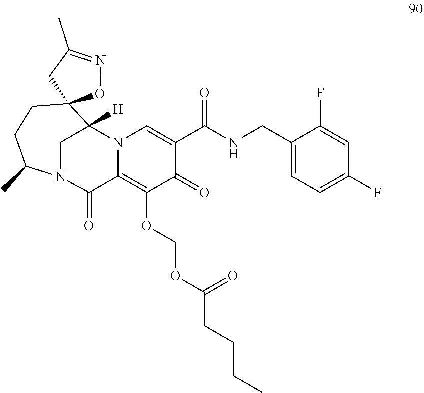 [SMILES: CCCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 82, except using chloromethyl pentanoate instead of chloromethyl 4-methylpentanoate and Intermediate B instead of Intermediate C in Step 2. MS (m/z) 601.24 [M+H]⁺. 1H NMR (400 MHz, Chloroform-d) δ 10.29 (s, 1H), 8.35 (s, 1H), 7.33 (q, J=7.8 Hz, 1H), 6.92-6.67 (m, 2H), 5.95 (d, J=6.1 Hz, 1H), 5.74 (d, J=6.2 Hz, 1H), 4.83 (h, J=7.0 Hz, 1H), 4.60 (qd, J=15.2, 5.3 Hz, 2H), 4.07 (s, 1H), 3.72 (d, J=14.9 Hz, 1H), 3.60 (d, J=15.0 Hz, 1H), 3.04 (d, J=17.2 Hz, 1H), 2.55 (d, J=17.4 Hz, 1H), 2.35 (t. J=7.5 Hz, 2H), 2.05 (s, 3H), 1.91 (dd, J=10.5, 6.2 Hz, 4H), 1.56 (p, J=7.3 Hz, 2H), 1.31 (h, J=7.4 Hz, 2H), 1.20 (d, J=6.6 Hz, 3H), 0.87 (t, J=7.3 Hz, 3H).

### Example 91: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl hexanoate (91)

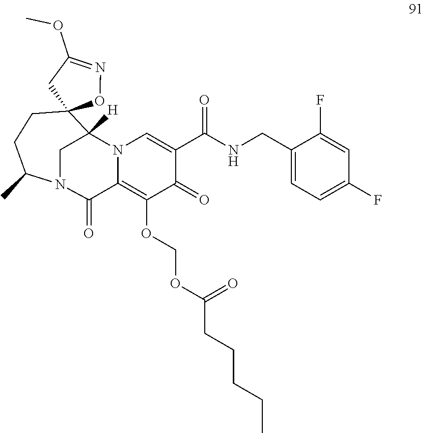 [SMILES: CCCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 82, except using hexanoic acid instead of 4-methylpentanoic acid in Step 1. MS (m/z) 631.242 [M+H]+. 1H NMR (400 MHz, Chloroform-d) δ 10.34 (t, J=5.9 Hz, 1H), 8.61 (s, 1H), 7.37 (td, J=8.4, 6.4 Hz, 1H), 6.90-6.75 (m, 2H), 5.99 (d, J=6.2 Hz, 1H), 5.77 (d, J=6.3 Hz, 1H), 4.86 (dt, J=9.4, 6.9 Hz, 1H), 4.68 (dd, J=15.1, 6.0 Hz, 1H), 4.57 (dd, J=15.1, 5.6 Hz, 1H), 4.35 (d, J=2.4 Hz, 1H), 3.86 (s, 3H), 3.74 (dd, J=15.1, 1.9 Hz, 1H), 3.61 (dd, J=15.1, 2.7 Hz, 1H), 3.11 (d, J=17.1 Hz, 1H), 2.64 (d, J=17.1 Hz, 1H), 2.37 (t, J=7.6 Hz, 2H), 2.08-1.87 (m, 3H), 1.66-1.53 (m, 3H), 1.36-1.25 (m, 4H), 1.22 (d, J=6.7 Hz, 3H), 0.96-0.81 (m, 3H).

### Example 92: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl hexanoate (92)

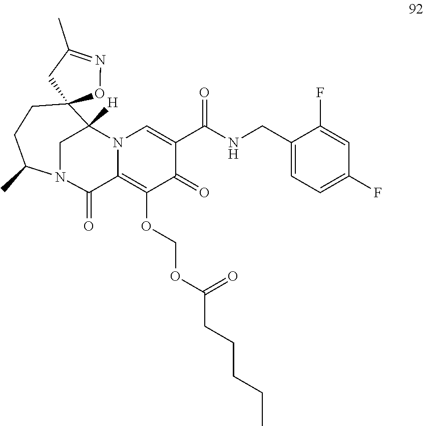 [SMILES: CCCCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 91, except using Intermediate B instead of Intermediate C. MS (m/z) 615.247 [M+H]+. 1H NMR (400 MHz, Chloroform-d) δ 10.32 (t, J=5.9 Hz 11H), 8.36 (s, 1H), 7.37 (td, J=8.6, 6.5 Hz, 1H), 6.94-6.77 (m, 2H), 6.00 (d, J=6.2 Hz, 1H), 5.79 (d, J=6.3 Hz, 1H), 4.96-4.78 (m, 1H), 4.75-4.53 (m, 2H), 4.06 (d, J=2.2 Hz, 1H), 3.76 (dd, J=15.1, 1.9 Hz, 1H), 3.62 (dd, J=15.0, 2.7 Hz, 1H), 3.06 (d, J=17.8 Hz, 1H), 2.65-2.52 (m, 1H), 2.38 (t, 3=7.6 Hz, 2H), 2.10 (s, 3H), 1.98-1.86 (m, 3H), 1.66-1.56 (m, 3H), 1.38-1.27 (m, 4H), 1.23 (d, J=6.7 Hz, 3H), 0.95-0.80 (m, 3H).

### Example 93: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl heptanoate (93)

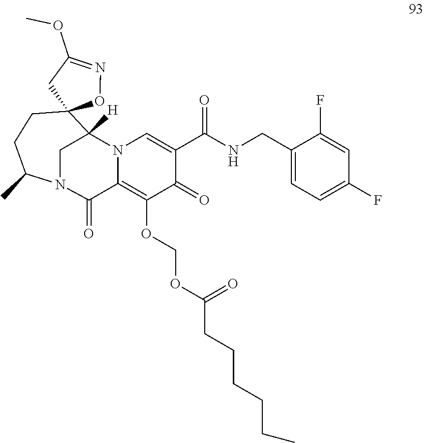 [SMILES: CCCCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 82, except using heptanoic acid instead of 4-methylpentanoic acid in Step 1. MS (m/z) 645.031 [M+H]+. 1H NMR (400 MHz, Chloroform-d) δ 10.33 (t, J=6.0 Hz, 1H), 8.50 (s, 1H), 7.38 (td, J=8.4, 6.4 Hz, 1H), 6.93-6.72 (m, 2H), 6.00 (d, J=6.2 Hz, 1H), 5.79 (d, J=6.2 Hz, 1H), 4.94-4.80 (m, 1H), 4.68 (dd, J=15.1, 6.1 Hz, 1H), 4.60 (dd, J=15.2, 5.8 Hz, 1H), 4.24 (s, 1H), 3.90 (s, 3H), 3.75 (dd, J=15.1, 1.9 Hz, 1H), 3.61 (dd, J=15.1, 2.7 Hz, 1H), 3.10 (d, J=17.1 Hz, 1H), 2.67 (d, J=17.2 Hz, 1H), 2.38 (t, J=7.5 Hz, 2H), 2.06-1.91 (m, 3H), 1.61 (p, J=7.9 Hz, 3H), 1.37-1.26 (m, 6H), 1.23 (d, J=6.7 Hz, 3H), 0.93-0.84 (m, 3H).

### Example 94: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl heptanoate (94)

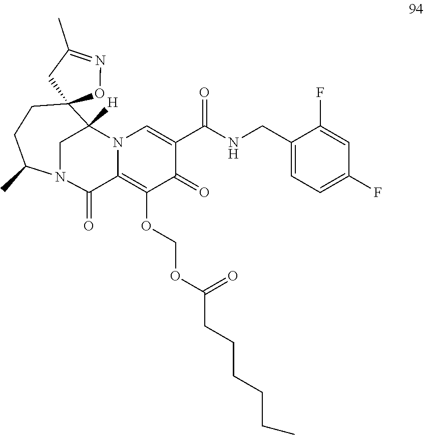 [SMILES: CCCCCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 93, except using Intermediate B instead of Intermediate C. MS (m/z) 629.024 [M+H]+. 1H NMR (400 MHz, Chloroform-d) δ 10.32 (t, J=6.0 Hz, 1H), 8.37 (s, 1H), 7.36 (td, J=8.6, 6.6 Hz, 1H), 6.91-6.75 (m, 2H), 5.99 (d, J=6.2 Hz, 1H), 5.77 (d, J=6.2 Hz, 1H), 4.86 (dp, J=13.5, 7.0 Hz, 1H), 4.71-4.54 (m, 2H), 4.08 (d, J=2.3 Hz, 1H), 3.75 (dd, J=15.0, 1.9 Hz, 1H), 3.62 (dd, J=15.1, 2.7 Hz, 1H), 3.06 (d, J=17.7 Hz, 1H), 2.62-2.53 (m, 1H), 2.37 (t, J=7.6 Hz, 2H), 2.09 (s, 3H), 1.98-1.84 (m, 3H), 1.66-1.53 (m, 3H), 1.37-1.25 (m, 6H), 1.23 (d, J=6.7 Hz, 3H), 0.88 (t, J=6.8 Hz, 3H).

### Example 95: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl octanoate (95)

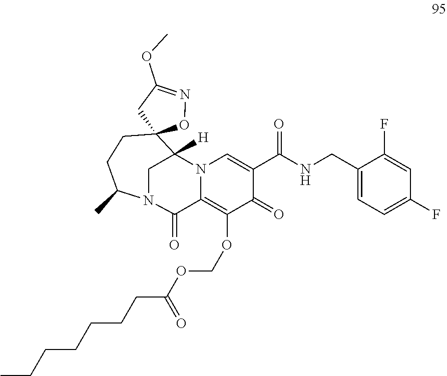 [SMILES: CCCCCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 18, except using chloromethyl octanoate instead of chloromethyl pivalate and Intermediate C instead of Intermediate B. MS (m/z) 659.0 [M+H]⁺. δ 10.34 (t, J=5.9 Hz, 1H), 8.59 (s, 1H), 7.37 (td, J=8.4, 6.3 Hz, 1H), 6.99-6.63 (m, 2H), 6.00 (d, J=6.2 Hz, 1H), 5.77 (d, J=6.3 Hz, 1H), 4.97-4.78 (m, 1H), 4.73-4.47 (m, 2H), 4.33 (d, J=2.3 Hz, 1H), 3.87 (s, 3H), 3.74 (dd, J=15.1, 1.9 Hz, 1H), 3.61 (dd, J=15.1, 2.7 Hz, 1H), 3.10 (d, J=17.1 Hz, 1H), 2.64 (d, J=17.2 Hz, 1H), 2.37 (t, J=7.5 Hz, 2H), 2.09-1.84 (m, 3H), 1.69-1.48 (m, 3H), 1.34-1.25 (m, 8H), 1.22 (d, J=6.7 Hz, 3H), 0.95-0.77 (m, 3H).

### Example 96: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl octanoate (96)

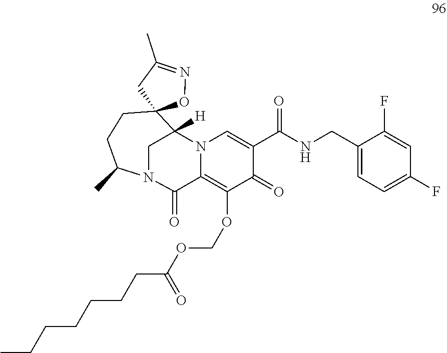 [SMILES: CCCCCCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 18, except using chloromethyl octanoate instead of chloromethyl pivalate. MS (m/z) 643.0 [M+H]⁺. 1H NMR (400 MHz, Chloroform-d) δ 10.32 (t, J=6.0 Hz, 1H), 8.34 (s, 1H), 7.37 (td, J=8.6, 6.5 Hz, 1H), 6.90-6.70 (m, 2H), 6.00 (d, J=6.3 Hz, 1H), 5.79 (d, J=6.3 Hz, 1H), 4.87 (dp, J=14.8, 7.2 Hz, 1H), 4.64 (qd, J=15.1, 5.8 Hz, 2H), 4.04 (d, J=2.1 Hz, 1H), 3.76 (dd, J=15.0, 1.9 Hz, 1H), 3.62 (dd, J=15.1, 2.7 Hz, 1H), 3.06 (d, J=17.8 Hz, 1H), 2.60 (dd, J=17.7, 1.3 Hz, 1H), 2.38 (t, J=7.5 Hz, 2H), 2.10 (s, 3H), 1.99-1.85 (m, 3H), 1.62-1.56 (m, 3H), 1.34-1.21 (m, 11H), 0.97-0.83 (m, 3H).

### Example 97: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl nonanoate (97)

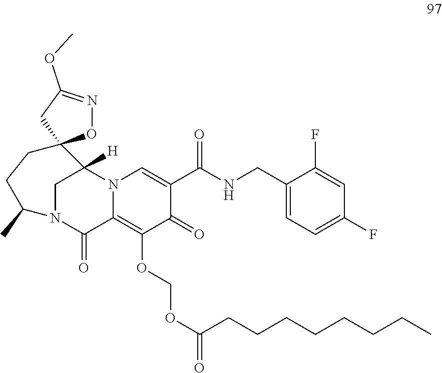 [SMILES: CCCCCCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 11, except using chloromethyl nonanoate instead of chloromethyl methyl carbonate. MS (m/z) 673.05 [M+H]+. 1H NMR (400 MHz, Chloroform-d) δ 10.33 (t, J=5.9 Hz, 1H), 8.55 (s, 1H), 7.37 (td, J=8.4, 6.3 Hz, 1H), 6.90-6.77 (m, 2H), 6.00 (d, J=6.2 Hz, 1H), 5.78 (d, J=6.3 Hz, 1H), 4.86 (dq, J=9.3, 7.0 Hz, 1H), 4.68 (dd, J=15.2, 6.1 Hz, 1H), 4.59 (dd, J=15.1, 5.8 Hz, 1H), 4.29 (d, J=2.3 Hz, 1H), 3.88 (s, 3H), 3.74 (dd, J=15.1, 1.9 Hz, 1H), 3.61 (dd, J=15.1, 2.7 Hz, 1H), 3.10 (d, J=17.1 Hz, 1H), 2.66 (d, J=17.2 Hz, 1H), 2.37 (t, J=7.6 Hz, 2H), 2.06-1.96 (m, 1H), 1.94 (td. J=9.5, 9.1, 4.0 Hz, 2H), 1.70 (s, 1H), 1.66-1.56 (m, 3H), 1.35-1.19 (m, 12H), 0.89 (t, J=6.7 Hz, 3H).

### Example 98: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl nonanoate (98)

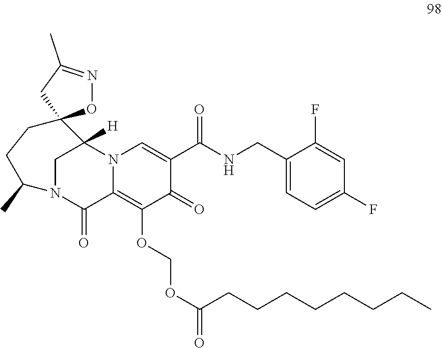 [SMILES: CCCCCCCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 11, except using chloromethyl nonanoate instead of chloromethyl methyl carbonate, and Intermediate B instead of Intermediate C. MS (m/z) 657.09 [M+H]+. 1H NMR (400 MHz, Chloroform-d) δ 10.31 (t, J=5.9 Hz, 1H), 8.36 (s, 1H), 7.36 (td, J=8.7, 6.5 Hz, 1H), 6.89-6.76 (m, 2H), 5.99 (d, J=6.3 Hz, 1H), 5.77 (d, J=6.3 Hz, 1H), 4.87 (p, J=7.9, 7.4 Hz, 1H), 4.63 (qd, J=15.2, 5.9 Hz, 2H), 4.08 (d, J=2.2 Hz, 1H), 3.75 (dd, J=15.1, 1.9 Hz, 1H), 3.62 (dd, J=15.1, 2.8 Hz, 1H), 3.06 (d, J=17.7 Hz, 1H), 2.58 (d, J=17.7 Hz, 1H), 2.37 (t, J=7.6 Hz, 2H), 2.08 (s, 3H), 1.98-1.84 (m, 3H), 1.79 (s, 1H), 1.59 (td, J=11.0, 9.0, 5.0 Hz, 3H), 1.35-1.19 (m, 12H), 0.88 (t, J=6.6 Hz, 3H).

### Example 99: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl decanoate (99)

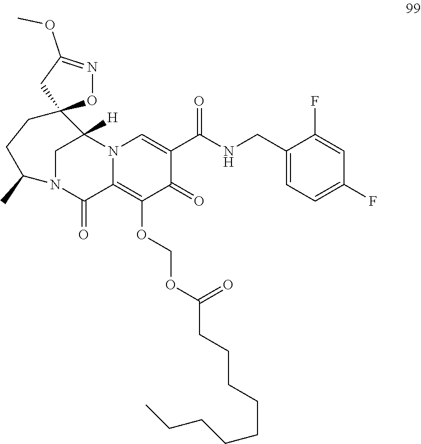 [SMILES: CCCCCCCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 82, except using decanoic acid instead of 4-methylpentanoic acid in Step 1. MS (m/z) 687.324 [M+H]+. 1H NMR (400 MHz, Chloroform-d) δ 10.33 (t, J=5.9 Hz, 1H), 8.48 (s, 1H), 7.46-7.33 (m, 1H), 6.93-6.75 (m, 2H), 6.00 (d, J=6.2 Hz, 1H), 5.79 (d, J=6.2 Hz, 1H), 4.99-4.78 (m, 1H), 4.64 (qd, J=15.2, 5.5 Hz, 2H), 4.23 (s, 1H), 3.91 (s, 3H), 3.75 (dd, J=15.1, 1.9 Hz, 1H), 3.61 (dd, J=15.1, 2.7 Hz, 1H), 3.09 (d, J=17.1 Hz, 1H), 2.67 (d, J=17.2 Hz, 1H), 2.38 (t, J=7.6 Hz, 2H), 2.08-1.91 (m, 3H), 1.66-1.56 (m, 3H), 1.33-1.21 (m, 15H), 0.89 (t, J=6.8 Hz, 3H).

### Example 100: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl decanoate (100)

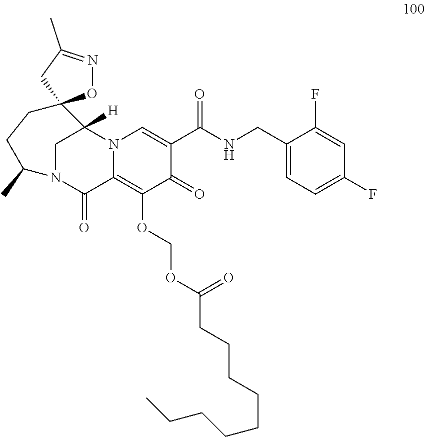 [SMILES: CCCCCCCCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 99, except using Intermediate B instead of Intermediate C. MS (m/z) 671.328 [M+H]+. 1H NMR (400 MHz, Chloroform-d) δ 10.38-10.26 (m, 1H), 8.34 (s, 1H), 7.43-7.32 (m, 1H), 6.90-6.77 (m, 2H), 6.01 (d, J=6.2 Hz, 1H), 5.79 (d, J=6.2 Hz, 1H), 4.95-4.81 (m, 1H), 4.73-4.56 (m, 2H), 4.04 (s, 1H), 3.77 (dd, J=15.0, 1.8 Hz, 1H), 3.62 (dd, J=15.0, 2.7 Hz, 1H), 3.06 (d, J=17.8 Hz, 1H), 2.60 (d, J=17.8 Hz, 1H), 2.39 (t, J=7.6 Hz, 2H), 2.11 (s, 3H), 2.01-1.85 (m, 3H), 1.64-1.56 (m, 3H), 1.32-1.22 (m, 15H), 0.90 (t, J=6.8 Hz, 3H).

### Example 101: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl dodecanoate (101)

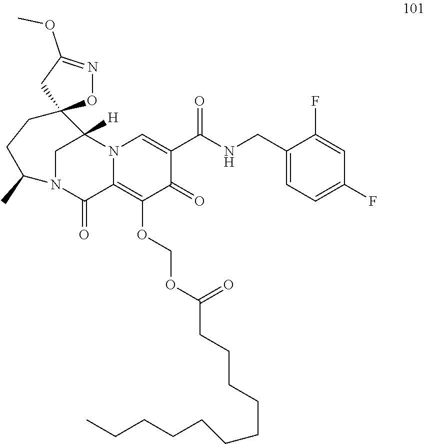 [SMILES: CCCCCCCCCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 82, except using dodecanoic acid instead of 4-methylpentanoic acid in Step 1. MS (m/z) 715.347 [M+H]+. 1H NMR (400 MHz, Chloroform-d) δ 10.32 (t, J=6.0 Hz, 1H), 8.44 (s, 1H), 7.46-7.33 (m, 1H), 6.94-6.74 (m, 2H), 6.00 (d, J=6.2 Hz, 1H), 5.80 (d, J=6.3 Hz, 1H), 4.97-4.80 (m, 1H), 4.68 (dd, J=15.1, 6.0 Hz, 1H), 4.61 (dd, J=15.1, 5.8 Hz, 1H), 4.18 (s, 1H), 3.92 (s, 3H), 3.76 (dd, J=15.1, 1.9 Hz, 1H), 3.61 (dd, J=15.1, 2.7 Hz, 1H), 3.09 (d, J=17.2 Hz, 1H), 2.69 (d, J=17.2 Hz, 1H), 2.38 (t, J=7.5 Hz, 2H), 2.09-1.90 (m, 3H), 1.66-1.57 (m, 4H), 1.34-1.26 (m, 15H), 1.23 (d, J=6.7 Hz, 3H), 0.90 (t, J=6.8 Hz, 3H).

### Example 102: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl dodecanoate (102)

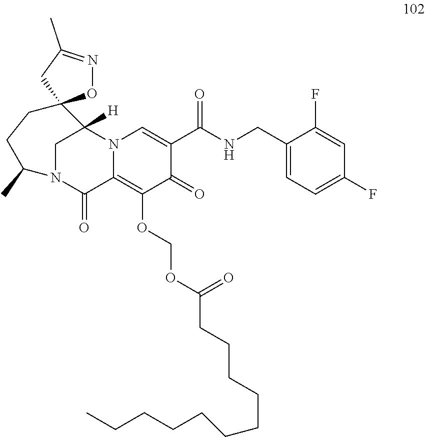 [SMILES: CCCCCCCCCCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 101, except using Intermediate B instead of Intermediate C. MS (m/z) 699.353 [M+H]+. 1H NMR (400 MHz, Chloroform-d) δ 10.31 (t, J=5.9 Hz, 1H), 8.33 (s, 1H), 7.44-7.32 (m, 1H), 6.91-6.76 (m, 2H), 6.01 (d, J=6.3 Hz, 1H), 5.79 (d, J=6.3 Hz, 1H), 4.95-4.79 (m, 1H), 4.68 (dd, J=15.1, 6.0 Hz, 1H), 4.61 (dd, J=15.0, 5.7 Hz, 1H), 4.04 (d, J=2.2 Hz, 1H), 3.77 (dd, J=15.1, 1.9 Hz, 1H), 3.62 (dd, J=15.0, 2.7 Hz, 1H), 3.05 (d, J=17.7 Hz, 1H), 2.60 (d, J=17.8 Hz, 1H), 2.38 (t, J=7.6 Hz, 2H), 2.10 (s, 3H), 2.00-1.86 (m, 3H), 1.65-1.56 (m, 4H), 1.34-1.25 (m, 15H), 1.23 (d, J=6.7 Hz, 3H), 0.90 (t, J=6.8 Hz, 3H).

### Example 103: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl tetradecanoate (103)

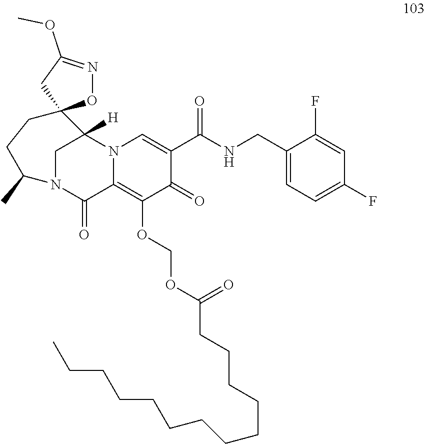 [SMILES: CCCCCCCCCCCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 82, except using tetradecanoic acid instead of 4-methylpentanoic acid in Step 1. MS (in/z) 743.1[M+H]⁺. 1H NMR (400 MHz, Chloroform-d) δ 10.32 (t, J=6.0 Hz, 1H), 8.46 (s, 1H), 7.38 (q, J=8.6, 8.1 Hz, 1H), 6.93-6.78 (m, 2H), 6.00 (d, J=6.2 Hz, 1H), 5.79 (d, J=6.3 Hz, 1H), 4.87 (h, J=6.9 Hz, 1H), 4.64 (qd, J=15.1, 5.9 Hz, 2H), 4.20 (s, 1H), 3.91 (s, 3H), 3.76 (dd, J=15.1, 1.9 Hz, 1H), 3.61 (dd, J=15.1, 2.7 Hz, 1H), 3.09 (d, J=17.2 Hz, 1H), 2.68 (d, J=17.2 Hz, 1H), 2.38 (t, J=7.6 Hz, 2H), 2.08-1.88 (m, 3H), 1.60 (d, J=10.3 Hz, 5H), 1.31-1.22 (m, 21H), 0.90 (t, J=6.7 Hz, 3H).

### Example 104: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl palmitate (104)

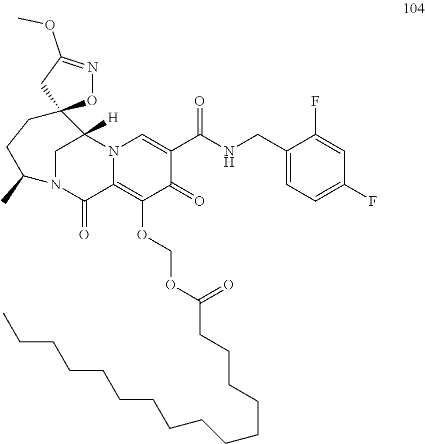 [SMILES: CCCCCCCCCCCCCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 82, except using palmitic acid instead of 4-methylpentanoic acid in Step 1. MS (m/z) 771.409 [M+H]+. 1H NMR (400 MH z, Chloroform-d) δ 10.32 (t, J=6.0 Hz, 1H), 8.44 (s, 1H), 7.38 (td, J=8.4, 6.4 Hz, 1H), 6.93-6.74 (m, 2H), 6.00 (d, J=6.2 Hz, 1H), 5.80 (d, J=6.3 Hz, 1H), 4.96-4.80 (m, 1H), 4.68 (dd, 3=15.1, 6.0 Hz, 1H), 4.61 (dd, J=15.1, 5.8 Hz, 1H), 4.19 (s, 1H), 3.92 (s, 3H), 3.76 (dd, J=15.2, 1.9 Hz, 1H), 3.61 (dd, J=15.1, 2.7 Hz, 1H), 3.09 (d, J=17.2 Hz, 1H), 2.68 (d, J=17.2 Hz, 1H), 2.38 (t, J=7.6 Hz, 2H), 2.08-1.89 (m, 3H), 1.67-1.56 (m, 4H), 1.27 (d, J=3.9 Hz, 23H), 1.23 (d, J=6.7 Hz, 3H), 0.94-0.85 (in, 3H).

### Example 105: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl palmitate (105)

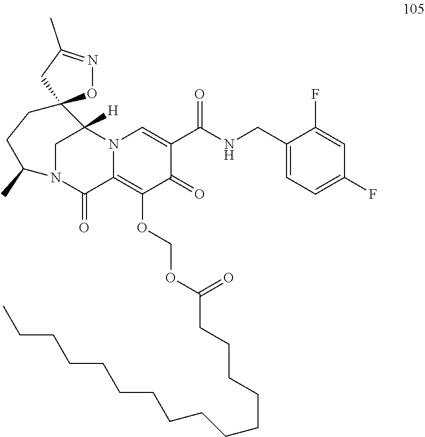 [SMILES: CCCCCCCCCCCCCCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 104, except using Intermediate B instead of Intermediate C. MS (m/z) 755.415 [M+H]+. 1H NMR (400 MHz, Chloroform-d) δ 10.32 (t, J=6.0 Hz, 1H), 8.34 (s, 1H), 7.37 (td, J=8.6, 6.6 Hz, 1H), 6.91-6.75 (m, 2H), 6.00 (d, J=6.2 Hz, 1H), 5.79 (d, J=6.3 Hz, 1H), 4.94-4.81 (m, 1H), 4.68 (dd, J=15.1, 6.0 Hz, 1H), 4.61 (dd, J=15.1, 5.8 Hz, 1H), 4.05 (s, 1H), 3.76 (dd, J=15.1, 1.9 Hz, 1H), 3.62 (dd, J=15.0, 2.7 Hz, 1H), 3.05 (d, J=17.7 Hz, 1H), 2.60 (d, J=17.8 Hz, 1H), 2.38 (t, J=7.6 Hz, 2H), 2.10 (s, 3H), 2.01-1.86 (m, 3H), 1.69-1.54 (m, 3H), 1.32-1.22 (m, 27H), 0.90 (t, J=6.8 Hz, 3H).

### Example 106: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl stearate (106)

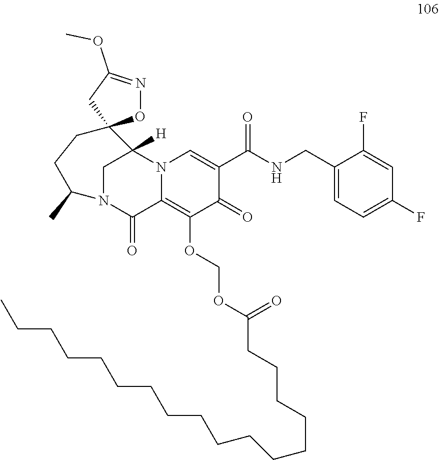 [SMILES: CCCCCCCCCCCCCCCCCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 82, except using stearic acid instead of 4-methylpentanoic acid in Step 1. MS (m/z) 799.442 [M+H]+. 1H NMR (400 MHz, Chloroform-d) δ 10.32 (t, J=6.0 Hz, 1H), 8.46 (s, 1H), 7.38 (td, J=8.4, 6.3 Hz, 1H), 6.91-6.77 (m, 2H), 6.00 (d, J=6.2 Hz, 1H), 5.79 (d, J=6.3 Hz, 1H), 4.95-4.80 (m, 1H), 4.68 (dd, J=15.1, 6.0 Hz, 1H), 4.60 (dd, J=15.2, 5.8 Hz, 1H), 4.20 (s, 1H), 3.91 (s, 3H), 3.75 (dd, J=15.1, 1.9 Hz, 1H), 3.61 (dd, J=15.1, 2.7 Hz, 1H), 3.09 (d, J=17.2 Hz, 1H), 2.68 (d, J=17.2 Hz, 1H), 2.38 (t, J=7.6 Hz, 2H), 2.05-1.90 (m, 3H), 1.67-1.55 (m, 4H), 1.27 (d, J=4.6 Hz, 27H), 1.23 (d, J=6.7 Hz, 3H), 0.93-0.86 (m, 3H).

### Example 107: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl stearate (107)

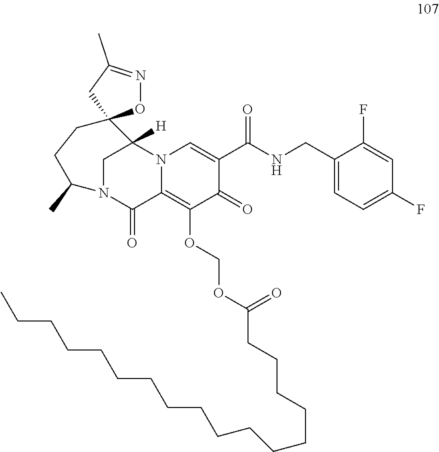 [SMILES: CCCCCCCCCCCCCCCCCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 106, except using Intermediate B instead of Intermediate C. MS (m/z) 783.447 [M+H]+. 1H NMR (400 MHz, Chloroform-d) δ 10.31 (t, J=6.0 Hz, 1H), 8.33 (s, 1H), 7.37 (td, J=8.5, 6.5 Hz, 1H), 6.91-6.75 (m, 2H), 6.01 (d, J=6.2 Hz, 1H), 5.79 (d, J=6.2 Hz, 1H), 4.94-4.81 (m, 1H), 4.68 (dd, J=15.1, 6.0 Hz, 1H), 4.61 (dd, J=15.1, 5.8 Hz, 1H), 4.04 (d, J=2.1 Hz, 1H), 3.77 (dd, J=15.0, 1.9 Hz, 1H), 3.62 (dd, J=15.0, 2.7 Hz, 1H), 3.05 (d, J=17.7 Hz, 1H), 2.60 (d, J=17.8 Hz, 1H), 2.38 (t, J=7.6 Hz, 2H), 2.11 (s, 3H), 1.99-1.85 (m, 3H), 1.65-1.55 (m, 4H), 1.31-1.25 (m, 27H), 1.24 (d, J=6.7 Hz, 3H), 0.90 (t, J=6.8 Hz, 3H).

### Example 108: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl isobutyrate (108)

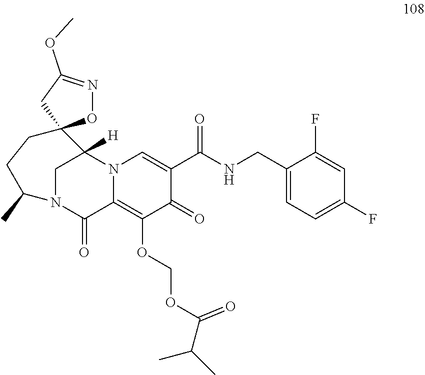 [SMILES: COC1=NO[C@@]2(CC[C@H](C)N3C[C@H]2n2cc(C(=O)NCc4ccc(F)cc4F)c(=O)c(OCOC(=O)C(C)C)c2C3=O)C1]

The title compound was prepared in a similar manner to Example 18, except using chloromethyl isobutyrate instead of chloromethyl pivalate and Intermediate C instead of Intermediate B. MS (m/z) 603.0 [M+H]+. 1H NMR (400 MHz, MeOD) δ 8.64 (s, 1H), 7.57-7.40 (m, 1H), 7.19-6.86 (m, 2H), 5.86 (d, J=6.4 Hz, 1H), 5.68 (d, J=6.3 Hz, 1H), 4.70-4.57 (m, 3H), 3.92 (s, 3H), 3.87-3.71 (m, 2H), 3.25-3.13 (m, 1H), 2.78 (d, J=17.1 Hz, 1H), 2.68-2.52 (m, 3H), 2.14-1.81 (m, 3H), 1.65-1.46 (m, 1H), 1.26 (d, J=6.7 Hz, 3H), 1.20-0.96 (m, 6H).

### Example 109: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl isobutyrate (109)

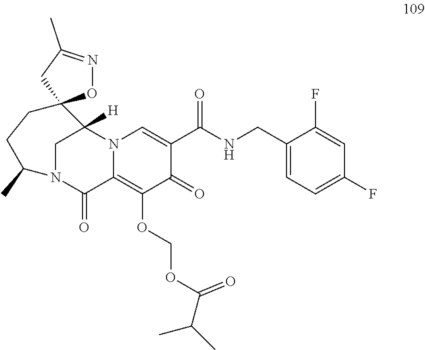 [SMILES: CC1=NO[C@@]2(CC[C@H](C)N3C[C@H]2n2cc(C(=O)NCc4ccc(F)cc4F)c(=O)c(OCOC(=O)C(C)C)c2C3=O)C1]

The title compound was prepared in a similar manner to Example 18, except using chloromethyl isobutyrate instead of chloromethyl pivalate. MS (m/z) 587.0 [M+H]+. ¹H NMR (400 MHz, MeOD) δ 8.60 (s, 1H), 7.57-7.38 (m, 1H), 7.08-6.96 (m, 2H), 5.94-5.77 (m, 1H), 5.77-5.57 (m, 1H), 4.73-4.59 (m, 2H), 4.59-4.43 (m, 1H), 4.01-3.63 (m, 2H), 3.27-3.10 (m, 1H), 2.81-2.47 (m, 3H), 2.13-2.02 (m, 3H), 2.00-1.89 (m, 4H), 1.69-1.43 (m, 1H), 1.26 (d, J=6.7 Hz, 3H), 1.22-1.10 (m, 6H).

### Example 110: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-methylbutanoate (110)

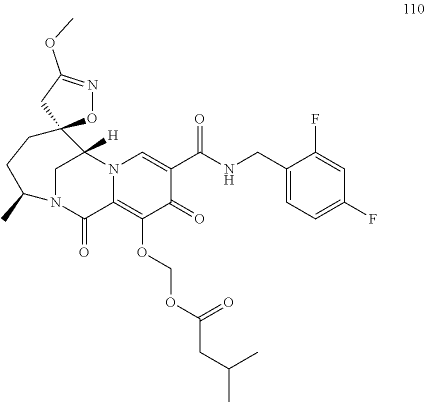 [SMILES: COC1=NO[C@@]2(CC[C@H](C)N3C[C@H]2n2cc(C(=O)NCc4ccc(F)cc4F)c(=O)c(OCOC(=O)CC(C)C)c2C3=O)C1]

The title compound was prepared in a similar manner to Example 18, except using chloromethyl 3-methylbutanoate instead of chloromethyl pivalate and Intermediate C instead of Intermediate B. MS (m/z) 617.0 [M+H]⁺. 1H NMR (400 MHz, Chloroform-d) δ 10.33 (t, J=5.9 Hz, 1H), 8.51 (s, 1H), 7.38 (td, J=8.4, 6.4 Hz, 1H), 6.90-6.77 (m, 2H), 5.99 (d, J=6.2 Hz, 1H), 5.79 (d, J=6.3 Hz, 1H), 4.92-4.81 (m, 1H), 4.68 (dd, J=15.1, 6.1 Hz, 1H), 4.59 (dd, J=15.1, 5.8 Hz, 1H), 4.26 (s, 1H), 3.89 (s, 3H), 3.75 (dd, J=15.1, 1.9 Hz, 1H), 3.61 (dd, J=15.1, 2.7 Hz, 1H), 3.09 (d, J=17.1 Hz, 1H), 2.66 (d, J=17.1 Hz, 1H), 2.32-2.19 (m, 2H), 2.16-1.96 (m, 2H), 1.94 (td, J=9.6, 8.7, 3.2 Hz, 2H), 1.67-1.55 (m, 1H), 1.22 (d, J=6.7 Hz, 3H), 0.95 (dd, J=6.6, 2.7 Hz, 6H).

### Example 111: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 3-methylbutanoate (111)

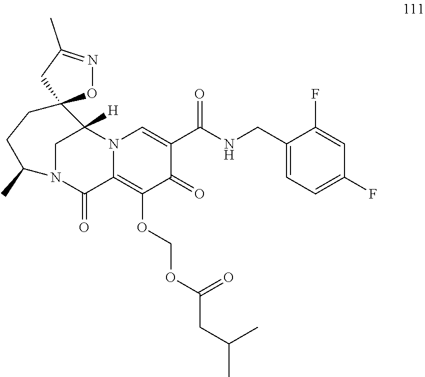 [SMILES: CC1=NO[C@@]2(CC[C@H](C)N3C[C@H]2n2cc(C(=O)NCc4ccc(F)cc4F)c(=O)c(OCOC(=O)CC(C)C)c2C3=O)C1]

The title compound was prepared in a similar manner to Example 18, except using chloromethyl 3-methylbutanoate instead of chloromethyl pivalate. MS (m/z) 601.0 [M+H]⁺. 1H NMR (400 MHz, Chloroform-d) δ 10.31 (t, J=6.0 Hz, 1H), 8.34 (s, 1H), 7.37 (td, J=8.6, 6.5 Hz, 1H), 6.90-6.77 (m, 2H), 5.99 (d, J=6.2 Hz, 1H), 5.79 (d, J=6.2 Hz, 1H), 4.86 (h, J=7.1, 6.6 Hz, 1H), 4.67 (dd, J=15.1, 6.0 Hz, 1H), 4.60 (dd, J=15.1, 5.9 Hz, 1H), 4.06 (d, J=2.3 Hz, 1H), 3.76 (dd, J=15.1, 1.9 Hz, 1H), 3.63 (dd, J=15.0, 2.7 Hz, 1H), 3.10-3.00 (m, 1H), 2.64-2.54 (m, 1H), 2.27 (dd, J=7.1, 1.1 Hz, 2H), 2.10 (s, 3H), 2.16-2.01 (m, 1H), 1.99-1.84 (m, 3H), 1.69-1.54 (m, 1H), 1.23 (d, J=6.7 Hz, 3H), 0.95 (dd, J=6.7, 2.6 Hz, 6H).

### Example 112: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 4-methylpentanoate (112)

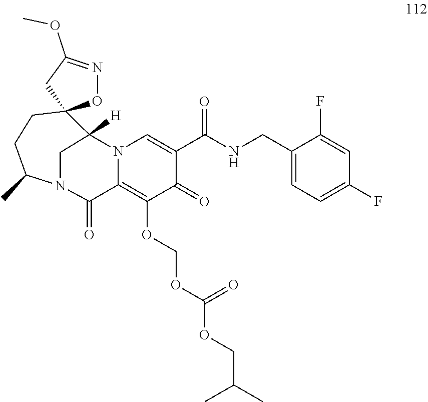 [SMILES: COC1=NO[C@@]2(CC[C@H](C)N3C[C@H]2n2cc(C(=O)NCc4ccc(F)cc4F)c(=O)c(OCOC(=O)OCC(C)C)c2C3=O)C1]

The title compound was prepared in a similar manner to Example 82, except using Intermediate B instead of Intermediate C in Step 2. MS (m/z) 614.994 [M+H]+. 1H NMR (400 MHz, Chloroform-d) δ 10.33 (t, J=6.0 Hz, 1H), 8.37 (s, 1H), 7.37 (td, J=8.6, 6.6 Hz, 1H), 6.91-6.73 (m, 2H), 6.00 (d, J=6.2 Hz, 1H), 5.80 (d, J=6.3 Hz, 1H), 4.94-4.81 (m, 1H), 4.64 (qd. J=15.1, 5.8 Hz, 2H), 4.07 (s, 1H), 3.76 (dd, J=15.1, 1.9 Hz, 1H), 3.63 (dd, J=15.0, 2.7 Hz, 1H), 3.06 (d, J=17.8 Hz, 1H), 2.59 (dd, J=17.8, 1.3 Hz, 1H), 2.43-2.35 (m, 2H), 2.10 (s, 3H), 1.98-1.87 (m, 3H), 1.63-1.48 (m, 4H), 1.24 (d, J=6.7 Hz, 3H), 0.89 (d, J=6.4 Hz, 6H).

### Example 113: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 5-methylhexanoate (113)

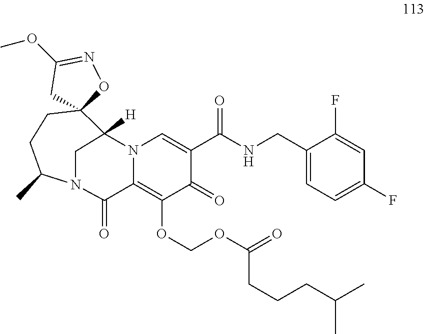 [SMILES: COC1=NO[C@@]2(CC[C@H](C)N3C[C@H]2n2cc(C(=O)NCc4ccc(F)cc4F)c(=O)c(OCOC(=O)CCCC(C)C)c2C3=O)C1]

The title compound was prepared in a similar manner to Example 18, except using chloromethyl 5-methylhexanoate instead of chloromethyl pivalate and Intermediate C instead of Intermediate B. MS (m-z) 645.981 [M+H]⁺. ¹H NMR (400 MHz, Chloroform-d) δ 10.32 (t, J=6.0 Hz, 1H), 8.45 (s, 1H), 7.47-7.33 (m, 1H), 6.92-6.75 (m, 2H), 6.01 (d, J=6.2 Hz, 1H), 5.80 (d, J=6.3 Hz, 1H), 4.88 (td, J=8.3, 6.5 Hz, 1H), 4.64 (qd, J=15.1, 5.9 Hz, 2H), 4.19 (s, 1H), 3.91 (s, 3H), 3.76 (dd, J=15.1, 1.9 Hz, 1H), 3.61 (dd, J=15.1, 2.7 Hz, 1H), 3.09 (d, J=17.2 Hz, 1H), 2.68 (d, J=17.2 Hz, 1H), 2.37 (t, J=7.6 Hz, 2H), 2.06-1.98 (m, 1H), 1.98-1.90 (m, 2H), 1.66-1.55 (m, 5H), 1.23 (d, J=6.6 Hz, 4H), 0.88 (d, J=6.7 Hz, 6H).

### Example 114: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 6-methylheptanoate (114)

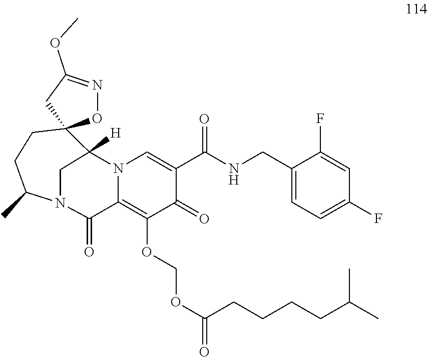 [SMILES: COC1=NO[C@@]2(CC[C@H](C)N3C[C@H]2n2cc(C(=O)NCc4ccc(F)cc4F)c(=O)c(OCOC(=O)CCCCC(C)C)c2C3=O)C1]

The title compound was prepared in a similar manner to Example 82, except using 6-methylheptanoic acid instead of in Step 1. MS (m/z) 659.05 [M+H]+. 1H NMR (400 MHz, Chloroform-d) δ 10.35 (t, J=5.9 Hz, 1H), 8.69 (s, 1H), 7.36 (td, J=8.4, 6.3 Hz, 1H), 6.82 (tdd, J=11.5, 8.5, 2.6 Hz, 2H), 5.99 (d, J=6.3 Hz, 1H), 5.75 (d, J=6.3 Hz, 1H), 4.86 (dt, J=9.5, 6.6 Hz, 1H), 4.68 (dd, J=15.1, 6.1 Hz, 1H), 4.56 (dd, J=15.2, 5.7 Hz, 1H), 4.43 (s, 1H), 3.83 (s, 3H), 3.72 (dd, J=15.1, 1.9 Hz, 1H), 3.61 (dd, J=15.0, 2.7 Hz, 1H), 3.11 (d, J=17.0 Hz, 1H), 2.61 (d, J=17.1 Hz, 1H), 2.37 (t, J=7.6 Hz, 2H), 2.04-1.85 (m, 3H), 1.60 (d, J=7.3 Hz, 1H), 1.60-1.44 (m, 3H), 1.30 (dddd, J=12.0, 9.6, 7.8, 4.4 Hz, 2H), 1.25-1.10 (m, 5H), 0.85 (d, J=6.6 Hz, 6H).

### Example 115: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 6-methylheptanoate (115)

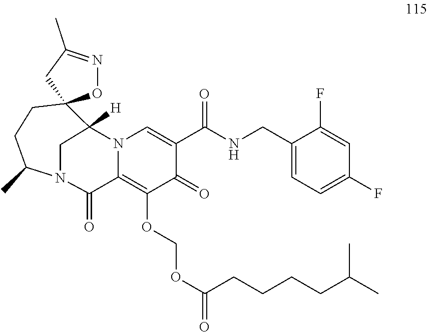 [SMILES: CC1=NO[C@@]2(CC[C@H](C)N3C[C@H]2n2cc(C(=O)NCc4ccc(F)cc4F)c(=O)c(OCOC(=O)CCCCC(C)C)c2C3=O)C1]

The title compound was prepared in a similar manner to Example 82, except using 6-methylheptanoic acid instead of 4-methylpentanoic acid in Step 1 and Intermediate B instead of Intermediate C in Step 2. MS (m/z) 643.9 [M+H]+. 1H NMR (400 MHz, Chloroform-d) δ 10.33 (t, J=6.0 Hz, 1H), 8.36 (s, 1H), 7.37 (td, J=8.6, 6.5 Hz, 1H), 6.90-6.77 (m, 2H), 6.00 (d, J=6.3 Hz, 1H), 5.79 (d, J=6.3 Hz, 1H), 4.87 (q, J=7.9 Hz, 1H), 4.64 (qd, J=15.1, 5.9 Hz, 2H), 4.07 (d, J=2.3 Hz, 1H), 3.76 (dd, J=15.0, 1.9 Hz, 1H), 3.62 (dd, J=15.0, 2.7 Hz, 1H), 3.06 (d, J=17.7 Hz, 1H), 2.64-2.54 (m, 1H), 2.38 (t, J=7.6 Hz, 2H), 2.10 (s, 3H), 1.99-1.85 (m, 3H), 1.67-1.45 (m, 4H), 1.38-1.29 (m, 1H), 1.34-1.12 (m, 6H), 0.87 (d, J=6.6 Hz, 6H).

### Example 116: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 7-methyloctanoate (116)

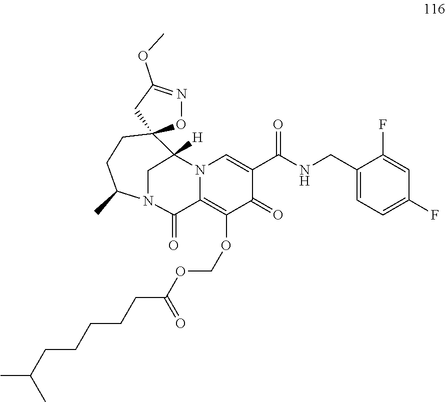 [SMILES: COC1=NO[C@@]2(CC[C@H](C)N3C[C@H]2n2cc(C(=O)NCc4ccc(F)cc4F)c(=O)c(OCOC(=O)CCCCCC(C)C)c2C3=O)C1]

The title compound was prepared in a similar manner to Example 82, except using 7-methyloctanoic acid instead of 4-methylpentanoic acid in Step 1. MS (m/z) 673.0 [M+H]⁺. 1H NMR (400 MHz, Chloroform-d) δ 10.34 (t, J=5.9 Hz, 1H), 8.64 (s, 1H), 7.36 (td, J=8.5, 6.4 Hz, 1H), 6.95-6.65 (m, 2H), 5.99 (d, J=6.3 Hz, 1H), 5.76 (d, J=6.2 Hz, 1H), 4.85 (ddt, J=14.8, 12.1, 6.1 Hz, 1H), 4.68 (dd, J=15.1, 6.1 Hz, 1H), 4.57 (dd, J=15.1, 5.7 Hz, 1H), 4.38 (d, J=2.3 Hz, 1H), 3.85 (s, 3H), 3.73 (dd, J=15.1, 1.9 Hz, 1H), 3.61 (dd, J=15.1, 2.7 Hz, 1H), 3.11 (d, J=17.0 Hz, 1H), 2.63 (d, J=17.1 Hz, 1H), 2.37 (t, J=7.6 Hz, 2H), 2.04-1.86 (m, 3H), 1.66-1.54 (m, 3H), 1.50 (dq, J=13.2, 6.6 Hz, 1H), 1.32-1.25 (m, 4H), 1.22 (d, J=6.7 Hz, 3H), 1.14 (qd, J=5.9, 5.3, 3.1 Hz, 2H), 0.86 (s, 3H), 0.85 (s, 3H).

### Example 117: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 7-methyloctanoate (117)

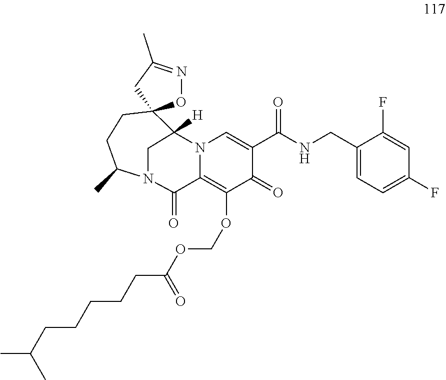 [SMILES: CC1=NO[C@@]2(CC[C@H](C)N3C[C@H]2n2cc(C(=O)NCc4ccc(F)cc4F)c(=O)c(OCOC(=O)CCCCCC(C)C)c2C3=O)C1]

The title compound was prepared in a similar manner to Example 82, except using 7-methyloctanoic acid instead of 4-methylpentanoic acid in Step 1 and Intermediate B instead of Intermediate C in Step 2. MS (m/z) 657.0 [M+H]⁺. 1H NMR (400 MHz, Chloroform-d) δ 10.32 (t, J=6.0 Hz, 1H), 8.34 (s, 1H), 7.37 (td, J=8.7, 6.6 Hz, 1H), 6.90-6.78 (m, 2H), 6.01 (d, J=6.3 Hz, 1H), 5.79 (d, J=6.2 Hz, 1H), 4.87 (h, J=7.0 Hz, 1H), 4.64 (qd, J=15.1, 5.9 Hz, 2H), 4.05 (d, J=2.3 Hz, 1H), 3.77 (dd, J=15.0, 1.9 Hz, 1H), 3.62 (dd, J=15.0, 2.7 Hz, 1H), 3.11-3.01 (m, 1H), 2.60 (dd, J=17.8, 1.3 Hz, 1H), 2.39 (t, J=7.6 Hz, 2H), 2.10 (s, 2H), 2.13-1.99 (m, 1H), 2.00-1.87 (m, 3H), 1.68-1.43 (m, 4H), 1.35-1.25 (m, J=3.3, 2.7 Hz, 4H), 1.24 (d, J=6.7 Hz, 3H), 1.16 (dddd, J=10.2, 8.3, 5.8, 2.9 Hz, 2H), 0.87 (d, J=6.6 Hz, 6H).

### Example 118: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (5-methylhexyl) carbonate (118)

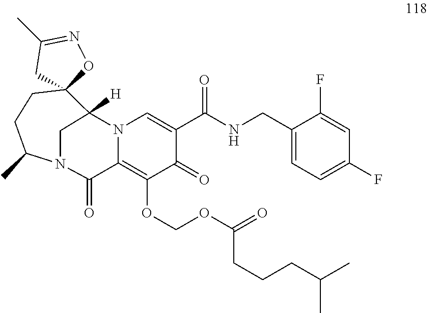 [SMILES: CC1=NO[C@@]2(CC[C@H](C)N3C[C@H]2n2cc(C(=O)NCc4ccc(F)cc4F)c(=O)c(OCOC(=O)CCCC(C)C)c2C3=O)C1]

The title compound was prepared in a similar manner to Example 18, except using chloromethyl 5-methylhexanoate instead of chloromethyl pivalate. MS (m/z) 629.981 [M+H]⁺. ¹H NMR (400 MHz, Chloroform-d) δ 10.31 (t, J=5.9 Hz, 1H), 8.34 (s, 1H), 7.37 (td, J=8.6, 6.6 Hz, 1H), 6.92-6.73 (m, 2H), 6.01 (d, J=6.3 Hz, 1H), 5.79 (d, J=6.3 Hz, 1H), 4.87 (d, J=7.4 Hz, 1H), 4.65 (dt, J=14.3, 7.4 Hz, 2H), 4.05 (d, J=2.2 Hz, 1H), 3.76 (dd, J=15.0, 1.9 Hz, 1H), 3.62 (dd, J=15.1, 2.7 Hz, 1H), 3.06 (d, J=17.8 Hz, 1H), 2.68-2.50 (m, 1H), 2.37 (t, J=7.6 Hz, 2H), 2.10 (s, 3H), 1.94 (d, J=7.2 Hz, 2H), 1.66-1.54 (m, 5H), 1.27-1.17 (m, 5H), 0.88 (d, J=6.6 Hz, 6H).

### Example 119: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 8-methylnonanoate (119)

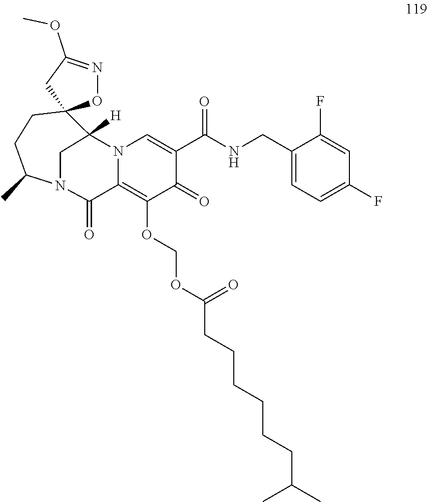 [SMILES: COC1=NO[C@@]2(CC[C@H](C)N3C[C@H]2n2cc(C(=O)NCc4ccc(F)cc4F)c(=O)c(OCOC(=O)CCCCCCC(C)C)c2C3=O)C1]

The title compound was prepared in a similar manner to Example 82, except using 8-methylnonanoic acid instead of 4-methylpentanoic acid in Step 1. MS (m/z) 687.6 [M+H]+. 1H NMR (400 MHz, DMSO-d6) δ 10.30 (t, J=5.9 Hz, 1H), 8.74 (s, 1H), 7.42 (td, J=8.7, 6.6 Hz, 1H), 7.24 (ddd, J=10.5, 9.3, 2.6 Hz, 1H), 7.17-6.90 (m, 1H), 5.76 (d, J=6.2 Hz, 1H), 5.59 (d. J=6.3 Hz, 1H), 4.81-4.45 (m, 4H), 3.82 (s, 3H), 3.67 (d, J=2.3 Hz, 2H), 3.06 (d, J=16.8 Hz, 1H), 2.75 (d, J=16.9 Hz, 1H), 2.27 (td, J=7.3, 1.3 Hz, 2H), 2.09-1.66 (m, 3H), 1.48 (pd, J=7.0, 3.5 Hz, 3H), 1.43-1.06 (m, 12H), 0.84 (d, J=6.6 Hz, 6H).

### Example 120: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl 8-methylnonanoate (120)

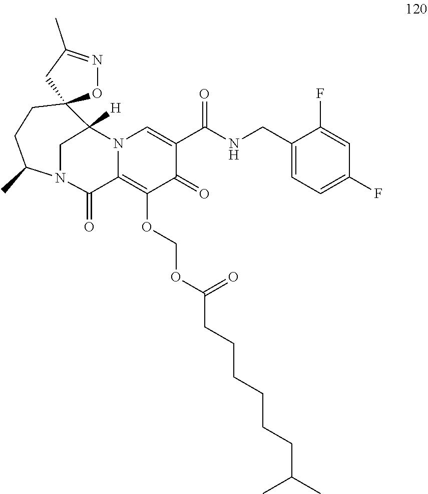 [SMILES: CC1=NO[C@@]2(CC[C@H](C)N3C[C@H]2n2cc(C(=O)NCc4ccc(F)cc4F)c(=O)c(OCOC(=O)CCCCCCC(C)C)c2C3=O)C1]

The title compound was prepared in a similar manner to Example 82, except using 8-methylnonanoic acid instead of 4-methylpentanoic acid in Step 1 and Intermediate B instead of Intermediate C in Step 2. MS (m/z) 671.5 [M+H]+. 1H NMR (400 MHz, DMSO-d6) δ 10.30 (t, J=6.0 Hz, 1H), 8.69 (s, 1H), 7.42 (td, J=8.7, 6.6 Hz, 1H), 7.25 (ddd, J=10.5, 9.3, 2.6 Hz, 1H), 7.17-7.00 (m, 1H), 5.78 (d, J=6.3 Hz, 1H), 5.59 (d, J=6.2 Hz, 1H), 4.73-4.45 (m, 4H), 3.68 (d, J=2.3 Hz, 2H), 3.01 (d, J=17.5 Hz, 1H), 2.63 (d, J=17.6 Hz, 1H), 2.39-2.20 (m, 2H), 1.95 (s, 3H), 1.77 (dt, J=20.9, 7.8 Hz, 3H), 1.48 (pd, J=7.0, 3.8 Hz, 3H), 1.40-1.03 (m, 12H), 0.84 (d, J=6.6 Hz, 6H).

### Example 121: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl carbonate (121)

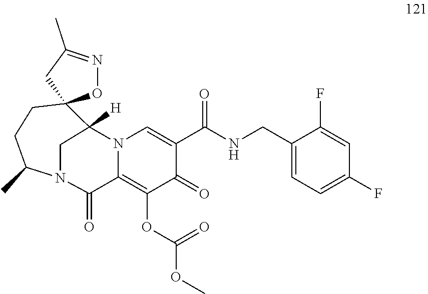 [SMILES: COC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using methyl carbonochloridate instead of 2-methoxyethyl carbonochloridate and Intermediate B instead of Intermediate C. MS (m/z) 545.0 [M+H]⁺. ¹H NMR (400 MHz, Methanol-d4) δ 8.69 (s, 1H), 7.53-7.37 (m, 1H), 7.08-6.87 (m, 2H), 4.82-4.48 (m, 4H), 4.02-3.89 (m, 3H), 3.89-3.77 (m, 2H), 3.16 (d, J=17.8 Hz, 1H), 2.71 (d, J=17.9 Hz, 1H), 2.09-1.89 (m, 6H), 1.68-1.46 (m, 1H), 1.27 (d, J=6.7 Hz, 3H).

### Example 122: Preparation of (03'S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl methyl carbonate (122)

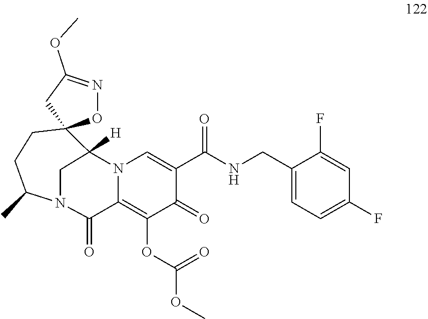 [SMILES: COC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 1, except using methyl carbonochloridate instead of 2-methoxyethyl carbonochloridate. MS (m/z) 561.0 [M+H]⁺. ¹H NMR (400 MHz, Methanol-d4) δ 8.73 (t, J=1.5 Hz, 1H), 7.53-7.33 (m, 1H), 7.21-6.87 (m, 2H), 4.84-4.57 (m, 4H), 3.95-3.85 (m, 5H), 3.86-3.75 (m, 2H), 3.18 (d, J=17.1 Hz, 1H), 2.95 (d, J=52.9 Hz, 1H), 2.78 (d, J=17.1 Hz, 1H), 2.17-1.88 (m, 3H), 1.66-1.48 (m, 1H), 1.27 (d, J=6.7 Hz, 3H).

### Example 123: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ethyl carbonate (123)

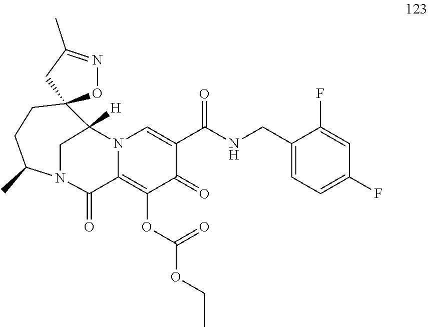 [SMILES: CCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using ethyl chloroformate instead of 2-methoxyethyl carbonochloridate and Intermediate B instead of Intermediate C. MS (m/z) 559.20 [M+H]⁺. 1H NMR (400 MHz, Chloroform-d) S 10.14 (t, J=5.9 Hz, 1H), 8.42 (s, 1H), 7.32 (td, J=8.7, 6.5 Hz, 1H), 6.86-6.73 (m, 2H), 4.79 (dq, J=9.8, 6.8 Hz, 1H), 4.64 (dd, J=15.1, 6.1 Hz, 1H), 4.54 (dd, J=15.1, 5.7 Hz, 1H), 4.35 (q, J=7.1 Hz, 2H), 3.77 (dd, J=15.1, 1.9 Hz, 1H), 3.67 (dd, J=15.2, 2.7 Hz, 1H), 3.07-2.93 (m, 1H), 2.58 (dd, J=17.7, 1.3 Hz, 1H), 2.07 (s, 3H), 1.97-1.81 (m, 3H), 1.73 (s, 1H), 1.59 (ddt, J=17.5, 8.2, 4.6 Hz, 1H), 1.39 (t, J=7.1 Hz, 3H), 1.22 (d, J=4.7 Hz, 3H).

### Example 124: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ethyl carbonate (124)

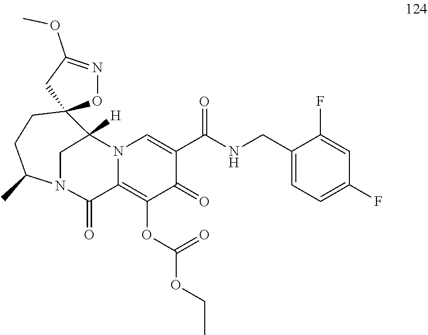 [SMILES: CCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 1, except using ethyl chloroformate instead of 2-methoxyethyl carbonochloridate. MS (m/z) 575.20 [M+H]⁺. 1H NMR (400 MHz, Chloroform-d) δ 10.20 (t, J=5.9 Hz, 1H), 8.80 (s, 1H), 7.33 (td, J=8.4, 6.3 Hz, 1H), 6.88-6.72 (m, 2H), 4.81 (dq, J=10.2, 6.8 Hz, 1H), 4.68 (dd, J=15.1, 6.2 Hz, 1H), 4.56-4.43 (m, 2H), 4.35 (q. J=7.1 Hz, 2H), 3.81 (s, 3H), 3.76 (dd, J=15.2, 1.8 Hz, 1H), 3.67 (dd, J=15.1, 2.6 Hz, 1H), 3.08 (d, J=17.0 Hz, 1H), 2.61 (d, J=17.1 Hz, 1H), 2.02-1.82 (m, 3H), 1.60 (ddd, J=15.3, 10.5, 2.1 Hz, 1H), 1.39 (t, J=7.1 Hz, 3H), 1.24-1.19 (m, 3H).

### Example 125: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl propyl carbonate (125)

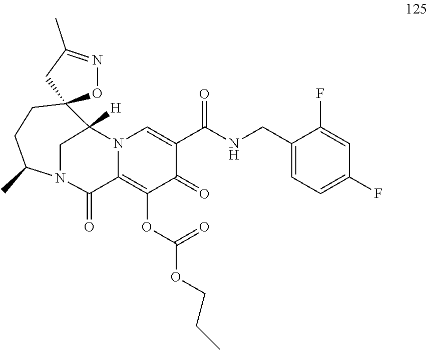 [SMILES: CCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using propyl carbonochloridate instead of 2-methoxyethyl carbonochloridate and Intermediate B instead of Intermediate C. MS (m/z) 573.0 [M+H]⁺. ¹H NMR (400 MHz, Methanol-d4) δ 8.68 (s, 1H), 7.55-7.32 (m, 1H), 7.07-6.88 (m, 2H), 4.79-4.45 (m, 4H), 4.36-4.16 (m, 2H), 3.94-3.73 (m, 2H), 3.23-3.10 (m, 1H), 2.81-2.62 (m, 1H), 2.15-1.89 (m, 6H), 1.88-1.69 (m, 2H), 1.66-1.48 (m, 1H), 1.27 (d, J=6.7 Hz, 3H), 1.03 (t, J=7.4 Hz, 3H).

### Example 126: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl propyl carbonate (126)

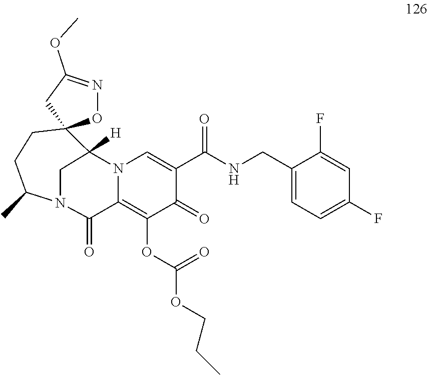 [SMILES: CCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 1, except using propyl carbonochloridate instead of 2-methoxyethyl carbonochloridate. MS (m/z) 589.0 [M+H]⁺. ¹H NMR (400 MHz, Methanol-d4) δ 8.73 (s, 1H), 7.56-7.36 (m, 1H), 7.04-6.90 (m, 2H), 4.83-4.67 (m, 1H), 4.65 (s, 3H), 4.36-4.19 (m, 2H), 3.92 (s, 3H), 3.87-3.72 (m, 2H), 3.25-3.11 (m, 1H), 2.77 (d, J=17.1 Hz, 1H), 2.15-1.85 (m, 3H), 1.85-1.68 (m, 2H), 1.68-1.48 (m, 1H), 1.27 (d, J=6.7 Hz, 3H), 1.03 (t, J=7.4 Hz, 3H).

### Example 127: Preparation of butyl butyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate (127)

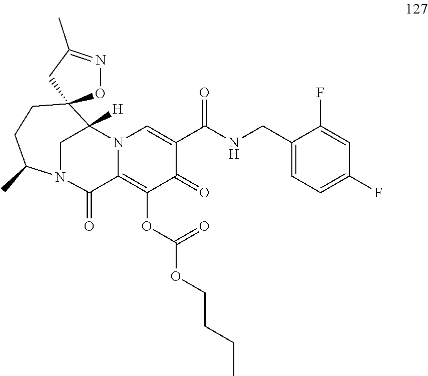 [SMILES: CCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using butyl carbonochloridate instead of 2-methoxyethyl carbonochloridate, and Intermediate B instead of Intermediate C. MS (m/z) 587.0 [M+H]⁺. 1H NMR (400 MHz, Chloroform-d) δ 10.15 (t, J=6.0 Hz, 1H), 8.49 (s, 1H), 7.32 (td, J=8.7, 6.4 Hz, 1H), 6.96-6.66 (m, 2H), 4.78 (dq, J=9.9, 6.8 Hz, 1H), 4.64 (dd, J=15.1, 6.1 Hz, 1H), 4.53 (dd, J=15.1, 5.7 Hz, 1H), 4.28 (t. J=6.8 Hz, 2H), 4.20 (d, J=2.3 Hz, 1H), 3.82-3.54 (m, 2H), 3.08-2.96 (m, 1H), 2.96-2.80 (m, 3H), 2.57 (dd, J=17.7, 1.3 Hz, 1H), 1.95-1.81 (m, 3H), 1.81-1.64 (m, 2H), 1.58 (ddd, J=15.0, 8.8, 3.8 Hz, 1H), 1.50-1.34 (m, 2H), 1.22 (d, J=6.6 Hz, 3H), 0.93 (t, J=7.4 Hz, 3H).

### Example 128: Preparation of butyl ((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate (128)

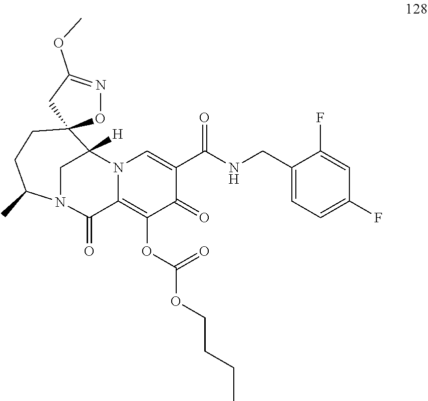 [SMILES: CCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 1, except using butyl carbonochloridate instead of 2-methoxyethyl carbonochloridate. MS (m/z) 603.0 [M+H]⁺. 1H NMR (400 MHz, Chloroform-d) δ 10.24 (t, J=5.9 Hz, 1H), 8.82 (s, 1H), 7.36 (td, J=8.4, 6.3 Hz, 1H), 6.89-6.76 (m, 2H), 4.84 (dp, J=10.2, 6.8 Hz, 1H), 4.71 (dd, J=15.1, 6.2 Hz, 1H), 4.58-4.46 (m, 2H), 4.32 (t, J=6.8 Hz, 2H), 3.84 (s, 3H), 3.79 (dd, J=15.1, 1.9 Hz, 1H), 3.68 (dd, J=15.1, 2.7 Hz, 1H), 3.10 (d, J=17.1 Hz, 1H), 2.63 (d, J=17.1 Hz, 1H), 2.08-1.85 (m, 3H), 1.82-1.56 (m, 3H), 1.54-1.40 (m, 2H), 1.26 (t, J=6.4 Hz, 3H), 0.97 (t, J=7.4 Hz, 3H).

### Example 129: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl pentyl carbonate (129)

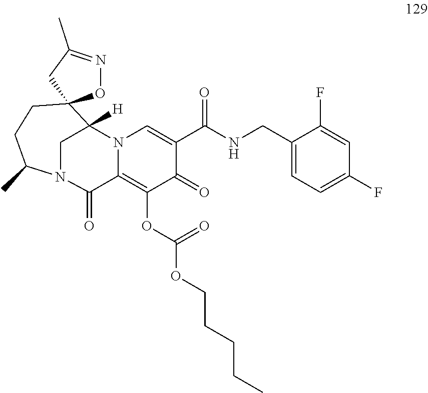 [SMILES: CCCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 2, except using n-pentanol instead of 2-(2-methoxyethoxy)ethanol in Step 1 and using Intermediate B instead of Intermediate C in Step 2. MS (m/z) 601.25 [M+H]⁺. 1H NMR (400 MHz, Chloroform-d) δ 10.16 (t, J=5.9 Hz, 1H), 8.39 (s, 1H), 7.34 (td, J=8.6, 6.5 Hz, 1H), 6.88-6.74 (m, 2H), 4.81 (dt, J=16.6, 6.9 Hz, 1H), 4.66 (dd, J=15.1, 6.0 Hz, 1H), 4.56 (dd, J=15.1, 5.5 Hz, 1H), 4.30 (t, J=6.8 Hz, 2H), 4.06 (d, J=2.4 Hz, 1H), 3.79 (dd, J=15.1, 1.9 Hz, 1H), 3.66 (dd, J=15.1, 2.7 Hz, 1H), 3.03 (d, J=17.8 Hz, 1H), 2.60 (dd, J=17.8, 1.3 Hz, 1H), 2.09 (d, J=1.1 Hz, 3H), 2.00-1.83 (m, 3H), 1.77 (p, J=7.0 Hz, 2H), 1.67-1.58 (m, 1H), 1.46-1.29 (m, 4H), 1.24 (d, J=6.7 Hz, 3H), 0.91 (t, J=7.0 Hz, 3H).

### Example 130: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl pentyl carbonate (130)

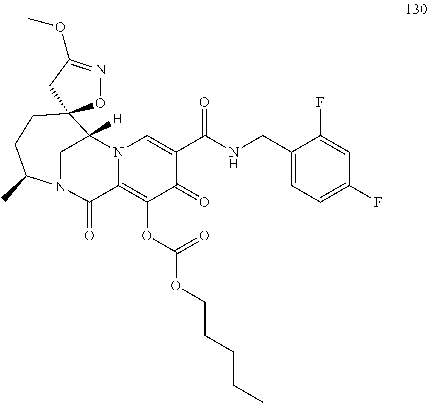 [SMILES: CCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 2, except using n-pentanol instead of 2-(2-methoxyethoxy)ethanol in Step 1. MS (m/z) 617.24 [M+H]⁺. 1H NMR (400 MHz, Chloroform-d) δ 1H NMR (400 MHz, Chloroform-d) δ 10.17 (t, J=5.8 Hz, 1H), 8.54 (s, 1H), 7.41-7.30 (m, 1H), 6.87-6.73 (m, 2H), 4.82 (dt, J=9.9, 6.8 Hz, 1H), 4.67 (dd, J=15.1, 6.0 Hz, 1H), 4.55 (dd, J=15.0, 5.5 Hz, 1H), 4.30 (t, J=6.8 Hz, 2H), 4.25 (s, 1H), 3.89 (s, 3H), 3.78 (dd, J=15.1, 1.8 Hz, 1H), 3.65 (dd, J=15.2, 2.7 Hz, 1H), 3.06 (d, J=17.1 Hz, 1H), 2.67 (d, J=17.2 Hz, 1H), 2.06-1.85 (m, 3H), 1.77 (p, J=7.0 Hz, 2H), 1.68-1.58 (m, 1H), 1.45-1.30 (m, 4H), 1.23 (d, J=6.7 Hz, 3H), 0.91 (t, J=7.1 Hz, 3H).

### Example 131: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl hexyl carbonate (131)

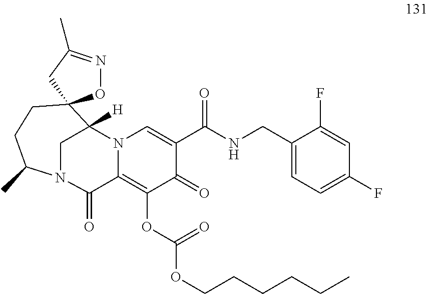 [SMILES: CCCCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using hexyl carbonochloridate instead of 2-methoxyethyl carbonochloridate and Intermediate B instead of Intermediate C. MS (m/z) 614.92 [M+H]⁺. 1H NMR (400 MHz, Chloroform-d) δ 10.16 (t, J=6.0 Hz, 1H), 8.49 (s, 1H), 7.33 (q, J=7.9 Hz, 1H), 6.80 (q, J=9.3, 8.4 Hz, 2H), 4.80 (q, J=7.7 Hz, 1H), 4.65 (dd, J=15.3, 6.1 Hz, 1H), 4.54 (dd, J=15.2, 5.6 Hz, 1H), 4.28 (t, J=6.8 Hz, 2H), 4.20 (s, 1H), 3.76 (d, J=15.1 Hz, 1H), 3.69 (d, J=15.1 Hz, 1H), 3.05 (d, J=17.7 Hz, 1H), 2.57 (d, J=17.7 Hz, 1H), 2.05 (s, 3H), 1.90 (dd, J=23.3, 13.5 Hz, 3H), 1.74 (d, J=7.6 Hz, 2H), 1.58 (dd, J=17.0, 7.9 Hz, 1H), 1.41-1.19 (m, 9H), 0.89 (d, J=6.2 Hz, 3H).

### Example 132: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl hexyl carbonate (132)

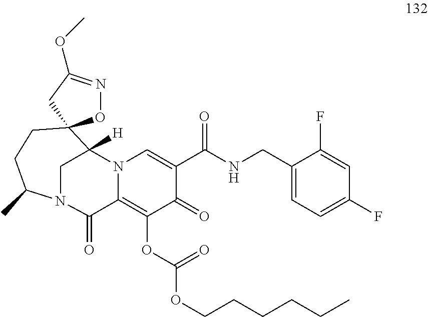 [SMILES: CCCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar method as Example 1, except using hexyl carbonochloridate instead of 2-methoxyethyl carbonochloridate. MS (m/z) 630.95 [M+H]⁺. 1H NMR (400 MHz, Chloroform-d) δ 10.25 (t, J=5.9 Hz, 1H), 8.90 (s, 1H), 7.36 (td, J=8.4, 6.3 Hz, 1H), 6.88-6.75 (m, 2H), 4.84 (dt, J=10.2, 6.6 Hz, 1H), 4.72 (dd, J=15.1, 6.2 Hz, 1H), 4.59-4.46 (m, 2H), 4.30 (t, J=6.8 Hz, 2H), 3.84-3.73 (m, 4H), 3.69 (dd, J=15.1, 2.7 Hz, 1H), 3.11 (d, J=17.0 Hz, 1H), 2.61 (d, J=17.1 Hz, 1H), 1.95 (dtt, J=21.9, 13.9, 5.8 Hz, 3H), 1.83-1.72 (m, 2H), 1.67-1.55 (m, 1H), 1.43 (dd, J=10.8, 4.8 Hz, 2H), 1.42-1.28 (m, 4H), 1.25 (d, J=6.7 Hz, 3H), 0.94-0.84 (m, 3H).

### Example 133: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl heptyl carbonate (133)

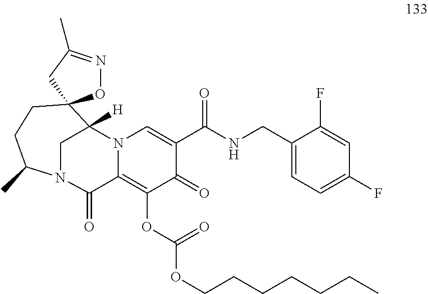 [SMILES: CCCCCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 2, except using heptan-1-ol instead of using 2-(2-methoxyethoxy)ethanol in Step 1, and using Intermediate B instead of Intermediate C in Step 2. MS (m/z) 628.94 [M+H]⁺. 1H NMR (400 MHz, Chloroform-d) δ 10.17 (t, J=5.9 Hz, 1H), 8.47 (s, 1H), 7.34 (td, J=8.7, 6.4 Hz, 1H), 6.88-6.75 (m, 2H), 4.81 (dq, J=9.4, 6.8 Hz, 1H), 4.66 (dd, J=15.1, 6.0 Hz, 1H), 4.55 (dd, J=15.1, 5.6 Hz, 1H), 4.30 (t, J=6.8 Hz, 2H), 4.17 (d, J=2.2 Hz, 1H), 3.78 (dd, J=15.2, 1.9 Hz, 1H), 3.69 (dd, J=15.1, 2.7 Hz, 1H), 3.05 (d, J=17.7 Hz, 1H), 2.59 (d, J=17.7 Hz, 1H), 2.07 (s, 3H), 2.03-1.72 (m, 5H), 1.60 (ddd, J=14.9, 8.9, 3.7 Hz, 1H), 1.40 (qd, J=8.5, 8.0, 2.8 Hz, 2H), 1.38-1.25 (m, 6H), 1.24 (d, J=6.7 Hz, 3H), 0.89 (t, J=6.6 Hz, 3H).

### Example 134: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl heptyl carbonate (134)

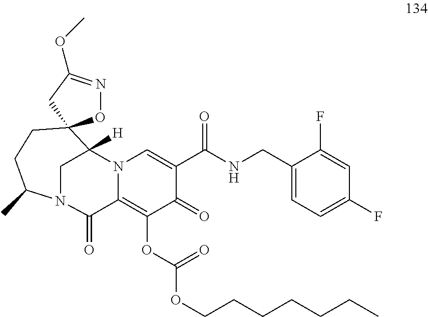 [SMILES: CCCCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 2, except using heptan-1-ol instead of 2-(2-methoxyethoxy)ethanol in Step 1. MS (m/z) 644.9 [M+H]⁺. 1H NMR (400 MHz, Chloroform-d) δ 10.21 (t, J=5.9 Hz, 1H), 8.65 (s, 1H), 7.36 (q, J=7.8 Hz, 1H), 6.83 (q, J=10.2, 9.4 Hz, 2H), 4.84 (dp, J=13.8, 7.4 Hz, 1H), 4.70 (dd, J=15.2, 5.8 Hz, 1H), 4.56 (dd, J=15.2, 5.3 Hz, 1H), 4.32 (dd, J=12.9, 6.1 Hz, 3H), 3.89 (s, 3H), 3.80 (d, J=15.1 Hz, 1H), 3.67 (d, J=15.2 Hz, 1H), 3.09 (d, J=17.1 Hz, 1H), 2.67 (d, J=17.1 Hz, 1H), 2.02 (dd, J=15.1, 5.9 Hz, 1H), 1.96 (t, J=10.3 Hz, 2H), 1.78 (p, J=7.1 Hz, 2H), 1.43 (t, J=7.7 Hz, 1H), 1.39-1.22 (m, 1H), 0.89 (q, J=11.0, 8.5 Hz, 3H).

### Example 135: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl octyl carbonate (135)

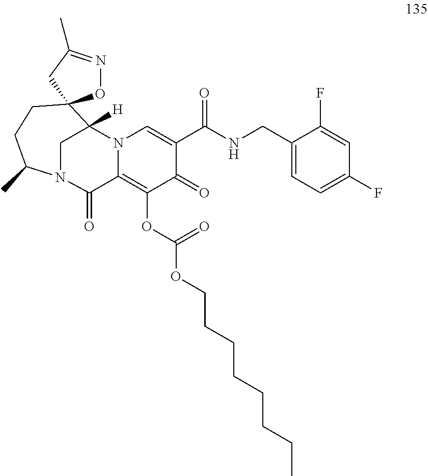 [SMILES: CCCCCCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using octyl carbonochloridate instead of 2-methoxyethyl carbonochloridate and Intermediate B instead of Intermediate C. MS (m/z) 643.0 [M+H]⁺. 1H NMR (400 MHz, Methanol-d4) δ 8.70 (s, 1H), 7.44 (td, J=8.5, 6.3 Hz, 1H), 7.04-6.79 (m, 2H), 4.74 (dtd, J=13.4, 9.5, 8.1, 5.5 Hz, 1H), 4.68-4.51 (m, 3H), 4.26 (tt, J=7.8, 3.9 Hz, 2H), 3.86 (qd, J=15.2, 2.3 Hz, 2H), 3.22-3.09 (m, 1H), 2.70 (dd, J=17.9, 1.2 Hz, 1H), 2.04 (s, 3H), 2.00-1.85 (m, 3H), 1.73 (dt, J=8.3, 6.5 Hz, 2H), 1.61-1.25 (m, 14H), 1.03-0.83 (m, 3H).

### Example 136: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl octyl carbonate (136)

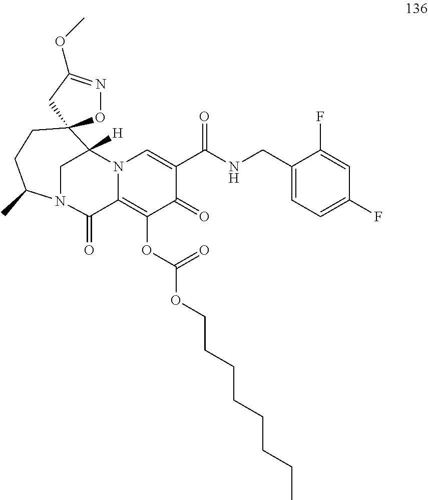 [SMILES: CCCCCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 1, except using octyl carbonochloridate instead of 2-methoxyethyl carbonochloridate. MS (m/z) 659.0 [M+H]⁺. 1H NMR (400 MHz, Methanol-d4) δ 8.91 (s, 1H), 7.44 (td, J=8.5, 6.4 Hz, 1H), 7.03-6.88 (m, 2H), 4.82-4.67 (m, 2H), 4.62 (s, 2H), 4.27 (tt, J=7.5, 3.8 Hz, 2H), 3.90 (dd, J=15.3, 2.7 Hz, 1H), 3.84 (s, 3H), 3.19 (d, J=17.0 Hz, 1H), 2.73 (d, J=17.1 Hz, 1H), 2.04 (dd, J=15.4, 6.2 Hz, 1H), 2.02-1.86 (m, 2H), 1.73 (dt, J=8.3, 6.5 Hz, 2H), 1.56 (ddd, J=15.6, 10.7, 2.0 Hz, 1H), 1.50-1.34 (m, 2H), 1.37-1.23 (m, 12H), 0.96-0.88 (m, 3H).

### Example 137: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl nonyl carbonate (137)

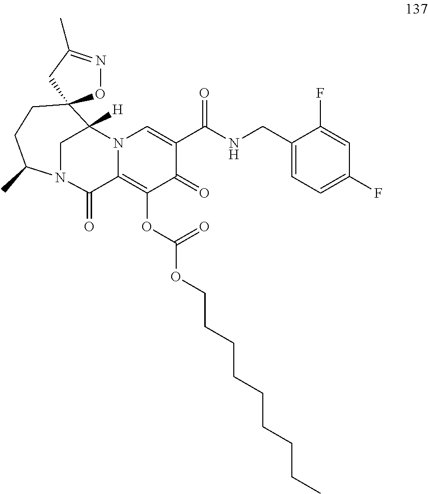 [SMILES: CCCCCCCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using nonyl carbonochloridate instead of 2-methoxyethyl carbonochloridate and Intermediate B instead of Intermediate C. MS (m/z) 657.1 [M+H]⁺. 1 H NMR (400 MHz, Chloroform-d) δ 10.17 (t, J=5.9 Hz, 1H), 8.46 (s, 1H), 7.34 (td, J=8.7, 6.4 Hz, 1H), 6.88-6.75 (m, 2H), 4.81 (dq, J=9.4, 6.7 Hz, 1H), 4.67 (dd, J=15.1, 6.2 Hz, 1H), 4.56 (dd, J=15.1, 5.7 Hz, 1H), 4.30 (t, J=6.8 Hz, 2H), 4.16 (d, J=2.2 Hz, 1H), 3.78 (dd, J=15.2, 1.9 Hz, 1H), 3.69 (dd, J=15.1, 2.7 Hz, 1H), 3.05 (d, J=17.7 Hz, 1H), 2.64-2.54 (m, 1H), 2.08 (s, 3H), 2.00-1.84 (m, 3H), 1.83-1.70 (m, 2H), 1.61 (ddd, J=15.0, 9.0, 3.7 Hz, 1H), 1.41 (qd, J=6.8, 3.8 Hz, 2H), 1.37-1.24 (m, 10H), 1.24 (d, J=6.7 Hz, 3H), 0.93-0.83 (m, 3H).

### Example 138: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl nonyl carbonate (138)

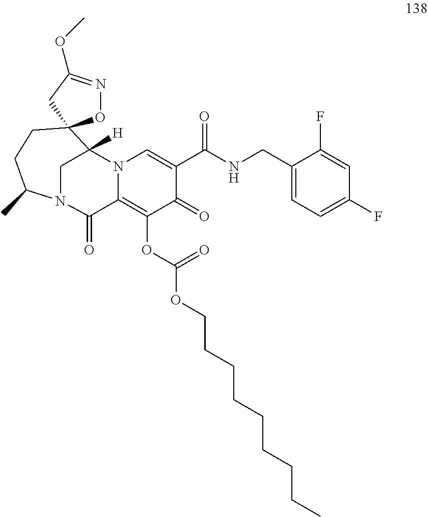 [SMILES: CCCCCCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 1, except using nonyl carbonochloridate instead of 2-methoxyethyl carbonochloridate. MS (m/z) 673.0 [M+H]⁺. 1H NMR (400 MHz, Chloroform-d) δ 10.25 (t, J=5.9 Hz, 1H), 8.95-8.84 (m, 1H), 7.36 (td, J=8.3, 6.2 Hz, 1H), 6.89-6.75 (m, 2H), 4.84 (dt, J=10.3, 6.6 Hz, 1H), 4.72 (dd, J=15.1, 6.3 Hz, 1H), 4.61-4.47 (m, 2H), 4.30 (t, J=6.8 Hz, 2H), 3.85-3.74 (m, 4H), 3.69 (dd, J=15.2, 2.7 Hz, 1H), 3.11 (d, J=17.0 Hz, 1H), 2.61 (dt, J=17.1, 3.1 Hz, 1H), 2.05-1.85 (m, 3H), 1.75 (dt, J=15.6, 8.0 Hz, 3H), 1.61 (dd, J=15.2, 10.8 Hz, 1H), 1.48-1.36 (m, 2H), 1.28 (ddd, J=16.8, 9.6, 4.7 Hz, 12H), 0.89 (t, J=6.7 Hz, 3H).

### Example 139: Preparation of decyl ((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate (139)

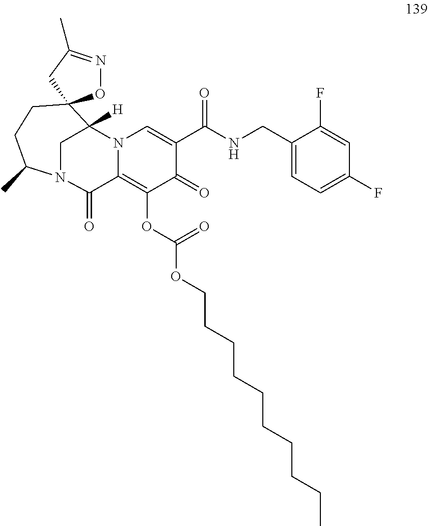 [SMILES: CCCCCCCCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using decyl carbonochloridate instead of 2-methoxyethyl carbonochloridate and Intermediate B instead of Intermediate C. MS (m/z) 671.1 [M+H]⁺. 1H NMR (400 MHz, Chloroform-d) δ 10.17 (t, J=6.0 Hz, 1H), 8.47 (d, J=4.0 Hz, 1H), 7.39-7.28 (m, 1H), 6.87-6.75 (m, 2H), 4.82 (q, J=7.6 Hz, 1H), 4.66 (dd, J=15.2, 6.2 Hz, 1H), 4.55 (dd, J=15.3, 5.7 Hz, 1H), 4.29 (td, J=6.9, 1.8 Hz, 2H), 4.17 (d, J=5.9 Hz, 1H), 3.78 (dd, J=15.2, 2.4 Hz, 1H), 3.69 (dd, J=15.1, 2.7 Hz, 1H), 3.05 (d, J=17.7 Hz, 1H), 2.59 (d, J=17.7 Hz, 1H), 2.07 (d, J=3.3 Hz, 3H), 2.03-1.80 (m, 3H), 1.84-1.70 (m, 2H), 1.60 (ddt, J=12.4, 6.5, 3.2 Hz, 1H), 1.41 (t, J=7.6 Hz, 2H), 1.26 (q, J=9.4, 6.5 Hz, 15H), 0.88 (t, J=6.6 Hz, 3H).

### Example 140: Preparation of decyl ((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl) carbonate (140)

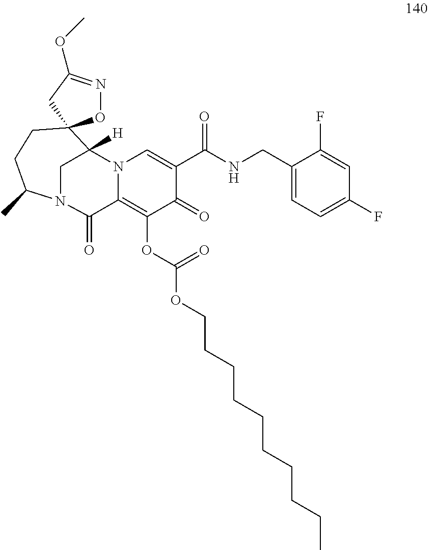 [SMILES: CCCCCCCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 1, except using decyl carbonochloridate instead of 2-methoxyethyl carbonochloridate. MS (m/z) 687.0 [M+H]⁺. 1H NMR (400 MHz, Chloroform-d) δ 10.21 (t, J=6.0 Hz, 1H), 8.62 (s, 1H), 7.36 (td, J=8.4, 6.3 Hz, 1H), 6.90-6.77 (m, 2H), 4.91-4.77 (m, 1H), 4.70 (dd, J=15.1, 6.2 Hz, 1H), 4.56 (dd, J=15.1, 5.6 Hz, 1H), 4.36-4.27 (m, 3H), 3.89 (s, 3H), 3.80 (dd, J=15.1, 1.9 Hz, 1H), 3.67 (dd, J=15.2, 2.7 Hz, 1H), 3.09 (d, J=17.1 Hz, 1H), 2.68 (d, J=17.2 Hz, 1H), 2.08-1.88 (m, 3H), 1.78 (p, J=7.0 Hz, 2H), 1.70-1.61 (m, 1H), 1.43 (dq, J=11.1, 6.9 Hz, 2H), 1.28 (q, J=10.0, 6.7 Hz, 15H), 0.90 (t, J=6.7 Hz, 3H).

### Example 141: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl dodecyl carbonate (141)

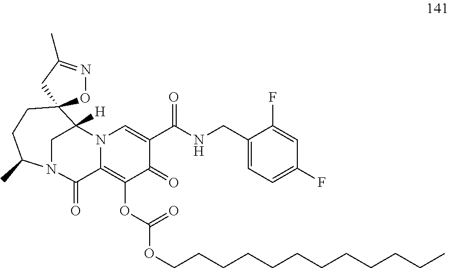 [SMILES: CCCCCCCCCCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using dodecyl carbonochloridate instead of 2-methoxyethyl carbonochloridate and using Intermediate B instead of Intermediate C. MS (m/z) 698.97 [M+H]⁺. 1H NMR (400 MHz, Chloroform-d) δ 10.18 (t, J=6.0 Hz, 1H), 8.46 (s, 1H), 7.34 (td, J=8.7, 6.4 Hz, 1H), 6.88-6.76 (m, 2H), 4.82 (dt, J=9.6, 6.6 Hz, 1H), 4.67 (dd, J=15.1, 6.2 Hz, 1H), 4.56 (dd, J=15.1, 5.7 Hz, 1H), 4.30 (t, J=6.8 Hz, 2H), 4.15 (d, J=2.2 Hz, 1H), 3.79 (dd, J=15.2, 1.9 Hz, 1H), 3.69 (dd, J=15.2, 2.7 Hz, 1H), 3.05 (d, J=17.7 Hz, 1H), 2.60 (d, J=17.8 Hz, 1H), 2.08 (s, 3H), 2.03-1.84 (m, 3H), 1.77 (dd, J=14.0, 6.6 Hz, 2H), 1.61 (ddd, J=14.9, 9.0, 3.7 Hz, 1H), 1.41 (dq, J=11.4, 6.7 Hz, 2H), 1.26 (q, J=8.0, 6.6 Hz, 19H), 0.89 (t, J=6.7 Hz, 3H).

### Example 142: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl dodecyl carbonate (142)

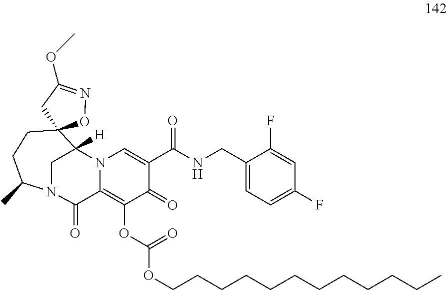 [SMILES: CCCCCCCCCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared following a similar method as Example 1, except using dodecyl carbonochloridate instead of 2-methoxyethyl carbonochloridate. MS (m/z) 714.99 [M+H]⁺. 1H NMR (400 MHz, Chloroform-d) δ 10.23 (t, J=5.9 Hz, 1H), 8.73 (s, 1H), 7.36 (td, J=8.4, 6.3 Hz, 1H), 6.89-6.76 (m, 2H), 4.83 (dq, J=10.1, 6.8 Hz, 1H), 4.71 (dd, J=15.1, 6.2 Hz, 1H), 4.54 (dd, J=15.1, 5.6 Hz, 1H), 4.42 (s, 1H), 4.31 (t, J=6.9 Hz, 2H), 3.87 (s, 3H), 3.79 (dd, J=15.2, 1.9 Hz, 1H), 3.68 (dd, J=15.1, 2.7 Hz, 1H), 3.10 (d, J=17.1 Hz, 1H), 2.65 (d, J=17.1 Hz, 1H), 2.07-1.82 (m, 4H), 1.77 (h, J=8.2, 7.6 Hz, 3H), 1.69-1.55 (m, 2H), 1.44 (q, J=5.6, 4.3 Hz, 2H), 1.43-1.22 (m, 18H), 0.90 (t, J=1.8 Hz, 3H).

### Example 143: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl tetradecyl carbonate (143)

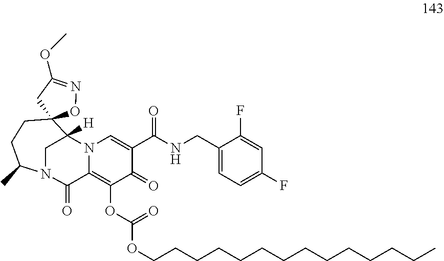 [SMILES: CCCCCCCCCCCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 2, except using tetradecan-1-ol instead of 2-(2-methoxyethoxy)ethanol in Step 1. MS (m/z) 742.9[M+H]⁺. 1H NMR (400 MHz, Chloroform-d) δ 10.20 (q, J=5.3 Hz, 1H), 8.70-8.43 (m, 1H), 7.37 (td, J=8.4, 6.3 Hz, 1H), 6.90-6.77 (m, 2H), 4.83 (dq, J=9.6, 6.8 Hz, 1H), 4.70 (dd, J=15.2, 6.1 Hz, 1H), 4.57 (ddd, J=15.0, 5.9, 2.3 Hz, 1H), 4.32 (t, J=6.8 Hz, 2H), 3.94-3.88 (m, 3H), 3.81 (dd, J=15.2, 1.8 Hz, 1H), 3.67 (dd, J=15.2, 2.7 Hz, 1H), 3.08 (dd, J=17.2, 1.8 Hz, 1H), 2.75-2.64 (m, 1H), 2.03 (dd, J=14.9, 5.7 Hz, 1H), 2.02-1.87 (m, 2H), 1.84-1.74 (m, 2H), 1.70-1.54 (m, 2H), 1.43 (tt, J=11.2, 6.7 Hz, 2H), 1.26 (d, J=13.1 Hz, 23H), 0.90 (t, J=6.8 Hz, 3H).

### Example 144: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl hexadecyl carbonate (144)

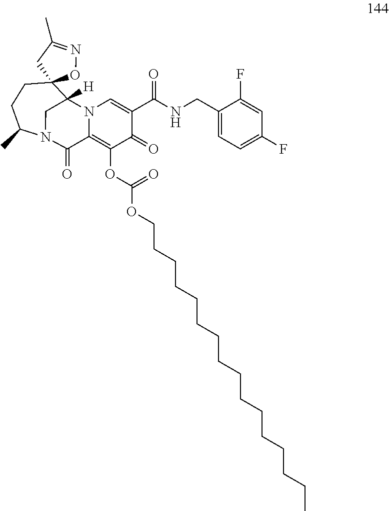 [SMILES: CCCCCCCCCCCCCCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 1, except using hexadecyl carbonochloridate instead of 2-methoxyethyl carbonochloridate and Intermediate B instead of Intermediate C. MS (m/z) 755.2 [M+H]⁺. 1H NMR (400 MHz, Chloroform-d) δ 10.17 (t, J=5.9 Hz, 1H), 8.63-8.33 (m, 1H), 7.34 (td, J=8.6, 6.5 Hz, 1H), 6.90-6.67 (m, 2H), 5.03-4.74 (m, 1H), 4.66 (dd, J=15.2, 6.1 Hz, 1H), 4.55 (dd, J=15.3, 5.7 Hz, 1H), 4.29 (td, J=6.9, 2.1 Hz, 2H), 4.18 (d, J=5.9 Hz, 1H), 3.89-3.55 (m, 2H), 3.05 (d, J=17.7 Hz, 1H), 2.59 (dd, J=17.7, 2.5 Hz, 1H), 2.07 (s, 3H), 2.00-1.80 (m, 3H), 1.83-1.70 (m, 2H), 1.60 (ddd, J=15.2, 8.1, 2.6 Hz, 1H), 1.48-1.16 (m, 29H), 0.95-0.76 (m, 3H).

### Example 145: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl hexadecyl carbonate (145)

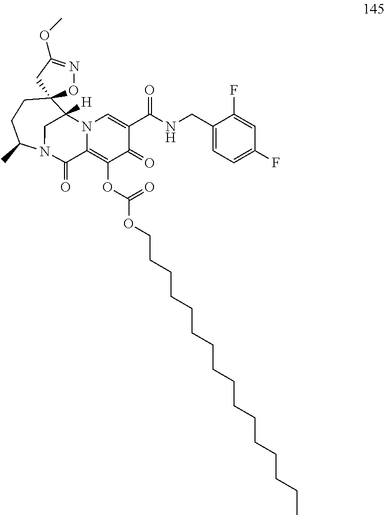 [SMILES: CCCCCCCCCCCCCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 1, except using hexadecyl carbonochloridate instead of 2-methoxyethyl carbonochloridate. MS (m/z) 771.4 [M+H]⁺. 1H NMR (400 MHz, Chloroform-d) δ 10.20 (t, J=5.9 Hz, 1H), 8.58 (s, 1H), 7.37 (td, J=8.4, 6.3 Hz, 1H), 6.90-6.77 (m, 2H), 4.84 (dt, J=10.1, 6.8 Hz, 1H), 4.70 (dd, J=15.1, 6.1 Hz, 1H), 4.57 (dd, J=15.1, 5.7 Hz, 1H), 4.36-4.25 (m, 3H), 3.91 (s, 3H), 3.80 (dd, J=15.1, 1.9 Hz, 1H), 3.67 (dd, J=15.1, 2.7 Hz, 1H), 3.08 (d, J=17.2 Hz, 1H), 2.69 (d, J=17.2 Hz, 1H), 2.08-1.93 (m, 2H), 1.97-1.87 (m, 1H), 1.78 (p, J=6.9 Hz, 2H), 1.71-1.56 (m, 1H), 1.47-1.22 (m, 29H), 0.94-0.86 (m, 3H).

### Example 146: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl octadecyl carbonate (146)

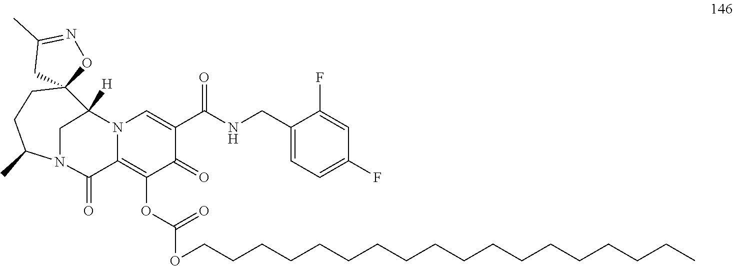 [SMILES: CCCCCCCCCCCCCCCCCCOC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 2, except using octadecan-1-ol instead of 2-(2-methoxyethoxy)ethanol in Step 1 and using Intermediate B instead of Intermediate C in Step 2. MS (m/z) 783.757 [M+H]⁺. ¹H NMR (400 MHz, Chloroform-d) δ 10.18 (s, 1H), 8.44 (s, 1H), 7.35 (d, J=7.7 Hz, 1H), 6.83 (d, J=8.9 Hz, 2H), 4.83 (d, J=7.7 Hz, 1H), 4.67 (t, J=7.6 Hz, 1H), 4.63-4.52 (m, 1H), 4.31 (t, J=6.9 Hz, 2H), 4.12 (s, 1H), 3.80 (d, J=15.0 Hz, 1H), 3.69 (d, J=15.1 Hz, 1H), 3.05 (d, J=17.7 Hz, 1H), 2.63 (s, 1H), 2.10 (s, 3H), 1.94 (d, J=9.3 Hz, 3H), 1.77 (s, 5H), 1.42 (s, 2H), 1.27 (s, 29H), 0.90 (d, J=6.2 Hz, 3H).

### Example 147: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl octadecyl carbonate (147)

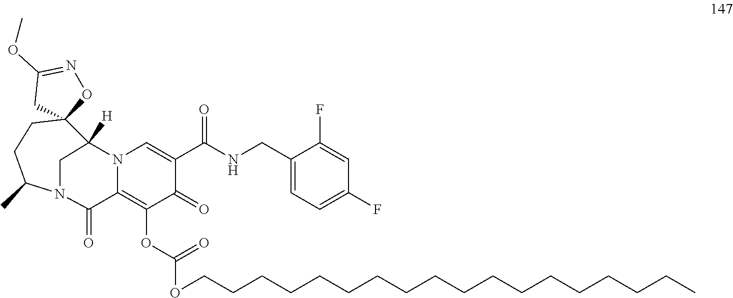 [SMILES: CCCCCCCCCCCCCCCCCCOC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 2, except using octadecan-1-ol instead of 2-(2-methoxyethoxy)ethanol in Step 1. MS (m/z) 799.4 [M+H]⁺. ¹H NMR (400 MHz, Chloroform-d) δ 10.20 (t, J=6.2 Hz, 1H), 8.57 (s, 1H), 7.37 (d, J=8.0 Hz, 1H), 6.83 (d, J=9.3 Hz, 2H), 4.85 (q, J=7.9 Hz, 1H), 4.70 (dd, J=15.4, 6.2 Hz, 1H), 4.57 (dd, J=15.8, 5.5 Hz, 1H), 4.31 (dd, J=15.1, 7.9 Hz, 3H), 3.91 (s, 3H), 3.81 (d, J=15.2 Hz, 1H), 3.67 (d, J=15.2 Hz, 1H), 3.08 (d, J=17.1 Hz, 1H), 2.70 (d, J=17.2 Hz, 1H), 2.11-1.89 (m, 3H), 1.78 (t, J=7.5 Hz, 2H), 1.59 (s, 3H), 1.48-1.40 (m, 2H), 1.28 (s, 29H), 0.90 (t, J=6.7 Hz, 3H).

### Example 148: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl isobutyl carbonate (148)

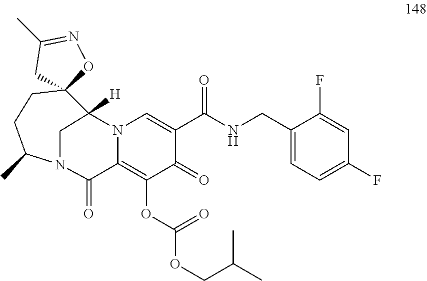 [SMILES: CC1=NO[C@@]2(CC[C@H](C)N3C[C@H]2n2cc(C(=O)NCc4ccc(F)cc4F)c(=O)c(OC(=O)OCC(C)C)c2C3=O)C1]

The title compound was prepared in a similar manner to Example 1, except using isobutyl carbonochloridate instead of 2-methoxyethyl carbonochloridate and using Intermediate B instead of Intermediate C. MS (m z) 587.992 [M+H]⁺. ¹H NMR (400 MHz, Chloroform-d) δ 10.18 (t, J=6.0 Hz, 1H), 8.42 (s, 1H), 7.45-7.32 (m, 1H), 6.93-6.75 (m, 2H), 4.84 (dd, J=6.9, 2.9 Hz, 1H), 4.69 (dd, J=15.1, 6.2 Hz, 1H), 4.57 (dd, J=15.1, 5.7 Hz, 1H), 4.15-4.07 (m, 3H), 3.81 (dd, J=15.1, 1.9 Hz, 1H), 3.68 (dd, J=15.1, 2.7 Hz, 1H), 3.05 (d, J=17.8 Hz, 1H), 2.67-2.57 (m, 1H), 2.12 (s, 3H), 2.00-1.92 (m, 2H), 1.62 (s, 2H), 1.26 (d, J=6.7 Hz, 4H), 1.06-0.99 (m, 6H).

### Example 149: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl isobutyl carbonate (149)

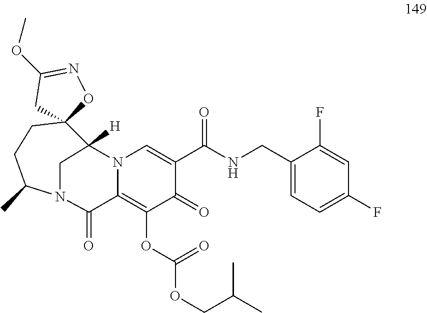 [SMILES: COC1=NO[C@@]2(CC[C@H](C)N3C[C@H]2n2cc(C(=O)NCc4ccc(F)cc4F)c(=O)c(OC(=O)OCC(C)C)c2C3=O)C1]

The title compound was prepared in a similar manner to Example 1, except using isobutyl carbonochloridate instead of 2-methoxyethyl carbonochloridate. MS (m/z) 603.896 [M+H]⁺. ¹H NMR (400 MHz, Chloroform-d) δ 10.20 (t, J=5.9 Hz, 1H), 8.60 (s, 1H), 7.37 (td, J=8.1, 6.0 Hz, 1H), 6.91-6.75 (m, 2H), 4.92-4.79 (m, 1H), 4.70 (dd, J=15.1, 6.2 Hz, 1H), 4.56 (dd, J=15.1, 5.6 Hz, 1H), 4.30 (s, 1H), 4.11 (dd, J=6.8, 3.1 Hz, 2H), 3.90 (s, 3H), 3.80 (dd, J=15.1, 1.9 Hz, 1H), 3.67 (dd, J=15.2, 2.8 Hz, 1H), 3.08 (d, J=17.1 Hz, 1H), 2.69 (d, J=17.2 Hz, 1H), 2.17-2.06 (m, 1H), 2.02-1.92 (m, 2H), 1.60 (s, 2H), 1.25 (d, J=6.7 Hz, 3H), 1.02 (dd, J=6.7, 1.0 Hz, 6H).

### Example 150: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl isopentyl carbonate (150)

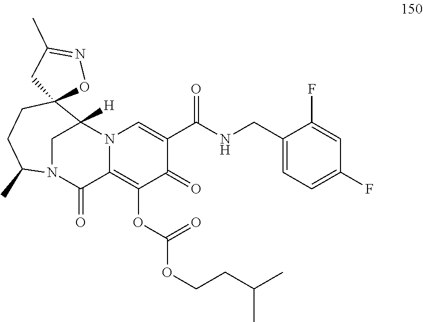 [SMILES: CC1=NO[C@@]2(CC[C@H](C)N3C[C@H]2n2cc(C(=O)NCc4ccc(F)cc4F)c(=O)c(OC(=O)OCCC(C)C)c2C3=O)C1]

The title compound was prepared in a similar manner to Example 1, except using isopentyl carbonochloridate instead of 2-methoxyethyl carbonochloridate, and Intermediate B instead of Intermediate C. MS (m/z) 600.94 [M+H]⁺. 1H NMR (400 MHz, Chloroform-d) δ 10.17 (t, J=5.9 Hz, 1H), 8.48 (s, 1H), 7.33 (td, J=8.7, 6.4 Hz, 1H), 6.87-6.75 (m, 2H), 4.81 (dt, J=9.8, 6.9 Hz, 1H), 4.66 (dd, J=15.1, 6.1 Hz, 1H), 4.55 (dd, J=15.1, 5.7 Hz, 1H), 4.33 (t, J=7.0 Hz, 2H), 4.19 (d, J=2.2 Hz, 1H), 3.77 (dd, J=15.2, 1.9 Hz, 1H), 3.69 (dd, J=15.1, 2.6 Hz, 1H), 3.10-3.00 (m, 1H), 2.58 (dd, J=17.7, 1.3 Hz, 1H), 2.06 (s, 3H), 2.02-1.83 (m, 3H), 1.77 (dt, J=13.4, 6.7 Hz, 1H), 1.65 (q, J=6.9 Hz, 2H), 1.65-1.54 (m, 1H), 1.23 (d, J=6.7 Hz, 3H), 0.94 (d, J=6.6 Hz, 6H).

### Example 151: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl isopentyl carbonate (151)

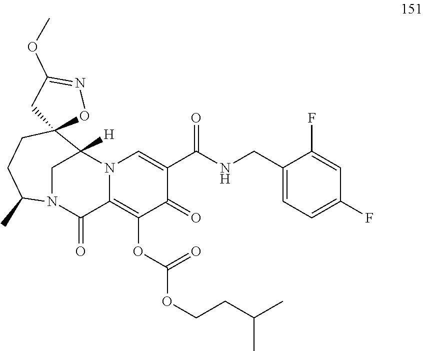 [SMILES: COC1=NO[C@@]2(CC[C@H](C)N3C[C@H]2n2cc(C(=O)NCc4ccc(F)cc4F)c(=O)c(OC(=O)OCCC(C)C)c2C3=O)C1]

The title compound was prepared in a similar manner to Example 1, except using isopentyl carbonochloridate instead of 2-methoxyethyl carbonochloridate, MS (m/z) 616.97 [M+H]⁺. 1H NMR (400 MHz, Chloroform-d) δ 10.26 (t, J=5.9 Hz, 1H), 9.00 (s, 1H), 7.35 (td, J=8.4, 6.3 Hz, 1H), 6.81 (ddt, J=12.3, 9.0, 4.1 Hz, 2H), 4.83 (dt, J=10.2, 6.5 Hz, 1H), 4.71 (dd, J=15.2, 6.2 Hz, 1H), 4.65 (s, 1H), 4.50 (dd, J=15.2, 5.4 Hz, 1H), 4.33 (t, J=7.0 Hz, 2H), 3.81-3.65 (m, 5H), 3.12 (d, J=17.0 Hz, 1H), 2.58 (d, J=17.0 Hz, 1H), 2.04-1.84 (m, 3H), 1.76 (dt, J=13.3, 6.7 Hz, 1H), 1.71-1.54 (m, 3H), 1.24 (d, J=6.6 Hz, 3H), 0.94 (d, J=6.6 Hz, 6H).

### Example 152: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (4-methylpentyl) carbonate (152)

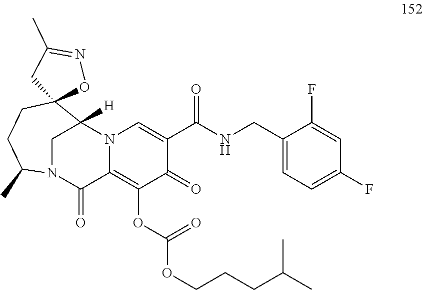 [SMILES: CC1=NO[C@@]2(CC[C@H](C)N3C[C@H]2n2cc(C(=O)NCc4ccc(F)cc4F)c(=O)c(OC(=O)OCCCC(C)C)c2C3=O)C1]

The title compound was prepared in a similar manner to Example 2, except using 4-methylpentan-1-ol instead of 2-(2-methoxyethoxy)ethanol in Step 1 and using Intermediate B instead of Intermediate C in Step 2. MS (m/z) 614.92 [M+H]⁺. 1H NMR (400 MHz, Chloroform-d) δ 10.17 (t, J=5.9 Hz, 1H), 8.48 (s, 1H), 7.34 (td, J=8.7, 6.4 Hz, 1H), 6.87-6.75 (m, 2H), 4.81 (dt, J=9.8, 6.8 Hz, 1H), 4.66 (dd, J=15.1, 6.1 Hz, 1H), 4.55 (dd, J=15.1, 5.6 Hz, 1H), 4.28 (t, J=6.9 Hz, 2H), 4.17 (t, J=2.2 Hz, 1H), 3.78 (dd, J=15.2, 1.9 Hz, 1H), 3.69 (dd, J=15.1, 2.6 Hz, 1H), 3.05 (d, J=17.7 Hz, 1H), 2.63-2.54 (m, 1H), 2.07 (s, 3H), 2.02-1.83 (m, 4H), 1.83-1.69 (m, 2H), 1.66-1.48 (m, 1H), 1.36-1.21 (m, 5H), 0.90 (d, J=6.6 Hz, 6H).

### Example 153: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (4-methylpentyl) carbonate (153)

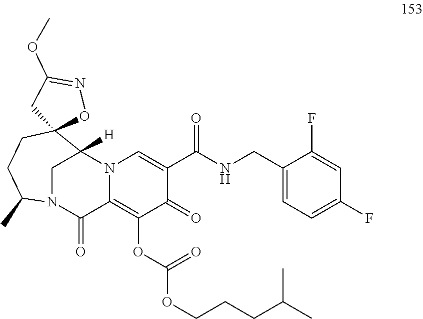 [SMILES: COC1=NO[C@@]2(CC[C@H](C)N3C[C@H]2n2cc(C(=O)NCc4ccc(F)cc4F)c(=O)c(OC(=O)OCCCC(C)C)c2C3=O)C1]

The title compound was prepared in a similar manner to Example 2, except using 4-methylpentan-1-ol instead of using 2-(2-methoxyethoxy)ethanol in Step 1. MS (m/z) 630.94[M+H]⁺. 1H NMR (400 MHz, Chloroform-d) δ 10.23 (t, J=5.9 Hz, 1H), 8.77 (s, 1H), 7.36 (td, J=8.4, 6.4 Hz, 1H), 6.89-6.76 (m, 2H), 4.91-4.77 (m, 1H), 4.71 (dd, J=15.1, 6.0 Hz, 1H), 4.54 (dd, J=15.1, 5.3 Hz, 1H), 4.45 (d, J=2.3 Hz, 1H), 4.30 (t, J=6.9 Hz, 2H), 3.85 (s, 3H), 3.83-3.74 (m, 1H), 3.68 (dd, J=15.1, 2.7 Hz, 1H), 3.10 (d, J=17.1 Hz, 1H), 2.64 (d, J=17.1 Hz, 1H), 2.07-1.89 (m, 4H), 1.80-1.70 (m, 1H), 1.70-1.51 (m, 2H), 1.37-1.26 (m, 2H), 1.25 (d, J=6.7 Hz, 3H), 0.91 (d, J=6.6 Hz, 6H).

### Example 154: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (5-methylhexyl) carbonate (154)

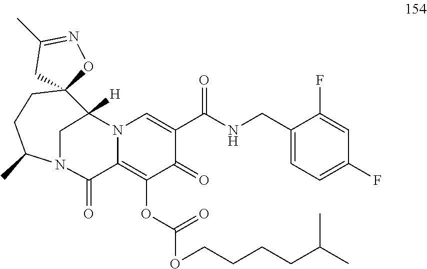 [SMILES: CC1=NO[C@@]2(CC[C@H](C)N3C[C@H]2n2cc(C(=O)NCc4ccc(F)cc4F)c(=O)c(OC(=O)OCCCCC(C)C)c2C3=O)C1]

The title compound was prepared in a similar manner to Example 2, except using 5-methylhexan-1-ol instead of 2-(2-methoxyethoxy)ethanol in Step 1 and using Intermediate B instead of Intermediate C in Step 2. MS (m/z) 629.9 [M+H]⁺. ¹H NMR (400 MHz, Chloroform-d) δ 10.18 (t, J=5.9 Hz, 1H), 8.42 (s, 1H), 7.35 (dd, J=8.5, 6.4 Hz, 1H), 6.91-6.75 (m, 2H), 4.84 (dd, J=7.2, 2.8 Hz, 1H), 4.69 (dd, J=15.1, 6.1 Hz, 1H), 4.58 (dd, J=15.1, 5.7 Hz, 1H), 4.32 (t, J=6.8 Hz, 2H), 4.09 (d, J=2.2 Hz, 1H), 3.81 (dd, J=15.2, 1.9 Hz, 1H), 3.70 (d, J=2.7 Hz, 1H), 3.05 (d, J=17.8 Hz, 1H), 2.67-2.55 (m, 1H), 2.11 (s, 3H), 2.01-1.86 (m, 3H), 1.84-1.70 (m, 2H), 1.61 (s, 2H), 1.57 (s, 1H), 1.43 (d, J=8.1 Hz, 2H), 1.24 (dd, J=9.5, 7.6 Hz, 4H), 0.90 (d, J=6.6 Hz, 6H).

### Example 155: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (5-methylhexyl) carbonate (155)

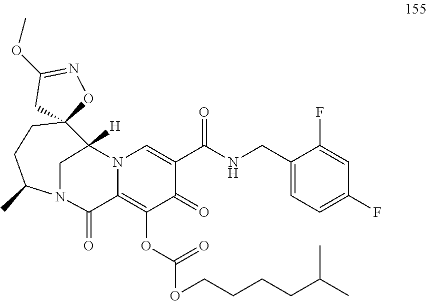 [SMILES: COC1=NO[C@@]2(CC[C@H](C)N3C[C@H]2n2cc(C(=O)NCc4ccc(F)cc4F)c(=O)c(OC(=O)OCCCCC(C)C)c2C3=O)C1]

The title compound was prepared in a similar manner to Example 2, except using 5-methylhexan-1-ol instead of 2-(2-methoxyethoxy)ethanol in Step 1. MS (m/z) 645.96 [M+H]⁺. ¹H NMR (400 MHz, Chloroform-d) δ 10.20 (t, J=6.0 Hz, 1H), 8.58 (s, 1H), 7.39 (d, J=8.0 Hz, 1H), 6.91-6.75 (m, 2H), 4.84 (dt, J=9.9, 6.8 Hz, 1H), 4.70 (dd, J=15.1, 6.2 Hz, 1H), 4.57 (dd, J=15.1, 5.6 Hz, 1H), 4.37-4.21 (m, 3H), 3.91 (s, 3H), 3.80 (dd, J=15.2, 1.8 Hz, 1H), 3.67 (dd, J=15.1, 2.7 Hz, 1H), 3.08 (d, J=17.2 Hz, 1H), 2.69 (d, J=17.2 Hz, 1H), 2.09-1.91 (m, 3H), 1.82-1.72 (m, 2H), 1.59 (s, 2H), 1.56 (d, J=6.6 Hz, 1H), 1.48-1.39 (m, 2H), 1.24 (t, J=8.0 Hz, 4H), 0.89 (d, J=6.6 Hz, 6H).

### Example 156: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (6-methylheptyl) carbonate (156)

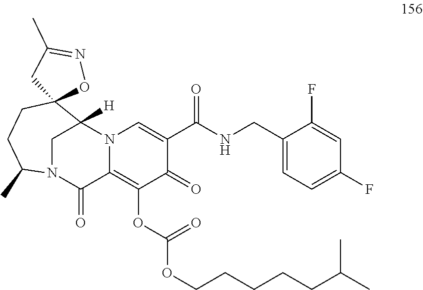 [SMILES: CC1=NO[C@@]2(CC[C@H](C)N3C[C@H]2n2cc(C(=O)NCc4ccc(F)cc4F)c(=O)c(OC(=O)OCCCCCC(C)C)c2C3=O)C1]

The title compound was prepared in a similar manner to Example 2, except using 6-methylheptan-1-ol instead of 2-(2-methoxyethoxy)ethanol in Step 1 and using Intermediate B instead of Intermediate C in Step 2. MS (m/z) 643.9 [M+H]⁺. 1H NMR (400 MHz, Chloroform-d) δ 10.17 (t, J=5.9 Hz, 1H), 8.48 (s, 1H), 7.33 (td, J=8.7, 6.4 Hz, 1H), 6.87-6.75 (m, 2H), 4.80 (dq, J=9.9, 6.8 Hz, 1H), 4.66 (dd, J=15.1, 6.1 Hz, 1H), 4.55 (dd, J=15.1, 5.7 Hz, 1H), 4.29 (t, J=6.8 Hz, 2H), 4.19 (d, J=2.2 Hz, 1H), 3.77 (dd, J=15.2, 1.9 Hz, 1H), 3.69 (dd, J=15.1, 2.6 Hz, 1H), 3.10-3.00 (m, 1H), 2.63-2.53 (m, 1H), 2.06 (s, 3H), 2.02-1.83 (m, 4H), 1.82-1.70 (m, 2H), 1.66-1.47 (m, 2H), 1.50-1.23 (m, 4H), 1.27-1.12 (m, 4H), 0.86 (d, J=6.6 Hz, 6H).

### Example 157: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (6-methylheptyl) carbonate (157)

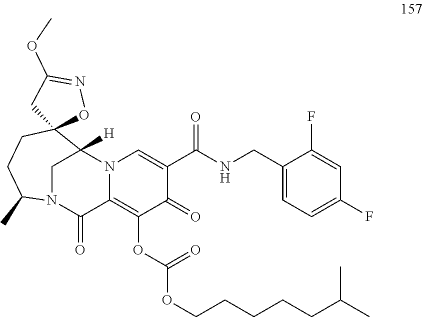 [SMILES: COC1=NO[C@@]2(CC[C@H](C)N3C[C@H]2n2cc(C(=O)NCc4ccc(F)cc4F)c(=O)c(OC(=O)OCCCCCC(C)C)c2C3=O)C1]

The title compound was prepared in a similar manner to Example 2, except using 6-methylheptan-1-ol instead of 2-(2-methoxyethoxy)ethanol in Step 1. MS (m/z) 659.9 [M+H]⁺. 1H NMR (400 MHz, Chloroform-d) δ 10.24 (t, J=5.9 Hz, 1H), 8.84 (s, 1H), 7.36 (td, J=8.4, 6.3 Hz, 1H), 6.89-6.76 (m, 2H), 4.84 (dp, J=10.2, 6.7 Hz, 1H), 4.72 (dd, J=15.1, 6.2 Hz, 1H), 4.58-4.48 (m, 2H), 4.31 (t, J=6.8 Hz, 2H), 3.84 (s, 3H), 3.78 (dd, J=15.2, 1.9 Hz, 1H), 3.68 (dd, J=15.1, 2.7 Hz, 1H), 3.11 (d, J=17.0 Hz, 1H), 2.63 (d, J=17.1 Hz, 1H), 2.06-1.85 (m, 3H), 1.84-1.72 (m, 2H), 1.68-1.47 (m, 2H), 1.48-1.25 (m, 4H), 1.28-1.13 (m, 5H), 0.88 (d, J=6.6 Hz, 6H).

### Example 158: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (7-methyloctyl) carbonate (158)

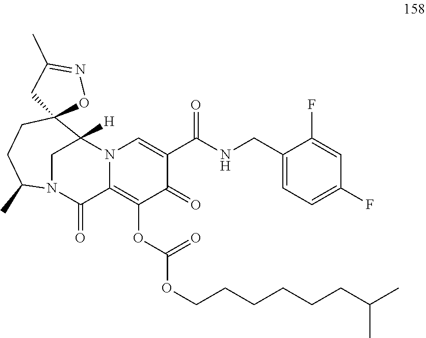 [SMILES: CC1=NO[C@@]2(CC[C@H](C)N3C[C@H]2n2cc(C(=O)NCc4ccc(F)cc4F)c(=O)c(OC(=O)OCCCCCCC(C)C)c2C3=O)C1]

The title compound was prepared in a similar manner to Example 2, except using 7-methyloctan-1-ol instead of 2-(2-methoxyethoxy)ethanol in Step 1 and using Intermediate B instead of Intermediate C in Step 2. MS (m/z) 657.07 [M+H]⁺. 1H NMR (400 MHz, Chloroform-d) δ 10.18 (t, J=6.0 Hz, 1H), 8.42 (s, 1H), 7.36 (td, J=8.7, 6.5 Hz, 1H), 6.90-6.77 (m, 2H), 4.84 (dt, J=9.7, 6.7 Hz, 1H), 4.69 (dd, J=15.1, 6.1 Hz, 1H), 4.58 (dd, J=15.1, 5.7 Hz, 1H), 4.32 (t, J=6.8 Hz, 2H), 4.08 (d, J=2.1 Hz, 1H), 3.81 (dd, J=15.2, 1.9 Hz, 1H), 3.68 (dd, J=15.1, 2.7 Hz, 1H), 3.05 (d, J=17.8 Hz, 1H), 2.62 (dd, J=17.8, 1.3 Hz, 1H), 2.11 (s, 3H), 2.05-1.86 (m, 3H), 1.79 (p, J=6.9 Hz, 2H), 1.70-1.38 (m, 2H), 1.44 (s, 2H), 1.40-1.22 (m, 7H), 1.25-1.12 (m, 2H), 0.88 (d, J=6.6 Hz, 6H).

### Example 159: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (7-methyloctyl) carbonate (159)

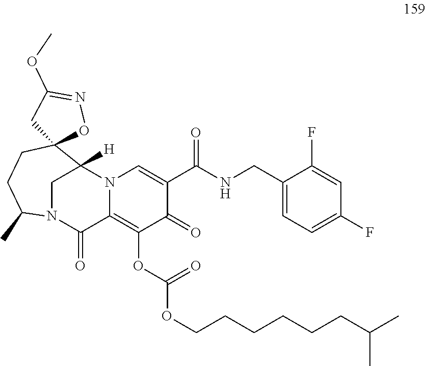 [SMILES: COC1=NO[C@@]2(CC[C@H](C)N3C[C@H]2n2cc(C(=O)NCc4ccc(F)cc4F)c(=O)c(OC(=O)OCCCCCCC(C)C)c2C3=O)C1]

The title compound was prepared in a similar manner to Example 2, except using 7-methyloctan-1-ol instead of 2-(2-methoxyethoxy)ethanol in Step 1. MS (m/z) 673.04 [M+H]⁺. 1H NMR (400 MHz, Chloroform-d) δ 10.21 (t, J=5.9 Hz, 1H), 8.67 (d, J=1.5 Hz, 1H), 7.36 (td, J=8.4, 6.3 Hz, 1H), 6.90-6.76 (m, 2H), 4.84 (dp, J=10.1, 6.8 Hz, 1H), 4.71 (dd, J=15.1, 6.2 Hz, 1H), 4.55 (dd, J=15.1, 5.6 Hz, 1H), 4.39-4.26 (m, 3H), 3.88 (s, 3H), 3.80 (dd, J=15.2, 1.9 Hz, 1H), 3.67 (dd, J=15.1, 2.7 Hz, 1H), 3.09 (d, J=17.1 Hz, 1H), 2.67 (d, J=17.2 Hz, 1H), 2.07-1.92 (m, 2H), 1.96-1.84 (m, 1H), 1.87-1.70 (m, 2H), 1.70-1.58 (m, 1H), 1.62 (s, 1H), 1.53 (dt, J=13.2.6.7 Hz, 1H), 1.50-1.39 (m, 2H), 1.42-1.26 (m, 3H), 1.25 (d, J=6.7 Hz, 3H), 1.17 (dtd, J=10.6, 5.4, 1.7 Hz, 2H), 0.88 (d, J=6.6 Hz, 6H).

### Example 160: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (8-methylnonyl) carbonate (160)

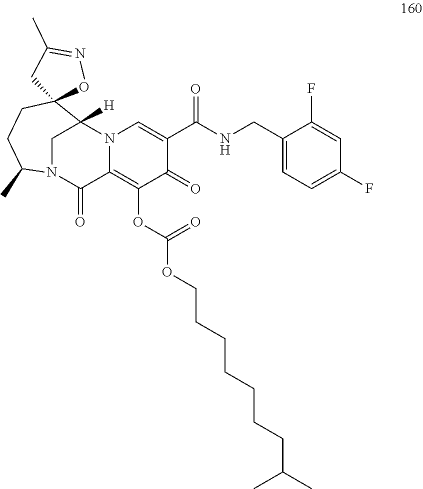 [SMILES: CC1=NO[C@@]2(CC[C@H](C)N3C[C@H]2n2cc(C(=O)NCc4ccc(F)cc4F)c(=O)c(OC(=O)OCCCCCCCC(C)C)c2C3=O)C1]

The title compound was prepared in a similar manner to Example 2, except using 8-methylnonan-1-ol instead of 2-(2-methoxyethoxy)ethanol in Step 1 and using Intermediate B instead of Intermediate C in Step 2. MS (m/z) 671.3 [M+H]⁺. 1H NMR (400 MHz, DMSO-d6) δ 10.13 (t, J=5.9 Hz, 1H), 8.84 (s, 1H), 7.42 (td. J=8.6, 6.6 Hz, 1H), 7.25 (ddd, J=10.5, 9.3, 2.6 Hz, 1H), 7.18-6.97 (m, 1H), 4.71 (d, J=2.7 Hz, 1H), 4.66-4.45 (m, 3H), 4.19 (dd, J=13.3, 6.6 Hz, 2H), 3.91-3.62 (m, 2H), 3.02 (d, J=17.4 Hz, 1H), 2.72-2.60 (m, 1H), 1.95 (s, 3H), 1.90-1.72 (m, 3H), 1.72-0.97 (m, 14H), 0.97-0.65 (m, 9H).

### Example 161: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (8-methylnonyl) carbonate (161)

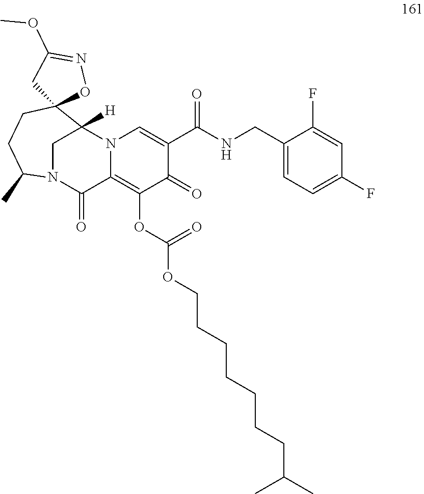 [SMILES: COC1=NO[C@@]2(CC[C@H](C)N3C[C@H]2n2cc(C(=O)NCc4ccc(F)cc4F)c(=O)c(OC(=O)OCCCCCCCC(C)C)c2C3=O)C1]

The title compound was prepared in a similar manner to Example 2, except using 8-methylnonan-1-ol instead of 2-(2-methoxyethoxy)ethanol in Step 1. MS (m/z) 687.7 [M+H]⁺. 1H NMR (400 MHz, DMSO-d6) δ 10.13 (t, J=5.9 Hz, 1H), 8.84 (s, 1H), 7.42 (td, J=8.6, 6.6 Hz, 1H), 7.25 (ddd, J=10.5, 9.3, 2.6 Hz, 1H), 7.18-6.97 (m, 1H), 4.71 (d, J=2.7 Hz, 1H), 4.66-4.45 (m, 3H), 4.19 (dd, J=13.3, 6.6 Hz, 2H), 3.91-3.62 (m, 2H), 3.02 (d, J=17.4 Hz, 1H), 2.72-2.60 (m, 1H), 1.95 (s, 3H), 1.90-1.72 (m, 3H), 1.72-0.97 (m, 14H), 0.97-0.65 (m, 9H).

### Example 162: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2,5,8,11,14,17,20-heptaoxadocosan-22-yl) carbonate (162)

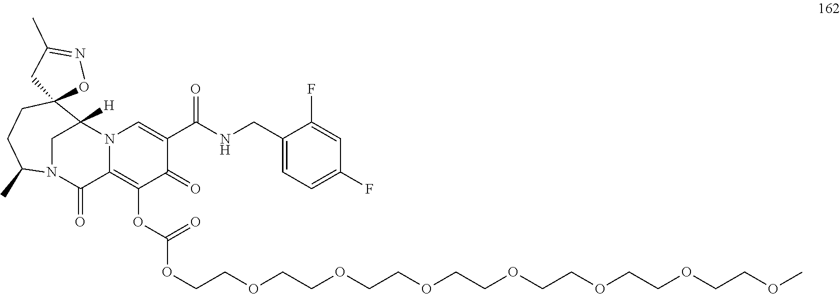

The title compound was prepared following a similar method as Example 2, except using 2,5,8,11,14,17,20-heptaoxadocosan-22-ol instead of 2-(2-methoxyethoxy)ethanol in Step 1 and using Intermediate B instead of Intermediate C in Step 2. MS (m/z) 853.2 [M+H]⁺. 1H NMR (400 MHz, Chloroform-d) δ 10.16 (t, J=6.0 Hz, 1H), 8.42 (s, 1H), 7.35 (td, J=8.7, 6.6 Hz, 1H), 6.95-6.73 (m, 2H), 4.81 (dq, J=9.2, 6.9 Hz, 1H), 4.73-4.51 (m, 2H), 4.50-4.39 (m, 2H), 4.32-4.23 (m, 1H), 4.10 (d, J=2.2 Hz, 1H), 3.85-3.78 (m, 3H), 3.75-3.68 (m, 9H), 3.67 (s, 3H), 3.65-3.59 (m, 3H), 3.56 (td, J=4.7, 1.7 Hz, 4H), 3.39 (d, J=1.5 Hz, 6H), 3.04 (d, J=17.8 Hz, 1H), 2.67-2.52 (m, 1H), 2.26 (m, 2H), 2.10 (s, 3H), 1.98-1.82 (m, 3H), 1.61 (ddd, J=14.8, 8.6, 3.9 Hz, 1H), 1.25 (d, J=6.7 Hz, 3H).

### Example 163: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (2,5,8,11,14,17,20-heptaoxadocosan-22-yl) carbonate (163)

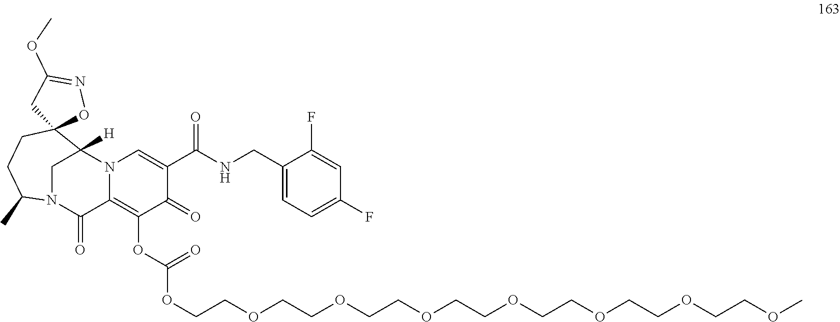

The title compound was prepared following a similar method as Example 2, except using 2,5,8,11,14,17,20-heptaoxadocosan-22-ol instead of 2-(2-methoxyethoxy)ethanol in Step 1. MS (m/z) 869.2 [M+H]⁺. 1H NMR (400 MHz, Chloroform-d) δ 10.19 (t, J=5.9 Hz, 1H), 8.66 (s, 1H), 7.35 (td, J=8.4, 6.3 Hz, 1H), 7.00-6.63 (m, 2H), 4.81 (dq, J=9.8, 6.9 Hz, 1H), 4.69 (dd, J=15.1, 6.1 Hz, 1H), 4.55 (dd, J=15.1, 5.7 Hz, 1H), 4.49-4.39 (m, 2H), 4.36 (d, J=2.4 Hz, 1H), 4.33-4.20 (m, 3H), 3.87 (s, 3H), 3.79-3.70 (m, 5H), 3.64-3.59 (m, 5H), 3.55 (ddd, J=6.3, 4.0, 1.7 Hz, 7H), 3.38 (d, J=1.5 Hz, 9H), 3.08 (d, J=17.1 Hz, 1H), 2.66 (d, J=17.1 Hz, 1H), 2.30 (s, 2H), 1.96 (tdq, J=18.4, 13.4, 6.6, 6.0 Hz, 3H), 1.61 (ddd, J=15.4, 10.2, 2.5 Hz, 1H), 1.24 (d, J=6.7 Hz, 3H).

### Example 164: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((S)-1-methoxypropan-2-yl) carbonate (164)

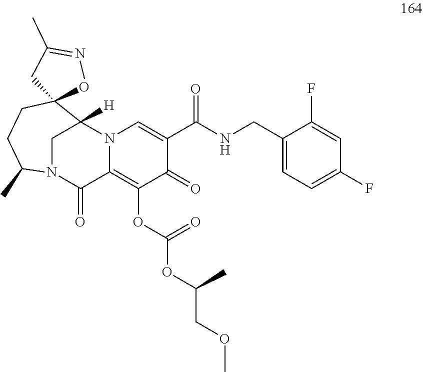 [SMILES: COC[C@H](C)OC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 2, except using (2S)-1-methoxypropan-2-ol instead of 2-(2-methoxyethoxy)ethanol in Step 1 and using Intermediate B instead of Intermediate C in Step 2. MS (m/z) 603.0 [M+H]⁺. 1H NMR (400 MHz, Chloroform-d) δ 10.19 (t, J=5.9 Hz, 1H), 8.43 (s, 1H), 7.35 (td, J=8.7, 6.5 Hz, 1H), 6.89-6.76 (m, 2H), 5.11-4.98 (m, 1H), 4.82 (dq, J=9.7, 6.7 Hz, 1H), 4.69 (dd, J=15.1, 6.1 Hz, 1H), 4.56 (dd, J=15.1, 5.7 Hz, 1H), 4.10 (d, J=2.2 Hz, 1H), 3.80 (dd, J=15.1, 1.9 Hz, 1H), 3.73-3.57 (m, 2H), 3.53 (dd, J=10.6, 4.7 Hz, 1H), 3.43 (s, 3H), 3.05 (d, J=17.8 Hz, 1H), 2.61 (dd, J=17.8, 1.3 Hz, 1H), 2.10 (s, 3H), 2.05-1.93 (m, 1H), 1.97-1.85 (m, 2H), 1.64 (ddd, J=14.9, 8.7, 3.8 Hz, 1H), 1.42 (d, J=6.4 Hz, 3H), 1.25 (d, J=6.6 Hz, 3H).

### Example 165: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((S)-1-methoxypropan-2-yl) carbonate (165)

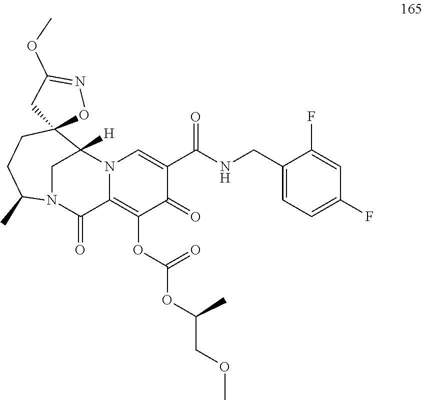 [SMILES: COC[C@H](C)OC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 2, except using (2S)-1-methoxypropan-2-ol instead of 2-(2-methoxyethoxy)ethanol in Step 1. MS (m/z) 619.0 [M+H]⁺. 1H NMR (400 MHz, Chloroform-d) δ 10.22 (t, J=6.0 Hz, 1H), 8.64 (s, 1H), 7.36 (td, J=8.4, 6.3 Hz, 1H), 6.89-6.64 (m, 2H), 5.14-4.94 (m, 1H), 4.84 (dt, J=10.2, 6.9 Hz, 1H), 4.71 (dd, J=15.1, 6.2 Hz, 1H), 4.55 (dd, J=15.1, 5.6 Hz, 1H), 3.89 (s, 3H), 3.79 (dd, J=15.1, 1.9 Hz, 1H), 3.62 (dd, J=10.5, 5.5 Hz, 1H), 3.54 (dd, J=10.5, 4.6 Hz, 1H), 3.44 (s, 3H), 3.09 (d, J=17.1 Hz, 1H), 2.67 (d, J=17.2 Hz, 1H), 2.17-1.87 (m, 3H), 1.72-1.56 (m, 3H), 1.43 (d, J=6.5 Hz, 3H), 1.25 (d, J=6.7 Hz, 3H).

### Example 166: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((S)-2-methoxypropyl) carbonate (166)

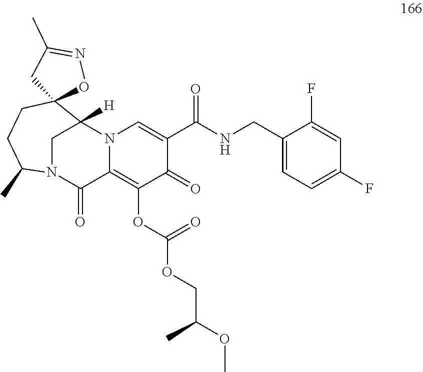 [SMILES: CO[C@@H](C)COC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 2, except using by (2S)-2-methoxypropan-1-ol instead of 2-(2-methoxyethoxy)ethanol in Step 1 and using Intermediate B instead of Intermediate C in Step 2. MS (m/z) 603.0 [M+H]⁺. 1H NMR (400 MHz, Chloroform-d) δ 10.17 (t, J=5.9 Hz, 1H), 8.43 (s, 1H), 7.35 (td, J=8.7, 6.5 Hz, 1H), 6.96-6.61 (m, 2H), 4.82 (dq, J=9.8, 6.8 Hz, 1H), 4.76-4.51 (m, 2H), 4.41-4.19 (m, 2H), 3.80 (dd, J=15.1, 1.9 Hz, 1H), 3.76-3.63 (m, 3H), 3.44 (s, 3H), 3.12-2.94 (m, 1H), 2.61 (dd, J=17.8, 1.2 Hz, 1H), 2.10 (d, J=1.1 Hz, 3H), 2.00-1.85 (m, 3H), 1.62 (ddd, J=14.9, 8.9, 3.9 Hz, 1H), 1.25 (dd, J=6.5, 5.2 Hz, 6H).

### Example 167: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((S)-2-methoxypropyl) carbonate (167)

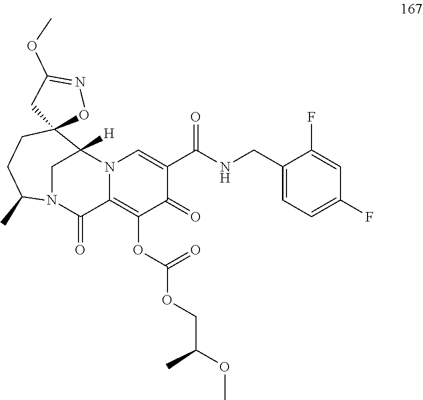 [SMILES: COC1=NO[C@@]2(CC[C@H](C)N3C[C@H]2n2cc(C(=O)NCc4ccc(F)cc4F)c(=O)c(OC(=O)OC[C@H](C)OC)c2C3=O)C1]

The title compound was prepared in a similar manner to Example 2, except using by (2S)-2-methoxypropan-1-ol instead of 2-(2-methoxyethoxy)ethanol in Step 1. MS (m/z) 619.0 [M+H]⁺. 1H NMR (400 MHz, Chloroform-d) δ 10.20 (t, J=5.9 Hz, 1H), 8.70 (s, 1H), 7.36 (td, J=8.4, 6.3 Hz, 1H), 6.95-6.73 (m, 2H), 4.83 (dq, J=9.7, 6.8 Hz, 1H), 4.71 (dd, J=15.1, 6.2 Hz, 1H), 4.55 (dd, J=15.1, 5.6 Hz, 1H), 4.34-4.14 (m, 2H), 3.87 (s, 3H), 3.80 (dd, J=15.2, 1.9 Hz, 1H), 3.72-3.64 (m, 2H), 3.44 (s, 3H), 3.09 (d, J=17.1 Hz, 1H), 2.66 (d, J=17.1 Hz, 1H), 2.08-1.86 (m, 3H), 1.63 (ddd, J=15.4, 10.3, 2.3 Hz, 2H), 1.25 (dd, J=6.5, 4.0 Hz, 6H).

### Example 168: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((R)-2-methoxypropyl) carbonate (168)

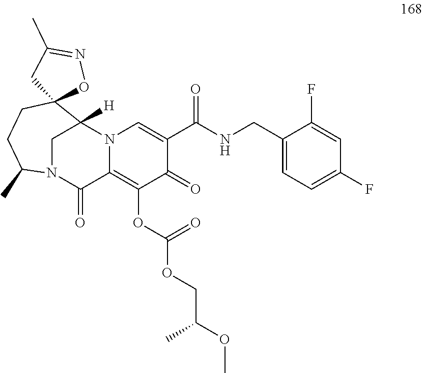 [SMILES: CO[C@H](C)COC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 2, except using (2R)-2-methoxypropan-1-ol instead of 1, 2-(2-methoxyethoxy)ethanol in Step 1 and using Intermediate B instead of Intermediate C in Step 2. MS (m/z) 603.0 [M+H]⁺. 1H NMR (400 MHz, Chloroform-d) δ 10.17 (t, J=6.0 Hz, 1H), 8.43 (s, 1H), 7.36 (td, J=8.6, 6.5 Hz, 1H), 6.89-6.77 (m, 2H), 4.83 (dt, J=9.8, 6.9 Hz, 1H), 4.68 (dd, J=15.1, 6.1 Hz, 1H), 4.58 (dd, J=15.1, 5.7 Hz, 1H), 4.34-4.21 (m, 2H), 4.10 (d, J=2.3 Hz, 1H), 3.81 (dd, J=15.1, 1.9 Hz, 1H), 3.77-3.64 (m, 2H), 3.45 (s, 3H), 3.10-3.00 (m, 1H), 2.61 (dd, J=17.8, 1.2 Hz, 1H), 2.11 (d, J=2.0 Hz, 1H), 2.11 (s, 2H), 2.01-1.86 (m, 3H), 1.68-1.56 (m, 1H), 1.29-1.10 (m, 6H).

### Example 169: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((R)-2-methoxypropyl) carbonate (169)

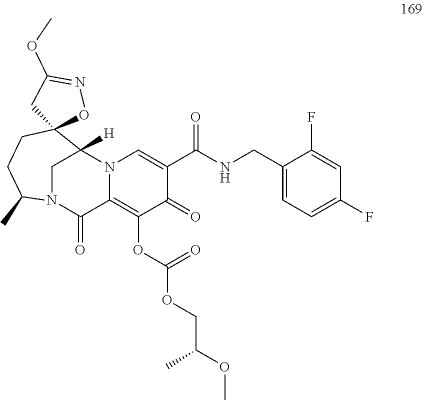 [SMILES: COC1=NO[C@@]2(CC[C@H](C)N3C[C@H]2n2cc(C(=O)NCc4ccc(F)cc4F)c(=O)c(OC(=O)OC[C@@H](C)OC)c2C3=O)C1]

The title compound was prepared in a similar manner to Example 2, except using (2R)-2-methoxypropan-1-ol instead of 1, 2-(2-methoxyethoxy)ethanol in Step 1. MS (m/z) 619.2 [M+H]⁺. 1H NMR (400 MHz, Chloroform-d) δ 10.20 (t, J=6.0 Hz, 1H), 8.68 (s, 1H), 7.36 (td, J=8.4, 6.3 Hz, 1H), 6.90-6.77 (m, 2H), 4.84 (dt, J=10.3, 6.9 Hz, 1H), 4.70 (dd, J=15.1, 6.2 Hz, 1H), 4.56 (dd, J=15.1, 5.6 Hz, 1H), 4.37 (s, 1H), 4.34-4.21 (m, 2H), 3.88 (s, 3H), 3.80 (dd, J=15.1, 1.9 Hz, 1H), 3.77-3.62 (m, 2H), 3.44 (s, 3H), 3.09 (d, J=17.1 Hz, 1H), 2.67 (d, J=17.1 Hz, 1H), 2.07-1.93 (m, 2H), 1.97-1.86 (m, 1H), 1.63 (ddd, J=15.4, 10.5, 2.3 Hz, 1H), 1.25 (dd, J=6.6, 5.5 Hz, 6H).

### Example 170: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((R)-1-methoxypropan-2-yl) carbonate (170)

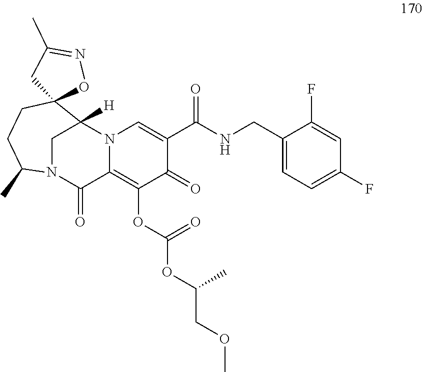 [SMILES: COC[C@@H](C)OC(=O)Oc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 2, except using (2R)-1-methoxypropan-2-ol instead of 2-(2-methoxyethoxy)ethanol in Step 1 and using Intermediate B instead of Intermediate C in Step 2. MS (m/z) 603.0 [M+H]⁺. 1H NMR (400 MHz, Chloroform-d) δ 10.19 (t, J=6.0 Hz, 1H), 8.42 (s, 1H), 7.36 (td, J=8.7, 6.5 Hz, 1H), 6.89-6.77 (m, 2H), 5.10-4.98 (m, 1H), 4.83 (dt, J=9.7, 6.7 Hz, 1H), 4.68 (dd, J=15.1, 6.2 Hz, 1H), 4.57 (dd, J=15.1, 5.7 Hz, 1H), 4.10 (d, J=2.2 Hz, 1H), 3.80 (dd, J=15.2, 1.9 Hz, 1H), 3.69 (dd, J=15.1, 2.7 Hz, 1H), 3.60 (dd, J=10.6, 5.7 Hz, 1H), 3.53 (dd, J=10.6, 4.6 Hz, 1H), 3.43 (s, 3H), 3.10-3.00 (m, 1H), 2.61 (dd, J=17.7, 1.3 Hz, 1H), 2.10 (d, J=1.0 Hz, 3H), 2.05-1.85 (m, 3H), 1.70-1.57 (m, 1H), 1.44 (d, J=6.5 Hz, 3H), 1.25 (d, J=6.7 Hz, 3H).

### Example 171: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl ((R)-1-methoxypropan-2-yl) carbonate (171)

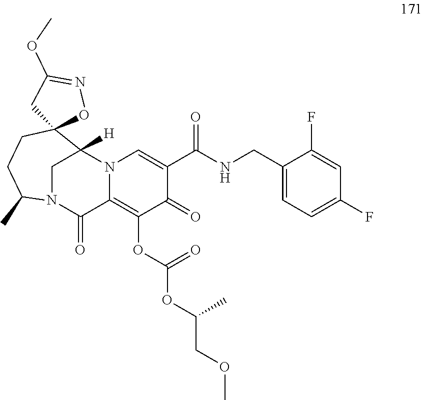 [SMILES: COC[C@@H](C)OC(=O)Oc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 2, except using (2R)-1-methoxypropan-2-ol instead of 2-(2-methoxyethoxy)ethanol in Step 1. MS (m/z) 619.0 [M+H]⁺. 1H NMR (400 MHz, Chloroform-d) δ 10.22 (t, J=5.9 Hz, 1H), 8.65 (s, 1H), 7.36 (td, J=8.4, 6.3 Hz, 1H), 6.96-6.69 (m, 2H), 5.16-4.94 (m, 1H), 4.84 (dt, J=10.2, 6.9 Hz, 1H), 4.71 (dd, J=15.1, 6.2 Hz, 1H), 4.55 (dd, J=15.1, 5.6 Hz, 1H), 4.35 (d, J=2.4 Hz, 1H), 3.88 (s, 3H), 3.79 (dd, J=15.1, 1.9 Hz, 1H), 3.68 (dd, J=15.1, 2.7 Hz, 1H), 3.63-3.49 (m, 2H), 3.39 (s, 1H), 3.09 (d, J=17.1 Hz, 1H), 2.67 (d, J=17.1 Hz, 1H), 2.16-1.82 (m, 3H), 1.44 (d, J=6.5 Hz, 3H), 1.26 (t, J=6.4 Hz, 4H), 1.15 (t, J=7.0 Hz, 2H).

### Example 172: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ethyl carbonate (172)

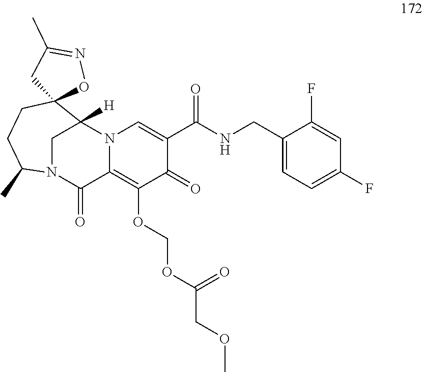 [SMILES: COCC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 12, except using chloromethyl ethyl carbonate instead of chloromethyl methyl carbonate. MS (m/z) 589.20 [M+H]⁺. ¹H NMR (400 MHz, Chloroform-d) δ 10.27 (t, J=5.7 Hz, 1H), 8.38 (d, J=32.3 Hz, 1H), 7.34 (q, J=7.7 Hz, 1H), 6.81 (q, J=8.9, 8.5 Hz, 2H), 6.01 (d, J=6.3 Hz, 1H), 5.81 (d, J=6.3 Hz, 1H), 4.83 (q, J=8.1, 7.7 Hz, 1H), 4.61 (qd, J=15.4, 5.4 Hz, 2H), 4.22 (p, J=6.7 Hz, 2H), 4.08 (d, J=20.1 Hz, 1H), 3.73 (d, J=15.3 Hz, 1H), 3.60 (d, J=15.0 Hz, 1H), 3.03 (d, J=17.3 Hz, 1H), 2.56 (d, J=17.3 Hz, 1H), 2.07 (s, 3H), 1.90 (q, J=16.5, 12.9 Hz, 3H), 1.61 (s, 1H), 1.30 (t, J=7.1 Hz, 3H), 1.20 (d, J=6.7 Hz, 3H).

### Example 173: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ethyl carbonate (173)

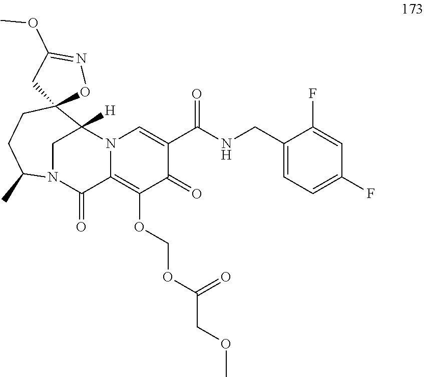 [SMILES: COCC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 11, except using chloromethyl ethyl carbonate instead of chloromethyl methyl carbonate. MS (m/z) 605.20 [M+H]⁺. ¹H NMR (400 MHz, Chloroform-d) δ 10.34-10.16 (m, 1H), 8.40 (s, 1H), 7.41-7.30 (m, 1H), 6.89-6.74 (m, 2H), 6.03 (d, J=6.4 Hz, 1H), 5.82 (d, J=6.4 Hz, 1H), 4.85 (h, J=7.1 Hz, 1H), 4.62 (qd, J=15.1, 5.7 Hz, 2H), 4.30-4.18 (m, 2H), 4.13 (d, J=13.2 Hz, 1H), 3.90 (s, 3H), 3.74 (dd, J=15.2, 1.8 Hz, 1H), 3.58 (dd, J=15.1, 2.6 Hz, 1H), 3.05 (d, J=17.2 Hz, 1H), 2.67 (d, J=17.2 Hz, 1H), 2.03-1.96 (m, 1H), 1.91 (dt, J=9.5, 5.2 Hz, 2H), 1.67-1.58 (m, 1H), 1.31 (t, J=7.1 Hz, 3H), 1.21 (d, J=6.7 Hz, 3H).

### Example 174: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl propyl carbonate (174)

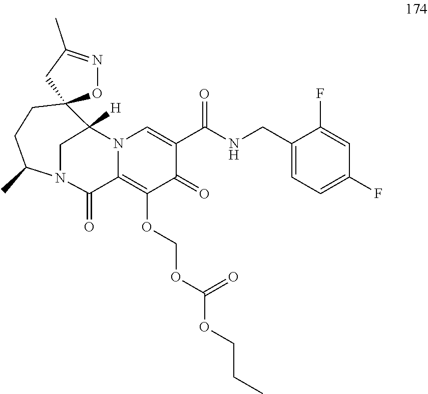 [SMILES: CCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was made in a similar manner to Example 12, except using chloromethyl propyl carbonate instead of chloromethyl methyl carbonate. MS (m/z) 603.4 [M+H]⁺. ¹H NMR (400 MHz, DMSO-d6) δ 10.28 (t, J=5.9 Hz, 1H), 8.70 (s, 1H), 7.43 (td, J=8.7, 6.6 Hz, 1H), 7.25 (ddd, J=10.5, 9.3, 2.6 Hz, 1H), 7.08 (tdd, J=8.5, 2.7, 1.0 Hz, 1H), 5.79 (d, J=6.5 Hz, 1H), 5.63 (d, J=6.5 Hz, 1H), 4.74-4.43 (m, 4H), 4.01 (td, J=6.6, 2.2 Hz, 2H), 3.69 (d, J=2.2 Hz, 2H), 3.01 (d, J=17.5 Hz, 1H), 2.64 (d, J=17.6 Hz, 1H), 1.95 (s, 3H), 1.88-1.70 (m, 3H), 1.58 (h, J=7.1 Hz, 2H), 1.45-1.23 (m, 1H), 1.16 (d, J=6.7 Hz, 3H), 0.87 (t, J=7.4 Hz, 3H).

### Example 175: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl propyl carbonate (175)

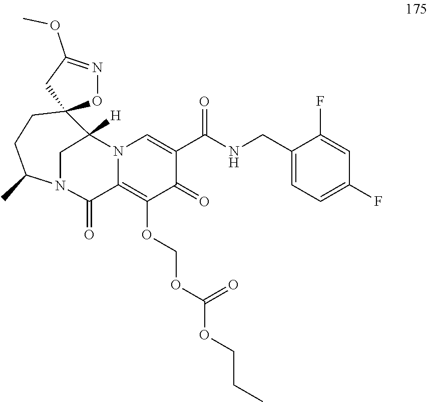 [SMILES: CCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 11, except using chloromethyl propyl carbonate instead of chloromethyl methyl carbonate. MS (m/z) 619.7 [M+H]⁺. ¹H NMR (400 MHz, DMSO-d6) δ 10.28 (t, J=5.9 Hz, 1H), 8.74 (s, 1H), 7.42 (td, J=8.7, 6.6 Hz, 1H), 7.25 (ddd, J=10.5, 9.4, 2.6 Hz, 1H), 7.08 (tdd, J=8.6, 2.6, 1.0 Hz, 1H), 5.77 (d, J=6.4 Hz, 1H), 5.62 (d, J=6.5 Hz, 1H), 4.71 (d, J=2.7 Hz, 1H), 4.69-4.46 (m, 3H), 4.09-3.93 (m, 2H), 3.82 (s, 3H), 3.68 (d, J=2.2 Hz, 2H), 3.05 (d, J=16.8 Hz, 1H), 2.76 (d, J=16.9 Hz, 11-1), 1.98-1.69 (m, 3H), 1.58 (h, J=7.1 Hz, 2H), 1.45-1.27 (m, 1H), 1.21-1.12 (m, 3H), 0.86 (t, J=7.4 Hz, 3H).

### Example 176: Preparation of butyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (176)

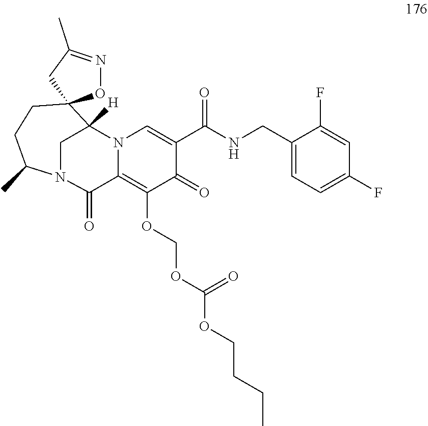 [SMILES: CCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 12, except using butyl chloromethyl carbonate instead of chloromethyl methyl carbonate. MS (m/z) 617.0 [M+H]⁺. ¹H NMR (400 MHz, Chloroform-d) δ 10.29 (t, J=6.0 Hz, 1H), 8.34 (s, 1H), 7.37 (td, J=8.6, 6.5 Hz, 1H), 6.92-6.76 (m, 2H), 6.06 (d, J=6.4 Hz, 1H), 5.87 (d, J=6.4 Hz, 1H), 5.00-4.80 (m, 1H), 4.73-4.48 (m, 2H), 4.20 (qt, J=10.6, 6.6 Hz, 2H), 4.04 (d, J=2.2 Hz, 1H), 3.76 (dd, J=15.1, 1.9 Hz, 1H), 3.61 (dd, J=15.1, 2.7 Hz, 1H), 3.13-2.97 (m, 1H), 2.60 (dd, J=17.8, 1.3 Hz, 1H), 2.11 (s, 3H), 2.00-1.82 (m, 3H), 1.73-1.63 (m, 3H), 1.43 (dq, J=14.6, 7.4 Hz, 2H), 1.24 (d, J=6.7 Hz, 3H), 0.94 (t, J=7.4 Hz, 3H).

### Example 177: Preparation of butyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (177)

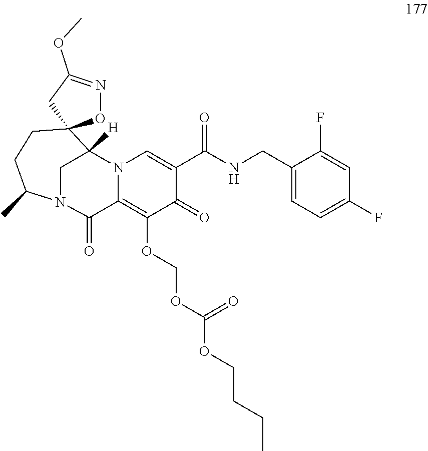 [SMILES: CCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 11, except using butyl chloromethyl carbonate instead of chloromethyl methyl carbonate. MS (m/z) 633.0 [M+H]⁺. ¹H NMR (400 MHz, Chloroform-d) δ 10.29 (t, J=6.0 Hz, 1H), 8.45 (s, 1H), 7.37 (td, J=8.4, 6.3 Hz, 1H), 6.91-6.78 (m, 2H), 6.06 (d, J=6.4 Hz, 1H), 5.87 (d, J=6.4 Hz, 1H), 4.92-4.81 (m, 1H), 4.69 (dd, J=15.1, 6.1 Hz, 1H), 4.60 (dd, J=15.1, 5.7 Hz, 1H), 4.28-4.12 (m, 3H), 3.92 (s, 3H), 3.75 (dd, J=15.1, 1.9 Hz, 1H), 3.60 (dd, J=15.1, 2.7 Hz, 1H), 3.08 (d, J=17.1 Hz, 1H), 2.69 (d, J=17.2 Hz, 1H), 2.06-1.97 (m, 1H), 2.00-1.87 (m, 2H), 1.75-1.60 (m, 3H), 1.43 (h, J=7.4 Hz, 2H), 1.23 (d, J=6.7 Hz, 3H), 0.94 (t, J=7.4 Hz, 3H).

### Example 178: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl octyl carbonate (178)

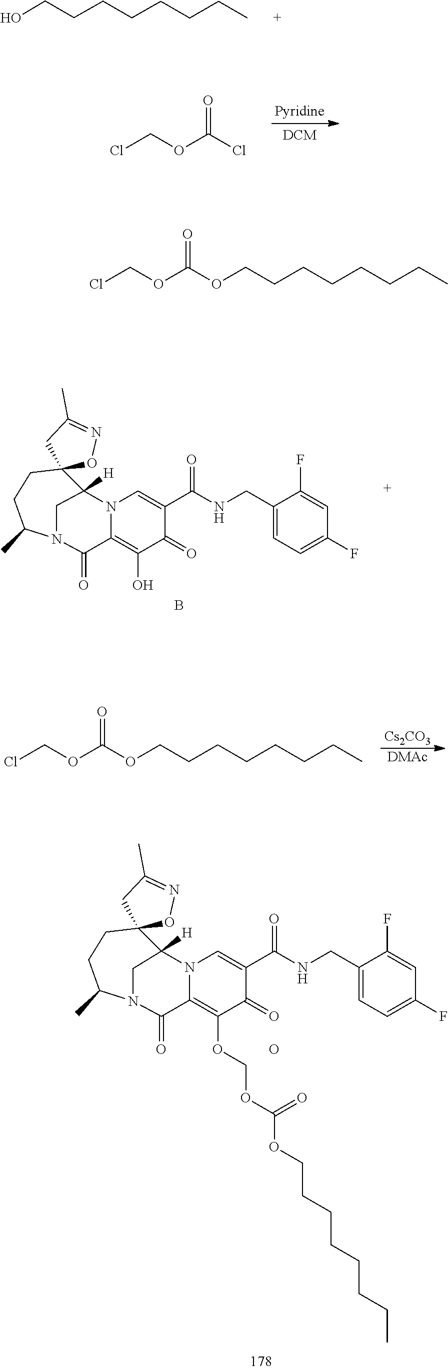

### Step 1: Synthesis of chloromethyl octyl carbonate

To a solution of octan-1-ol (2.5 g, 19.2 mmol) in DCM (20 mL) at 0° C. was added pyridine (2.32 mL, 28.8 mmol) followed by chloromethyl carbonochloridate (2.22 mL, 25 mmol). The resulting suspension was removed from cooling bath and allowed to warm to room temperature and stir for 1 hour. The reaction was rinsed with DCM. The filtrate was washed with 1 N HCl in water, dried over Na2SO4, filtered, and concentrated. The product was used directly in the next step.

Step 2. Synthesis of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl octyl carbonate:

To a mixture of Intermediate B (2 g, 4.11 mmol) and chloromethyl octyl carbonate (1.83 g, 8.22 mmol) in DMAc (30 mL) at room temperature was added cesium carbonate (1.61 g, 4.93 mmol). The resulting mixture was vigorously stirred at 35° C. for 16 hours. The reaction was then cooled to room temperature, diluted with DCM, washed sequentially with water and brine, dried over sodium sulfate, filtered and concentrated, purified by normal phase chromatography, eluted with 30-100% EtOAc/Hexane to provide the title compound. MS (m/z) 673.8 [M+H]⁺. ¹H NMR (400 MHz, DMSO-d6) δ 10.29 (t, J=6.0 Hz, 1H), 8.70 (s, 1H), 7.42 (td, J=8.7, 6.6 Hz, 1H), 7.25 (ddd, J=10.4, 9.3, 2.6 Hz, 1H), 7.08 (tdd, J=8.5, 2.6, 1.0 Hz, 1H), 5.80 (d, J=6.5 Hz, 1H), 5.62 (d, J=6.5 Hz, 1H), 4.70-4.46 (m, 4H), 4.03 (tt, J=6.8, 3.4 Hz, 2H), 3.69 (d, J=2.2 Hz, 2H), 3.01 (d, J=17.6 Hz, 1H), 2.64 (d, J=17.5 Hz, 1H), 1.95 (s, 3H), 1.86-1.66 (m, 3H), 1.55 (q, J=6.7 Hz, 2H), 1.42-1.19 (m, 1H), 1.16 (d, J=6.7 Hz, 3H), 0.91-0.76 (m, 3H).

### Example 179: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl pentyl carbonate (179)

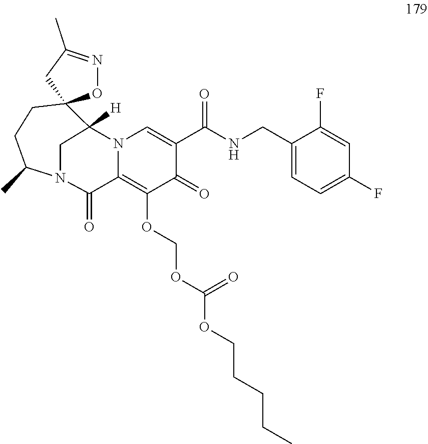 [SMILES: CCCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using 1-pentanol instead of octan-1-ol in Step 1. MS (m/z) 631.26 [M+H]⁺. ¹H NMR (400 MHz, Chloroform-d) δ 10.26 (s, 1H), 8.36 (s, 1H), 7.32 (q, J=8.0 Hz, 1H), 6.80 (q, J=9.7, 9.0 Hz, 2H), 6.00 (d, J=6.0 Hz, 1H), 5.81 (d, J=6.1 Hz, 1H), 4.99-4.75 (m, 1H), 4.59 (q, J=15.3 Hz, 2H), 4.14 (dq, J=21.6, 7.0 Hz, 3H), 3.71 (d, J=14.6 Hz, 1H), 3.59 (d, J=15.4 Hz, 1H), 3.03 (d, J=16.5 Hz, 1H), 2.55 (d, J=16.6 Hz, 1H), 2.03 (d, J=11.8 Hz, 3H), 1.96-1.78 (m, 3H), 1.73-1.60 (m, 2H), 1.57 (s, 1H), 1.32 (s, 4H), 1.19 (d, J=6.4 Hz, 3H), 0.88 (d, J=7.0 Hz, 3H).

### Example 180: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl pentyl carbonate (180)

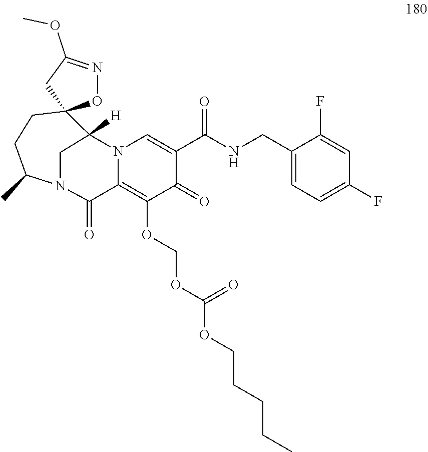 [SMILES: CCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using 1-pentanol instead of octan-1-ol in Step 1 and Intermediate C instead of Intermediate B in Step 2. MS (m/z) 647.26 [M+H]⁺. ¹H NMR (400 MHz, Chloroform-d) δ 10.29 (s, 1H), 8.58 (s, 1H), 7.34 (q, J=7.6 Hz, 1H), 6.81 (q, J=9.4, 9.0 Hz, 2H), 6.02 (d, J=5.9 Hz, 1H), 5.82 (d, J=6.1 Hz, 1H), 4.97-4.75 (m, 1H), 4.67 (d, J=14.5 Hz, 1H), 4.55 (d, J=15.0 Hz, 1H), 4.32 (s, 1H), 4.15 (dtd, J=17.0, 10.6, 6.5 Hz, 2H), 3.84 (s, 3H), 3.71 (d, J=14.0 Hz, 1H), 3.58 (d, J=15.1 Hz, 1H), 3.08 (d, J=16.4 Hz, 1H), 2.62 (d, J=16.3 Hz, 1H), 2.10-1.82 (m, 3H), 1.66 (h, J=6.1, 5.3 Hz, 3H), 1.32 (tt, J=10.6, 4.3 Hz, 4H), 1.20 (d, J=6.3 Hz, 3H), 0.88 (t, J=6.7 Hz, 3H).

### Example 181: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl hexyl carbonate (181)

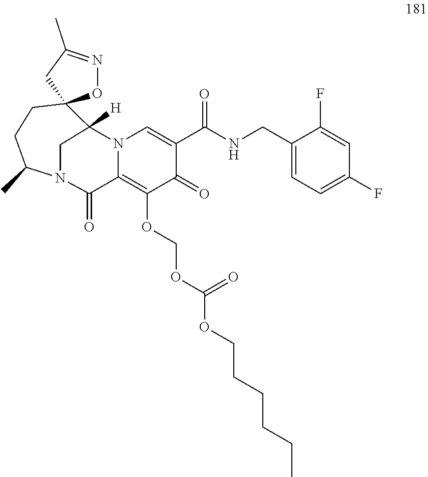 [SMILES: CCCCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was made in a similar manner to Example 178, except using hexan-1-ol instead of octan-1-ol in Step 1. MS (m/z) 645.2 [M+H]⁺. ¹H NMR (400 MHz, DMSO-d6) δ 10.28 (t, J=5.9 Hz, 1H), 8.70 (s, 1H), 7.42 (td, J=8.7, 6.6 Hz, 1H), 7.25 (ddd, J=10.5, 9.3, 2.6 Hz, 1H), 7.08 (tdd, J=8.6, 2.6, 1.0 Hz, 1H), 5.80 (d, J=6.5 Hz, 1H), 5.63 (d, J=6.5 Hz, 1H), 4.70-4.45 (m, 4H), 4.15-3.93 (m, 2H), 3.69 (d, J=2.2 Hz, 2H), 3.01 (d, J=17.5 Hz, 1H), 2.64 (d, J=17.5 Hz, 1H), 1.95 (s, 3H), 1.90-1.69 (m, 3H), 1.55 (dt, J=8.0, 6.2 Hz, 2H), 1.38-1.21 (m, 5H), 1.21-1.10 (m, 5H), 0.91-0.77 (m, 3H).

### Example 182: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl hexyl carbonate (182)

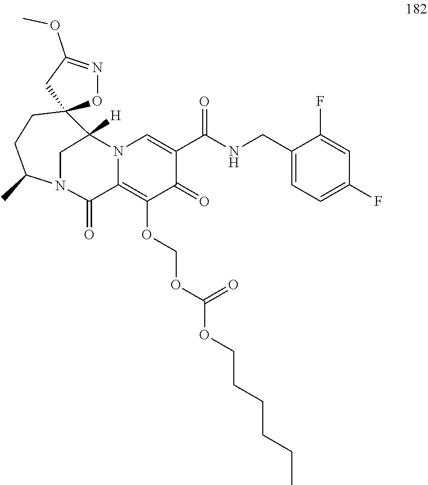 [SMILES: CCCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using hexan-1-ol instead of octan-1-ol in Step 1 and Intermediate C instead of Intermediate B in Step 2. MS (m/z) 661.4 [M+H]⁺. ¹H NMR (400 MHz, DMSO-d6) δ 10.28 (t, J=6.0 Hz, 1H), 8.75 (s, 1H), 7.42 (td, J=8.7, 6.6 Hz, 1H), 7.25 (ddd, J=10.5, 9.3, 2.6 Hz, 1H), 7.08 (tdd, J=8.6, 2.6, 1.0 Hz, 1H), 5.78 (d, J=6.5 Hz, 1H), 5.62 (d, J=6.4 Hz, 1H), 4.72 (d, J=2.1 Hz, 1H), 4.68-4.44 (m, 3H), 4.16-3.98 (m, 2H), 3.82 (s, 3H), 3.75-3.57 (m, 2H), 3.06 (d, J=16.8 Hz, 1H), 2.76 (d, J=16.9 Hz, 1H), 1.96-1.89 (m, 1H), 1.89-1.71 (m, 2H), 1.66-1.50 (m, 2H), 1.45-1.21 (m, 6H), 1.16 (d, J=6.7 Hz, 4H), 0.95-0.71 (m, 3H).

### Example 183: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl heptyl carbonate (183)

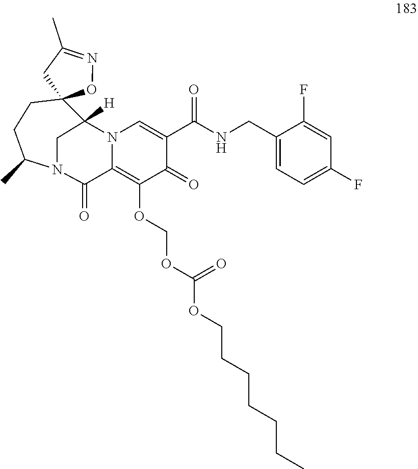 [SMILES: CCCCCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using heptan-1-ol instead of octan-1-ol. MS (m/z) 659.3 [M+H]⁺. ¹H NMR (400 MHz, DMSO-d6) δ 10.28 (t, J=6.0 Hz, 1H), 8.70 (s, 1H), 7.42 (td, J=8.7, 6.6 Hz, 1H), 7.25 (ddd, J=10.4, 9.3, 2.6 Hz, 1H), 7.21-7.02 (m, 1H), 5.80 (d, J=6.4 Hz, 1H), 5.62 (d, J=6.5 Hz, 1H), 4.71-4.46 (m, 4H), 4.04 (tt, J=6.9, 3.5 Hz, 2H), 3.69 (d, J=2.3 Hz, 2H), 3.01 (d, J=17.5 Hz, 1H), 2.64 (d, J=17.5 Hz, 1H), 1.95 (s, 3H), 1.90-1.66 (m, 3H), 1.56 (p, J=6.6 Hz, 2H), 1.38-1.20 (m, 9H), 1.16 (d, J=6.7 Hz, 3H), 0.97-0.76 (m, 3H).

### Example 184: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl heptyl carbonate (184)

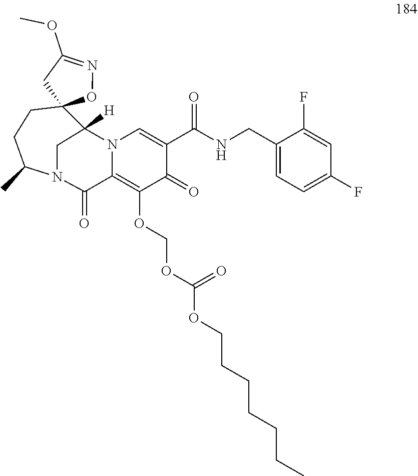 [SMILES: CCCCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using heptan-1-ol instead of octan-1-ol in Step 1 and Intermediate C instead of Intermediate B in Step 2. MS (m/z) 675.4 [M+H]⁺. ¹H NMR (400 MHz, DMSO-d6) δ 10.28 (t, J=5.9 Hz, 1H), 8.75 (s, 1H), 7.42 (td, J=8.7, 6.6 Hz, 1H), 7.25 (ddd, J=10.4, 9.3, 2.6 Hz, 1H), 7.07 (ddd, J=10.6, 8.3, 2.6 Hz, 1H), 5.78 (d, J=6.4 Hz, 1H), 5.62 (d, J=6.5 Hz, 1H), 4.71 (d, J=2.4 Hz, 1H), 4.68-4.48 (m, 3H), 4.04 (tt, J=7.3, 3.7 Hz, 2H), 3.82 (s, 3H), 3.75-3.60 (m, 2H), 3.06 (d, J=16.8 Hz, 1H), 2.75 (d, J=16.9 Hz, 1H), 2.02-1.86 (m, 1H), 1.79 (dt, J=10.4, 5.7 Hz, 2H), 1.56 (p, J=6.6 Hz, 2H), 1.43-1.19 (m, 9H), 1.16 (d, J=6.7 Hz, 3H), 0.97-0.76 (m, 3H).

### Example 185: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl octyl carbonate (185)

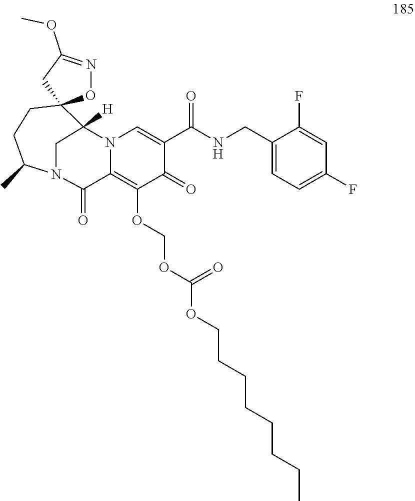 [SMILES: CCCCCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was made in a similar manner to Example 178, except using Intermediate C instead of Intermediate B in Step 2. MS (m/z) 689.5 [M+H]⁺. ¹H NMR (400 MHz, DMSO-d6) δ 10.28 (t, J=5.9 Hz, 1H), 8.75 (s, 1H), 7.42 (td, J=8.7, 6.6 Hz, 1H), 7.25 (ddd, J=10.5, 9.3, 2.6 Hz, 1H), 7.16-6.93 (m, 1H), 5.78 (d, J=6.4 Hz, 1H), 5.62 (d, J=6.5 Hz, 1H), 4.72 (s, 1H), 4.69-4.45 (m, 3H), 4.04 (tt, J=7.2, 3.6 Hz, 2H), 3.82 (s, 3H), 3.68 (d, J=2.3 Hz, 2H), 3.06 (d, J=16.8 Hz, 1H), 2.75 (d, J=16.9 Hz, 1H), 1.90 (d, J=13.1 Hz, 1H), 1.80 (d, J=8.0 Hz, 2H), 1.55 (q, J=6.8 Hz, 2H), 1.24 (br, 1H), 1.16 (d, J=6.7 Hz, 3H), 0.93-0.68 (m, 3H).

### Example 186: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl nonyl carbonate (186)

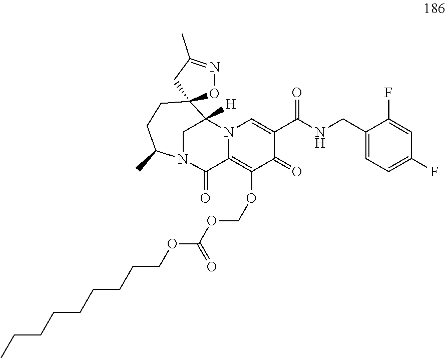 [SMILES: CCCCCCCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using nonan-1-ol instead of 1 octan-1-ol in Step 1. MS (m/z) 687.1 [M+H]⁺. ¹H NMR (400 MHz, Chloroform-d) δ 10.29 (t, J=6.0 Hz, 1H), 8.35 (s, 1H), 7.42-7.31 (m, 1H), 6.90-6.77 (m, 2H), 6.06 (dd, J=6.4, 1.4 Hz, 1H), 5.86 (dd, J=6.4, 1.5 Hz, 1H), 4.86 (dp, J=13.7, 7.0 Hz, 1H), 4.68 (dd, J=15.1, 6.2 Hz, 1H), 4.59 (dd, J=15.1, 5.8 Hz, 1H), 4.18 (qt, J=10.6, 6.6 Hz, 2H), 4.06 (d, J=2.4 Hz, 1H), 3.76 (dd, J=15.1, 1.9 Hz, 1H), 3.61 (dd, J=15.1, 2.7 Hz, 1H), 3.05 (d, J=17.7 Hz, 1H), 2.59 (d, J=17.8 Hz, 1H), 2.10 (s, 3H), 1.99-1.84 (m, 3H), 1.73-1.65 (m, 3H), 1.68-1.55 (m, 1H), 1.38 (h, J=6.7, 6.2 Hz, 2H), 1.28 (ddd, J=22.8, 16.2, 6.4 Hz, 12H), 0.89 (t, J=6.7 Hz, 3H).

### Example 187: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl nonyl carbonate (187)

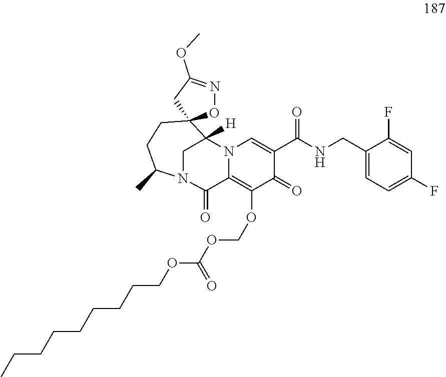 [SMILES: CCCCCCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using nonan-1-ol instead of 1 octan-1-ol in Step 1 and using Intermediate C instead of Intermediate B. MS (m/z) 703.0 [M+H]⁺. ¹H NMR (400 MHz, Chloroform-d) δ 10.30 (t, J=6.0 Hz, 1H), 8.51 (d, J=5.5 Hz, 1H), 7.37 (td, J=8.4, 6.3 Hz, 1H), 7.02-6.70 (m, 2H), 6.06 (d, J=6.4 Hz, 1H), 5.85 (d, J=6.4 Hz, 1H), 4.96-4.79 (m, 1H), 4.69 (dd, J=15.1, 6.1 Hz, 1H), 4.58 (dd, J=15.1, 5.7 Hz, 1H), 4.18 (qt, J=10.5, 6.6 Hz, 2H), 3.90 (s, 3H), 3.74 (dd, J=15.1, 1.9 Hz, 1H), 3.60 (dd, J=15.1, 2.7 Hz, 1H), 3.09 (d, J=17.1 Hz, 1H), 2.67 (d, J=17.2 Hz, 1H), 2.13-1.82 (m, 3H), 1.74-1.56 (m, 4H), 1.27 (td, J=21.6, 18.0, 7.7 Hz, 15H), 0.89 (t, J=6.7 Hz, 3H).

### Example 188: Preparation of decyl ((((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (188)

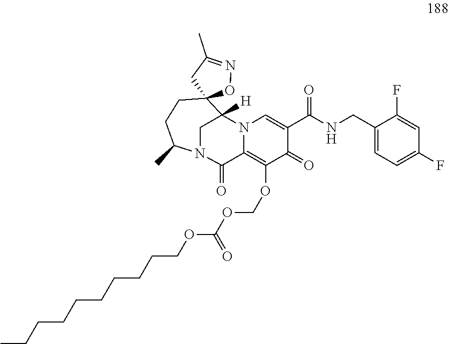 [SMILES: CCCCCCCCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using decan-1-ol instead of octan-1-ol. MS (m/z) 701.1 [M+H]⁺. ¹H NMR (400 MHz, Chloroform-d) δ 10.29 (t, J=6.0 Hz, 1H), 8.35 (s, 1H), 7.37 (q, J=8.0 Hz, 1H), 6.84 (q, J=9.0, 8.4 Hz, 2H), 6.06 (d, J=6.4 Hz, 1H), 5.86 (d, J=6.4 Hz, 1H), 4.93-4.81 (m, 1H), 4.68 (dd, J=15.2, 6.1 Hz, 1H), 4.60 (dd, J=15.2, 5.8 Hz, 1H), 4.18 (dtd, J=17.1, 10.6, 6.6 Hz, 2H), 4.05 (s, 1H), 3.76 (d, J=15.0 Hz, 1H), 3.61 (dd, J=15.2, 2.7 Hz, 1H), 3.05 (d, J=17.7 Hz, 1H), 2.60 (d, J=17.8 Hz, 1H), 2.10 (s, 3H), 1.99-1.84 (m, 3H), 1.74-1.56 (m, 4H), 1.38 (t, J=7.5 Hz, 2H), 1.26 (q, J=10.0, 6.6 Hz, 14H), 0.89 (t, J=6.6 Hz, 3H).

### Example 189: Preparation of decyl ((((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl) carbonate (189)

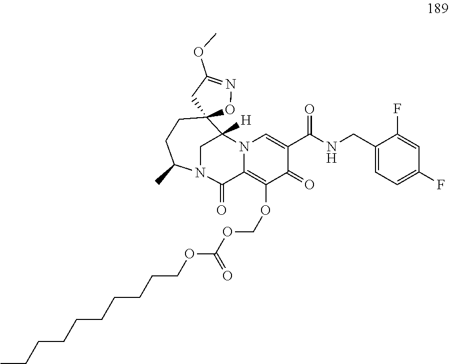 [SMILES: CCCCCCCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using decan-1-ol instead of octan-1-ol in Step 1 and using Intermediate C instead of Intermediate B in Step 2. MS (m/z) 717.1 [M+H]⁺. ¹H NMR (400 MHz, Chloroform-d) δ 10.31 (t, J=5.9 Hz, 1H), 8.55 (s, 1H), 7.37 (td, J=8.4, 6.3 Hz, 1H), 6.90-6.77 (m, 2H), 6.05 (d, J=6.4 Hz, 1H), 5.85 (d, J=6.4 Hz, 1H), 4.94-4.79 (m, 1H), 4.69 (dd, J=15.1, 6.1 Hz, 1H), 4.58 (dd, J=15.1, 5.7 Hz, 1H), 4.28 (d, J=2.4 Hz, 1H), 4.17 (qt, J=10.5, 6.7 Hz, 2H), 3.89 (s, 3H), 3.74 (dd, J=15.1, 1.9 Hz, 1H), 3.60 (dd, J=15.1, 2.7 Hz, 1H), 3.09 (d, J=17.1 Hz, 1H), 2.66 (d, J=17.2 Hz, 1H), 2.05-1.91 (m, 2H), 1.95-1.84 (m, 1H), 1.75-1.56 (m, 2H), 1.42-1.19 (m, 18H), 0.89 (t, J=6.8 Hz, 3H).

### Example 190: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl dodecyl carbonate (190)

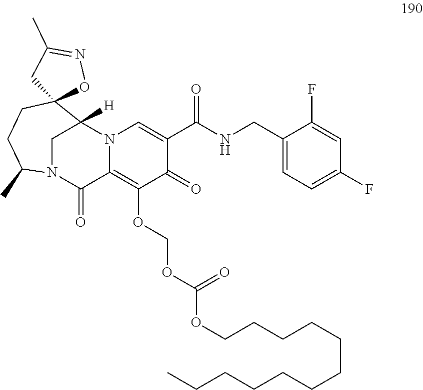 [SMILES: CCCCCCCCCCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using dodecan-1-ol instead of octan-1-ol. MS (m/z) 729.6 [M+H]⁺. ¹H NMR (400 MHz, DMSO-d6) δ 10.29 (t, J=5.9 Hz, 1H), 8.70 (s, 1H), 7.42 (td, J=8.7, 6.6 Hz, 1H), 7.25 (ddd, J=10.5, 9.3, 2.6 Hz, 1H), 7.16-6.95 (m, 1H), 5.80 (d, J=6.5 Hz, 1H), 5.62 (d, J=6.5 Hz, 1H), 4.74-4.44 (m, 4H), 4.04 (tt. J=6.5, 3.5 Hz, 2H), 3.69 (d, J=2.3 Hz, 2H), 3.01 (d, J=17.5 Hz, 1H), 2.73-2.54 (m, 1H), 1.95 (s, 3H), 1.88-1.70 (m, 3H), 1.55 (q, J=6.8 Hz, 2H), 1.24 (s, 19H), 1.16 (d, J=6.7 Hz, 3H), 0.96-0.80 (m, 3H).

### Example 191: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl dodecyl carbonate (191)

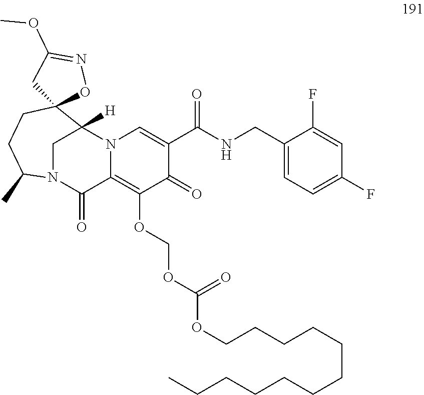 [SMILES: CCCCCCCCCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using dodecan-1-ol instead of octan-1-ol in Step 1 and using Intermediate C instead of Intermediate B in Step 2. MS (m/z) 745.6 [M+H]⁺. ¹H NMR (400 MHz, DMSO-d6) δ 10.28 (t. J=6.0 Hz, 1H), 8.75 (s, 1H), 7.42 (td, J=8.7, 6.6 Hz, 1H), 7.24 (ddd, J=10.5, 9.3, 2.6 Hz, 1H), 7.07 (ddd, J=10.7, 8.3, 2.6 Hz, 1H), 5.78 (d, J=6.4 Hz, 1H), 5.62 (d, J=6.4 Hz, 1H), 4.71 (d, J=2.4 Hz, 1H), 4.68-4.45 (m, 3H), 4.04 (tt, J=7.5, 3.8 Hz, 2H), 3.82 (s, 3H), 3.73-3.59 (m, 2H), 3.06 (d, J=16.8 Hz, 1H), 2.75 (d, J=16.9 Hz, 1H), 2.00-1.84 (m, 1H), 1.84-1.72 (m, 2H), 1.56 (p, J=6.7 Hz, 2H), 1.24 (s, 19H), 1.15 (d, J=6.6 Hz, 3H), 0.93-0.83 (m, 3H).

### Example 192: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl tetradecyl carbonate (192)

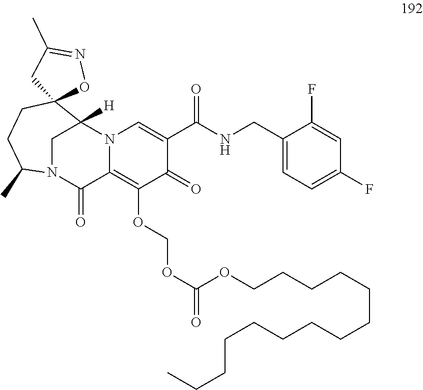 [SMILES: CCCCCCCCCCCCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using tetradecanol instead of octan-1-ol. MS (m/z) 757.0 [M+H]⁺. ¹H NMR (400 MHz, Methanol-d) δ 8.58 (s, 1H), 7.52-7.40 (m, 1H), 7.15-6.89 (m, 2H), 5.96-5.83 (m, 1H), 5.71-5.65 (m, 1H), 4.74-4.60 (m, 2H), 4.47 (s, 1H), 4.21-4.06 (m, 2H), 3.92-3.71 (m, 2H), 3.21-3.13 (m, 2H), 2.77-2.65 (m, 1H), 2.07 (s, 3H), 2.03-1.82 (m, 3H), 1.74-1.62 (m, 2H), 1.62-1.49 (m, 1H), 1.47-1.22 (m, 25H), 0.99-0.89 (m, 3H).

### Example 193: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl tetradecyl carbonate (193)

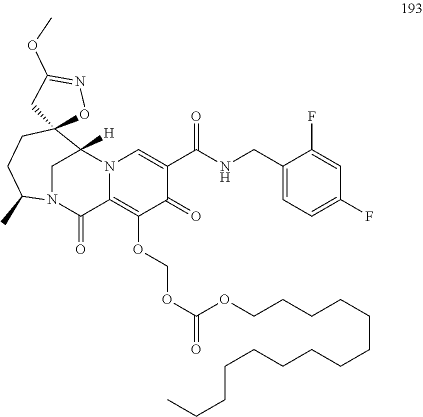 [SMILES: CCCCCCCCCCCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using tetradecanol instead of octan-1-ol in Step 1 and using Intermediate C instead of Intermediate B in Step 2. MS (m/z) 773.4 [M+H]⁺. ¹H NMR (400 MHz, Methanol-d) δ 8.62 (s, 1H), 7.54-7.34 (m, 1H), 7.11-6.82 (m, 2H), 5.90 (d, J=6.6 Hz, 1H), 5.68 (d, J=6.6 Hz, 1H), 4.72-4.44 (m, 4H), 4.22-4.05 (m, 2H), 3.92 (s, 3H), 3.88-3.68 (m, 2H), 3.23-3.16 (m, 1H), 2.86-2.67 (m, 1H), 2.13-1.84 (m, 4H), 1.73-1.45 (m, 3H), 1.45-1.20 (m, 24H), 0.98-0.86 (m, 3H).

### Example 194: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl hexadecyl carbonate (194)

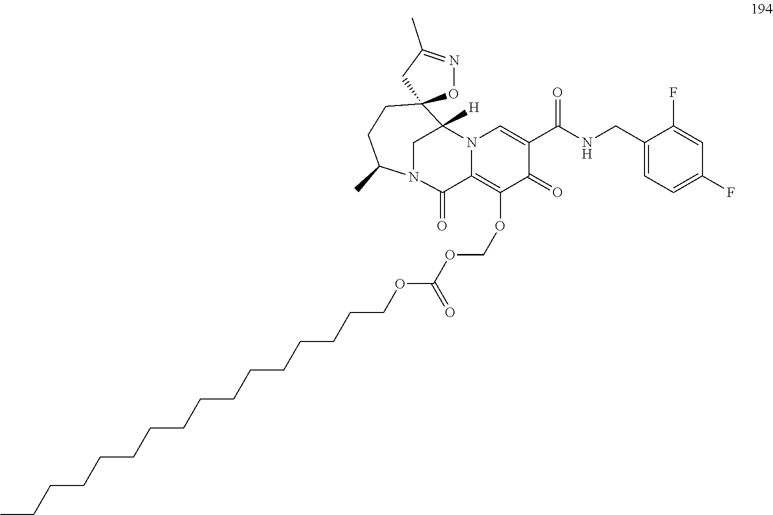 [SMILES: CCCCCCCCCCCCCCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using hexadecan-1-ol instead of octan-1-ol. MS (m/z) 785.1 [M+H]⁺. ¹H NMR (400 MHz, Chloroform-d) δ 10.29 (t, J=6.0 Hz, 1H), 8.34 (s, 1H), 7.37 (td, J=8.7, 6.6 Hz, 1H), 6.90-6.78 (m, 2H), 6.06 (d, J=6.4 Hz, 1H), 5.86 (d, J=6.4 Hz, 1H), 4.86 (dq, J=9.4, 6.9 Hz, 1H), 4.68 (dd, J=15.1, 6.1 Hz, 1H), 4.60 (dd, J=15.1, 5.8 Hz, 1H), 4.18 (qt, J=10.5, 6.6 Hz, 2H), 4.04 (d, J=2.2 Hz, 1H), 3.76 (dd, J=15.1, 1.9 Hz, 1H), 3.61 (dd, J=15.0, 2.7 Hz, 1H), 3.05 (d, J=17.8 Hz, 1H), 2.60 (d, J=17.8 Hz, 1H), 2.11 (s, 3H), 2.00-1.85 (m, 3H), 1.75-1.56 (m, 3H), 1.43-1.20 (m, 29H), 0.94-0.86 (m, 3H).

### Example 195: Preparation of (((3′S,5S,7′R)-10′-((2,4-difluorobenzyl)carbamoyl)-3-methoxy-3′-methyl-1′,11′-dioxo-1′,4′˜5′,11′-tetrahydro-3′H,4H,7′H-spiro[isoxazole-5,6′-[2,7]methanopyrido[1,2-a][1,4]diazonin]-12′-yl)oxy)methyl hexadecyl carbonate (195)

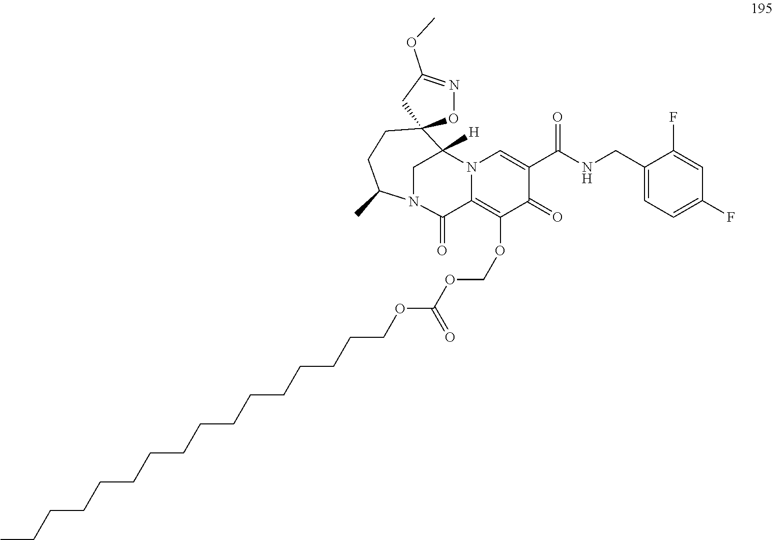 [SMILES: CCCCCCCCCCCCCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using hexadecan-1-ol instead of octan-1-ol in Step 1 and using Intermediate C instead of Intermediate B in Step 2. MS (m/z) 801.0 [M+H]⁺. ¹H NMR (400 MHz, Chloroform-d) δ 10.29 (t, J=5.9 Hz, 1H), 8.45 (s, 1H), 7.37 (td, J=8.4, 6.4 Hz, 1H), 6.90-6.78 (m, 2H), 6.06 (d, J=6.5 Hz, 1H), 5.86 (d, J=6.5 Hz, 1H), 4.87 (dt, J=9.8, 6.9 Hz, 1H), 4.69 (dd, J=15.1, 6.1 Hz, 1H), 4.60 (dd, J=15.1, 5.8 Hz, 1H), 4.18 (qt, J=10.5, 6.7 Hz, 3H), 3.92 (s, 3H), 3.75 (dd, J=15.1, 1.9 Hz, 1H), 3.60 (dd, J=15.1, 2.7 Hz, 1H), 3.08 (d, J=17.1 Hz, 1H), 2.69 (d, J=17.2 Hz, 1H), 2.06-1.87 (m, 3H), 1.75-1.61 (m, 3H), 1.25 (dd, J=17.8, 4.8 Hz, 29H), 0.90 (t, J=6.7 Hz, 3H).

### Example 196: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl octadecyl carbonate (196)

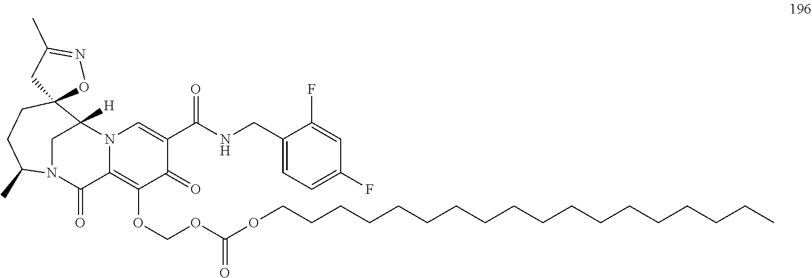 [SMILES: CCCCCCCCCCCCCCCCCCOC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using octadecan-1-ol instead of octan-1-ol. MS (m/z) 813.4 [M+H]⁺. ¹H NMR (400 MHz, Chloroform-d) δ 10.29 (t, J=6.0 Hz, 1H), 8.34 (s, 1H), 7.42-7.35 (m, 1H), 6.90-6.78 (m, 2H), 6.07 (d, J=6.5 Hz, 1H), 5.87 (d, J=6.4 Hz, 1H), 4.95-4.83 (m, 1H), 4.76-4.53 (m, 3H), 4.19 (tdd, J=10.5, 6.7, 3.9 Hz, 2H), 4.03 (d, J=2.5 Hz, 1H), 3.77 (dd, J=15.2, 1.9 Hz, 1H), 3.61 (dd, J=15.1, 2.7 Hz, 1H), 3.05 (d, J=17.8 Hz, 1H), 2.60 (d, J=17.8 Hz, 1H), 2.11 (s, 3H), 1.94 (dd, J=8.2, 3.1 Hz, 3H), 1.74-1.65 (m, 2H), 1.57 (s, 5H), 1.27 (d, J=3.1 Hz, 28H), 0.90 (t, J=6.7 Hz, 3H).

### Example 197: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl octadecyl carbonate (197)

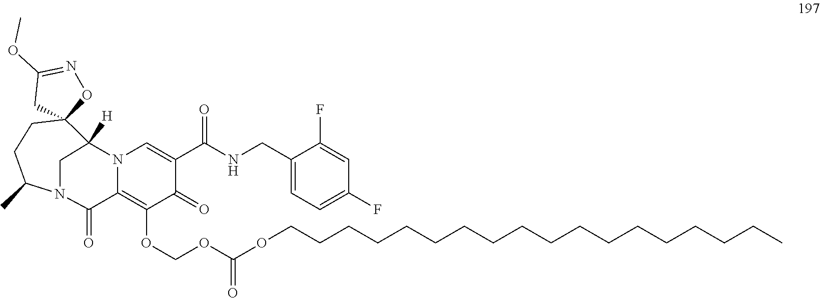 [SMILES: CCCCCCCCCCCCCCCCCCOC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using octadecan-1-ol instead of octan-1-ol in Step 1 and using Intermediate C instead of Intermediate B in Step 2. MS (m % z) 829.3 [M+H]⁺. ¹H NMR (400 MHz, Chloroform-d) δ 10.32 (t, J=6.0 Hz, 1H), 8.66 (s, 1H), 7.36 (d, J=7.6 Hz, 1H), 6.82 (q, J=10.3, 9.7 Hz, 2H), 6.04 (d, J=6.5 Hz, 1H), 5.83 (d, J=6.5 Hz, 1H), 4.85 (q, J=7.8 Hz, 1H), 4.69 (dd, J=15.2, 6.3 Hz, 1H), 4.55 (dd, J=15.2, 5.8 Hz, 1H), 4.40 (s, 1H), 4.16 (q, J=7.0 Hz, 2H), 3.85 (s, 3H), 3.72 (d, J=15.4 Hz, 1H), 3.60 (d, J=15.0 Hz, 1H), 3.10 (d, J=17.1 Hz, 1H), 2.62 (d, J=17.2 Hz, 1H), 2.07-1.84 (m, 3H), 1.67 (q, J=8.5, 7.9 Hz, 2H), 1.26 (s, 34H), 0.88 (t, J=6.7 Hz, 3H).

### Example 198: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl isopropyl carbonate (198)

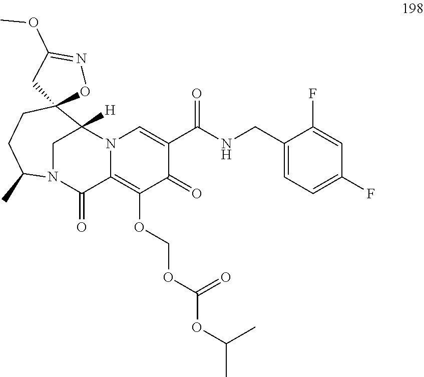 [SMILES: COC1=NO[C@@]2(CC[C@H](C)N3C[C@H]2n2cc(C(=O)NCc4ccc(F)cc4F)c(=O)c(OCOC(=O)OC(C)C)c2C3=O)C1]

The title compound was prepared in a similar manner to Example 11, except using chloromethyl isopropyl carbonate instead of 2-methoxyethyl carbonochloridate. MS (m/z) 619.8 [M+H]⁺. ¹H NMR (400 MHz, DMSO-d6) δ 10.28 (t, J=5.9 Hz, 1H), 8.74 (s, 1H), 7.42 (td, J=8.7, 6.6 Hz, 1H), 7.25 (ddd, J=10.5, 9.4, 2.6 Hz, 1H), 7.08 (tdd, J=8.6, 2.6, 1.0 Hz, 1H), 5.77 (d, J=6.4 Hz, 1H), 5.62 (d, J=6.5 Hz, 1H), 4.71 (d, J=2.7 Hz, 1H), 4.69-4.46 (m, 3H), 4.09-3.93 (m, 2H), 3.82 (s, 3H), 3.68 (d, J=2.2 Hz, 2H), 3.05 (d, J=16.8 Hz, 1H), 2.76 (d, J=16.9 Hz, 1H), 1.98-1.69 (m, 3H), 1.58 (h, J=7.1 Hz, 2H), 1.45-1.27 (m, 1H), 1.21-1.12 (m, 3H), 0.86 (t, J=7.4 Hz, 3H).

### Example 199: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl isobutyl carbonate (199)

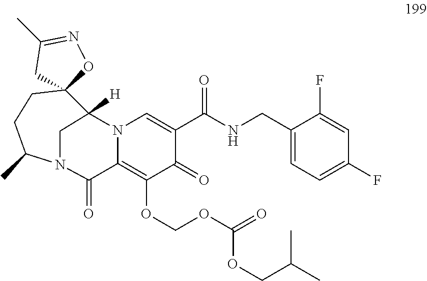 [SMILES: CC1=NO[C@@]2(CC[C@H](C)N3C[C@H]2n2cc(C(=O)NCc4ccc(F)cc4F)c(=O)c(OCOC(=O)OCC(C)C)c2C3=O)C1]

The title compound was prepared in a similar manner to Example 178, except using isobutyl alcohol instead of octan-1-ol in Step 1. MS (m/z) 617.97 [M+H]⁺. ¹H NMR (400 MHz, Chloroform-d) δ 10.29 (t, J=6.0 Hz, 1H), 8.35 (s, 1H), 7.38 (d, J=2.0 Hz, 1H), 6.90-6.74 (m, 2H), 6.05 (d, J=6.4 Hz, 1H), 5.88 (d, J=6.4 Hz, 1H), 4.92-4.83 (m, 1H), 4.64 (dd, J=20.7, 6.0 Hz, 2H), 4.07-3.93 (m, 3H), 3.76 (dd, J=15.1, 1.9 Hz, 1H), 3.62 (dd, J=15.0, 2.7 Hz, 1H), 3.05 (d, J=17.7 Hz, 1H), 2.66-2.55 (m, 1H), 2.11 (s, 3H), 1.98-1.87 (m, 3H), 1.63 (s, 2H), 1.23 (d, J=6.7 Hz, 3H), 0.96 (dd, J=6.7, 1.1 Hz, 6H).

### Example 200: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl isobutyl carbonate (200)

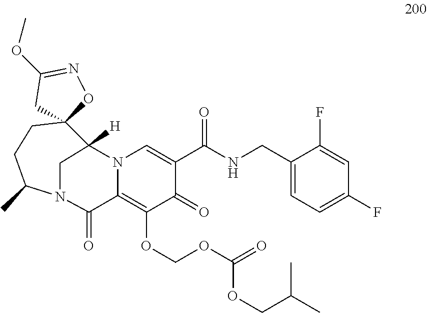 [SMILES: COC1=NO[C@@]2(CC[C@H](C)N3C[C@H]2n2cc(C(=O)NCc4ccc(F)cc4F)c(=O)c(OCOC(=O)OCC(C)C)c2C3=O)C1]

The title compound was prepared in a similar manner to Example 178, except using isobutyl alcohol instead of octan-1-ol in Step 1 and using Intermediate C instead of Intermediate B in Step 2. MS (m/z) 633.94[M+H]⁺. ¹H NMR (400 MHz, Chloroform-d) δ 10.30 (t, J=6.0 Hz, 1H), 8.47 (s, 1H), 7.36 (dd, J=8.8, 6.5 Hz, 1H), 6.94-6.80 (m, 2H), 6.05 (d, J=6.4 Hz, 1H), 5.88 (d, J=6.4 Hz, 1H), 4.85 (t, J=8.4 Hz, 1H), 4.77-4.54 (m, 2H), 4.20 (s, 1H), 4.03-3.93 (m, 2H), 3.91 (s, 3H), 3.75 (dd, J=15.2, 1.8 Hz, 1H), 3.60 (dd, J=15.1, 2.7 Hz, 1H), 3.08 (d, J=17.1 Hz, 1H), 2.70 (s, 1H), 2.04-1.99 (m, 1H), 1.93 (d, J=8.2 Hz, 2H), 1.61 (s, 2H), 1.23 (d, J=6.7 Hz, 3H), 1.02-0.91 (m, 6H).

### Example 201: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl isopentyl carbonate (201)

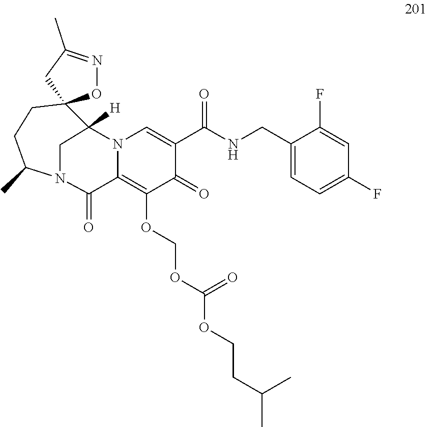 [SMILES: CC1=NO[C@@]2(CC[C@H](C)N3C[C@H]2n2cc(C(=O)NCc4ccc(F)cc4F)c(=O)c(OCOC(=O)OCCC(C)C)c2C3=O)C1]

The title compound was prepared in a similar manner to Example 178, except using 3-methylbutan-1-ol instead of octan-1-ol in Step 1. MS (m/z) 631.9 [M+H]⁺. ¹H NMR (400 MHz, DMSO-d6) δ 10.28 (t, J=6.0 Hz, 1H), 8.71 (s, 1H), 7.42 (td, J=8.7, 6.6 Hz, 1H), 7.25 (ddd, J=10.4, 9.3, 2.6 Hz, 1H), 7.08 (tdd, J=8.5, 2.6, 1.0 Hz, 1H), 5.80 (d, J=6.5 Hz, 1H), 5.63 (d, J=6.5 Hz, 1H), 4.72-4.48 (m, 4H), 4.08 (tt, J=7.8, 4.0 Hz, 2H), 3.69 (d, J=2.3 Hz, 2H), 3.17-2.86 (m, 1H), 2.79-2.58 (m, 1H), 1.95 (s, 3H), 1.78 (td, J=13.1, 12.6, 3.3 Hz, 3H), 1.63 (dh, J=13.3, 6.7 Hz, 1H), 1.47 (q, J=6.7 Hz, 2H), 1.29 (ddd, J=13.0, 10.3, 4.6 Hz, 1H), 1.16 (d, J=6.7 Hz, 3H), 0.87 (d, J=6.6 Hz, 6H).

### Example 202: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl isopentyl carbonate (202)

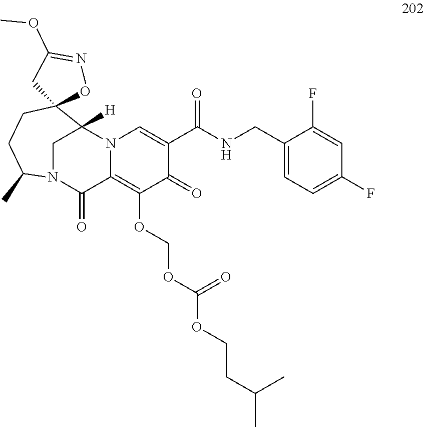 [SMILES: COC1=NO[C@@]2(CC[C@H](C)N3C[C@H]2n2cc(C(=O)NCc4ccc(F)cc4F)c(=O)c(OCOC(=O)OCCC(C)C)c2C3=O)C1]

The title compound was prepared in a similar manner to Example 178, except using 3-methylbutan-1-ol instead of octan-1-ol in Step 1 and using Intermediate C instead of Intermediate B in Step 2. MS (m/z) 647.7 [M+H]⁺. ¹H NMR (400 MHz, DMSO-d6) δ 10.28 (t, J=6.0 Hz, 1H), 8.75 (s, 1H), 7.42 (td, J=8.7, 6.6 Hz, 1H), 7.25 (ddd, J=10.5, 9.3, 2.6 Hz, 1H), 7.12-7.00 (m, 1H), 5.79 (d, J=6.5 Hz, 1H), 5.63 (d, J=6.5 Hz, 1H), 4.72 (d, J=2.4 Hz, 1H), 4.69-4.43 (m, 3H), 4.08 (tt, J=8.2, 4.2 Hz, 2H), 3.82 (s, 3H), 3.74-3.57 (m, 2H), 3.06 (d, J=16.9 Hz, 1H), 2.76 (d, J=16.9 Hz, 1H), 2.03-1.88 (m, 1H), 1.88-1.71 (m, 2H), 1.71-1.56 (m, J=6.8 Hz, 1H), 1.47 (q, J=6.7 Hz, 2H), 1.42-1.27 (m, 1H), 1.16 (d, J=6.7 Hz, 3H), 0.86 (d, J=6.6 Hz, 6H).

### Example 203: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-methylpentyl) carbonate (203)

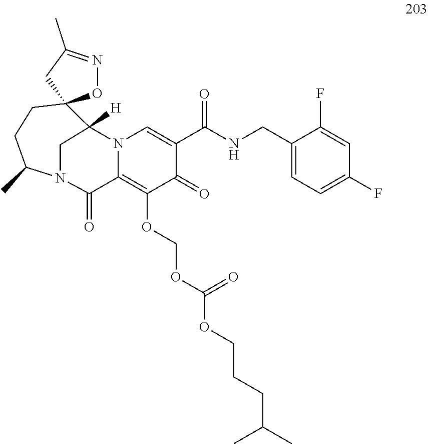 [SMILES: CC1=NO[C@@]2(CC[C@H](C)N3C[C@H]2n2cc(C(=O)NCc4ccc(F)cc4F)c(=O)c(OCOC(=O)OCCCC(C)C)c2C3=O)C1]

The title compound was prepared in a similar manner to Example 178, except using 4-methylpentan-1-ol instead of octan-1-ol in Step 1. MS (m/z) 645.6 [M+H]⁺. ¹H NMR (400 MHz, DMSO-d6) δ 10.28 (t, J=6.0 Hz, 1H), 8.71 (s, 1H), 7.42 (td, J=8.7, 6.6 Hz, 1H), 7.25 (ddd, J=10.5, 9.3, 2.6 Hz, 1H), 7.17-6.89 (n, 1H), 5.80 (d, J=6.5 Hz, 1H), 5.63 (d, J=6.5 Hz, 1H), 4.75-4.42 (m, 4H), 4.03 (tt, J=7.1, 3.6 Hz, 2H), 3.69 (d, J=2.3 Hz, 2H), 3.01 (d, J=17.5 Hz, 1H), 2.64 (d, J=17.6 Hz, 1H), 1.95 (s, 3H), 1.90-1.67 (m, 3H), 1.67-1.42 (m, 3H), 1.42-1.25 (m, 1H), 1.18 (dd, J=15.2, 6.8 Hz, 5H), 0.85 (d, J=6.6 Hz, 6H).

### Example 204: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (4-methylpentyl) carbonate (204)

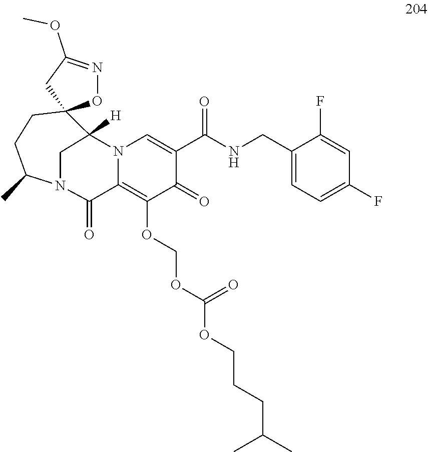 [SMILES: COC1=NO[C@@]2(CC[C@H](C)N3C[C@H]2n2cc(C(=O)NCc4ccc(F)cc4F)c(=O)c(OCOC(=O)OCCCC(C)C)c2C3=O)C1]

The title compound was prepared in a similar manner to Example 178, except using 4-methylpentan-1-ol instead of octan-1-ol in Step 1 and using Intermediate C instead of Intermediate B in Step 2. MS (m/z) 661.2 [M+H]⁺. ¹H NMR (400 MHz, DMSO-d6) δ 10.28 (t, J=6.0 Hz, 1H), 8.75 (s, 1H), 7.42 (td, J=8.7, 6.6 Hz, 1H), 7.25 (ddd, J=10.5, 9.3, 2.6 Hz, 1H), 7.13-7.03 (m, 1H), 5.78 (d, J=6.5 Hz, 1H), 5.62 (d, J=6.4 Hz, 1H), 4.71 (d, J=2.4 Hz, 1H), 4.69-4.58 (m, 1H), 4.55 (t, J=5.5 Hz, 2H), 4.03 (tt, J=7.7, 3.9 Hz, 2H), 3.82 (s, 3H), 3.71-3.65 (m, 2H), 3.06 (d, J=16.8 Hz, 1H), 2.76 (d, J=16.9 Hz, 1H), 1.96-1.86 (m, 1H), 1.84-1.70 (m, 2H), 1.63-1.53 (m, 2H), 1.57-1.44 (m, 1H), 1.33 (dt, J=15.5, 5.4 Hz, 1H), 1.18 (dd, J=16.0, 6.8 Hz, 5H), 0.85 (d, J=6.6 Hz, 6H).

### Example 205: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (5-methylhexyl) carbonate (205)

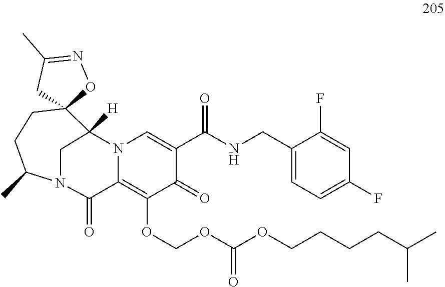 [SMILES: CC1=NO[C@@]2(CC[C@H](C)N3C[C@H]2n2cc(C(=O)NCc4ccc(F)cc4F)c(=O)c(OCOC(=O)OCCCCC(C)C)c2C3=O)C1]

The title compound was prepared in a similar manner to Example 178, except using 5-methylhexan-1-ol instead of octan-1-ol in Step 1. MS (m/z) 659.014 [M+H]⁺. ¹H NMR (400 MHz, Chloroform-d) δ 10.29 (t, J=6.0 Hz, 1H), 8.34 (s, 1H), 7.38 (t, J=7.7 Hz, 1H), 6.90-6.81 (m, 2H), 6.07 (d, J=6.4 Hz, 1H), 5.87 (d, J=6.4 Hz, 1H), 4.94-4.81 (m, 1H), 4.68 (dd, J=15.1, 6.1 Hz, 1H), 4.64-4.58 (m, 1H), 4.25-4.14 (m, 2H), 4.04 (d, J=2.2 Hz, 1H), 3.76 (dd, J=15.1, 1.9 Hz, 1H), 3.61 (dd, J=15.0, 2.7 Hz, 1H), 3.05 (d, J=17.8 Hz, 1H), 2.60 (d, J=17.8 Hz, 1H), 2.11 (d, J=2.8 Hz, 3H), 1.98-1.89 (m, 3H), 1.72-1.64 (m, 2H), 1.60 (s, 3H), 1.39 (t, J=8.0 Hz, 2H), 1.23 (t, J=6.3 Hz, 4H), 0.88 (d, J=6.6 Hz, 6H).

### Example 206: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (5-methylhexyl) carbonate (206)

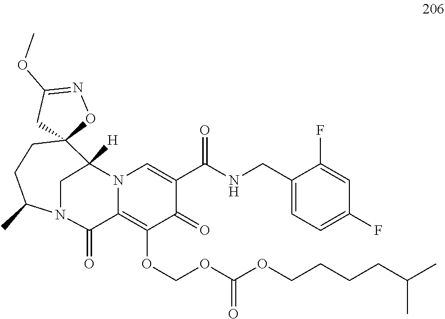 [SMILES: COC1=NO[C@@]2(CC[C@H](C)N3C[C@H]2n2cc(C(=O)NCc4ccc(F)cc4F)c(=O)c(OCOC(=O)OCCCCC(C)C)c2C3=O)C1]

The title compound was prepared in a similar manner to Example 178, except using 5-methylhexan-1-ol instead of octan-1-ol in Step 1 and using Intermediate C instead of Intermediate B in Step 2. MS (m/z) 675.026 [M+H]⁺. ¹H NMR (400 MHz, Chloroform-d) δ 10.29 (t, J=6.0 Hz, 1H), 8.46 (s, 1H), 7.46-7.33 (m, 1H), 6.90-6.75 (m, 2H), 6.06 (d, J=6.4 Hz, 1H), 5.86 (d, J=6.4 Hz, 1H), 4.86 (dd, J=10.0, 7.0 Hz, 1H), 4.76-4.66 (m, 1H), 4.62-4.53 (m, 1H), 4.21-4.17 (m, 2H), 3.91 (d, J=1.7 Hz, 3H), 3.75 (dd, J=15.1, 1.9 Hz, 1H), 3.60 (dd, J=15.1, 2.7 Hz, 1H), 3.08 (d, J=17.2 Hz, 1H), 2.68 (d, J=17.2 Hz, 1H), 2.06-1.99 (m, 1H), 1.93 (d, J=8.2 Hz, 2H), 1.71-1.64 (m, 2H), 1.61 (s, 3H), 1.43-1.35 (m, 2H), 1.21 (dd, J=11.7, 7.8 Hz, 5H), 0.89 (dd, J=6.6, 4.4 Hz, 6H).

### Example 207: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (6-methylheptyl) carbonate (207)

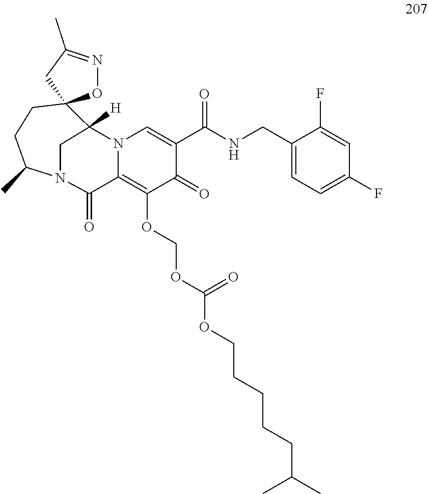 [SMILES: CC1=NO[C@@]2(CC[C@H](C)N3C[C@H]2n2cc(C(=O)NCc4ccc(F)cc4F)c(=O)c(OCOC(=O)OCCCCCC(C)C)c2C3=O)C1]

The title compound was prepared in a similar manner to Example 178, except using 6-methylheptan-1-ol instead of octan-1-ol in Step 1. MS (m/z) 673.4 [M+H]⁺. ¹H NMR (400 MHz, DMSO-d6) δ 10.29 (t, J=6.0 Hz, 1H), 8.70 (s, 1H), 7.42 (td, J=8.7, 6.6 Hz, 1H), 7.25 (ddd, J=10.5, 9.3, 2.6 Hz, 1H), 7.08 (td, J=8.6, 2.6 Hz, 1H), 5.80 (d, J=6.5 Hz, 1H), 5.62 (d, J=6.5 Hz, 1H), 4.72-4.45 (m, 4H), 4.04 (tt, J=6.6, 3.4 Hz, 2H), 3.69 (d, J=2.3 Hz, 2H), 3.01 (d, J=17.5 Hz, 1H), 2.64 (d, J=17.6 Hz, 1H), 1.95 (s, 3H), 1.87-1.68 (m, 3H), 1.51 (ddt, J=33.1, 13.2, 6.7 Hz, 3H), 1.26 (dq, J=7.4, 4.7, 3.6 Hz, 5H), 1.15 (dd, J=11.1, 5.7 Hz, 5H), 0.84 (d, J=6.6 Hz, 6H).

### Example 208: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (6-methylheptyl) carbonate (208)

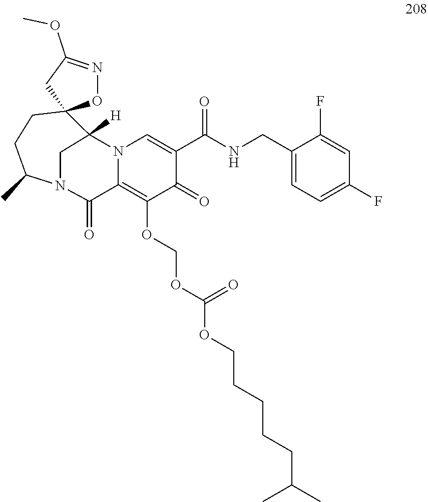 [SMILES: COC1=NO[C@@]2(CC[C@H](C)N3C[C@H]2n2cc(C(=O)NCc4ccc(F)cc4F)c(=O)c(OCOC(=O)OCCCCCC(C)C)c2C3=O)C1]

The title compound was prepared in a similar manner to Example 178, except using 6-methylheptan-1-ol instead of octan-1-ol in Step 1 and using Intermediate C instead of Intermediate B in Step 2. MS (m/z) 689.7 [M+H]⁺. ¹H NMR (400 MHz, DMSO-d6) δ 10.28 (t, J=6.0 Hz, 1H), 8.75 (s, 1H), 7.42 (td. J=8.7, 6.6 Hz, 1H), 7.25 (ddd, J=10.5, 9.3, 2.6 Hz, 1H), 7.15-6.93 (m, 1H), 5.78 (d, J=6.4 Hz, 1H), 5.62 (d, J=6.4 Hz, 1H), 4.71 (d, J=2.3 Hz, 1H), 4.69-4.47 (m, 3H), 4.04 (tt, J=7.0, 3.5 Hz, 2H), 3.82 (s, 3H), 3.75-3.54 (m, 2H), 3.06 (d, J=16.8 Hz, 1H), 2.75 (d, J=16.9 Hz, 1H), 2.02-1.87 (m, 1H), 1.87-1.73 (m, 2H), 1.51 (ddp, J=33.3, 13.5, 7.0 Hz, 3H), 1.41-1.21 (m, 5H), 1.14 (dd, J=11.3, 6.9 Hz, 5H), 0.84 (d, J=6.6 Hz, 6H).

### Example 209: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (7-methyloctyl) carbonate (209)

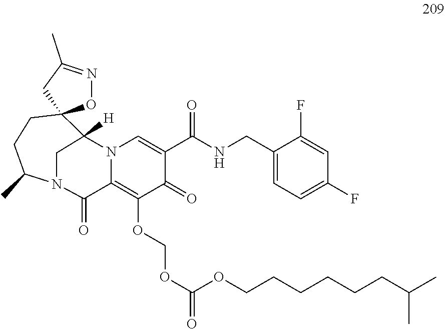 [SMILES: CC1=NO[C@@]2(CC[C@H](C)N3C[C@H]2n2cc(C(=O)NCc4ccc(F)cc4F)c(=O)c(OCOC(=O)OCCCCCCC(C)C)c2C3=O)C1]

The title compound was prepared in a similar manner to Example 178, except using 7-methyloctan-1-ol instead of octan-1-ol in Step 1. MS (m/z) 687.04 [M+H]⁺. ¹H NMR (400 MHz, Chloroform-d) δ 10.29 (t, J=6.0 Hz, 1H), 8.34 (s, 1H), 7.37 (td, J=8.6, 6.5 Hz, 1H), 6.91-6.77 (m, 2H), 6.07 (d, J=6.4 Hz, 1H), 5.87 (d, J=6.4 Hz, 1H), 4.92-4.81 (m, 1H), 4.74-4.53 (m, 2H), 4.27-4.16 (m, 1H), 4.21-4.06 (m, 1H), 4.03 (d, J=2.2 Hz, 1H), 3.76 (dd, J=15.1, 1.9 Hz, 1H), 3.61 (dd, J=15.1, 2.7 Hz, 1H), 3.05 (d, J=17.8 Hz, 1H), 2.60 (dd, J=17.7, 1.3 Hz, 1H), 2.11 (s, 3H), 2.01-1.84 (m, 3H), 1.74-1.47 (m, 6H), 1.45-1.08 (m, 9H), 0.88 (d, J=6.6 Hz, 6H).

### Example 210: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (7-methyloctyl) carbonate (210)

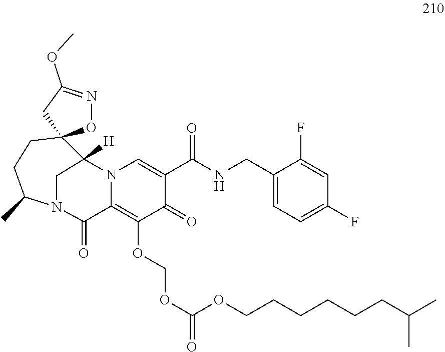 [SMILES: COC1=NO[C@@]2(CC[C@H](C)N3C[C@H]2n2cc(C(=O)NCc4ccc(F)cc4F)c(=O)c(OCOC(=O)OCCCCCCC(C)C)c2C3=O)C1]

The title compound was prepared in a similar manner to Example 178, except using 7-methyloctan-1-ol instead of octan-1-ol in Step 1 and using Intermediate C instead of Intermediate B in Step 2. MS (m/z) 703.03 [M+H]⁺. ¹H NMR (400 MHz, Chloroform-d) δ 10.29 (t, J=5.9 Hz, 1H), 8.45 (s, 1H), 7.37 (td, J=8.4, 6.4 Hz, 1H), 6.91-6.77 (m, 2H), 6.06 (d, J=6.4 Hz, 1H), 5.86 (d, J=6.4 Hz, 1H), 4.87 (dt, J=9.8, 6.9 Hz, 1H), 4.69 (dd, J=15.1, 6.1 Hz, 1H), 4.60 (dd, J=15.1, 5.8 Hz, 1H), 4.36-4.10 (m, 3H), 3.92 (s, 3H), 3.75 (dd, J=15.1, 1.9 Hz, 1H), 3.60 (dd, J=15.1, 2.7 Hz, 1H), 3.08 (d, J=17.2 Hz, 1H), 2.69 (d, J=17.2 Hz, 1H), 2.06-1.96 (m, 1H), 1.94 (t, J=9.0 Hz, 2H), 1.84-1.45 (m, 4H), 1.44 (s, 1H), 1.40-1.15 (m, 10H), 0.88 (d, J=6.6 Hz, 6H).

### Example 211: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (8-methylnonyl) carbonate (211)

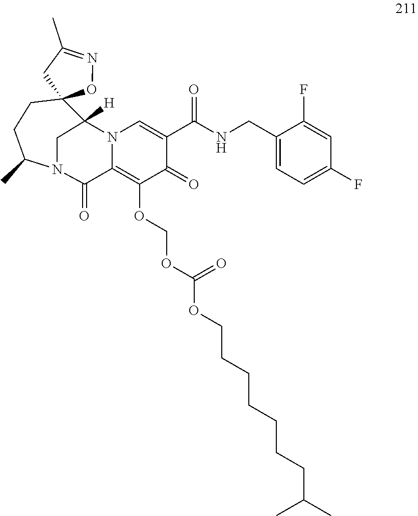 [SMILES: CC1=NO[C@@]2(CC[C@H](C)N3C[C@H]2n2cc(C(=O)NCc4ccc(F)cc4F)c(=O)c(OCOC(=O)OCCCCCCCC(C)C)c2C3=O)C1]

The title compound was prepared in a similar manner to Example 178, except using 8-methylnonan-1-ol instead of octan-1-ol in Step 1. MS (m/z) 701.7 [M+H]⁺. ¹H NMR (400 MHz, DMSO-d6) δ 10.29 (t, J=6.0 Hz, 1H), 8.70 (s, 1H), 7.42 (td, J=8.6, 6.6 Hz, 1H), 7.33-7.19 (m, 1H), 7.19-7.01 (m, 1H), 5.80 (d, J=6.5 Hz, 1H), 5.63 (t, J=5.7 Hz, 1H), 4.78-4.46 (m, 4H), 4.06 (dd, J=19.1, 6.2 Hz, 2H), 3.69 (d, J=2.3 Hz, 2H), 3.01 (d, J=17.6 Hz, 1H), 2.63 (d, J=17.5 Hz, 1H), 1.95 (s, 3H), 1.89-1.69 (m, 3H), 1.68-0.96 (m, 13H), 0.96-0.67 (m, 10H).

### Example 212: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (8-methylnonyl) carbonate (212)

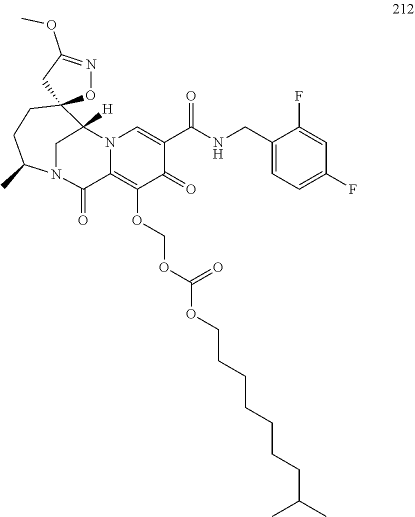 [SMILES: COC1=NO[C@@]2(CC[C@H](C)N3C[C@H]2n2cc(C(=O)NCc4ccc(F)cc4F)c(=O)c(OCOC(=O)OCCCCCCCC(C)C)c2C3=O)C1]

The title compound was prepared in a similar manner to Example 178, except using 8-methylnonan-1-ol instead of octan-1-ol in Step 1 and using Intermediate C instead of Intermediate B in Step 2. MS (m/z) 717.3 [M+H]⁺. ¹H NMR (400 MHz, DMSO-d6) δ 10.28 (t, J=6.0 Hz, 1H), 8.75 (s, 1H), 7.42 (td, J=8.7, 6.6 Hz, 1H), 7.25 (ddd, J=10.5, 9.3, 2.6 Hz, 1H), 7.07 (tt, J=8.5, 1.6 Hz, 1H), 5.78 (d, J=6.4 Hz, 1H), 5.62 (t, J=5.7 Hz, 1H), 4.76-4.48 (m, 5H), 4.16-3.95 (m, 2H), 3.82 (s, 3H), 3.68 (s, 2H), 3.06 (d, J=16.8 Hz, 1H), 2.75 (d, J=16.9 Hz, 1H), 1.97-1.73 (m, 3H), 1.70-0.94 (m, 12H), 0.93-0.65 (m, 10H).

### Example 213: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-methoxypropan-2-yl) carbonate (213)

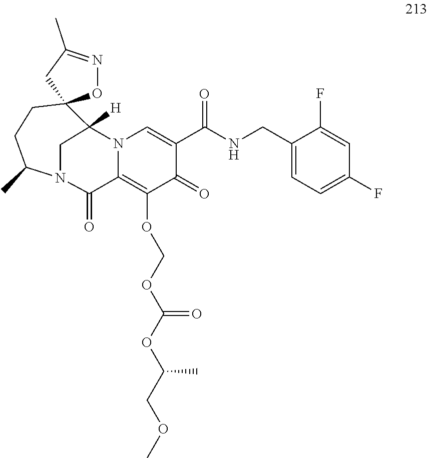 [SMILES: COC[C@@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using (2R)-1-methoxypropan-2-ol instead of octan-1-ol in Step 1. MS (m/z) 633.0 [M+H]⁺. ¹H NMR (400 MHz, Chloroform-d) δ 10.29 (t, J=5.9 Hz, 1H), 8.35 (s, 1H), 7.37 (td, J=8.6, 6.6 Hz, 1H), 6.90-6.78 (m, 2H), 6.08 (d, J=6.5 Hz, 1H), 5.83 (d, J=6.5 Hz, 1H), 5.02-4.80 (m, 2H), 4.68 (dd, J=15.1, 6.1 Hz, 1H), 4.60 (dd, J=15.1, 5.8 Hz, 1H), 4.04 (d, J=2.3 Hz, 1H), 3.76 (dd, J=15.1, 1.8 Hz, 1H), 3.63 (dd, J=15.1, 2.7 Hz, 1H), 3.53 (dd, J=10.6, 6.0 Hz, 1H), 3.45 (dd, J=10.6, 4.3 Hz, 1H), 3.37 (s, 3H), 3.05 (d, J=17.8 Hz, 1H), 2.64-2.55 (m, 1H), 2.10 (s, 3H), 2.03-1.85 (m, 3H), 1.65-1.55 (m, 1H), 1.31 (d, J=6.5 Hz, 3H), 1.24 (d, J=6.7 Hz, 3H).

### Example 214: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-1-methoxypropan-2-yl) carbonate (214)

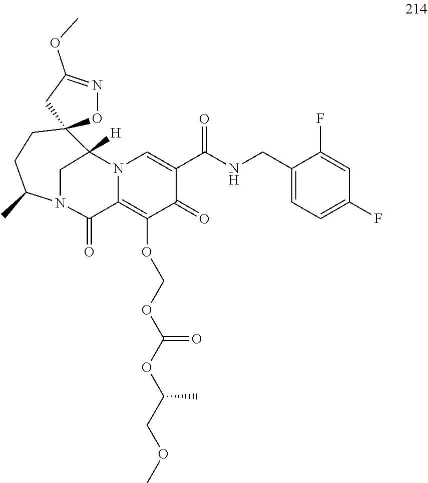 [SMILES: COC[C@@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using (2R)-1-methoxypropan-2-ol instead of octan-1-ol in Step 1 and using Intermediate C instead of Intermediate B in Step 2. MS (m/z) 649.0 [M+H]⁺. ¹H NMR (400 MHz, Chloroform-d) δ 10.29 (t. J=5.9 Hz, 1H), 8.43 (s, 1H), 7.38 (td, J=8.4, 6.3 Hz, 1H), 6.93-6.75 (m, 2H), 6.08 (d, J=6.4 Hz, 1H), 5.83 (d, J=6.4 Hz, 1H), 5.05-4.90 (m, 1H), 4.87 (dt, J=10.0, 7.0 Hz, 1H), 4.64 (qd, J=15.2, 5.7 Hz, 2H), 4.17 (s, 1H), 3.92 (s, 3H), 3.76 (dd, J=15.1, 1.9 Hz, 1H), 3.61 (dd, J=15.1, 2.7 Hz, 1H), 3.53 (dd, J=10.6, 6.1 Hz, 1H), 3.45 (dd, J=10.6, 4.4 Hz, 1H), 3.37 (s, 3H), 3.08 (d, J=17.2 Hz, 1H), 2.69 (d, J=17.3 Hz, 1H), 2.09-1.79 (m, 3H), 1.63 (ddd, J=15.2, 10.0, 2.9 Hz, 1H), 1.31 (d, J=6.5 Hz, 3H), 1.24 (d, J=6.7 Hz, 3H).

### Example 215: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-1-methoxypropan-2-yl) carbonate (215)

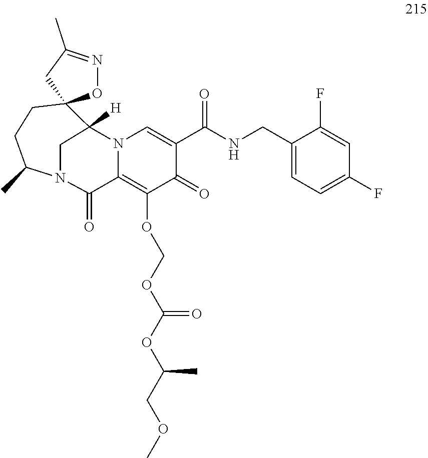 [SMILES: COC[C@H](C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using (2S)-1-methoxypropan-2-ol instead of octan-1-ol in Step 1. MS (m/z) 633.0 [M+H]⁺. 1H NMR (400 MHz, Chloroform-d) δ 10.28 (t, J=5.9 Hz, 1H), 8.36 (s, 1H), 7.43-7.32 (m, 1H), 6.93-6.72 (m, 2H), 6.01 (d, J=6.4 Hz, 1H), 5.92 (d, J=6.4 Hz, 1H), 4.96 (td, J=6.6, 4.4 Hz, 1H), 4.83 (dq, J=14.0, 7.1 Hz, 1H), 4.74-4.50 (m, 2H), 4.07 (s, 1H), 3.81-3.68 (m, 1H), 3.61 (dd. J=15.1, 2.6 Hz, 1H), 3.50 (dd, J=10.6, 6.2 Hz, 1H), 3.42 (dd, J=10.6, 4.2 Hz, 1H), 3.36 (s, 3H), 3.04 (d, J=17.7 Hz, 1H), 2.59 (d, J=17.8 Hz, 1H), 2.09 (s, 3H), 1.97-1.84 (m, 3H), 1.78 (s, 1H), 1.68-1.52 (m, 1H), 1.32 (d, J=6.5 Hz, 2H), 1.22 (d, J=6.7 Hz, 3H).

### Example 216: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-1-methoxypropan-2-yl) carbonate (216)

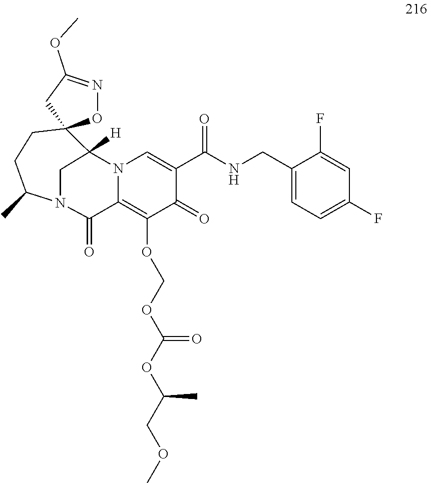 [SMILES: COC[C@H](C)OC(=O)OCOc1c2n(cc(C(=O)NCc3ccc(F)cc3F)c1=O)[C@@H]1CN(C2=O)[C@@H](C)CC[C@]12CC(OC)=NO2]

The title compound was prepared in a similar manner to Example 178, except using (2S)-1-methoxypropan-2-ol instead of octan-1-ol in Step 1 and using Intermediate C instead of Intermediate B in Step 2. MS (m/z) 649.0 [M+H]⁺. ¹H NMR (400 MHz, Chloroform-d) δ 10.30 (t, J=6.0 Hz, 1H), 8.52 (s, 1H), 7.37 (td, J=8.4, 6.3 Hz, 1H), 6.90-6.77 (m, 2H), 6.01 (d, J=6.4 Hz, 1H), 5.93 (d, J=6.4 Hz, 1H), 4.97 (pd, J=6.4, 4.2 Hz, 1H), 4.85 (dt, J=9.7, 6.8 Hz, 1H), 4.69 (dd, J=15.1, 6.2 Hz, 1H), 4.58 (dd, J=15.1, 5.7 Hz, 1H), 4.25 (s, 1H), 3.89 (s, 3H), 3.74 (dd, J=15.2, 1.9 Hz, 1H), 3.60 (dd, J=15.1, 2.6 Hz, 1H), 3.51 (dd, J=10.6, 6.2 Hz, 1H), 3.42 (dd, J=10.6, 4.2 Hz, 1H), 3.37 (s, 3H), 3.08 (d, J=17.1 Hz, 1H), 2.67 (d, J=17.2 Hz, 1H), 2.05-1.96 (m, 1H), 1.98-1.84 (m, 2H), 1.63 (ddd, J=15.3, 9.9, 3.0 Hz, 1H), 1.33 (d, J=6.4 Hz, 3H), 1.22 (d, J=6.7 Hz, 3H).

### Example 217: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-methoxypropyl) carbonate (217)

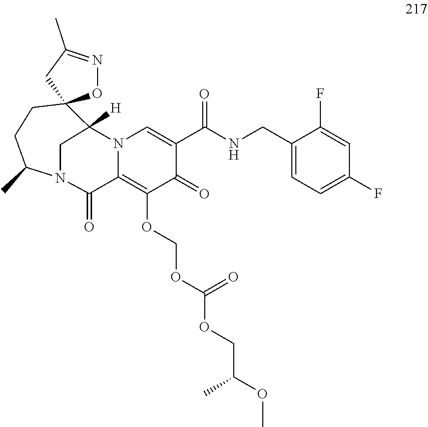 [SMILES: CO[C@H](C)COC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using (2R)-2-methoxypropan-1-ol instead of octan-1-ol in Step 1. MS (m/z) 633.2 [M+H]⁺. ¹H NMR (400 MHz, Chloroform-d) δ 10.28 (t, J=5.8 Hz, 1H), 8.35 (s, 1H), 7.37 (q, J=7.9 Hz, 1H), 6.84 (q, J=9.1, 8.6 Hz, 2H), 6.07 (d, J=6.5 Hz, 1H), 5.89 (d, J=6.4 Hz, 1H), 4.86 (q, J=7.7 Hz, 1H), 4.63 (qd, J=15.1, 5.8 Hz, 2H), 4.16 (tt, J=11.2, 6.3 Hz, 2H), 4.08 (d, J=18.2 Hz, 1H), 3.76 (d, J=15.1 Hz, 1H), 3.63 (dt, J=13.2, 6.0 Hz, 2H), 3.39 (s, 3H), 3.05 (d, J=17.7 Hz, 1H), 2.60 (d, J=17.8 Hz, 1H), 2.19 (s, 1H), 2.10 (s, 3H), 1.96-1.88 (m, 2H), 1.75-1.58 (m, 2H), 1.21 (dd, J=16.3, 6.5 Hz, 5H).

### Example 218: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((R)-2-methoxypropyl) carbonate (218)

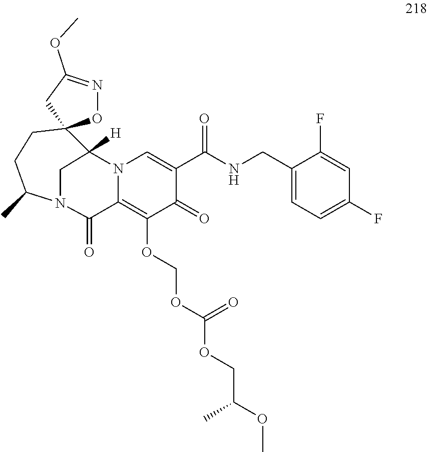 [SMILES: COC1=NO[C@@]2(CC[C@H](C)N3C[C@H]2n2cc(C(=O)NCc4ccc(F)cc4F)c(=O)c(OCOC(=O)OC[C@@H](C)OC)c2C3=O)C1]

The title compound was prepared in a similar manner to Example 178, except using (2R)-2-methoxypropan-1-ol instead of octan-1-ol in Step 1 and using Intermediate C instead of Intermediate B in Step 2. MS (m/z) 649.2 [M+H]⁺. ¹H NMR (400 MHz, Chloroform-d) δ 10.30 (t, J=5.9 Hz, 1H), 8.63 (s, 1H), 7.36 (td. J=8.4, 6.3 Hz, 1H), 6.89-6.76 (m, 2H), 6.05 (d, J=6.4 Hz, 1H), 5.86 (d, J=6.4 Hz, 1H), 4.85 (dt, J=9.7, 6.6 Hz, 1H), 4.69 (dd, J=15.2, 6.1 Hz, 1H), 4.56 (dd, J=15.1, 5.7 Hz, 1H), 4.36 (s, 1H), 4.14 (qd, J=11.1, 5.2 Hz, 2H), 3.86 (s, 3H), 3.73 (dd, J=15.2, 1.9 Hz, 1H), 3.61 (ddt, J=12.8, 6.6, 3.5 Hz, 2H), 3.38 (s, 3H), 3.09 (d, J=17.1 Hz, 1H), 2.64 (d, J=17.1 Hz, 1H), 2.04-1.83 (m, 3H), 1.60 (ddd, J=15.4, 10.2, 2.6 Hz, 1H), 1.20 (dd, J=15.9, 6.5 Hz, 6H).

### Example 219: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-methoxypropyl) carbonate (219)

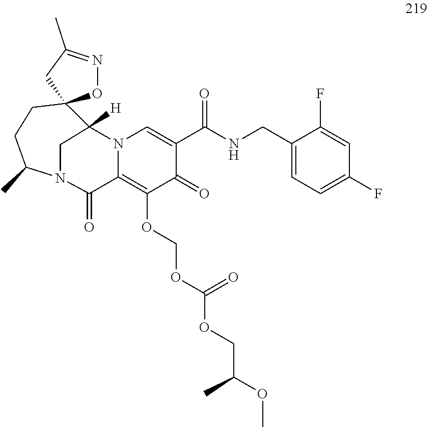 [SMILES: CO[C@@H](C)COC(=O)OCOc1c2n(cc(C(=O)NCc3ccc(F)cc3F)c1=O)[C@@H]1CN(C2=O)[C@@H](C)CC[C@]12CC(C)=NO2]

The title compound was prepared in a similar manner to Example 178, except using by (2S)-2-methoxypropan-1-ol instead of octan-1-ol in Step 1. MS (m/z) 633.0 [M+H]⁺. ¹H NMR (400 MHz, Chloroform-d) δ 10.28 (t, J=5.8 Hz, 1H), 8.35 (s, 1H), 7.37 (q, J=8.0 Hz, 1H), 6.95-6.72 (m, 2H), 6.07 (d, J=6.4 Hz, 1H), 5.89 (d, J=6.4 Hz, 1H), 4.85 (dq, J=13.8, 7.2 Hz, 1H), 4.64 (qd, J=15.2, 5.9 Hz, 2H), 4.25-4.09 (m, 2H), 4.04 (s, 1H), 3.76 (d, J=15.1 Hz, 1H), 3.69-3.55 (m, 2H), 3.39 (s, 3H), 3.05 (d, J=17.8 Hz, 1H), 2.60 (d, J=17.8 Hz, 1H), 2.11 (s, 3H), 2.02-1.84 (m, 3H), 1.64 (s, 3H), 1.24 (d, J=6.7 Hz, 2H), 1.20 (d, J=6.4 Hz, 2H).

### Example 220: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl ((S)-2-methoxypropyl) carbonate (220)

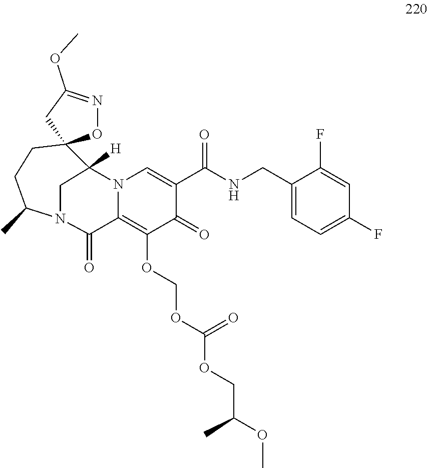 [SMILES: COC1=NO[C@@]2(CC[C@H](C)N3C[C@H]2n2cc(C(=O)NCc4ccc(F)cc4F)c(=O)c(OCOC(=O)OC[C@H](C)OC)c2C3=O)C1]

The title compound was prepared in a similar manner to Example 178, except using (2S)-2-methoxypropan-1-ol instead of octan-1-ol in Step 1 and using Intermediate C instead of Intermediate B in Step 2. MS (m/z) 649.0 [M+H]⁺. ¹H NMR (400 MHz, Chloroform-d) δ 10.30 (t, J=6.0 Hz, 1H), 8.56 (s, 1H), 7.37 (q, J=7.8 Hz, 1H), 6.84 (q, J=9.2 Hz, 2H), 6.06 (d, J=6.4 Hz, 1H), 5.87 (d, J=6.4 Hz, 1H), 4.86 (h, J=7.2 Hz, 1H), 4.69 (dd, J=15.1, 6.1 Hz, 1H), 4.57 (dd, J=15.2, 5.7 Hz, 1H), 4.29 (s, 1H), 4.14 (dp, J=11.3, 6.1 Hz, 2H), 3.88 (s, 3H), 3.74 (d, J=14.9 Hz, 1H), 3.62 (q, J=9.4, 7.7 Hz, 2H), 3.38 (s, 3H), 3.09 (d, J=17.1 Hz, 1H), 2.66 (d, J=17.2 Hz, 1H), 2.06-1.85 (m, 3H), 1.74 (s, 1H), 1.61 (dd, J=15.0, 9.5 Hz, 1H), 1.21 (dd, J=14.3, 6.5 Hz, 5H).

### Example 221: Preparation of (3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3-methoxybutan-2-yl) carbonate (221)

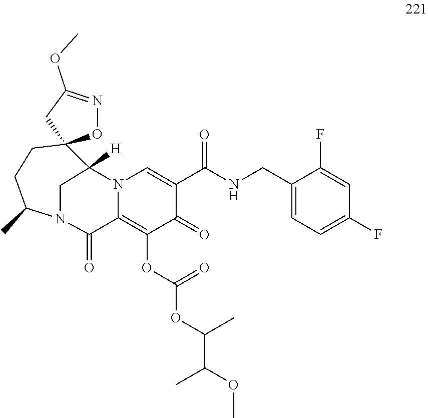 [SMILES: COC1=NO[C@@]2(CC[C@H](C)N3C[C@H]2n2cc(C(=O)NCc4ccc(F)cc4F)c(=O)c(OC(=O)OC(C)C(C)OC)c2C3=O)C1]

The title compound was made following the same method as Example 2, except using 3-methoxybutan-2-ol instead of 2-(2-methoxyethoxy)ethanol in Step 1. MS (m/z) 633.2 [M+H]⁺. 1H NMR (400 MHz, Chloroform-d) δ 10.23 (t, J=6.0 Hz, 1H), 8.74 (s, 1H), 7.36 (td, J=8.4, 6.3 Hz, 1H), 6.89-6.76 (m, 2H), 4.83 (dtq, J=8.2, 4.5, 2.3 Hz, 1H), 4.72 (dd, J=15.1, 6.3 Hz, 1H), 4.53 (dd, J=15.2, 5.6 Hz, 1H), 4.42 (d, J=2.6 Hz, 1H), 3.86 (s, 3H), 3.78 (dd, J=15.1, 1.7 Hz, 1H), 3.67 (dt, J=15.1, 2.5 Hz, 1H), 3.66-3.49 (m, 1H), 3.51-3.34 (m, 4H), 3.09 (d, J=17.1 Hz, 1H), 2.65 (d, J=17.1 Hz, 1H), 2.06-1.92 (m, 2H), 1.96-1.85 (m, 1H), 1.70-1.58 (m, 1H), 1.45-1.29 (m, 3H), 1.28-1.12 (m, 6H).

### Example 222: Preparation of (3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl (3-methoxybutan-2-yl) carbonate (222)

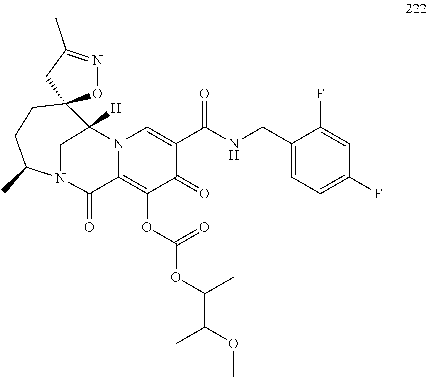 [SMILES: COC(C)C(C)OC(=O)Oc1c2n(cc(C(=O)NCc3ccc(F)cc3F)c1=O)[C@@H]1CN(C2=O)[C@@H](C)CC[C@]12CC(C)=NO2]

The title compound was made following the same method as Example 2, except using 3-methoxybutan-2-ol instead of 2(2-methoxyethoxy)ethanol in Step 1 and using Intermediate B instead of Intermediate C in Step 2. MS (m/z) 617.2 [M+H]⁺. 1H NMR (400 MHz, Chloroform-d) δ 10.19 (t, J=6.1 Hz, 1H), 8.42 (s, 1H), 7.36 (td, J=8.7, 6.5 Hz, 1H), 6.89-6.73 (m, 2H), 4.89-4.80 (m, 2H), 4.69 (dd, J=15.1, 6.2 Hz, 1H), 4.57 (dd, J=15.1, 5.7 Hz, 1H), 4.09 (d, J=2.2 Hz, 1H), 3.88-3.76 (m, 1H), 3.68 (dt, J=15.1, 2.6 Hz, 1H), 3.46-3.42 (m, 3H), 3.40 (d, J=2.3 Hz, 2H), 3.05 (d, J=17.9 Hz, 1H), 2.66-2.56 (m, 1H), 2.11 (s, 3H), 1.97-1.90 (m, 2H), 1.69-1.60 (m, 2H), 1.41 (dd, J=6.5, 5.5 Hz, 2H), 1.37-1.32 (m, 2H), 1.25 (d, J=6.7 Hz, 2H), 1.21 (dd, J=6.4, 2.2 Hz, 2H).

### Example 223: Preparation of (((3′S,5S,7′R)-10′-((2,4-difluorobenzyl)carbamoyl)-3,3′-dimethyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3-methoxybutan-2-yl) carbonate (223)

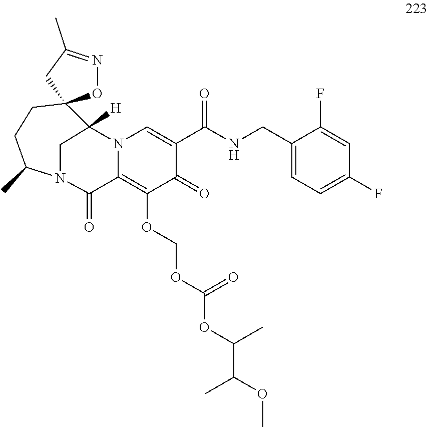 [SMILES: COC(C)C(C)OC(=O)OCOc1c2n(cc(C(=O)NCc3ccc(F)cc3F)c1=O)[C@@H]1CN(C2=O)[C@@H](C)CC[C@]12CC(C)=NO2]

The title compound was made following the same method as Example 178, except using 3-methoxybutan-2-ol instead of octan-1-ol in Step 1. MS (m/z) 647.2 [M+H]⁺. 1H NMR (400 MHz, Chloroform-d) δ 10.29 (t, J=6.0 Hz, 1H), 8.35 (d, J=1.3 Hz, 1H), 7.42-7.31 (m, 1H), 6.90-6.78 (m, 2H), 6.11-6.00 (m, 1H), 5.98-5.80 (m, 1H), 4.91-4.83 (m, 1H), 4.81 (ddt, J=17.1, 6.6, 3.7 Hz, 1H), 4.74-4.63 (m, 1H), 4.59 (dt, J=15.1, 4.8 Hz, 1H), 4.05 (s, 1H), 3.76 (dd, J=15.0, 1.8 Hz, 1H), 3.63 (ddt, J=14.9, 11.8, 2.7 Hz, 1H), 3.50-3.32 (m, 4H), 3.05 (d, J=17.8 Hz, 1H), 2.59 (dd, J=17.4, 3.7 Hz, 1H), 2.10 (s, 2H), 2.03-1.84 (m, 3H), 1.68-1.54 (m, 2H), 1.40-1.20 (m, 6H), 1.15 (t, J=6.6 Hz, 3H).

### Example 224: Preparation of (((3′S,5S,7′R)-10′-((2,4-difluorobenzyl)carbamoyl)-3-methoxy-3′-methyl-1′,11′-dioxo-1′,4′,5′,11′-tetrahydro-3′H,4H,7′H-spiro[isoxazole-5,6′-[2,7]methanopyrido[1,2-a][1,4]diazonin]-12′-yl)oxy)methyl (3-methoxybutan-2-yl) carbonate (224)

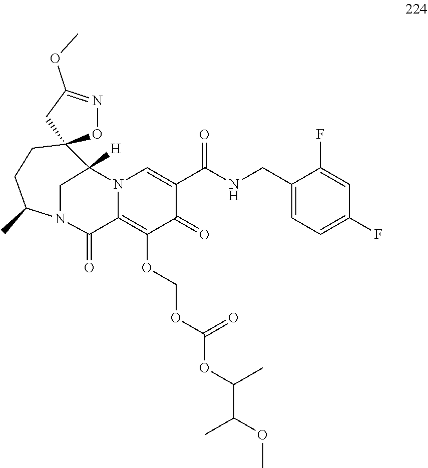 [SMILES: COC1=NO[C@@]2(CC[C@H](C)N3C[C@H]2n2cc(C(=O)NCc4ccc(F)cc4F)c(=O)c(OCOC(=O)OC(C)C(C)OC)c2C3=O)C1]

The title compound was made following the same method as Example 178, except using 3-methoxybutan-2-ol instead of octan-1-ol in Step 1 and using Intermediate C instead of was Intermediate B in Step 2. MS (m/z) 663.2 [M+H]⁺. 1H NMR (400 MHz, Chloroform-d) δ 10.29 (t, J=6.0 Hz, 1H), 8.46 (d, J=2.5 Hz, 1H), 7.37 (td, J=8.4, 6.5 Hz, 1H), 6.91-6.75 (m, 2H), 6.15-6.00 (m, 1H), 5.94-5.79 (m, 1H), 4.93-4.77 (m, 2H), 4.76-4.49 (m, 2H), 4.19 (d, J=2.5 Hz, 1H), 3.91 (s, 3H), 3.75 (dd, J=15.1, 1.8 Hz, 1H), 3.69-3.53 (m, 1H), 3.46-3.39 (m, 1H), 3.40-3.31 (m, 3H), 3.08 (d, J=17.2 Hz, 1H), 2.74-2.53 (m, 1H), 2.10-1.87 (m, 3H), 1.70-1.57 (m, 3H), 1.32-1.28 (m, 2H), 1.26-1.21 (m, 3H), 1.16 (dd, J=6.4, 0.8 Hz, 2H).

### Example A: HIV MT-4 Antiviral and Cytotoxicity Assay

### Antiviral Assay in MT-4 Cells

Compounds were tested in a high-throughput 384-well assay format for their ability to inhibit the replication of HIV-1 (IIIB) in MT-4 cells. Compounds were serially diluted (1.3) in DMSO on 384-well polypropylene plates and further diluted 200-fold into complete RPMI media (10% FBS, 1% P/S) using the Biotek Micro Flow and Labcyte ECHO acoustic dispenser. Each plate contained up to 8 test compounds, with negative (No Drug Control) and 5 μM AZT positive controls. MT-4 cells were pre-infected with 10 μL of either RPMI (mock-infected) or a fresh 1:250 dilution of HIV-1 IIIB concentrated virus stock. Infected and uninfected MT-4 cells were further diluted in complete RPMI media and added to each plate using a Micro Flow dispenser. After 5 days incubation in a humidified and temperature controlled incubator (37° C.), Cell Titer Glo (Promega) was added to the assay plates and chemiluminescence read using an Envision plate-reader. EC₅₀ values were defined as the compound concentration that causes a 50% decrease in luminescence signal, and were calculated using a sigmoidal dose-response model to generate curve fits. The EC₅₀ data for exemplary compounds is shown in Table 1.

### Cytotoxicity Assay in MT-4 Cells

Assays were performed as above except uninfected MT-4 cells were added to each well containing test compound. In addition, 10 M puromycin was added to the last column of each assay plate to assess a base level of cytotoxicity. The CC₅₀ data for exemplary compounds is shown in Table 1.

TABLE 1

| Example No. | MT4 EC₅₀ (nM) | MT4 CC₅₀ (nM) |
|---|---|---|
| 1 | 1.478 | 10867 |
| 2 | 0.79 | 11548 |
| 3 | 1.061 | 16277 |
| 4 | 15.433 | 50000 |
| 5 | 4.085 | 39755 |
| 6 | 9.476 | 50000 |
| 7 | 7.46 | 50000 |
| 8 | 0.632 | 11725 |
| 9 | 1.607 | 19554 |
| 10 | 6.401 | 31743 |
| 11 | 8.337 | 36803 |
| 12 | 3.805 | 25896 |
| 13 | 4.836 | 13428 |
| 14 | 6.464 | 50000 |
| 15 | 6.359 | 50000 |
| 16 | 1.296 | 11250 |
| 17 | 1.28 | 10184 |
| 18 | 2.764 | 20140 |
| 19 | 1.53 | 12312 |
| 20 | 11.49 | 34973 |
| 21 | 26.385 | 50000 |
| 22 | 0.992 | 12546 |
| 23 | 2.795 | 27289 |
| 24 | 5.165 | 31654 |
| 25 | 1.901 | 16165 |
| 26 | 2.622 | 22588 |
| 27 | 4.5 | 50000 |
| 28 | 1.717 | 11645 |
| 29 | 1.615 | 12320 |
| 30 | 1.3 | 12627 |
| 31 | 2.393 | 15656 |
| 32 | 1.177 | 31728 |
| 33 | 1.418 | 22624 |
| 34 | 0.218 | 16308 |
| 35 | 0.203 | 14084 |
| 36 | 0.345 | 19573 |
| 37 | 0.403 | 17376 |
| 38 | 0.331 | 25661 |
| 39 | 0.75 | 23657 |
| 40 | 0.592 | 25072 |
| 41 | 0.319 | 11491 |
| 42 | 1.944 | 40586 |
| 43 | 1.966 | 21061 |
| 44 | 0.477 | 24524 |
| 45 | 0.373 | 22218 |
| 46 | 2.055 | 36181 |
| 47 | 2.009 | 22635 |
| 48 | | 22405 |
| 49 | | 15076 |
| 50 | | 21748 |
| 51 | | 20041 |
| 52 | 0.604 | 24654 |
| 53 | 1.439 | 24350 |
| 54 | 0.504 | 19442 |
| 55 | 0.313 | 16390 |
| 56 | 1.236 | 34719 |
| 57 | 1.536 | 23077 |
| 58 | 0.284 | 23932 |
| 59 | 0.459 | 15377 |
| 60 | 0.539 | 21002 |
| 61 | 0.733 | 20611 |
| 62 | | |
| 63 | | |
| 64 | 2.483 | 38092 |
| 65 | 3.008 | 21768 |
| 66 | 0.316 | 22337 |
| 67 | 0.196 | 18855 |
| 68 | 1.094 | 16537 |
| 69 | 0.54 | 42170 |
| 70 | 1.023 | 17561 |
| 71 | 1.793 | 19500 |
| 72 | | 10791 |
| 73 | 0.797 | 20246 |
| 74 | | 26318 |
| 75 | 1.603 | 16115 |
| 76 | 0.518 | 46037 |
| 77 | 0.658 | 50000 |
| 78 | | 16247 |
| 79 | | 19515 |
| 80 | 1.302 | 19813 |
| 81 | 1.442 | 18111 |
| 82 | 2.518 | 17292 |
| 83 | 2.69 | 15998 |
| 84 | 3.293 | 19877 |
| 85 | 2.252 | 28392 |
| 86 | 2.526 | 21020 |
| 87 | 1.804 | 16389 |
| 88 | 1.784 | 20435 |
| 89 | 0.2 | 15784 |
| 90 | 0.176 | 18150 |
| 91 | 1.861 | 37257 |
| 92 | 2.139 | 21488 |
| 93 | 2.63 | 19514 |
| 94 | 2.734 | 17632 |
| 95 | 0.998 | 50000 |
| 96 | 0.562 | 50000 |
| 97 | 4.589 | 50000 |
| 98 | 3.865 | 50000 |
| 99 | 0.471 | 50000 |
| 100 | 0.246 | 50000 |
| 101 | 5.789 | 50000 |
| 102 | 4.956 | 50000 |
| 103 | | |
| 104 | 11.704 | 50000 |
| 105 | 22.455 | 50000 |
| 106 | 27.211 | 50000 |
| 107 | 32.32 | 50000 |
| 108 | | 21748 |
| 109 | | 15516 |
| 110 | 1.497 | 16379 |
| 111 | 1.952 | 13807 |
| 112 | 2.14 | 12244 |
| 113 | 1.78 | 20281 |
| 114 | 3.892 | 19722 |
| 115 | 3.625 | 16250 |
| 116 | 4.17 | 27165 |
| 117 | 2.841 | 50000 |
| 118 | 2.189 | 18166 |
| 119 | 2.874 | 43782 |
| 120 | 2.04 | 42327 |
| 121 | 1.172 | 18912 |
| 122 | | 19809 |
| 123 | 1.728 | 14147 |
| 124 | 0.898 | 17074 |
| 125 | | 18517 |
| 126 | | 21913 |
| 127 | 0.201 | 13325 |
| 128 | 0.21 | 19289 |
| 129 | 1.785 | 21737 |
| 130 | 1.83 | 31681 |
| 131 | 0.136 | 19849 |
| 132 | 0.153 | 17834 |
| 133 | 0.819 | 16698 |
| 134 | 0.516 | 25688 |
| 135 | 0.409 | 14207 |
| 136 | 0.496 | 23476 |
| 137 | 2.07 | 10173 |
| 138 | 2.144 | 14138 |
| 139 | 0.995 | 13223 |
| 140 | 3.362 | 15871 |
| 141 | 2.638 | 16541 |
| 142 | 3.644 | 36370 |
| 143 | | |
| 144 | 6.903 | 50000 |
| 145 | 5.928 | 50000 |
| 146 | 7.561 | 50000 |
| 147 | 7.158 | 50000 |
| 148 | 1.847 | 16134 |
| 149 | 2.167 | 15935 |
| 150 | 0.153 | 16030 |
| 151 | 0.17 | 18522 |
| 152 | 0.573 | 14883 |
| 153 | 0.648 | 23586 |
| 154 | 1.719 | 17200 |
| 155 | 1.19 | 17511 |
| 156 | 1.996 | 14698 |
| 157 | 2.325 | 15443 |
| 158 | | 16794 |
| 159 | | 35906 |
| 160 | 1.637 | 12617 |
| 161 | 1.593 | 40633 |
| 162 | 2.419 | 27774 |
| 163 | 2.082 | 37636 |
| 164 | | |
| 165 | | |
| 166 | | |
| 167 | | |
| 168 | | |
| 169 | | |
| 170 | | |
| 171 | | |
| 172 | 1.941 | 26791 |
| 173 | 4.752 | 37887 |
| 174 | 2.405 | 18117 |
| 175 | 2.057 | 23336 |
| 176 | 0.284 | 22956 |
| 177 | 0.366 | 23944 |
| 178 | 2.637 | 15781 |
| 179 | 0.527 | 50000 |
| 180 | 0.274 | 50000 |
| 181 | 0.262 | 14724 |
| 182 | 0.29 | 47866 |
| 183 | 0.358 | 11425 |
| 184 | 0.441 | 9978 |
| 185 | 5.145 | 50000 |
| 186 | 2.242 | 50000 |
| 187 | 3.251 | 43856 |
| 188 | 1.55 | 38503 |
| 189 | 3.54 | 50000 |
| 190 | 0.495 | 50000 |
| 191 | 1.665 | 50000 |
| 192 | | 50000 |
| 193 | | 50000 |
| 194 | 16.694 | 50000 |
| 195 | 33.528 | 50000 |
| 196 | 18.41 | 50000 |
| 197 | 41.217 | 50000 |
| 198 | 6.288 | 50000 |
| 199 | 3.869 | 15498 |
| 200 | 1.897 | 15191 |
| 201 | 4.473 | 16948 |
| 202 | 2.708 | 21585 |
| 203 | 3.559 | 26403 |
| 204 | 3.821 | 29840 |
| 205 | 2.473 | 23065 |
| 206 | 2.44 | 37250 |
| 207 | 0.564 | 11292 |
| 208 | 0.594 | 21308 |
| 209 | | 50000 |
| 210 | | 50000 |
| 211 | 3.733 | 36728 |
| 212 | 8.603 | 50000 |
| 213 | 8.29 | 50000 |
| 214 | 6.253 | 50000 |
| 215 | | 50000 |
| 216 | | 50000 |
| 217 | | 24570 |
| 218 | 3.086 | 32463 |
| 219 | | 33869 |
| 220 | | 41624 |

### Example B: FaSSIF and PBS Thermodynamic Solubility Assay

Approximately 7 mg of each test compound as a dry powder was placed in a vial. Aliquots were weighed out for each assay media at 2 hour and 24 hour time points, to be analyzed. The appropriate buffer (FaSSIF or PBS) was added to each vial such that the final dose concentration of 5 mg/mL was achieved. Samples were then vortexed for 5-10 seconds. Following a 2-hour or a 24-hour incubation on a rotary shaker (200 RPM) at ambient temperature (22.3-23.8° C.), the samples were vacuum filtered through a Millipore solubility filter plate with 0.45 μM polycarbonate filter membrane and the filtrates were collected in a 96 well polypropylene plate. The plate was sealed with a pierceable heat seal and analyzed by HPLC-UV.

An Agilent 1290 UHPLC equipped with a micro-well plate autosampler, quaternary HPLC pump, and diode array detector was used for analysis. Each filtrate (1.5 L) was injected onto the column (AQUASIL C18, 5 μM, 50×2.1 mm) and eluted using a gradient of 10-100% MeCN/water containing 0.1% formic acid. A system QC (0.01 mg/mL Caffeine in DMSO) was injected every 12-15 injections. Data was collected at 214, 254 and 280 nm. Results are reported using data obtained at 280 nm. The resulting peak areas were plotted against the known concentrations from the calibration and the filtrates are quantified with respect to the linear regression using Agilent OpenLab Intelligent Reporting Software. The results are reported electronically in mg/mL.

TABLE 2

| | Thermodynamic FaSSIF | Thermodynamic PBS |
|---|---|---|
| | Solubility @ 24 h | Solubility |
| Example No. | (mg/mL) | (mg/mL) |
| 3 | 0.47 | nd |
| 5 | 0.168 | 0.039 |
| 6 | <LOD | nd |
| 7 | 0.002 | nd |
| 8 | 0.486 | nd |
| 10 | 0.035 | 0.015 |
| 11 | 0.14 | nd |
| 12 | 0.03 | nd |
| 15 | nd | <LOD |
| 16 | 0.27 | 0.372 |
| 17 | nd | <LOD |
| 19 | 0.083 | nd |
| 20 | 0.07 | nd |
| 21 | 0.15 | nd |
| 22 | 0.07 | 0.034 |
| 24 | 0.008 | nd |
| Intermediate B | 0.002 | nd |
| Intermediate C | 0.002 | nd |

FaSSIF = Fasted-state simulated intestinal fluid.
LOD = Limit of detection
Nd = not determined

### Example C: Rat Parenteral Pharmacokinetic Studies

Doses are expressed as “mg-eq”, referring to the mass (in mg) of Intermediate B or Intermediate C equivalents (eq) as a fixed dose or relative to body weight (in kg).

### Parenteral Formulation Preparation

Each compound was dissolved in 100% NMP in a glass vial to form an 83.33 mg-eq/mL solution and allowed to equilibrate to ambient temperature for 15 mins. The solution was vortexed for 20 seconds and observed to ensure all compound is dissolved. The clear solution was filtered with a sterile 0.22 um PTFE syringe filter using a glass syringe into a separate sterile vial. 0.3 mL/kg was drawn into a glass syringe for parenteral injection (25 mg-eq/kg) within 4 hours of filtration.

### Rat PK Dosing

Male Wistar Han Intact Rats (250-350 g) were received from an approved vendor and allowed to acclimate to the Test Facility for at least 2 days. Subcutaneous (SC) doses were administered via injection into the dorsal surface between the shoulders and point of the hip in accordance with facility procedures. All animals were observed at dosing and each scheduled sample collection. Each injection site was evaluated at each collection time point and observations recorded. Serial blood samples (through Week 52) were collected into K₂EDTA tubes and stored on wet ice until processed. Whole blood was processed to plasma by centrifugation (3500 rpm for 10 minutes at 5° C.) within 30 minutes of collection. Plasma samples were transferred into Micronic 96 well tubes and stored at −80° C. as soon as possible and remained at −80° C. until shipped for bioanalysis.

### Bioanalysis of Plasma Samples:

To a 10 μL aliquot of each plasma sample with exception of the matrix blanks, 60 μL of 100 ng/mL Chrysin and Glyburide in acetonitrile (ACN) was added. The matrix blank samples received 60 μL of acetonitrile only. The precipitated proteins were removed by centrifugation and 50 μL of supernatant was transferred into a clean 96 well plate. A 50 μL aliquot of water was added to each sample. An aliquot of 0.5-5 μL was injected into an Applied Biosystems API-6500+LC/MS/MS system, using a Waters XSelect HSS T3 column and eluting with a gradient of water and acetonitrile (containing 0.1% formic acid).

AUC_{τ} was calculated as area under the plasma concentration vs. time curve from day 0 to a specified time point (τ). AUC_{inf} was calculated as area under the plasma concentration vs. time curve from day 0 to infinity.

Bioavailability (% F) was calculated by comparing plasma concentration via parenteral dose vs. plasma concentration via IV dose (intravenous) using the following equation:

$$\% F = \frac{S C AUCinf}{I V AUCinf} \times \frac{I V Dose}{S C Dose} \times 100$$

TABLE 3
Injectable Bioavailability of Intermediate B
or Intermediate C in Rats Following a 25 mg-
eq/kg Subcutaneous Injection of Test Compound.

| | | | Rat Injectable % F of Intermediate |
|---|---|---|---|
| | Compound | Compound | B or Intermediate C at Day 364 |
| Entry | Dosed | Measured | (25 mg-eq/kg NMP Solution) |
| 1 | 5 | Intermediate C | 99.9 |
| 2 | 8 | Intermediate B | 90.4 |
| 3 | 10 | Intermediate B | 74.7 |
| 4 | 15 | Intermediate B | 20.6 |
| 5 | 16 | Intermediate B | 52 |
| 6 | 17 | Intermediate B | 66.1 |
| 7 | 22 | Intermediate B | 81.3 |

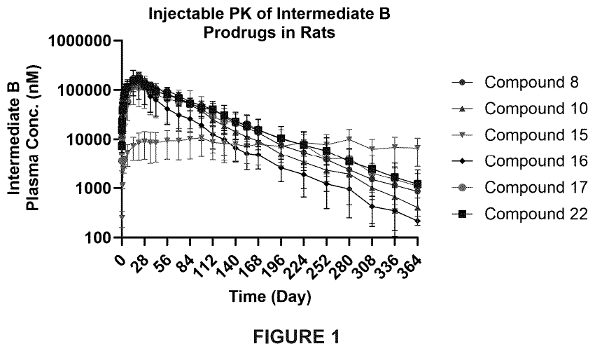

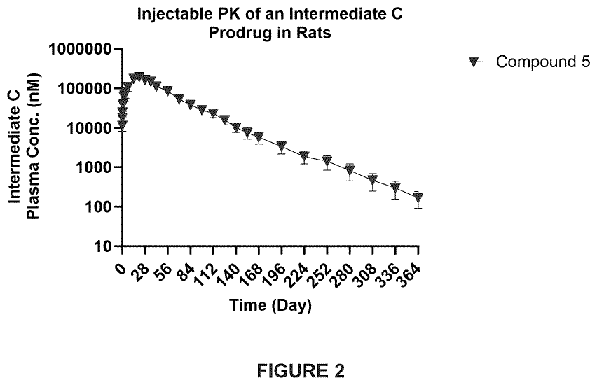

FIG. 1 shows an overlay of the plasma concentrations of Intermediate B measured over time following a single subcutaneous injection of Compounds 8, 10, 15, 16, 17, or 22 in rats. FIG. 2 shows plasma concentrations of Intermediate C measured over time following a single subcutaneous injection of Compound 5 in rats. The compounds disclosed herein generally demonstrate long-acting pharmacokinetic properties.

### Example D: Dog PO Pharmacokinetic Studies

### Dog PK Dosing

Male beagle dogs were fasted overnight. Food was returned approximately 4 hours postdose. In some instances, each animal received a single 6 μg/kg intramuscular injection of pentagastrin approximately 30 minutes prior to test article administration to stimulate gastric secretion. The intramuscular dose was administered in a thigh muscle using a needle and syringe.

Solution or suspension oral doses were administered at a dose of 4 mg-eq/kg via gavage and the dosing tube was flushed with ˜10 mL water prior to removal. Solid or capsule oral doses were administered at a dose of 40 mg-eq fixed, by hand by deep throat deposition. Following each dose, the animals were offered approximately 5 mL of water to assist in swallowing. This was done by depositing ˜1-3 mL at a time into the back of the throat and holding the mouth closed until swallowing was observed. This was repeated until all 5 mL of water has been given. Once swallowed, 25 mL water was administered by gavage for a total of 30 mL water administered with each dose. All animals were observed at dosing and each scheduled collection.

Serial blood samples (through 168 h) were collected into pre-chilled K₂EDTA with the appropriate volume of 40 mM dichlorvos added to result in a final dichlorvos concentration of 2 mM and stored on wet ice until processed. Whole blood was processed to plasma by centrifugation (3500 rpm for 10 minutes at 5° C.) within 30 minutes of collection. Plasma samples were transferred into Micronic 96 well tubes and stored at −70° C. as soon as possible and remained at −70° C. until shipped for bioanalysis.

### Bioanalysis of Plasma Samples

To a 20 μL aliquot of each plasma sample with exception of the matrix blanks, 120 μL of 100 ng/mL Carbamazepine and Chrysin in acetonitrile (ACN) was added. The matrix blank samples received 120 μL of acetonitrile only. The precipitated proteins were removed by centrifugation and 100 μL of supernatant was transferred into a clean 96-well plate. A 100 μL aliquot of water was added to each sample. An aliquot of 2-2.5 μL was injected into an Applied Biosystems API-6500 LC/MS/MS system, eluting with a gradient of water and acetonitrile (containing 0.1% formic acid).

AUC_{inf} was calculated as area under the plasma concentration vs. time curve from 0 h to infinity.

Bioavailability (% F) was calculated by comparing plasma concentration via PO dose (oral) vs. plasma concentration via IV dose (intravenous) using the following equation:

$$\% F = \frac{P O AUCinf}{I V AUCinf} \times \frac{I V Dose}{P O Dose} \times 100$$

TABLE 4
Oral Bioavailability of Intermediate B or Intermediate C in
Dog Following a 4 mg-eq/kg Solution Dose of Test Compound.

| | | | Dog % F of Intermediate B or |
|---|---|---|---|
| | | Compound | Intermediate C |
| Entry | Compound Dosed | Measured | (4 mg-eq/kg solution) |
| 1 | 5 | Intermediate C | 15.3 |
| 2 | 8 | Intermediate B | 5.4 |
| 3 | 11 | Intermediate C | 14.4 |
| 4 | 22 | Intermediate B | 17.7 |

TABLE 5
Oral Bioavailability of Intermediate C in Dog Following
a 40 mg-eq Powder-in-Capsule Fixed Dose of Test Compound.

| | | | Dog % F of Intermediate B |
|---|---|---|---|
| | | Compound | or Intermediate C |
| Entry | Compound Dosed | Measured | (40 mg-eq PIC) |
| 1 | 5 | Intermediate C | 12.6 |
| 2 | 11 | Intermediate C | 16.2 |

All of the U.S. patents. U.S. patent application publications. U.S. patent applications, foreign patents, foreign patent applications and non-patent publications referred to in this specification are incorporated herein by reference, in their entirety to the extent non inconsistent with the present description.

From the forgoing it will be appreciated that, although specific embodiments have been described herein for purposes of illustration, various modifications may be made without deviating from the spirit and scope of the disclosure. Accordingly, the disclosure is not limited except as by the appended claims.

1. A compound of Formula I: 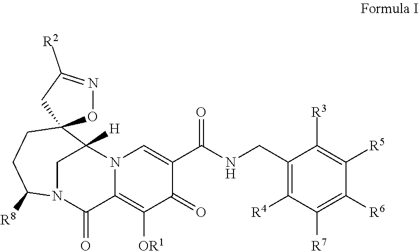
or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
wherein m is 0 or 1;
n is an integer between 0 and 10, inclusive;
p is 0 or 1;
R^{1A} is H or C₁₋₃alkyl;
R^{1B} is H or C₁₋₃alkyl;
each R^{C} is independently H or C₁₋₃alkyl;
each R^{1D} is independently H or C₁₋₃alkyl;
each R^{1E} is independently H or C₁₋₃alkyl;
each R^{1F} is independently H or C₁₋₃alkyl;
R^{1G} is C₃₋₆ cycloalkyl, C₁₋₂₀alkyl or C₁₋₂₀alkenyl; wherein the C₃₋₆ cycloalkyl, C₁₋₂₀alkyl or C₁₋₂₀alkenyl is optionally substituted with a COOH, C₃₋₆ cycloalkyl, C₁₋₃ alkoxy, or —NHCOOR¹H;
each R¹H is C₁₋₃alkyl;
R² is C₁₋₃ alkyl or C₁₋₃ alkoxy;
each R³, R⁴, R⁵, R⁶ and R⁷ is independently H or halo; and
R⁸ is H or C₁₋₃alkyl.

2. A compound of Formula I: 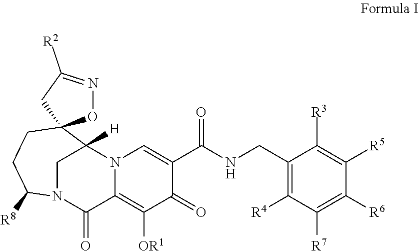
or a pharmaceutically acceptable salt thereof, wherein:
R¹ is —(CR^{1A}R¹BO)ₘCO(O—CR^{1C}R^{1D}—CR^{1E}R^{1F})ₙ—(O)ₚR^{1G};
wherein m is 0 or 1;
n is 0, 1, 2 or 3; and
p is 0 or 1;
R^{1A} is H or C₁₋₃alkyl;
R^{1B} is H or C₁₋₃alkyl;
each R^{C} is independently H or C₁₋₃alkyl;
each R^{1D} is independently H or C₁₋₃alkyl;
each R^{1E} is independently H or C₁₋₃alkyl;
each R^{1F} is independently H or C₁₋₃alkyl;
R^{1G} is C₃₋₆ cycloalkyl, C₁₋₂₀alkyl or C₁₋₂₀alkenyl; wherein the C₃₋₆ cycloalkyl, C₁₋₂₀alkyl or C₁₋₂₀alkenyl is optionally substituted with a COOH or C₃₋₆ cycloalkyl;
R² is C₁₋₃ alkyl or C₁₋₃ alkoxy;
each R³, R⁴, R⁵, R⁶ and R⁷ is independently H or halo; and
R⁸ is H or C₁₋₃alkyl.

3. The compound of claim 1, or the pharmaceutically acceptable salt thereof, wherein R⁴, R⁵ and R⁷ are each H.

4. The compound of claim 1, or the pharmaceutically acceptable salt thereof, wherein R³ and R⁶ are each independently a halo.

5. (canceled)

6. The compound of claim 1, or the pharmaceutically acceptable salt thereof, wherein R⁸ is C₁₋₃alkyl.

7. (canceled)

8. The compound of claim 1, or the pharmaceutically acceptable salt thereof, wherein R² is methyl or methoxy.

9.-18. (canceled)

19. The compound of a claim 1, or the pharmaceutically acceptable salt thereof, wherein the compound is of a Formula Ia:

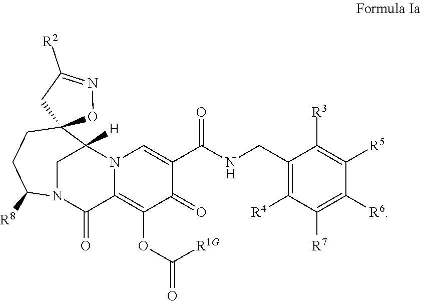

20. The compound of a claim 1, or the pharmaceutically acceptable salt thereof, wherein the compound is of a Formula Ib:

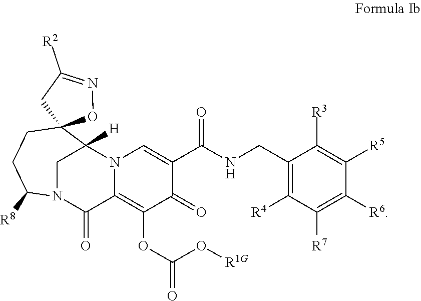

21. The compound of claim 1, or the pharmaceutically acceptable salt thereof, wherein the compound is of a Formula Ic: 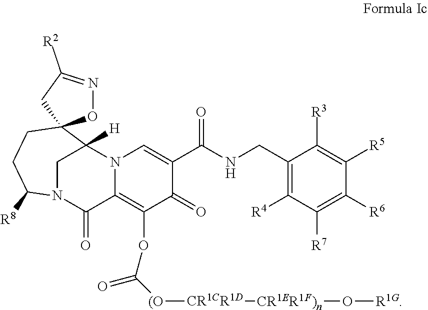
wherein n is an integer between 1 and 7, inclusive.

22. (canceled)

23. The compound of claim 1, or the pharmaceutically acceptable salt thereof, wherein the compound is of a Formula Id: 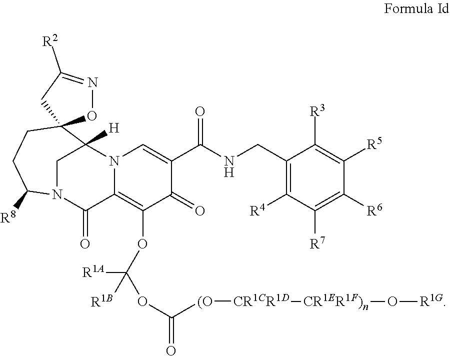
wherein n is 0, 1, 2, or 3.

24. (canceled)

25. The compound of claim 1, or the pharmaceutically acceptable salt thereof, wherein the compound is of a Formula Ie:

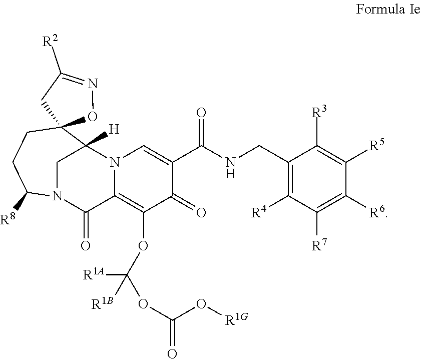

26. The compound of claim 1, or the pharmaceutically acceptable salt thereof, wherein the compound is of a Formula If.

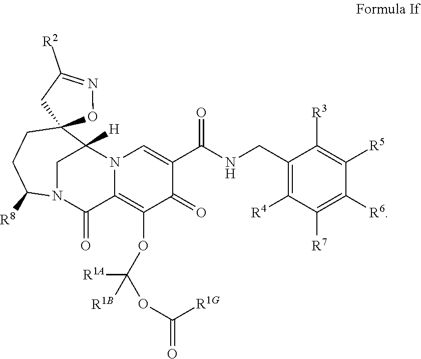

27.-38. (canceled)

39. The compound of claim 1, or the pharmaceutically acceptable salt thereof, wherein R^{1G} is C₅₋₆ cycloalkyl, C₁₋₂₀alkyl or C₁₋₂₀alkenyl; wherein the C₁₋₂₀alkyl or C₁₋₂₀alkenyl is optionally substituted with COOH, C₅₋₆ cycloalkyl, C₁₋₃ alkoxy, or —NHCOOR^{1H}.

40.-42. (canceled)

43. The compound of claim 1, wherein R^{1G} is C₁₋₂₀alkyl optionally substituted with a COOH, C₃₋₆ cycloalkyl, C₁₋₃ alkoxy, or —NHCOOR^{1H}.

44.-45. (canceled)

46. The compound of claim 1, or the pharmaceutically acceptable salt thereof, wherein R^{1G} is C₁₋₂₀alkenyl optionally substituted with a COOH.

47. (canceled)

48. The compound of claim 1, or the pharmaceutically acceptable salt thereof, wherein R^{1G} is C₃₋₆ cycloalkyl.

49. (canceled)

50. The compound of claim 1, or the pharmaceutically acceptable salt thereof, wherein R^{1G} is selected from the group consisting of —CH₃, —CH(CH₃)₂, —C(CH₃)₃, —(CH₂)₃CH₃, C₁₃H₂₇, C₁₅H₃₁, C₁₇H₃₅, C₁₇H₃₁, —CHCHCOOH, cyclopentyl, C₂H₅, C₃H₇, C₄H₉, C₅H₁₁, C₆H₁₃, C₇H₁₅, C₈H₁₇, C₉H₁₉, C₁₀H₂₁C₁₁H₂₃, C₁₂H₂₅, C₁₄H₂₉, C₁₅H₃₁, C₁₆H₃₃, C₁₈H₃₇, —(CH₂)₂(cyclopentyl), —(CH₂)₅OCH₃, —(CH₂)₃COOH, —(CH₂)₅COOH, —(CH₂)₁₆COOH, —CH(CH₃)NHCOOCH₃, —CH(C₃H₇)NHCOOCH₃, C₁₃H₂₅, and C₁₉H₂₉.

51.-53. (canceled)

54. The compound of claim 1, or the pharmaceutically acceptable salt thereof, wherein R¹ is selected from the group consisting of:

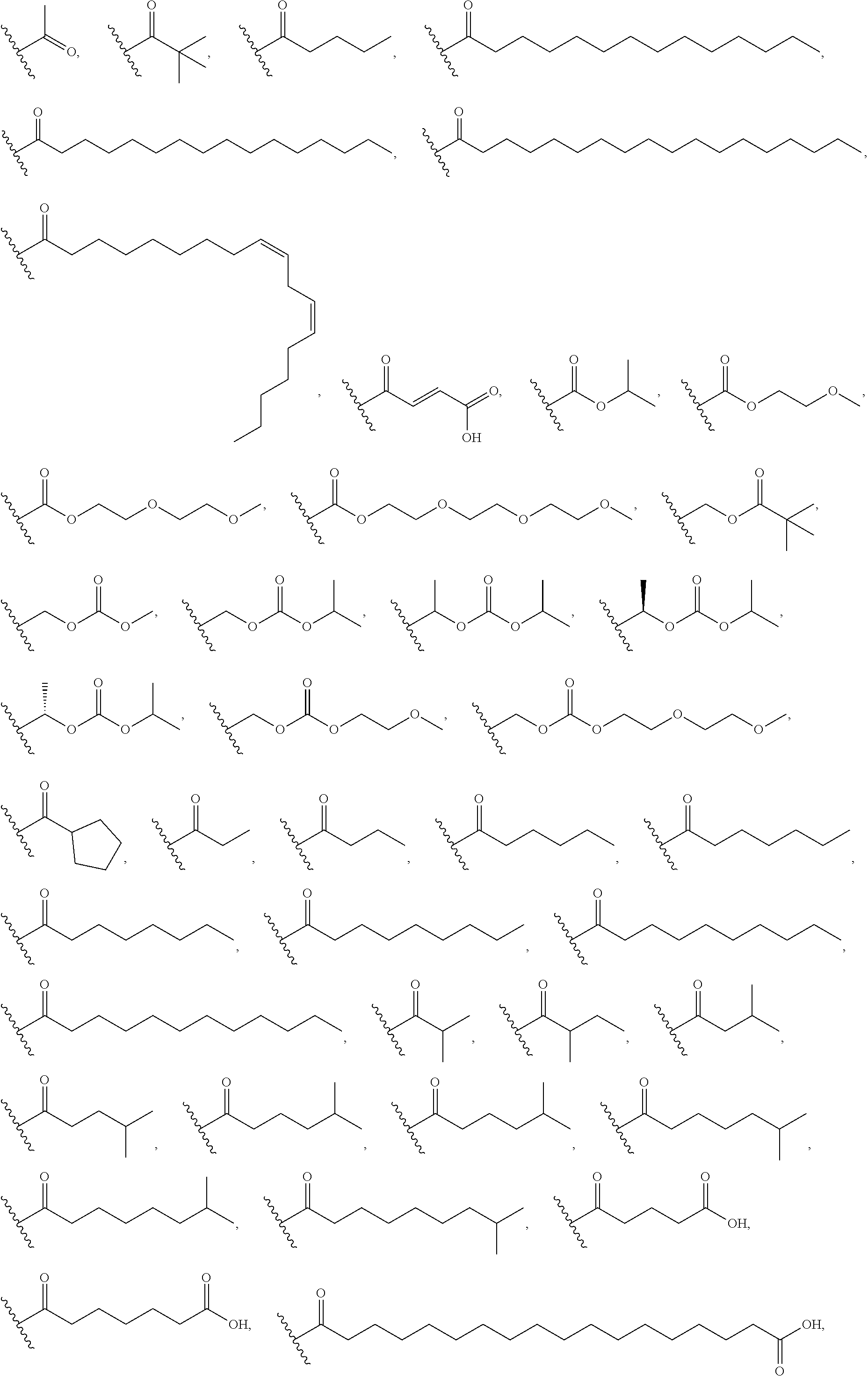

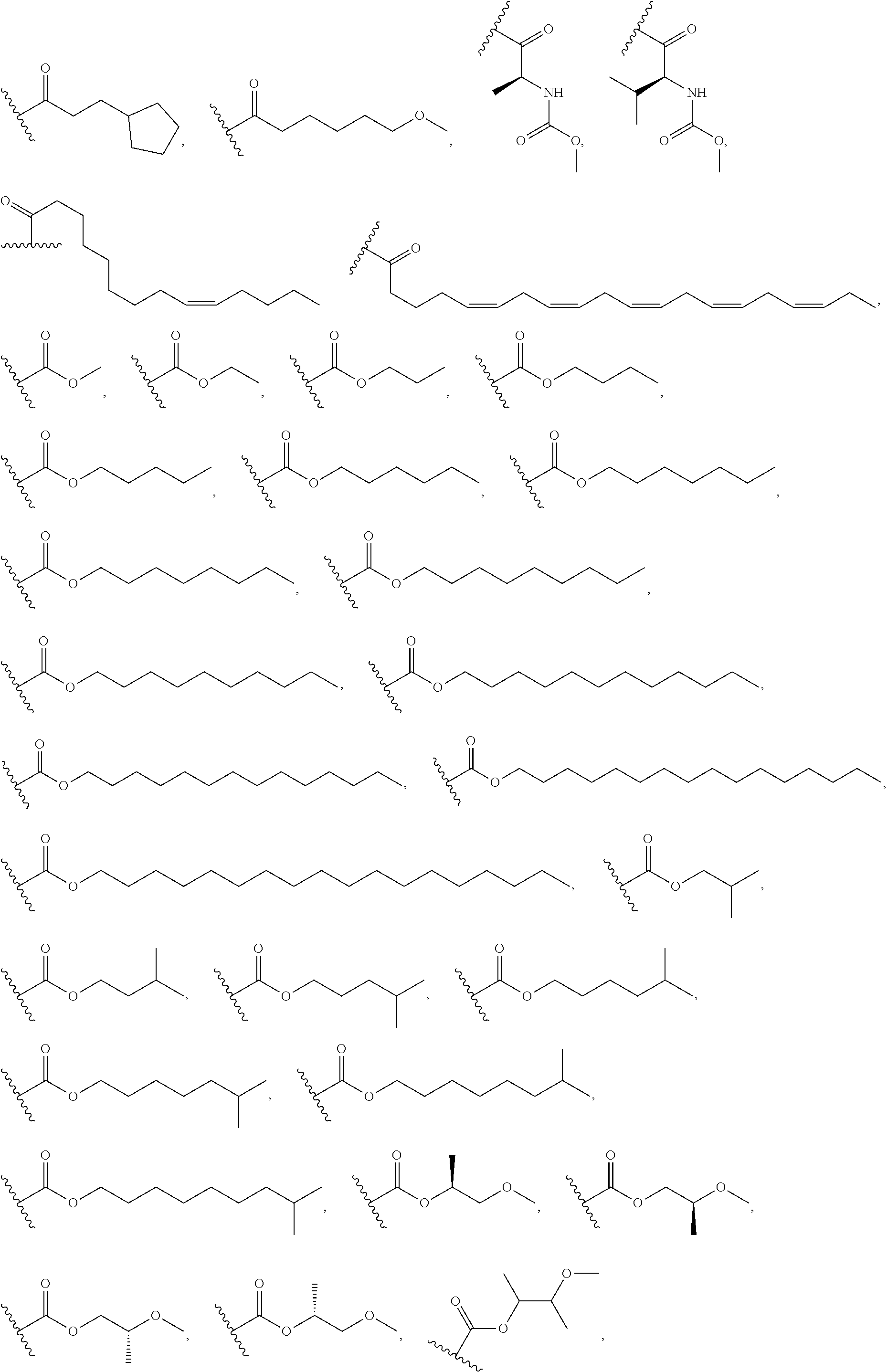

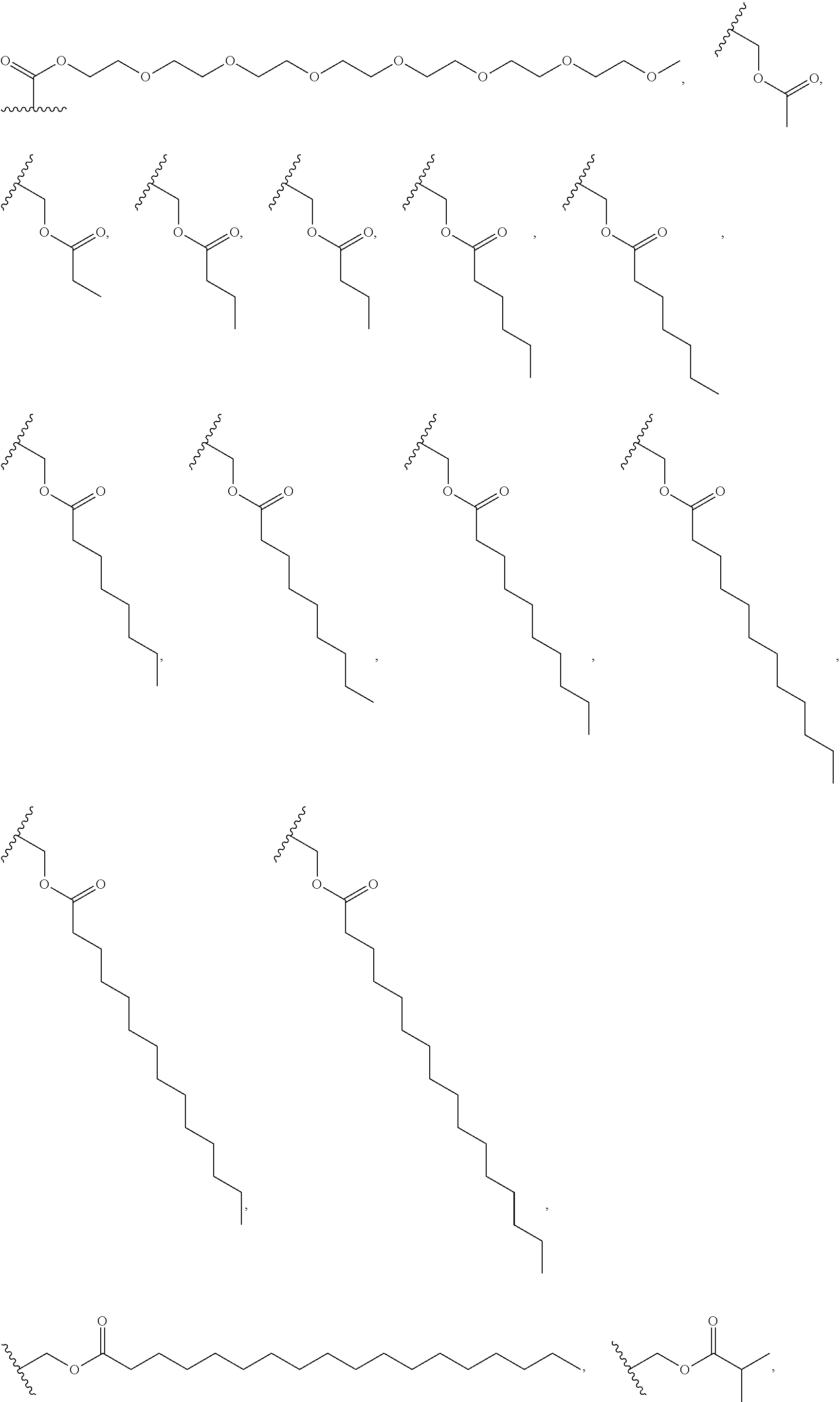

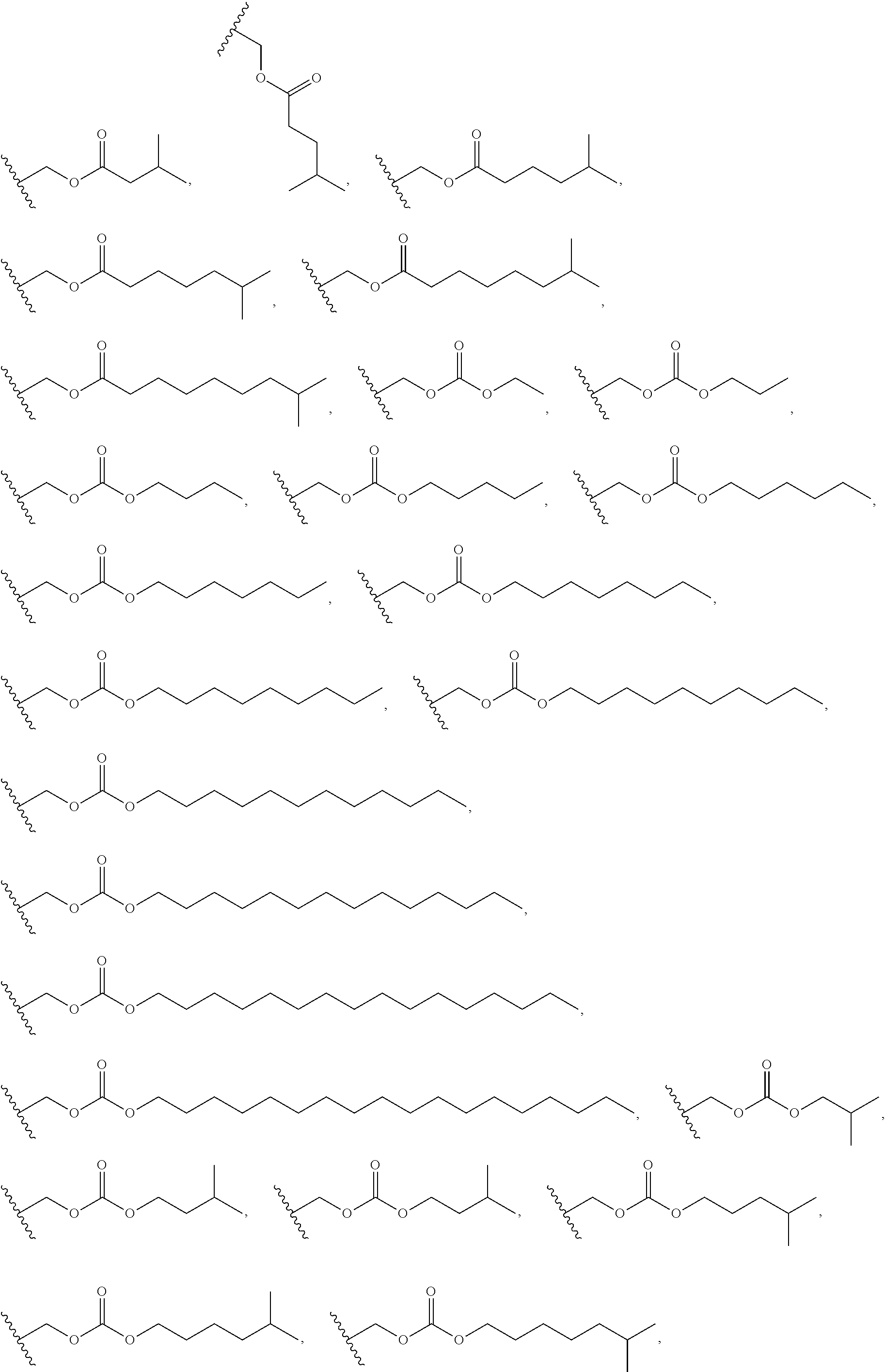

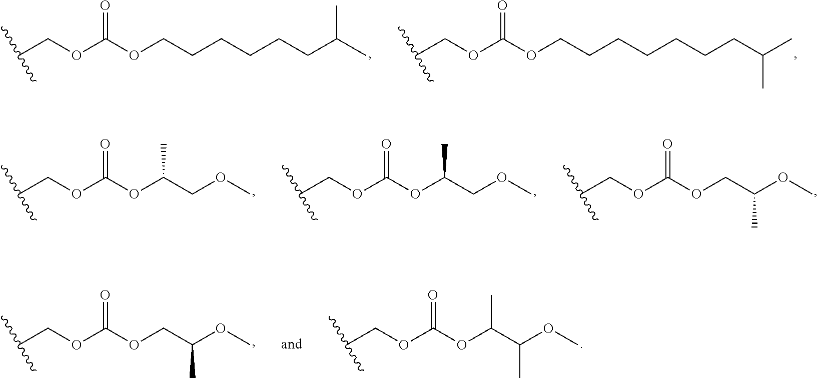

55.-65. (canceled)

66. A pharmaceutical composition comprising a therapeutically effective amount of a compound of claim 1, or pharmaceutically acceptable salt thereof, and a pharmaceutically acceptable excipient.

67.-76. (canceled)

77. A method of treating an HIV infection in a human having or at risk of having the infection, comprising administering to the human a therapeutically effective amount of a compound of claim 1 or pharmaceutically acceptable salt thereof.

78.-86. (canceled)
